(19) United States
(12) Patent Application Publication
(10) Pub. No.: US 2026/0223850 A1
(43) Pub. Date: Aug. 6, 2026

(54) SUBSTITUTED PYRROLIDONE COMPOUND, PREPARATION METHOD THEREFOR, COMPOSITION AND APPLICATION THEREOF

(71) Applicant: QINGDAO KINGAGROOT CHEMICAL COMPOUND CO., LTD., Qingdao, Shandong (CN)

(72) Inventors: Lei LIAN, Qingdao, Shandong (CN); Xuegang PENG, Qingdao, Shandong (CN); Rongbao HUA, Qingdao, Shandong (CN)

(21) Appl. No.: 19/155,661

(22) PCT Filed: Feb. 28, 2024
(86) PCT No.: PCT/CN2024/078965

§ 371 Date: Aug. 12, 2025

(30) Foreign Application Priority Data
Mar. 15, 2023 (CN) 202310251077.5
Dec. 22, 2023 (CN) 202311782018.7

(51) Int. Cl. A01N 43/56; A01N 37/18; A01N 37/20; A01N 37/28; A01N 43/08; A01N 43/40; A01N 43/54; A01N 43/653; A01N 43/78; A01N 43/80; A01N 43/82; A01N 43/90; A01N 57/12; A01P 3/00; A01P 7/04; A01P 13/00; C07D 207/277; C07D 231/38; C07D 401/14; C07D 403/12; C07D 403/14; C07D 405/14; C07D 409/14; C07D 413/14; C07D 417/14

(52) U.S. Cl. CPC A01N 43/56; A01N 37/18; A01N 37/20; A01N 37/28; A01N 43/08; A01N 43/40; A01N 43/54; A01N 43/653; A01N 43/78; A01N 43/80; A01N 43/82; A01N 43/90; A01N 57/12; A01P 3/00; A01P 7/04; A01P 13/00; C07D 207/277; C07D 231/38; C07D 401/14; C07D 403/12; C07D 403/14; C07D 405/14; C07D 409/14; C07D 413/14; C07D 417/14

(57) ABSTRACT

Disclosed is a substituted pyrrolidone compound, a preparation method therefor, and a composition and an application thereof. The substituted pyrrolidone compound is shown in the general formula I.

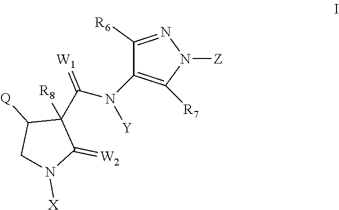

wherein, Q represents

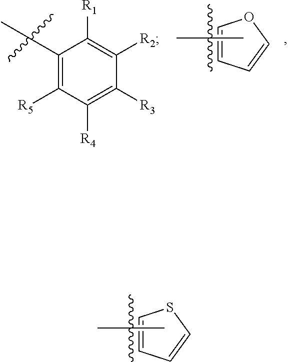

or the like that is unsubstituted or substituted by at least one R₁₁; X and Y each independently represent hydrogen, alkyl, alkenyl, alkynyl, etc.; Z represents alkyl, alkenyl, alkynyl, etc.; W₁ and W₂ each independently represent O or S; R₆ represents hydrogen, hydroxyl, halogen, etc.; R₇ represents hydrogen, halogen, etc.; R₈ represents hydrogen, alkyl, alkenyl, alkynyl, etc. The compound has excellent herbicidal activity against gramineous weeds and the like, and is safe and has high selectivity to crops, but also has good control activity against various fungi such as tomato gray mold, rice sheath blight, apple ring rot, etc. and agricultural pests such as lepidopterans (e.g., Spodoptera frugiperda, Mythimna separata, etc.).

### TECHNICAL FIELD

The present invention belongs to the technical field of pesticides and particularly relates to a substituted pyrrolidone compound, a preparation method therefor, and a composition and an application thereof.

### BACKGROUND ART

Weed control is one of the most important links in the course of achieving high-efficiency agriculture. Although various herbicides are available in the market, the herbicidal properties against harmful plants and the selectivity to crops of these known compounds are not completely satisfactory. In addition, due to the continuous expansion of the market, issues such as resistance of weeds and service life and economy of drugs, and people's increasing attention to the environment, scientists are expected to continuously study and further develop new efficient, safe and economical herbicide varieties with different modes of action. Furthermore, in recent years, owing to the long-term use of pest control agents such as insecticides or fungicides, diseases and pests have acquired drug resistance and become difficult to prevent or eliminate by currently used insecticides or fungicides. Besides, some of the known pest control agents are highly toxic, or some of which damage the ecosystem by their long-term residue. In this context, although a large number of insecticides are known, it is still necessary to develop new pest control agents with low toxicity and low residue.

### CONTENTS OF THE INVENTION

The present invention provides a substituted pyrrolidone compound, a preparation method therefor, and a composition and an application thereof. The compound not only has excellent herbicidal activity against gramineous weeds and the like, and is safe and has high selectivity to crops, but also has good control activity against various fungi such as tomato gray mold, rice sheath blight, apple ring rot, etc. and agricultural pests such as lepidopterans (e.g., Spodoptera frugiperda, Mythimna separata, etc.).

The technical scheme adopted in the present invention is as follows:
a substituted pyrrolidone compound is as shown in the general formula I:

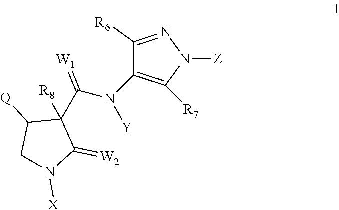

wherein, Q represents

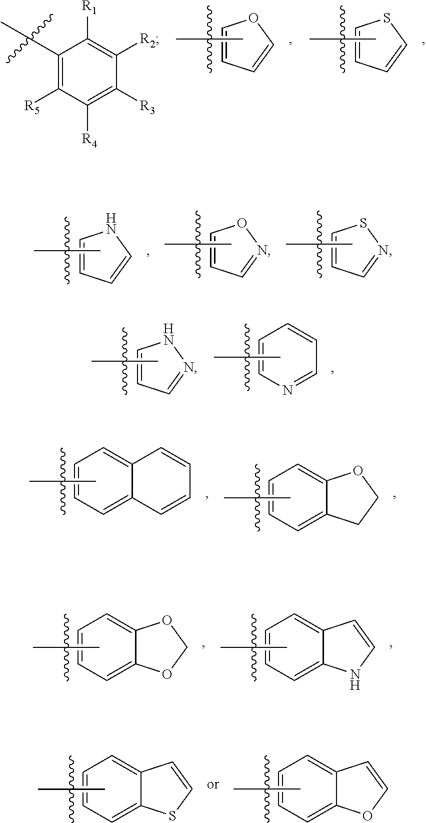

that is unsubstituted or substituted by at least one R₁₁;
X and Y each independently represent hydrogen, alkyl, alkenyl, alkynyl, haloalkyl, haloalkenyl, haloalkynyl, cycloalkyl, cycloalkylalkyl, aryl, arylalkyl, heterocyclyl, heterocyclylalkyl, hydroxyl, hydroxylalkyl, mercapto, mercaptoalkyl, nitro, cyanoalkyl, formyl, trialkylsilyl, —N(R₂₁)₂, —OR₂₂, —SR₂₂, —(SO)R₂₂, —(CO)R₂₂, —(CO)OR₂₂, —(SO₂)R₂₂, —(SO₂)N(R₂₁)₂, -alkylene-N(R₂₁)₂, -alkylene-OR₂₂, -alkylene-SR₂₂, -alkylene-(CO)R₂₂, -alkylene-(CO)OR₂₂, -alkylene-(SO₂)R₂₂, or -alkylene-(SO₂)N(R₂₁)₂;
Z represents alkyl, alkenyl, alkynyl, haloalkyl, haloalkenyl, haloalkynyl, cycloalkyl, cycloalkylalkyl, aryl, arylalkyl, heterocyclyl, heterocyclylalkyl, hydroxyl, hydroxylalkyl, mercapto, mercaptoalkyl, nitro, cyanoalkyl, formyl, trialkylsilyl, —N(R₂₁)₂, —OR₂₂, —SR₂₂, —(SO)R₂₂, —(CO)R₂₂, —(CO)OR₂₂, —(SO₂)R₂₂, —(SO₂)N(R₂₁)₂, -alkylene-N(R₂₁)₂, -alkylene-OR₂₂, -alkylene-SR₂₂, -alkylene-(CO)R₂₂, -alkylene-(CO)OR₂₂, -alkylene-(SO₂)R₂₂, or -alkylene-(SO₂)N(R₂₁)₂;
W₁ and W₂ each independently represent O or S;
R₆ represents hydrogen, hydroxyl, halogen, alkyl, cycloalkyl, alkoxy, or haloalkyl;
R₇ represents hydrogen, halogen, nitro, cyano, formyl, carboxyl, hydroxyl, hydroxylalkyl, mercapto, mercaptoalkyl, alkyl, alkenyl, alkynyl, haloalkyl, haloalkenyl, haloalkynyl, —OR₂₂, —SR₂₂, —(SO)R₂₂, —(SO₂)R₂₂, —CR₂₃=N—O—R₂₂, -alkylene-OR₂₂, -alkylene-SR₂₂, -alkylene-(SO₂)R₂₂, cycloalkyl, cycloalkenyl, aryl, heterocyclyl, —(CO)R₂₂, —(CO)N(R₂₁)₂, —(CO)OR₂₂, —N(R₂₁)₂, -alkylene-(CO)R₂₂, -alkylene-(CO)N(R₂₁)₂, -alkylene-(CO)OR₂₂, or -alkylene-N(R₂₁)₂;
R₈ represents hydrogen, alkyl, alkenyl, alkynyl, haloalkyl, haloalkenyl, haloalkynyl, cycloalkyl, or cycloalkylalkyl;
R₁, R₂, R₃, R₄ and R₅ each independently represent hydrogen, halogen, nitro, cyano, cyanothio, hydroxyl, mercapto, carboxyl, sulfo, formyl, haloformyl, azido, alkyl, alkenyl, alkynyl, cycloalkyl, cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —PO(OR₂₂)₂, —OR₂₂, —(CO)R₂₂, —SR₂₂, —(SO)R₂₂, —(SO₂)R₂₂, —Si(R₂₂)₃, —O(CO)R₂₂, —O—(SO₂)R₂₂, —S(CO)R₂₂, —(SO₂)OR₂₂, —O(CO)OR₂₂, —(CO)(CO)OR₂₂, —(CO)OR₂₂, —O—N═C(R₂₃)₂, —CR₂₃=N—OH, or —CR₂₃=N—O—R₂₂; wherein, the “alkyl”, “alkenyl” or “alkynyl” is optionally substituted by at least one group selected from halogen, nitro, cyano, hydroxyl, mercapto, carboxyl, cycloalkyl, cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —OR₂₂, —(CO)R₂₂, —SR₂₂, —(SO)R₂₂, —(SO₂)R₂₂, —O(CO)H, —O(CO)R₂₂, —O—(SO₂)R₂₂, —(CO)OR₂₂, —O(CO)OR₂₂, —Si(R₂₂)₃, —O(CO)(CO)OH, —O(CO)(CO)OR₂₂, —O-alkylene-(CO)OH, and —O-alkylene-(CO)OR₂₂;
R₁₁ each independently represents halogen, nitro, cyano, cyanothio, hydroxyl, mercapto, carboxyl, sulfo, formyl, haloformyl, azido, alkyl, alkenyl, alkynyl, cycloalkyl, cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —PO(OR₂₂)₂, —OR₂₂, —(CO)R₂₂, —SR₂₂, —(SO)R₂₂, —(SO₂)R₂₂, —Si(R₂₂)₃, —O(CO)R₂₂, —O—(SO₂)R₂₂, —S(CO)R₂₂, —(SO₂)OR₂₂, —O(CO)OR₂₂, —(CO)(CO)OR₂₂, —(CO)OR₂₂, —O—N═C(R₂₃)₂, —CR₂₃=N—OH, or —CR₂₃=N—O—R₂₂; wherein, the “alkyl”, “alkenyl” or “alkynyl” is optionally substituted by at least one group selected from halogen, nitro, cyano, hydroxyl, mercapto, carboxyl, cycloalkyl, cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —OR₂₂, —(CO)R₂₂, —SR₂₂, —(SO)R₂₂, —(SO₂)R₂₂, —O(CO)H, —O(CO)R₂₂, —O—(SO₂)R₂₂, —(CO)OR₂₂, —O(CO)OR₂₂, —Si(R₂₂)₃, —O(CO)(CO)OH, —O(CO)(CO)OR₂₂, —O-alkylene-(CO)OH, and —O— alkylene-(CO)OR₂₂;
R₂₁ each independently represents hydrogen, alkyl, alkenyl, alkynyl, cycloalkyl, cycloalkenyl, aryl, heterocyclyl, —OR₂₂, —(CO)R₂₂, —(CO)OR₂₂, -alkylene-(CO)OR₂₂, —(SO₂)R₂₂, —(SO₂)OR₂₂, -alkylene-(SO₂)R₂₂, —(CO)N(R₂₄)₂, or —(SO₂)N(R₂₄)₂;
R₂₂ each independently represents alkyl, alkenyl, alkynyl, cycloalkyl, cycloalkenyl, aryl, or heterocyclyl, wherein, the “alkyl”, “alkenyl” or “alkynyl” is optionally substituted by at least one group selected from halogen, cyano, trialkylsilyl, cycloalkyl, cycloalkenyl, aryl, heterocyclyl, —OR₂₅, —SR₂₅, —O(CO)R₂₅, —(CO)R₂₅, —(CO)OR₂₅, and —O(CO)OR₂₅;
R₂₃ each independently represents hydrogen, halogen, alkoxy, alkoxyalkyl, alkyl, alkenyl, alkynyl, cycloalkyl, cycloalkylalkyl, cycloalkenyl, cycloalkenylalkyl, aryl, arylalkyl, heterocyclyl, or heterocyclylalkyl;
R₂₄ each independently represents hydrogen, alkyl, alkenyl, alkynyl, alkoxy, alkylsulfonyl, cycloalkyl, cycloalkylalkyl, cycloalkenyl, or cycloalkenylalkyl;
or N(R₂₁)₂ and N(R₂₄)₂ each independently represent heterocyclyl with a nitrogen atom at 1-position;
R₂₅ each independently represents hydrogen, alkyl, alkenyl, alkynyl, cycloalkyl, haloalkyl, haloalkenyl, haloalkynyl, phenyl or phenyl that is substituted by at least one group selected from the following: halogen, cyano, nitro, alkyl, haloalkyl, alkoxy, haloalkoxy, alkoxycarbonyl, alkylthio, alkylsulfonyl, phenoxy and phenoxy that is substituted by at least one group selected from halogen, cyano, nitro, alkyl, haloalkyl, alkoxy and haloalkoxy;
the foregoing “cycloalkyl”, “cycloalkenyl”, “heterocyclyl” or “aryl” is optionally substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₀, —SR₁₀, —(CO)OR₁₀, —(SO₂)R₁₀, —N(R₁₀)₂ and —O-alkylene-(CO)OR₁₀, or two adjacent carbon atoms form a fused ring with —OCH₂CH₂— or —OCH₂O— that is unsubstituted or substituted by halogen;
R₁₀ each independently represents hydrogen, alkyl, haloalkyl, phenyl, or phenyl that is substituted by at least one group selected from halogen, cyano, nitro, alkyl, haloalkyl, alkoxycarbonyl, alkylthio, alkylsulfonyl, alkoxy, and haloalkoxy.

Preferably, X and Y each independently represent hydrogen, C1-C8 alkyl, C2-C8 alkenyl, C2-C8 alkynyl, halo C1-C8 alkyl, halo C2-C8 alkenyl, halo C2-C8 alkynyl, C3-C8 cycloalkyl, C3-C8 cycloalkyl C1-C8 alkyl, aryl, aryl C1-C8 alkyl, heterocyclyl, heterocyclyl C1-C8 alkyl, hydroxyl, hydroxyl C1-C8 alkyl, mercapto, mercapto C1-C8 alkyl, nitro, cyano C1-C8 alkyl, formyl, tri C1-C8 alkylsilyl, —N(R₂₁)₂, —OR₂₂, —SR₂₂, —(SO)R₂₂, —(CO)R₂₂, —(CO)OR₂₂, —(SO₂)R₂₂, —(SO₂)N(R₂₁)₂, —(C1-C8 alkylene)-N(R₂₁)₂, —(C1-C8 alkylene)-OR₂₂, —(C1-C8 alkylene)-SR₂₂, —(C1-C8 alkylene)-(CO)R₂₂, —(C1-C8 alkylene)-(CO)OR₂₂, —(C1-C8 alkylene)-(SO₂)R₂₂, or —(C1-C8 alkylene)-(SO₂)N(R₂₁)₂;
Z represents C1-C8 alkyl, C2-C8 alkenyl, C2-C8 alkynyl, halo C1-C8 alkyl, halo C2-C8 alkenyl, halo C2-C8 alkynyl, C3-C8 cycloalkyl, C3-C8 cycloalkyl C1-C8 alkyl, aryl, aryl C1-C8 alkyl, heterocyclyl, heterocyclyl C1-C8 alkyl, hydroxyl, hydroxyl C1-C8 alkyl, mercapto, mercapto C1-C8 alkyl, nitro, cyano C1-C8 alkyl, formyl, tri C1-C8 alkylsilyl, —N(R₂₁)₂, —OR₂₂, —SR₂₂, —(SO)R₂₂, —(CO)R₂₂, —(CO)OR₂₂, —(SO₂)R₂₂, —(SO₂)N(R₂₁)₂, —(C1-C8 alkylene)-N(R₂₁)₂, —(C1-C8 alkylene)-OR₂₂, —(C1-C8 alkylene)-SR₂₂, —(C1-C8 alkylene)-(CO)R₂₂, —(C1-C8 alkylene)-(CO)OR₂₂, —(C1-C8 alkylene)-(SO₂)R₂₂, or —(C1-C8 alkylene)-(SO₂)N(R₂₁)₂;
R₆ represents hydrogen, hydroxyl, halogen, C1-C8 alkyl, C3-C8 cycloalkyl, C1-C8 alkoxy, or halo C1-C8 alkyl;
R₇ represents hydrogen, halogen, nitro, cyano, formyl, carboxyl, hydroxyl, hydroxyl C1-C8 alkyl, mercapto, mercapto C1-C8 alkyl, C1-C8 alkyl, C2-C8 alkenyl, C2-C8 alkynyl, halo C1-C8 alkyl, halo C2-C8 alkenyl, halo C2-C8 alkynyl, —OR₂₂, —SR₂₂, —(SO)R₂₂, —(SO₂)R₂₂, —CR₂₃=N—O—R₂₂, —(C1-C8 alkylene)-OR₂₂, —(C1-C8 alkylene)-SR₂₂, —(C1-C8 alkylene)-(SO₂)R₂₂, C3-C8 cycloalkyl, C3-C8 cycloalkenyl, aryl, heterocyclyl, —(CO)R₂₂, —(CO)N(R₂₁)₂, —(CO)OR₂₂, —N(R₂₁)₂, —(C1-C8 alkylene)-(CO)R₂₂, —(C1-C8 alkylene)-(CO)N(R₂₁)₂, —(C1-C8 alkylene)-(CO)OR₂₂, or —(C1-C8 alkylene)-N(R₂₁)₂;
R₈ represents hydrogen, C1-C8 alkyl, C2-C8 alkenyl, C2-C8 alkynyl, halo C1-C8 alkyl, halo C2-C8 alkenyl, halo C2-C8 alkynyl, C3-C8 cycloalkyl, or C3-C8 cycloalkyl C1-C8 alkyl;
R₁, R₂, R₃, R₄ and R₈ each independently represent hydrogen, halogen, nitro, cyano, cyanothio, hydroxyl, mercapto, carboxyl, sulfo, formyl, haloformyl, azido, C1-C8 alkyl, C2-C8 alkenyl, C2-C8 alkynyl, C3-C8 cycloalkyl, C3-C8 cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —PO(OR₂₂)₂, —OR₂₂, —(CO)R₂₂, —SR₂₂, —(SO)R₂₂, —(SO₂)R₂₂, —Si(R₂₂)₃, —O(CO)R₂₂, —O—(SO₂)R₂₂, —S(CO)R₂₂, —(SO₂)OR₂₂, —O(CO)OR₂₂, —(CO)(CO)OR₂₂, —(CO)OR₂₂, —O—N═C(R₂₃)₂, —CR₂₃=N—OH, or —CR₂₃=N—O—R₂₂; wherein, the “C1-C8 alkyl”, “C2-C8 alkenyl” or “C2-C8 alkynyl” is optionally substituted by at least one group selected from halogen, nitro, cyano, hydroxyl, mercapto, carboxyl, C3-C8 cycloalkyl, C3-C8 cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —OR₂₂, —(CO)R₂₂, —SR₂₂, —(SO)R₂₂, —(SO₂)R₂₂, —O(CO)H, —O(CO)R₂₂, —O—(SO₂)R₂₂, —(CO)OR₂₂, —O(CO)OR₂₂, —Si(R₂₂)₃, —O(CO)(CO)OH, —O(CO)(CO)OR₂₂, —O—(C1-C8 alkylene)-(CO)OH, and —O—(C1-C8 alkylene)-(CO)OR₂₂;
R₁₁ each independently represents halogen, nitro, cyano, cyanothio, hydroxyl, mercapto, carboxyl, sulfo, formyl, haloformyl, azido, C1-C8 alkyl, C2-C8 alkenyl, C2-C8 alkynyl, C3-C8 cycloalkyl, C3-C8 cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —PO(OR₂₂)₂, —OR₂₂, —(CO)R₂₂, —SR₂₂, —(SO)R₂₂, —(SO₂)R₂₂, —Si(R₂₂)₃, —O(CO)R₂₂, —O—(SO₂)R₂₂, —S(CO)R₂₂, —(SO₂)OR₂₂, —O(CO)OR₂₂, —(CO)(CO)OR₂₂, —(CO)OR₂₂, —O—N═C(R₂₃)₂, —CR₂₃=N—OH, or —CR₂₃=N—O—R₂₂; wherein, the “C1-C8 alkyl”, “C2-C8 alkenyl” or “C2-C8 alkynyl” is optionally substituted by at least one group selected from halogen, nitro, cyano, hydroxyl, mercapto, carboxyl, C3-C8 cycloalkyl, C3-C8 cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —OR₂₂, —(CO)R₂₂, —SR₂₂, —(SO)R₂₂, —(SO₂)R₂₂, —O(CO)H, —O(CO)R₂₂, —O—(SO₂)R₂₂, —(CO)OR₂₂, —O(CO)OR₂₂, —Si(R₂₂)₃, —O(CO)(CO)OH, —O(CO)(CO)OR₂₂, —O—(C1-C8 alkylene)-(CO)OH, and —O—(C1-C8 alkylene)-(CO)OR₂₂;
R₂₁ each independently represents hydrogen, C1-C8 alkyl, C2-C8 alkenyl, C2-C8 alkynyl, C3-C8 cycloalkyl, C3-C8 cycloalkenyl, aryl, heterocyclyl, —OR₂₂, —(CO)R₂₂, —(CO)OR₂₂, —(C1-C8 alkylene)-(CO)OR₂₂, —(SO₂)R₂₂, —(SO₂)OR₂₂, —(C1-C8 alkylene)-(SO₂)R₂₂, —(CO)N(R₂₄)₂, or —(SO₂)N(R₂₄)₂;

R₂₂ each independently represents C1-C8 alkyl, C2-C8 alkenyl, C2-C8 alkynyl, C3-C8 cycloalkyl, C3-C8 cycloalkenyl, aryl, or heterocyclyl, wherein, the “C1-C8 alkyl”, “C2-C8 alkenyl” or “C2-C8 alkynyl” is optionally substituted by at least one group selected from halogen, cyano, tri C1-C8 alkylsilyl, C3-C8 cycloalkyl, C3-C8 cycloalkenyl, aryl, heterocyclyl, —OR₂₅, —SR₂₅, —O(CO)R₂₅, —(CO)R₂₅, —(CO)OR₂₅, and —O(CO)OR₂₅;
R₂₃ each independently represents hydrogen, halogen, C1-C8 alkoxy, C1-C8 alkoxy C1-C8 alkyl, C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, aryl, aryl C1-C8 alkyl, heterocyclyl, or heterocyclyl C1-C8 alkyl;
R₂₄ each independently represents hydrogen, C1-C8 alkyl, C2-C8 alkenyl, C2-C8 alkynyl, C1-C8 alkoxy, C1-C8 alkylsulfonyl, C3-C8 cycloalkyl, C3-C8 cycloalkyl C1-C8 alkyl, C3-C8 cycloalkenyl, or C3-C8 cycloalkenyl C1-C8 alkyl;
or N(R₂₁)₂ and N(R₂₄)₂ each independently represent heterocyclyl with a nitrogen atom at 1-position;
R₂₅ each independently represents hydrogen, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, phenyl or phenyl that is substituted by at least one group selected from the following: halogen, cyano, nitro, C1-C8 alkyl, halo C1-C8 alkyl, C1-C8 alkoxy, halo C1-C8 alkoxy, C1-C8 alkoxycarbonyl, C1-C8 alkylthio, C1-C8 alkylsulfonyl, phenoxy and phenoxy that is substituted by at least one group selected from halogen, cyano, nitro, C1-C8 alkyl, halo C1-C8 alkyl, C1-C8 alkoxy and halo C1-C8 alkoxy;
the foregoing “C3-C8 cycloalkyl”, “C3-C8 cycloalkenyl”, “heterocyclyl” or “aryl” is optionally substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₀, —SR₁₀, —(CO)OR₁₀, —(SO₂)R₁₀, —N(R₁₀)₂ and —O—(C1-C8 alkylene)-(CO)OR₁₀, or two adjacent carbon atoms form a fused ring with —OCH₂CH₂— or —OCH₂O— that is unsubstituted or substituted by halogen;
R₁₀ each independently represents hydrogen, C1-C8 alkyl, halo C1-C8 alkyl, phenyl, or phenyl that is substituted by at least one group selected from halogen, cyano, nitro, C1-C8 alkyl, halo C1-C8 alkyl, C1-C8 alkoxycarbonyl, C1-C8 alkylthio, C1-C8 alkylsulfonyl, C1-C8 alkoxy, and halo C1-C8 alkoxy.

More preferably, X and Y each independently represent hydrogen, C1-C6 alkyl, C2-C6 alkenyl, C2-C6 alkynyl, halo C1-C6 alkyl, halo C2-C6 alkenyl, halo C2-C6 alkynyl, C3-C6 cycloalkyl, C3-C6 cycloalkyl C1-C6 alkyl, aryl, aryl C1-C6 alkyl, heterocyclyl, heterocyclyl C1-C6 alkyl, hydroxyl, hydroxyl C1-C6 alkyl, mercapto, mercapto C1-C6 alkyl, nitro, cyano C1-C6 alkyl, formyl, tri C1-C6 alkylsilyl, —N(R₂₁)₂, —OR₂₂, —SR₂₂, —(SO)R₂₂, —(CO)R₂₂, —(CO)OR₂₂, —(SO₂)R₂₂, —(SO₂)N(R₂₁)₂, —(C1-C6 alkylene)-N(R₂₁)₂, —(C1-C6 alkylene)-OR₂₂, —(C1-C6 alkylene)-SR₂₂, —(C1-C6 alkylene)-(CO)R₂₂, —(C1-C6 alkylene)-(CO)OR₂₂, —(C1-C6 alkylene)-(SO₂)R₂₂, or —(C1-C6 alkylene)-(SO₂)N(R₂₁)₂;
Z represents C1-C6 alkyl, C2-C6 alkenyl, C2-C6 alkynyl, halo C1-C6 alkyl, halo C2-C6 alkenyl, halo C2-C6 alkynyl, C3-C6 cycloalkyl, C3-C6 cycloalkyl C1-C6 alkyl, aryl, aryl C1-C6 alkyl, heterocyclyl, heterocyclyl C1-C6 alkyl, hydroxyl, hydroxyl C1-C6 alkyl, mercapto, mercapto C1-C6 alkyl, nitro, cyano C1-C6 alkyl, formyl, tri C1-C6 alkylsilyl, —N(R₂₁)₂, —OR₂₂, —SR₂₂, —(SO)R₂₂, —(CO)R₂₂, —(CO)OR₂₂, —(SO₂)R₂₂, —(SO₂)N(R₂₁)₂, —(C1-C6 alkylene)-N(R₂₁)₂, —(C1-C6 alkylene)-OR₂₂, —(C1-C6 alkylene)-SR₂₂, —(C1-C6 alkylene)-(CO)R₂₂, —(C1-C6 alkylene)-(CO)OR₂₂, —(C1-C6 alkylene)-(SO₂)R₂₂, or —(C1-C6 alkylene)-(SO₂)N(R₂₁)₂;
R₆ represents hydrogen, hydroxyl, halogen, C1-C6 alkyl, C3-C6 cycloalkyl, C1-C6 alkoxy, or halo C1-C6 alkyl;
R₇ represents hydrogen, halogen, nitro, cyano, formyl, carboxyl, hydroxyl, hydroxyl C1-C6 alkyl, mercapto, mercapto C1-C6 alkyl, C1-C6 alkyl, C2-C6 alkenyl, C2-C6 alkynyl, halo C1-C6 alkyl, halo C2-C6 alkenyl, halo C2-C6 alkynyl, —OR₂₂, —SR₂₂, —(SO)R₂₂, —(SO₂)R₂₂, —CR₂₃=N—O—R₂₂, —(C1-C6 alkylene)-OR₂₂, —(C1-C6 alkylene)-SR₂₂, —(C1-C6 alkylene)-(SO₂)R₂₂, C3-C6 cycloalkyl, C3-C6 cycloalkenyl, aryl, heterocyclyl, —(CO)R₂₂, —(CO)N(R₂₁)₂, —(CO)OR₂₂, —N(R₂₁)₂, —(C1-C6 alkylene)-(CO)R₂₂, —(C1-C6 alkylene)-(CO)N(R₂₁)₂, —(C1-C6 alkylene)-(CO)OR₂₂, or —(C1-C6 alkylene)-N(R₂₁)₂;
R₈ represents hydrogen, C1-C6 alkyl, C2-C6 alkenyl, C2-C6 alkynyl, halo C1-C6 alkyl, halo C2-C6 alkenyl, halo C2-C6 alkynyl, C3-C6 cycloalkyl, or C3-C6 cycloalkyl C1-C6 alkyl;
R₁, R₂, R₃, R₄ and R₈ each independently represent hydrogen, halogen, nitro, cyano, cyanothio, hydroxyl, mercapto, carboxyl, sulfo, formyl, haloformyl, azido, C1-C6 alkyl, C2-C6 alkenyl, C2-C6 alkynyl, C3-C6 cycloalkyl, C3-C6 cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —PO(OR₂₂)₂, —OR₂₂, —(CO)R₂₂, —SR₂₂, —(SO)R₂₂, —(SO₂)R₂₂, —Si(R₂₂)₃, —O(CO)R₂₂, —O—(SO₂)R₂₂, —S(CO)R₂₂, —(SO₂)OR₂₂, —O(CO)OR₂₂, —(CO)(CO)OR₂₂, —(CO)OR₂₂, —O—N═C(R₂₃)₂, —CR₂₃=N—OH, or —CR₂₃=N—O—R₂₂; wherein, the “C1-C6 alkyl”, “C2-C6 alkenyl” or “C2-C6 alkynyl” is optionally substituted by at least one group selected from halogen, nitro, cyano, hydroxyl, mercapto, carboxyl, C3-C6 cycloalkyl, C3-C6 cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —OR₂₂, —(CO)R₂₂, —SR₂₂, —(SO)R₂₂, —(SO₂)R₂₂, —O(CO)H, —O(CO)R₂₂, —O—(SO₂)R₂₂, —(CO)OR₂₂, —O(CO)OR₂₂, —Si(R₂₂)₃, —O(CO)(CO)OH, —O(CO)(CO)OR₂₂, —O—(C1-C6 alkylene)-(CO)OH, and —O—(C1-C6 alkylene)-(CO)OR₂₂;
R₁₁ each independently represents halogen, nitro, cyano, cyanothio, hydroxyl, mercapto, carboxyl, sulfo, formyl, haloformyl, azido, C1-C6 alkyl, C2-C6 alkenyl, C2-C6 alkynyl, C3-C6 cycloalkyl, C3-C6 cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —PO(OR₂₂)₂, —OR₂₂, —(CO)R₂₂, —SR₂₂, —(SO)R₂₂, —(SO₂)R₂₂, —Si(R₂₂)₃, —O(CO)R₂₂, —O—(SO₂)R₂₂, —S(CO)R₂₂, —(SO₂)OR₂₂, —O(CO)OR₂₂, —(CO)(CO)OR₂₂, —(CO)OR₂₂, —O—N═C(R₂₃)₂, —CR₂₃=N—OH, or —CR₂₃=N—O—R₂₂; wherein, the “C1-C6 alkyl”, “C2-C6 alkenyl” or “C2-C6 alkynyl” is optionally substituted by at least one group selected from halogen, nitro, cyano, hydroxyl, mercapto, carboxyl, C3-C6 cycloalkyl, C3-C6 cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —OR₂₂, —(CO)R₂₂, —SR₂₂, —(SO)R₂₂, —(SO₂)R₂₂, —O(CO)H, —O(CO)R₂₂, —O—(SO₂)R₂₂, —(CO)OR₂₂, —O(CO)OR₂₂, —Si(R₂₂)₃, —O(CO)(CO)OH, —O(CO)(CO)OR₂₂, —O—(C1-C6 alkylene)-(CO)OH, and —O—(C1-C6 alkylene)-(CO)OR₂₂;
R₂₁ each independently represents hydrogen, C1-C6 alkyl, C2-C6 alkenyl, C2-C6 alkynyl, C3-C6 cycloalkyl, C3-C6 cycloalkenyl, aryl, heterocyclyl, —OR₂₂, —(CO)R₂₂, —(CO)OR₂₂, —(C1-C6 alkylene)-(CO)OR₂₂, —(SO₂)R₂₂, —(SO₂)OR₂₂, —(C1-C6 alkylene)-(SO₂)R₂₂, —(CO)N(R₂₄)₂, or —(SO₂)N(R₂₄)₂;
R₂₂ each independently represents C1-C6 alkyl, C2-C6 alkenyl, C2-C6 alkynyl, C3-C6 cycloalkyl, C3-C6 cycloalkenyl, aryl, or heterocyclyl, wherein, the “C1-C6 alkyl”, “C2-C6 alkenyl” or “C2-C6 alkynyl” is optionally substituted by at least one group selected from halogen, cyano, tri C1-C6 alkylsilyl, C3-C6 cycloalkyl, C3-C6 cycloalkenyl, aryl, heterocyclyl, —OR₂₅, —SR₂₅, —O(CO)R₂₅, —(CO)R₂₅, —(CO)OR₂₅, and —O(CO)OR₂₅;
R₂₃ each independently represents hydrogen, halogen, C1-C6 alkoxy, C1-C6 alkoxy C1-C6 alkyl, C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, aryl, aryl C1-C6 alkyl, heterocyclyl, or heterocyclyl C1-C6 alkyl;
R₂₄ each independently represents hydrogen, C1-C6 alkyl, C2-C6 alkenyl, C2-C6 alkynyl, C1-C6 alkoxy, C1-C6 alkylsulfonyl, C3-C6 cycloalkyl, C3-C6 cycloalkyl C1-C6 alkyl, C3-C6 cycloalkenyl, or C3-C6 cycloalkenyl C1-C6 alkyl;
or N(R₂₁)₂ and N(R₂₄)₂ each independently represent

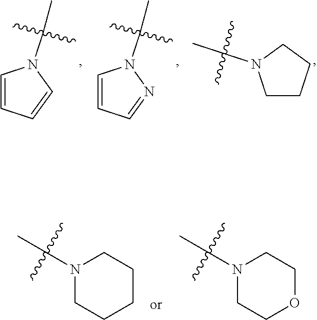 [SMILES: *n1cccc1 ; *n1cccn1 ; *N1CCCC1 ; *N1CCCCC1 ; *N1CCOCC1]

that is unsubstituted or substituted by at least one group selected from oxo, C1-C6 alkyl, and C1-C6 alkoxycarbonyl;
R₂₅ each independently represents hydrogen, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, phenyl or phenyl that is substituted by at least one group selected from the following: halogen, cyano, nitro, C1-C6 alkyl, halo C1-C6 alkyl, C1-C6 alkoxy, halo C1-C6 alkoxy, C1-C6 alkoxycarbonyl, C1-C6 alkylthio, C1-C6 alkylsulfonyl, phenoxy and phenoxy that is substituted by at least one group selected from halogen, cyano, nitro, C1-C6 alkyl, halo C1-C6 alkyl, C1-C6 alkoxy and halo C1-C6 alkoxy;
the foregoing “C3-C6 cycloalkyl”, “C3-C6 cycloalkenyl”, “heterocyclyl” or “aryl” is optionally substituted by at least one group selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₀, —SR₁₀, —(CO)OR₁₀, —(SO₂)R₁₀, —N(R₁₀)₂ and —O—(C1-C6 alkylene)-(CO)OR₁₀, or two adjacent carbon atoms form a fused ring with —OCH₂CH₂— or —OCH₂O— that is unsubstituted or substituted by halogen;
R₁₀ each independently represents hydrogen, C1-C6 alkyl, halo C1-C6 alkyl, phenyl, or phenyl that is substituted by at least one group selected from halogen, cyano, nitro, C1-C6 alkyl, halo C1-C6 alkyl, C1-C6 alkoxycarbonyl, C1-C6 alkylthio, C1-C6 alkylsulfonyl, C1-C6 alkoxy, and halo C1-C6 alkoxy.

In the definitions of the compounds represented by the above-mentioned general formula and all of the following structural formulas, the technical terms used, whether used alone or used in compound words, represent the following substituent groups: an alkyl group which has more than two carbon atoms and may be linear or branched. For example, the alkylene in the compound word “-alkylene-(CO)OR₂₂” may be —CH₂—, —CH₂CH₂—, —CH(CH₃)—, —C(CH₃)₂—, etc. An alkyl group is, for example, C1 alkyl: methyl; C2 alkyl: ethyl; C3 alkyl: propyl such as n-propyl or isopropyl; C4 alkyl: butyl such as n-butyl, isobutyl, tert-butyl, or 2-butyl; C5 alkyl: pentyl such as n-pentyl; C6 alkyl: hexyl such as n-hexyl, isohexyl, or 1,3-dimethylbutyl. Similarly, alkenyl is, for example, vinyl, allyl, 1-methylprop-2-en-1-yl, 2-methylprop-2-en-1-yl, but-2-en-1-yl, but-3-en-1-yl, 1-methylbut-3-en-1-yl, or 1-methylbut-2-en-1-yl. Alkynyl is, for example, ethynyl, propargyl, but-2-yn-1-yl, but-3-yn-1-yl, or 1-methylbut-3-yn-1-yl. A multiple bond may be at any position of each unsaturated group. Cycloalkyl is a saturated carbocyclic ring system having for example three to six carbon atoms such as cyclopropyl, cyclobutyl, cyclopentyl, or cyclohexyl. Similarly, cycloalkenyl is monocyclic alkenyl having for example three to six carbon ring members such as cyclopropenyl, cyclobutenyl, cyclopentenyl, or cyclohexenyl, wherein a double bond can be at any position. A halogen is fluorine, chlorine, bromine, or iodine.

Unless otherwise specified, the “aryl” of the present invention includes, but is not limited to, phenyl, naphthyl,

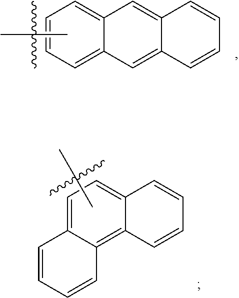

the “heterocyclyl” not only includes, but is not limited to, saturated or unsaturated non-aromatic cyclic groups

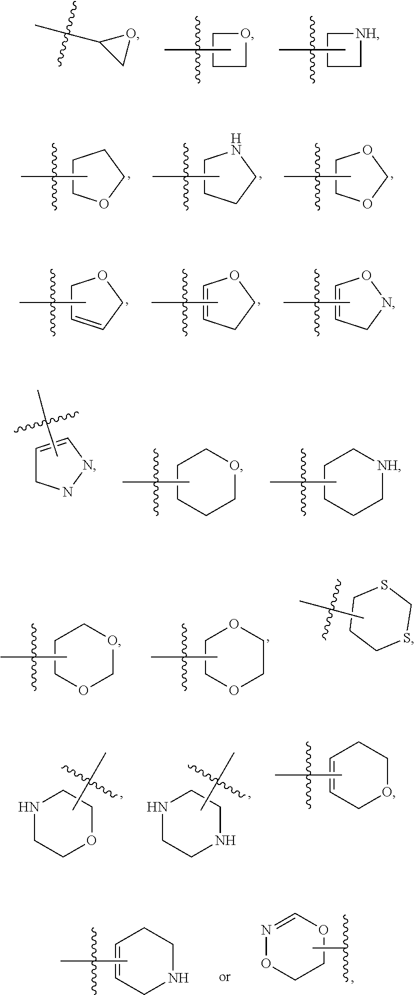

etc., but also includes, but is not limited to, heteroaryl, that is an aromatic cyclic group having for example 3 to 6 ring atoms and also optionally fused with a benzo ring. One to four (for example, 1, 2, 3, or 4) heteroatoms of the ring atoms are selected from oxygen, nitrogen, and sulfur, for example

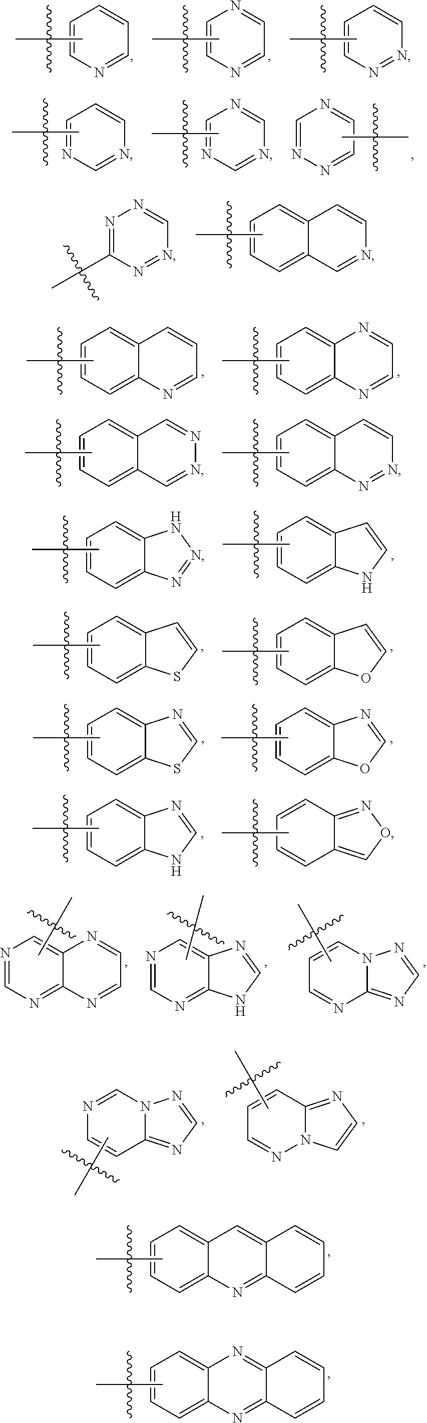

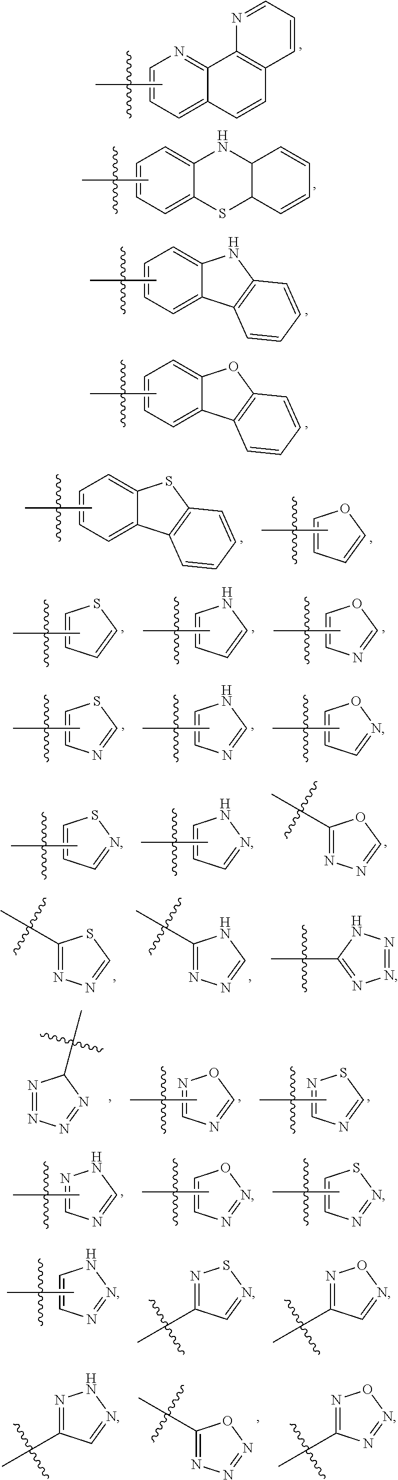

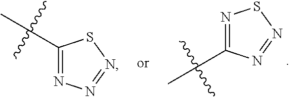 [SMILES: *c1nnns1 ; "or" ; *c1nnsn1 ; "."]

If one group is substituted by a group, which should be understood to mean that the group is substituted by one or more the same or different groups selected from the mentioned groups. In addition, the same or different substitution characters contained in the same or different substituent groups are independently selected, which may be the same or different. This is also applicable to a ring system formed by different atoms and units. Meanwhile, the scope of the claims will exclude those compounds chemically unstable under standard conditions which are known to a person skilled in the art.

In addition, unless specifically defined, the term “substituted by at least one group” herein refers to being substituted by for example 1, 2, 3, 4, or 5 groups; a group (including heterocyclyl, aryl, etc.) without being specified a linking site may be linked at any site, including a site connected to C or N; if it is substituted, the substituent group may also substitute at any site as long as the valence bond theory is complied with. For example, the heteroaryl

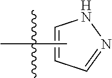

substituted by 1 methyl may represent

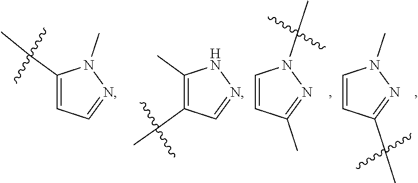

etc.

The present invention provides a substituted pyrrolidone compound with chiral centers as set forth in Formula I′:

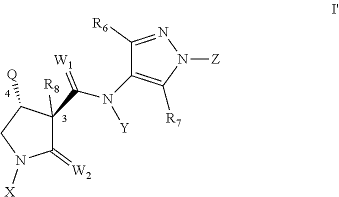

wherein, the definitions of substituents Q, R₆, R₇, R₈, W₁, W₂, X, Y and Z are as described above.

In one specific embodiment, based on the contents of stereoisomers that have R- and S-configuration at position 3, it has a stereochemical purity of 60%-100% (S), preferably 70%-100%, more preferably 80%-100%, further preferably 90%-100%, even further preferably 95%-100%; and based on the contents of stereoisomers that have R- and S-configuration at position 4, it has a stereochemical purity of 60%-100% (S or R, depending on the type of the substituent Q), preferably 70%-100%, more preferably 80%-100%, further preferably 90%-100%, even further preferably 95%-100%.

Among them, “stereochemical purity” refers to the percentage of the amount of the stereoisomers to the total amount of stereoisomers with chiral centers.

In the present invention, the stereochemical configurations at positions 3 and 4 in Formula I′ are respectively determined according to the Cahn-Ingold-Prelog system. However, the subject matter of the present invention also relates to all the stereoisomers at other positions encompassed by Formula I and I′, and mixtures thereof. Such compounds of Formula I and I′ contain, for example, one or more additional asymmetric carbon atoms or other double bonds that are not specified in Formula I and I′. It is to be understood that the present invention comprises pure isomers and mixtures thereof of pure isomers enriched to varying degrees, wherein, the asymmetric carbon atom at the labeled position 3 is in S-configuration, and the asymmetric carbon atom at the labeled position 4 is in S-configuration or R-configuration; or in the mixture, a compound or a compound of the same chemical structure has the configuration at the labeled position, or is present in such a proportion where the compound with the configuration is predominantly present (at least 60% having the configuration); meanwhile, other asymmetric carbon atoms may exist in racemic forms or may be resolved to varying degrees. As long as the stereochemical configuration at a labeled position is eligible, possible stereoisomers defined by particular spatial forms such as enantiomers, diastereoisomers, Z- and E-isomers, are all included in Formula I and I′, and may be obtained from mixtures of stereoisomers using conventional methods, or may be prepared by stereoselective reactions in combination with the use of stereochemically pure starting materials.

The present invention also includes any keto-enol tautomeric form, a mixture and salt thereof, if various functional groups are present.

Stereoisomers are obtainable from the mixtures prepared by optical resolution. Stereoisomers may also be selectively prepared by employing stereoselective reactions and using optically active starting materials and/or auxiliaries. For optical resolution, conventional methods can often be employed (see Textbooks of Stereochemistry), such as those described below for resolving the mixtures into diastereoisomers, for example, physical methods, e.g., crystallization, chromatography, particularly column chromatography and high pressure liquid chromatography, distillation methods carried out under reduced pressure as needed, extraction and other methods. Chromatographic separation on a chiral solid phase is usually adopted to separate residual mixtures of enantiomers. Suitable for a preparative amount or for use on an industrial scale are methods such as crystallization of diastereomeric salts, which can be obtained from compounds using optically active acids, and if acidic groups are present, optically active bases can be utilized as needed.

A method for preparing the substituted pyrrolidone compound includes the following steps:

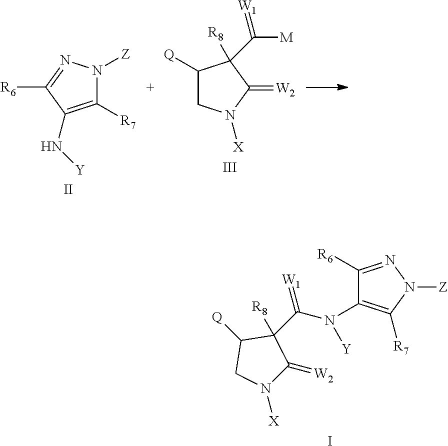

wherein, M represents OH or halogen, and the definitions of substituents Q, R₆, R₇, R₈, W₁, W₂, X, Y and Z are as described above.

Preferably, the reaction is carried out in the presence of a solvent; more preferably, a condensing agent and/or a base is added during the reaction.

In one specific embodiment, the base is selected from at least one of inorganic bases (e.g., K₂CO₃, Na₂CO₃, Cs₂CO₃, NaHCO₃, KHCO₃, KF, CsF, KI, NaI, K₃PO₄, K₂HPO₄, NaOH, KOH, NaH, KH, etc.) and organic bases (e.g., DMAP, pyrazole, triethylamine, DIEA, potassium trimethylsilanolate, AcOK, AcONa, MeONa, EtONa, t-BuONa, etc.).

In one specific embodiment, the solvent is selected from at least one of DMF, DMA, methanol, ethanol, acetonitrile, dichloroethane, DMSO, dioxane, dichloromethane (DCM), and ethyl acetate.

In one specific embodiment, the condensing agent is selected from at least one of Py-BOP, Py-AOP, EDCI, HOBT (1-hydroxybenzotriazole), DCC, HBTU, and HATU.

In addition, the compound as shown in the general formula I can be prepared with reference to the methods set forth in WO2016196593A1, WO2015084796A1, CN115504920A, etc.

An intermediate is as shown in the foregoing Formula II or Formula III.

A herbicidal composition comprises a herbicidally effective amount of at least one of the substituted pyrrolidone compounds, preferably, also comprising a formulation auxiliary.

A method for controlling weeds comprises applying a herbicidally effective amount of at least one of the substituted pyrrolidone compounds or the herbicidal composition to a plant or a weedy area.

At least one of the substituted pyrrolidone compounds or the herbicidal composition has use in controlling weeds; preferably, the substituted pyrrolidone compound is used for preventing or eliminating weeds among useful crops, and the useful crops are transgenic crops or crops treated by genome editing techniques.

A compound of Formula I or I′ of the present invention has outstanding herbicidal activity against a broad spectrum of monocotyledonous and dicotyledonous harmful plants of economic importance. The active substances of the present invention also act effectively on perennial weeds which grow from root stocks, rhizomes, or other perennial organs and are difficult to control. In this context, it is generally immaterial whether the substances are applied before sowing, before emergence, or after emergence. Representative examples of monocotyledonous and dicotyledonous weed florae which can be controlled by a compound of the present invention may be specifically mentioned, without limiting to certain species. Examples of weed species on which the active substances act efficiently include the monocotyledons such as annual Avena, Lolium, Alopecurus, Phalaris, Echinochloa, Digitaria, Setaria, and Cyperus, as well as perennial Agropyron, Cynodon, Imperata and Sorghum, and perennial Cyperus.

In the case of the dicotyledonous weed species, the spectrum of action extends to species such as annual Galium, Viola, Veronica, Lamium, Stellaria, Amaranthus, Sinapis, Ipomoea, Sida, Matricaria, and Abutilon, as well as perennial weeds Convolvulus, Cirsium, Rumex, and Artemisia. The active substances of the present invention effectively control harmful plants such as Echinochloa, Sagittaria, Alisma, Eleocharis, Scirpus, and Cyperus in the specific condition of paddy rice growing. If a compound of the present invention is applied to soil surface prior to germination, the weed seedlings are either prevented completely from emerging, or the weeds stop growing when reaching the cotyledon stage and eventually died completely after three to four weeks. In particular, a compound of the present invention exhibits excellent activity against Apera spica venti, Matsumurella chinense, Fallopia convolvulus, Stellaria media, Veronica hederifolia, Veronica persica, Viola tricolor, Amaranthus, Galium, and Kochia.

Although a compound of the present invention has excellent herbicidal activity against monocotyledonous and dicotyledonous weeds, there is no damage at all to crop plants of economic importance such as wheat, barley, rye, rice, maize, sugarbeet, cotton and soybean, or the damage is negligible. In particular, they have excellent compatibility with cereals such as wheat, barley, and maize, in particular wheat. Therefore, a compound of the present invention is highly suitable for selectively controlling undesired plants in plantings for agricultural or ornamental use.

Owing to their herbicidal properties, these active substances may be employed for controlling harmful plants in plantings of genetically engineered plants that are known or to be introduced. Transgenic plants usually have advantageous traits, for example, resistance to certain pesticides, in particular to certain herbicides; resistance to plant diseases or pathogenic microorganisms of plant diseases such as certain insects or microorganisms including fungi, bacteria, or viruses. Other particular traits relate to the following conditions of the product, for example, quantity, quality, storage stability, composition, and special ingredients. Thus, it is known that the obtained transgenic plant product has an increased starch content, or modified starch quality, or a different fatty acid composition.

A compound of Formula I or I′ of the present invention or salts thereof are preferably used in plantings of transgenic crops and ornamental plants of economic importance, for example, cereals such as wheat, barley, rye, oats, millet, rice, manioc, and maize; or in plantings of vegetable plants such as sugarbeet, cotton, soybean, rapeseed, potato, tomato, pea and the like. A compound of Formula I or I′ is preferably used as a herbicide in plantings of useful plants which are resistant, or have been made resistant by genetic engineering, against the toxic effects of the herbicide.

Conventional ways for breeding a plant which has a modified trait compared to known plants include, for example, conventional breeding methods and breeding of mutant strains. In other words, a novel plant having an improved trait may be generated with the aid of genetic engineering methods (see, e.g., EP0221044A, EP0131624A). For example, several methods have been described:
changing crop plants using genetic engineering to modify the starch synthesis in plants (e.g., WO 92/11376, WO 92/14827, WO 91/19806);
transgenic crop plants resistant to certain herbicides, including glufosinate herbicides (e.g., EP0242236A, EP0242246A), or glyphosate herbicides (WO 92/00377), or sulfonylurea herbicides (EP0257993A, U.S. Pat. No. 5,013,659A);
transgenic crop plants, for example cotton, able to produce Bacillus thuringiensis toxins (Bt toxins) which impart resistance to certain pests that invade the plants (EP0142924A, EP0193259A);
transgenic crop plants having a modified fatty acid composition (WO 91/13972).

Numerous molecular biology techniques which allow preparation of a transgenic plant having a modified trait are known (see, e.g., Sambrook et al., 1989, Molecular Cloning, A Laboratory Manual, 2^{nd} edition, Cold Spring Harbor Laboratory Press, Cold Spring Harbor, N.Y.; or Winnacker, “Gene und Klone” [Genes and Clones], VCH Weinheim, 2^{nd} edition, 1996; or Christou, “Trends in Plant Science”, 1 (1996) 423-431). In order to carry out genetic engineering manipulations, it is possible to introduce a nucleic acid molecule into a plasmid, allowing mutagenesis or a sequence change to occur by recombination of the DNA sequence. Using the above-mentioned standard processes, it is possible to, for example, substitute bases, remove partial sequences, or add natural or synthetic sequences. To link DNA fragments with each other, it is possible to attach adaptors or linkers to the fragments.

A plant cell having a gene product of reduced activity may be prepared by the following methods, for example, by expressing at least one appropriate antisense-RNA and sense-RNA to achieve a cosuppression effect, or by expressing at least one appropriately constructed ribozyme which specifically cleaves the transcripts of the above-mentioned gene product.

To this end, it is possible to employ a DNA molecule which comprises all the coding sequences of the gene product including any flanking sequence that may be present and a DNA molecule which comprises only parts of the coding sequences that must be long enough to cause an antisense effect in the cells. It is also possible to use sequences which have a high degree of homology but are not entirely identical to the coding sequences of the gene product.

When nucleic acid molecules are expressed in a plant, the synthesized protein may be localized in any desired compartment of the plant cell. However, to achieve localization in a certain compartment, it is possible, for example, to link the coding region with the DNA sequences to ensure localization in the certain location. Such sequences are known to a person skilled in the art (see, e.g., Braun et al., EMBO J., 11 (1992), 3219-3227; Wolter et al., Proc. Natl. Acad. Sci. USA, 85 (1988), 846-850; Sonnewald et al., Plant J., 1 (1991), 95-106).

The transgenic plant cells can be recombined onto a whole plant using known techniques. A transgenic plant may be of any desired plant species, i.e., a monocotyledonous and dicotyledonous plant. In this manner, it is possible to obtain a transgenic plant having a modified trait by overexpression, suppression, or inhibition of a homologous (=natural) gene or gene sequence, or by expression of a heterologous (=foreign) gene or gene sequence.

When using the active substances of the present invention in a transgenic crop, in addition to the inhibitory effect against harmful plants which can be observed in other crops, there are frequently special effects on the corresponding transgenic crop, for example, improving or broadening the spectrum of weeds which can be controlled, modifying the application amount during application, excellent combination of the drug resistance of the preferred transgenic crop and herbicide performance, as well as effects on the growth and the yield of the transgenic crop plant. The present invention therefore also provides use of the compounds as herbicides for controlling harmful plants among transgenic crop plants.

In addition, a compound of the present invention is able to significantly regulate the growth of a crop plant. These compounds are employed for the targeted control of plant constituents and for facilitating harvesting by engaging in plant metabolism in a regulating manner, for example by provoking desiccation and stunted growth. Furthermore, they are also suitable for regulating and inhibiting undesirable plant growth without destroying the growth of crop plants. Inhibition of plant growth plays a key role in many monocotyledonous and dicotyledonous crops because it can reduce or completely prevent lodging hereby.

A compound of the present invention may be applied in customary formulations in the form of wettable powders, emulsifiable concentrates, sprayable solutions, dusts, or granules. The present invention therefore also provides a herbicidal composition comprising a compound of Formula I or I′. A compound of Formula I or I′ may be formulated in numerous ways depending on the prevailing physical parameters in biology and/or chemistry. Examples of suitable formulation options are: wettable powders (WP), water-soluble powders (SP), water-soluble concentrates, emulsifiable concentrates (EC), emulsions such as oil-in-water and water-in-oil emulsions (EW), sprayable solutions, suspension concentrates (SC), oil dispersions (OD), oil- or water-based dispersions, oil-miscible solutions, dust powders (DP), capsule suspensions (CS), seed-dressing compositions, granules for broadcasting and soil application, spray granules, coating granules and adsorption granules, water-dispersible granules (WG), water-soluble granules (SG), ULV (Ultra-low volume) formulations, microcapsules, and waxes. These individual formulation types are known and described in the following literature, for example, Winnacker-Küchler, “Chemische Technologie” [Chemical Technology], Volume 7, C. Hauser Verlag Munich, 4^{th} edition, 1986; Wade van Valkenburg, “Pesticide Formulations”, Marcel Dekker, N.Y., 1973; K. Martens, “Spray Drying” Handbook, 3^{rd} edition, 1979, G. Goodwin Ltd. London.

Necessary formulation auxiliaries such as inert materials, surfactants, solvents and other additives are likewise known and described in the following documents, for example, Watkins, “Handbook of Insecticide Dust Diluents and Carriers”, 2^{nd} edition, Dorland Books, Caldwell N.J.; H. v. Olphen, “An Introduction to Clay Colloid Chemistry”, 2^{nd} edition, J. Wiley & Sons, N.Y.; C. Marsden, “Solvents Guide”, 2^{nd} edition, Interscience, N.Y. 1963; “McCutcheon's Detergents and Emulsifiers Annual”, MC Publ. Corp., Ridgewood N.J.; Sisley and Wood, “Encyclopedia of Surface Active Agents”, Chem. Publ. Co. Inc., N.Y., 1964; Schönfeldt, “Grenzflüchenaktive Äthylenoxidaddkte” [Surface-active ethylene oxide adducts], Wiss. Verlagagesell., Stuttgart, 1976; Winnacker-Kuchler, “Chemische Technologie” [Chemical Technology], Volume 7, C. Hauser Verlag Munich, 4^{th} edition, 1986.

Wettable powders are uniformly dispersible in water which contain, in addition to the active substance, a diluent or inert substance, ionic and nonionic surfactant (wetting agent, dispersant), for example polyethoxylated alkyl phenol, polyethoxylated fatty alcohol, polyethoxylated fatty amine, fatty alcohol polyglycol ether sulfate, alkane sulfonate, alkylphenyl sulfonate, sodium lignosulfonate, sodium 2,2′-dinaphthylmethane-6,6′-disulfonats, sodium dibutylnaphthalenesulfonate, or sodium methyl oleoyl taurate. To prepare the wettable powders, the herbicidally active substance is finely ground, for example, in a customary apparatus such as hammer mill, fan mill and air-jet mill, and mixed with formulation auxiliaries simultaneously or sequentially.

Emulsifiable concentrates are prepared by dissolving the active substance in an organic solvent, for example butanol, cyclohexanone, dimethylformamide, xylene, or aromatic compounds with relatively high boiling point, or hydrocarbons or mixtures of the solvents, with the addition of one or more ionic and/or nonionic surfactants (emulsifiers). Examples of emulsifiers which can be used are calcium alkylarylsulfonates such as calcium dodecylbenzene sulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters such as sorbitan fatty acid esters, or polyoxyethylene sorbitan esters such as polyoxyethylene sorbitan fatty acid esters.

Dusts are obtained by grinding the active substance with finely-divided solid substances, for example talc, natural clays such as kaolin, bentonite and pyrophyllite, or diatomaceous earth. Water- or oil-based suspensions may be prepared by the following methods, for example, by wet milling using a commercially customary bead mill, with or without the addition of the surfactants mentioned above in the case of another formulation type.

Emulsions such as oil-in-water emulsions (EW) may be prepared by means of stirrers, colloid mills and/or static mixers using aqueous organic solvents, and, if desired, the surfactants as mentioned above in the case of another formulation type may be added.

Granules may be prepared by the following methods that either spraying the active substance onto the adsorptive and granulating with inert materials, or concentrating the active substance onto the surface of carriers such as sand and kaolinite and granulating inert materials by means of adhesive binders such polyvinyl alcohol, sodium polyacrylate, or mineral oils. Suitable active substances may also be granulated in the manner which is customary for the preparation of fertilizer granules. If desired, fertilizers may be mixed in. Water-dispersible granules are prepared by customary methods such as spray-drying, fluidized-bed granulation, disk granulation, or mixing using high-speed mixers and extrusion without solid inert materials.

For the preparation methods of granules using disk, fluidized-bed, extruder and spray, see the following processes in, for example, “Spray-Drying Handbook” 3^{rd} edition, 1979, G. Goodwin Ltd., London; J. E. Browning, “Agglomeration”, Chemical and Engineering, 1967, pages 147 ff; “Perry's Chemical Engineer's Handbook”, 5^{th} edition, McGraw-Hill, New York 1973, pp. 8-57. For further details on the formulation of crop protection products, see, for example G. C. Klingman, “Weed Control as a Science”, John Wiley and Sons Inc., New York, 1961, pages 81-96 and J. D. Freyer, S. A. Evans, “Weed Control Handbook”, 5^{th} edition, Blackwell Scientific Publications, Oxford, 1968, pages 101-103.

An agrochemical formulation generally contains from 0.1 to 99% by weight, in particular from 0.1 to 95%, of the active substance of Formula I or I′. In a wettable powder, the concentration of the active substance is, for example, from about 10 to 99% by weight, and the remainder to 100% by weight consists of customary formulation constituents. In an emulsifiable concentrate, the concentration of the active substance may be from about 1 to 90%, preferably from 5 to 80%, by weight. A dust formulation contains from 1 to 30% by weight of the active substance, preferably from 5 to 20% by weight of the active substance in usual cases, while a sprayable solution contains from about 0.05 to 80% by weight, preferably from 2 to 50%, of the active substance. In the case of water-dispersible granules, the content of the active substance mainly depends on whether the active substance is liquid or solid and on auxiliaries, fillers, etc. that are used during granulation. In water-dispersible granules, the content of the active substance, for example, is between 1 and 95% by weight, preferably between 10 and 80% by weight.

In addition, formulations of the active substances may include tackifiers, wetting agents, dispersants, emulsifiers, penetrants, preservatives, antifreeze agents, solvents, fillers, carriers, colorants, defoamers, evaporation suppressors as well as pH and viscosity modifiers which are usually customary in all cases.

Based on these formulations, it is also possible to produce mixtures with other pesticidally active substances, for example insecticides, acaricides, herbicides and fungicides, and also with safeners, fertilizers and/or plant growth regulators, in a manner of pre-mix or tank mix.

In mixed formulations or tank mix formulations, suitable active substances which can be mixed with the active substances of the present invention are the known substances described, for example in World Herbicide New Product Technology Handbook, China Agricultural Science and Farming Techniques Press, 2010. 9 and in the literature cited herein. For example, the herbicidally active substances mentioned below may be mixed with a compound of Formula I or I′ (noted that the compounds are either named by the “common name” in accordance with the International Organization for Standardization (ISO) or by the chemical names, accompanied with a customary code number if appropriate): acetochlor, butachlor, alachlor, propisochlor, metolachlor, S-metolachlor, pretilachlor, propachlor, ethachlor, napropamide, R-left handed napropamide, propanil, mefenacet, diphenamid, diflufenican, ethaprochlor, beflubutamid, bromobutide, dimethenamid, dimethenamid-P, etobenzanid, flufenacet, thenylchlor, metazachlor, isoxaben, flamprop-M-methyl, flamprop-M-propyl, allidochlor, pethoxamid, chloranocryl, cypromid, mefluidide, monalide, delachlor, prynachlor, terbuchlor, xylachlor, dimethachlor, cisanilide, trimexachlor, clomeprop, propyzamide, pentanochlor, carbetamide, benzoylprop-ethyl, cyprazole, butenachlor, tebutam, benzipram, quinonamid, dichlofluanid, naproanilide, diethatyl-ethyl, naptalam, flufenacet, benzadox, chlorthiamid, chlorophthalimide, isocarbamide, picolinafen, atrazine, simazine, prometryn, cyanatryn, simetryn, ametryn, propazine, dipropetryn, SSH-108, terbutryn, terbuthylazine, triaziflam, cyprazine, proglinazine, trietazine, prometon, simetone, aziprotryne, desmetryn, dimethametryn, procyazine, mesoprazine, sebuthylazine, secbumeton, terbumeton, methoprotryne, cyanatryn, ipazine, chlorazine, atraton, pendimethalin, eglinazine, cyanuric acid, indaziflam, chlorsulfuron, metsulfuron-methyl, bensulfuron-methyl, chlorimuron-ethyl, tribenuron-methyl, thifensulfuron-methyl, pyrazosulfuron-ethyl, mesosulfuron, iodosulfuron-methyl sodium, foramsulfuron, cinosulfuron, triasulfuron, sulfometuron methyl, nicosulfuron, ethametsulfuron-methyl, amidosulfuron, ethoxysulfuron, cyclosulfamuron, rimsulfuron, azimsulfuron, flazasulfuron, monosulfuron, monosulfuron-ester, flucarbazone-sodium, flupyrsulfuron-methyl, halosulfuron-methyl, oxasulfuron, imazosulfuron, primisulfuron, propoxycarbazone, prosulfuron, sulfosulfuron, trifloxysulfuron, triflusulfuron-methyl, tritosulfuron, sodium metsulfuron methyl, flucetosulfuron, HNPC-C9908, orthosulfamuron, propyrisulfuron, metazosulfuron, acifluorfen, fomesafen, lactofen, fluoroglycofen, oxyfluorfen, chlornitrofen, aclonifen, ethoxyfen-ethyl, bifenox, nitrofluorfen, chlomethoxyfen, fluorodifen, fluoronitrofen, furyloxyfen, nitrofen, TOPE, DMNP, PPG1013, AKH-7088, halosafen, chlortoluron, isoproturon, linuron, diuron, dymron, fluometuron, benzthiazuron, methabenzthiazuron, cumyluron, ethidimuron, isouron, tebuthiuron, buturon, chlorbromuron, methyldymron, phenobenzuron, SK-85, metobromuron, metoxuron, afesin, monuron, siduron, fenuron, fluothiuron, neburon, chloroxuron, noruron, isonoruron, 3-cyclooctyl-1, thiazfluron, tebuthiuron, difenoxuron, parafluron, methylamine tribunil, karbutilate, trimeturon, dimefuron, monisouron, anisuron, methiuron, chloreturon, tetrafluron, phenmedipham, phenmedipham-ethyl, desmedipham, asulam, terbucarb, barban, propham, chlorpropham, rowmate, swep, chlorbufam, carboxazole, chlorprocarb, fenasulam, BCPC, CPPC, carbasulam, butylate, benthiocarb, vemolate, molinate, triallate, dimepiperate, esprocarb, pyributicarb, cycloate, avadex, EPTC, ethiolate, orbencarb, pebulate, prosulfocarb, tiocarbazil, CDEC, dimexano, isopolinate, methiobencarb, 2,4-D butyl ester, MCPA-Na, 2,4-D isooctyl ester, MCPA isooctyl ester, 2,4-D sodium salt, 2,4-D dimethyla mine salt, MCPA-thioethyl, MCPA, 2,4-D propionic acid, high 2,4-D propionic acid salt, 2,4-D butyric acid, MCPA propionic acid, MCPA propionic acid salt, MCPA butyric acid, 2,4,5-D, 2,4,5-D propionic acid, 2,4,5-D butyric acid, MCPA amine salt, dicamba, erbon, chlorfenac, saison, TBA, chloramben, methoxy-TBA, diclofop-methyl, fluazifop-butyl, fluazifop-P-butyl, haloxyfop-methyl, haloxyfop-P, quizalofop-ethyl, quizalofop-P-ethyl, fenoxaprop-ethy, fenoxaprop-P-ethyl, propaquizafop, cyhalofop-butyl, metamifop, clodinafop-propargyl, fenthiapropethyl, chloroazifop-propynyl, poppenate-methyl, trifopsime, isoxapyrifop, paraquat, diquat, oryzalin, ethalfluralin, isopropalin, nitralin, profluralin, prodinamine, benfluralin, fluchloraline, dinitramina, dipropalin, chlomidine, methalpropalin, dinoprop, glyphosate, anilofos, glufosinate ammonium, amiprophos-methyl, sulphosate, piperophos, bialaphos-sodium, bensulide, butamifos, phocarb, 2,4-DEP, H-9201, zytron, imazapyr, imazethapyr, imazaquin, imazamox, imazamox ammonium salt, imazapic, imazamethabenz-methyl, fluroxypyr, fluroxypyr isooctyl ester, clopyralid, picloram, trichlopyr, dithiopyr, haloxydine, 3,5,6-trichloro-2-pyridinol, thiazopyr, fluridone, aminopyralid, diflufenzopyr, triclopyr-butotyl, Clio-dinate, sethoxydim, clethodim, cycloxydim, alloxydim, clefoxydim, butroxydim, tralkoxydim, tepraloxydim, buthidazole, metribuzin, hexazinone, metamitron, ethiozin, ametridione, amibuzin, bromoxynil, bromoxynil octanoate, ioxynil octanoate, ioxynil, dichlobenil, diphenatrile, pyraclonil, chloroxynil, iodobonil, flumetsulam, florasulam, penoxsulam, metosulam, cloransulam-methyl, diclosulam, pyroxsulam, benfuresate, bispyribac-sodium, pyribenzoxim, pyriftalid, pyriminobac-methyl, pyrithiobac-sodium, benzobicylon, mesotrione, sulcotrione, tembotrione, tefuryltrione, bicyclopyrone, ketodpiradox, isoxaflutole, clomazone, fenoxasulfone, methiozolin, fluazolate, pyraflufen-ethyl, pyrazolynate, difenzoquat, pyrazoxyfen, benzofenap, nipyraclofen, pyrasulfotole, topramezone, pyroxasulfone, cafenstrole, flupoxam, aminotriazole, amicarbazone, azafenidin, carfentrazone-ethyl, sulfentrazone, bencarbazone, benzfendizone, butafenacil, bromacil, isocil, lenacil, terbacil, flupropacil, cinidon-ethyl, flumiclorac-pentyl, flumioxazin, S-23121, MK-129, flumezin, pentachlorophenol, dinoseb, dinoterb, dinoterb acetate, dinosam, DNOC, chloronitrophene, medinoterb acetate, dinofenate, oxadiargyl, oxadiazon, pentoxazone, flufenacet, fluthiacet-methyl, fentrazamide, flufenpyr-ethyl, pyrazon, brompyrazon, metflurazon, kusakira, dimidazon, oxapyrazon, norflurazon, pyridafol, quinclorac, quinmerac, bentazone, pyridate, oxaziclomefone, benazolin-ethyl, clomazone, cinmethylin, ZJ0702, pyribambenz-propyl, indanofan, sodium chlorate, dalapon, trichloroacetic acid, monochloroacetic acid, hexachloroacetone, flupropanate, cyperquat, bromofenoxim, epronaz, methazole, flurtamone, benfuresate, ethofumesate, tioclorim, chlorthal, fluorochloridone, tavron, acrolein, bentranil, tridiphane, chlorfenpropmethyl, thidiarizonaimin, phenisopham, busoxinone, methoxyphenone, saflufenacil, clacyfos, chloropon, alorac, diethamquat, etnipromid, iprymidam, ipfencarbazone, thiencarbazone-methyl, pyrimisulfan, chlorflurazole, tripropindan, sulglycapin, prosulfalin, cambendichlor, aminocyclopyrachlor, rodethanil, benoxacor, fenclorim, flurazole, fenchlorazole-ethyl, cloquintocet-mexyl, oxabetrinil, MG/91, cyometrinil, DKA-24, mefenpyr-diethyl, furilazole, fluxofenim, isoxadifen-ethyl, dichlormid, halauxifen-methyl, DOW848, UBH-509, D489, LS 82-556, KPP-300, NC-324, NC-330, KH-218, DPX-N8189, SC-0744, DOWC0535, DK-8910, V-53482, PP-600, MBH-001, KIH-9201, ET751, KIH-6127 and KIH-2023.

In one specific embodiment, the active substance (the additional herbicide in the foregoing (iii)) is selected from one or more of the following compounds:
(1) VLCFA inhibitors: pretilachlor (CAS: 51218-49-6), butachlor (CAS: 23184-66-9), mefenacet (CAS: 73250-68-7), acetochlor (CAS: 34256-82-1), anilofos (CAS: 64249-01-0);
(2) HPPD inhibitors:

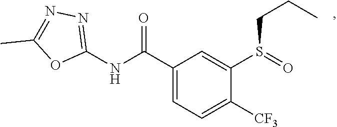 [SMILES: CCC[S@@](=O)c1cc(C(=O)Nc2nnc(C)o2)ccc1C(F)(F)F]

tefuryltrione (CAS: 473278-76-1), benzobicyclon (CAS: 156963-66-5), bipyrazone (CAS: 1622908-18-2);
(3) PPO inhibitors: oxadiazon (CAS: 19666-30-9), pyraclonil (CAS: 158353-15-2), oxyfluorfen (CAS: 42874-03-3), oxadiargyl (CAS: 39807-15-3), pentoxazone (CAS: 110956-75-7)

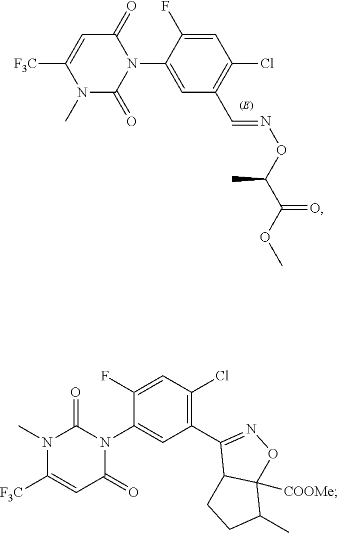 [SMILES: COC(=O)[C@@H](C)O/N=C/c1cc(-n2c(=O)cc(C(F)(F)F)n(C)c2=O)c(F)cc1Cl ; COC(=O)C12ON=C(c3cc(-n4c(=O)cc(C(F)(F)F)n(C)c4=O)c(F)cc3Cl)C1CCC2C]

(4) Synthetic hormones: halauxifen-methyl (CAS: 943831-98-9), florpyrauxifen-benzyl (CAS: 1390661-72-9),

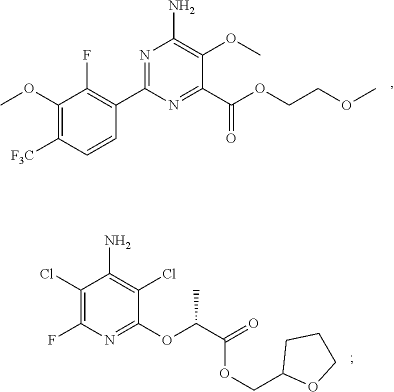

(5) PSII inhibitors: propanil (CAS: 709-98-8), bentazone (CAS: 25057-89-0), simetryn (CAS: 1014-70-6);
(6) DOXP inhibitors: clomazone (CAS: 81777-89-1),

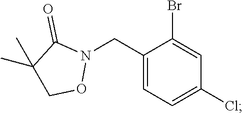 [SMILES: CC1(C)CON(Cc2ccc(Cl)cc2Br)C1=O]

(7) PDS inhibitors: bixlozone (CAS: 81777-95-9),

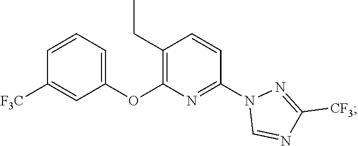 [SMILES: CCc1ccc(-n2cnc(C(F)(F)F)n2)nc1Oc1cccc(C(F)(F)F)c1]

(8) FAT inhibitors: cinmethylin (CAS: 87818-31-3),;

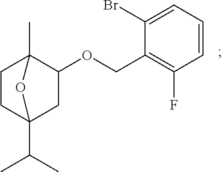 [SMILES: CC(C)C12CCC(C)(O1)C(OCc1c(F)cccc1Br)C2]

(9) Other herbicides: oxaziclomefone (CAS: 153197-14-9).

In another specific embodiment, the weight ratio of the effective ingredient (i) and the additional herbicide in (iii) in the composition is 1:100-100:1, 1:80-80:1, 1:50-50:1, 1:30-30:1, 1:20-20:1, 1:10-10:1, 1:5-1:1, or 1:1-5:1.

For use, the commercially available formulations are, if needed, diluted in customary manners, for example dilute with water in the cases of wettable powders, emulsifiable concentrates, dispersions, and water-dispersible granules. For products in the form of dusts, granules for soil application, or solutions for broadcasting and spray, usually no further dilution with inert materials is required prior to use. The required application amount of a compound of Formula I or I′ varies with the external conditions such as temperature, humidity, the nature of the used herbicide and the like. It may have a large variation range, for example, between 0.001 and 1.0 kg a.i./ha, or more active substances, but preferably between 0.005 and 750 g a.i./ha, in particular between 0.005 and 250 g a.i./ha.

Furthermore, another embodiment of the present application is a fungicidal composition which comprises a disease-inhibiting and phytologically acceptable amount of a compound of Formula I or I′; preferably, in one specific embodiment, also comprising a formulation auxiliary; in another specific embodiment, further comprising another active ingredient.

Another embodiment of the present application is use of a compound of Formula I or I′ or the above-mentioned fungicidal composition in preventing and controlling a phytopathogenic fungus, for protecting a plant from infestation by a phytopathogenic microorganism or for treating a plant which is attacked by a phytopathogenic microorganism. The use includes applying a compound of Formula I or I′ or a composition of the compound to soil, a plant, a part of a plant, leaves, and/or roots.

A compound of the present application may also be combined with another fungicide to form a fungicidal mixture. A compound of the present application is usually applied in combination with another fungicide to prevent and control a broader spectrum of undesired diseases. When applied in combination with another fungicide, the presently claimed compound may be formulated with another fungicide, tank-mixed with another fungicide, or applied sequentially with another fungicide. The other fungicides may include 2-(thiocyanatomethylthio)-benzothiazole, 2-phenylphenol, 8-hydroxyquinoline sulfate, ametoctradin, amisulbrom, antimycin, Ampelomyces quisqualis, azaconazole, azoxystrobin, Bacillus subtilis, Bacillus subtilis strain QST713, benalaxyl, benomyl, benthiavalicarb-isopropyl, benzylaminobenzene-sulfonate (BABS) salt, bicarbonates, biphenyl, bismerthiazol, bitertanol, bixafen, blasticidin-S, borax, Bordeaux mixture, boscalid, bromuconazole, bupirimate, calcium polysulfide, captafol, captan, carbendazim, carboxin, carpropamid, carvone, chlazafenone, chloroneb, chlorothalonil, chlozolinate, Coniothyrium minitans, copper hydroxide, copper octanoate, copper oxychloride, copper sulfate, copper sulfate (tribasic), cuprous oxide, cyazofamid, cyflufenamid, cymoxanil, cyproconazole, cyprodinil, dazomet, debacarb, diammonium ethylenebis-(dithiocarbamate), dichlofluanid, dichlorophen, diclocymet, diclomezine, dicloran, diethofencarb, difenoconazole, difenzoquat ion, diflumetorim, dimethomorph, dimoxystrobin, diniconazole, diniconazole-M, dinobuton, dinocap, diphenylamine, dithianon, dodemorph, dodemorph acetate, dodine, dodine free base, edifenphos, enestrobin, enestroburin, epoxiconazole, ethaboxam, ethoxyquin, etridiazole, famoxadone, fenamidone, fenarimol, fenbuconazole, fenfuram, fenhexamid, fenoxanil, fenpiclonil, fenpropidin, fenpropimorph, fenpyrazamine, fentin, fentin acetate, fentin hydroxide, ferbam, ferimzone, fluazinam, fludioxonil, flumorph, fluopicolide, fluopyram, fluoroimide, fluoxastrobin, fluquinconazole, flusilazole, flusulfamide, flutianil, flutolanil, flutriafol, fluxapyroxad, folpet, formaldehyde, fosetyl, fosetyl-aluminium, fuberidazole, furalaxyl, furametpyr, guazatine, guazatine acetates, GY-81, hexachlorobenzene, hexaconazole, hymexazol, imazalil, imazalil sulfate, imibenconazole, iminoctadine, iminoctadine triacetate, iminoctadine tris(albesilate), iodocarb, ipconazole, ipfenpyrazolone, iprobenfos, iprodione, iprovalicarb, isoprothiolane, isopyrazam, isotianil, kasugamycin, kasugamycin hydrochloride hydrate, kresoxim-methyl, laminarin, mancopper, mancozeb, mandipropamid, maneb, mefenoxam, mepanipyrim, mepronil, meptyl-dinocap, mercuric chloride, mercuric oxide, mercurous chloride, metalaxyl, metalaxyl-M, metam, metam-ammonium, metam-potassium, metam-sodium, metconazole, methasulfocarb, methyl iodide, methyl isothiocyanate, metiram, metominostrobin, metrafenone, mildiomycin, myclobutanil, nabam, nitrothal-isopropyl, nuarimol, octhilinone, ofurace, oleic acid (fatty acids), orysastrobin, oxadixyl, oxine-copper, oxpoconazole fumarate, oxycarboxin, pefurazoate, penconazole, pencycuron, penflufen, pentachlorophenol, pentachlorophenyl laurate, penthiopyrad, phenylmercury acetate, phosphonic acid, phthalide, picoxystrobin, polyoxin B, polyoxins, polyoxorim, potassium bicarbonate, potassium hydroxyquinoline sulfate, probenazole, prochloraz, procymidone, propamocarb, propamocarb hydrochloride, propiconazole, propineb, proquinazid, prothioconazole, pyraclostrobin, pyrametostrobin, pyraoxystrobin, pyrazophos, pyribencarb, pyributicarb, pyrifenox, pyrimethanil, pyriofenone, pyroquilon, quinoclamine, quinoxyfen, quintozene, Reynoutria sachalinensis extract, sedaxane, silthiofam, simeconazole, sodium 2-phenylphenoxide, sodium bicarbonate, sodium pentachlorophenoxide, spiroxamine, sulfur, SYP-Z048, tar oils, tebuconazole, tebufloquin, tecnazene, tetraconazole, thiabendazole, thifluzamide, thiophanate-methyl, thiram, tiadinil, tolclofos-methyl, tolylfluanid, triadimefon, triadimenol, triazoxide, tricyclazole, tridemorph, trifloxystrobin, triflumizole, triforine, triticonazole, validamycin, valifenalate, valiphenal, vinclozolin, zineb, ziram, zoxamide, Candida oleophila, Fusarium oxysporum, Gliocladium spp., Phlebiopsis gigantea, Streptomyces griseoviridis, Trichoderma spp., (RS)—N-(3,5-dichlorophenyl)-2-(methoxymethyl)-succinimide, 1,2-dichloropropane, 1,3-dichloro-1,1,3,3-tetrafluoroacetone hydrate, 1-chloro-2,4-dinitronaphthalene, 1-chloro-2-nitropropane, 2-(2-heptadecyl-2-imidazolin-1-yl)ethanol, 2,3-dihydro-5-phenyl-1,4-dithi-ine-1,1,4,4-tetraoxide, 2-methoxyethylmercury acetate, 2-methoxyethylmercury chloride, 2-methoxyethylmercury silicate, 3-(4-chlorophenyl)-5-methylrhodanine, 4-(2-nitroprop-1-enyl)phenyl thiocyanateme, ampropylfos, anilazine, azithiram, barium polysulfide, Bayer 32394, benodanil, benquinox, bentaluron, benzamacril; benzamacril-isobutyl, benzamorf, binapacryl, bis(methylmercury) sulfate, bis(tributyltin) oxide, buthiobate, cadmium calcium copper zinc chromate sulfate, carbamorph, CECA, chlobenthiazone, chloraniformethan, chlorfenazole, chlorquinox, climbazole, copper bis(3-phenylsalicylate), copper zinc chromate, cufraneb, cupric hydrazinium sulfate, cuprobam, cyclafuramid, cypendazole, cyprofuram, decafentin, dichlone, dichlozoline, diclobutrazol, dimethirimol, dinocton, dinosulfon, dinoterbon, dipyrithione, ditalimfos, dodicin, drazoxolon, EBP, ESBP, etaconazole, etem, ethirim, fenaminosulf, fenapanil, fenitropan, fluotrimazole, furcarbanil, furconazole, furconazole-cis, furmecyclox, furophanate, glyodine, griseofulvin, halacrinate, Hercules 3944, hexylthiofos, ICIA0858, isopamphos, isovaledione, mebenil, mecarbinzid, metazoxolon, methfuroxam, methylmercury dicyandiamide, metsulfovax, milneb, mucochloric anhydride, myclozolin, N-3,5-dichlorophenyl-succinimide, N-3-nitrophenylitaconimide, natamycin, N-ethylmercurio-4-toluenesulfonanilide, nickel bis(dimethyldithiocarbamate), OCH, phenylmercury dimethyldithiocarbamate, phenylmercury nitrate, phosdiphen, prothiocarb; prothiocarb hydrochloride, pyracarbolid, pyridinitril, pyroxychlor, pyroxyfur, quinacetol; quinacetol sulfate, quinazamid, quinconazole, rabenzazole, salicylanilide, SSF-109, sultropen, tecoram, thiadifluor, thicyofen, thiochlorfenphim, thiophanate, thioquinox, tioxymid, triamiphos, triarimol, triazbutil, trichlamide, urbacid, zarilamid, and any combinations thereof.

Another embodiment of the present application is a method for preventing and controlling a harmful fungus, comprising treating the fungus or a material, a plant, soil or a seed to be protected against the fungal infestation with a compound of Formula I or I′ or the above-mentioned fungicidal composition.

The compounds have been found to have significant fungicidal action, particularly for agricultural use. Many of the compounds are particularly effective for use with agricultural crops and horticultural plants.

It will be understood by one skilled in the art that the efficacy of the compounds for the foregoing fungus establishes the general utility of the compounds as fungicides.

The compounds have activity against a broad range of fungal pathogens. Exemplary pathogens may include, but are not limited to, causing agent of the following diseases: wheat leaf blotch (Mycosphaerella graminicola), wheat brown rust (Puccinia triticina), wheat stripe rust (Puccinia striiformis), scab of apple (Venturia inaequalis), powdery mildew of grapevine (Uncinula necator), barley scald (Rhynchosporium secalis), blast of rice (Pyricularia oryzae), rust of soybean (Phakopsora pachyrhizi), glume blotch of wheat (Leptosphaeria nodorum), powdery mildew of wheat (Blumeria graminis f. sp. tritici), powdery mildew of barley (Blumeria graminis f. sp. hordei), powdery mildew of cucurbits (Erysiphe cichoracearum), anthracnose of cucurbits (Glomerella lagenarium), leaf spot of beet (Cercospora beticola), early blight of tomato (Alternaria solani), and spot blotch of barley (Cochliobolus sativus). The exact amount of the active substance to be applied is dependent not only on the specific active substance being applied, but also on the particular action desired, the fungal species to be controlled and the growth stage thereof, as well as the part of the plant or other product to be contacted with the compound. Thus, all the compounds and formulations containing the compounds may not be equally effective at similar concentrations or may not be able to act against the same fungal species.

The compounds are effective in use with plants in a disease-inhibiting and phytologically acceptable amount. The term “disease-inhibiting and phytologically acceptable amount” refers to an amount of a compound that kills or inhibits the plant disease for which control is desired but is not significantly toxic to the plant. This amount is generally from about 0.1 to about 1000 ppm (parts per million), preferably 1 to 500 ppm. The exact concentration of the compound required varies with the fungal disease to be controlled, the type of formulation employed, the method of application, the particular plant species, climate conditions, and the like. A suitable application rate is typically in the range from about 0.10 to about 4 pounds/acre (about 0.01 to 0.45 grams per square meter, g/m²).

Furthermore, another embodiment of the present application is an insecticidal composition comprising a biologically effective amount of a compound of Formula I or I′; preferably, in one specific embodiment, also comprising a formulation auxiliary; in another specific embodiment, further comprising another active ingredient.

Another embodiment of the present application is use of a compound of Formula I or I′ or the above-mentioned insecticidal composition in preventing and controlling a pest insect.

Another embodiment of the present application is a method for preventing and controlling a pest insect, comprising exposing the pest insect or its environment to a biologically effective amount of a compound of Formula I or I′ or the above-mentioned insecticidal composition.

In one specific embodiment, a biologically effective amount of a compound of the present invention or a composition as defined above is used for treating a pest insect, its food source, its habitat or its breeding place; or a cultivated plant, plant propagation material (e.g., seeds), soil, area, material or environment where a pest insect grows or is likely to grow; or a material, cultivated plant, plant propagation material (e.g., seeds), soil, surface or space to be protected against attack or infestation by a pest insect.

The term “pest control” refers to inhibiting the development of a pest insect (including death, decreased food intake, and/or mating disruption), and related expressions can be defined similarly.

Additionally, a compound described herein may be combined with another pesticide, including insecticide, nematocide, acaricide, arthropodicide, bactericide or a combination thereof, that is compatible with the compound of the present application in the medium selected for application and not antagonistic to the activity of the compound of the present application to form an pesticidal mixture. A compound of the present application may be applied in conjunction with one or more other pesticides to control a wider variety of undesirable pests. When used in conjunction with another pesticide, the presently claimed compound may be formulated with another pesticide, tank-mixed with another pesticide or applied sequentially with another pesticide. Typical insecticides include, but are not limited to: 1,2-dichloropropane, abamectin, acephate, acetamiprid, acethion, acetoprole, acrinathrin, acrylonitrile, alanycarb, aldicarb, aldoxycarb, aldrin, allethrin, allosamidin, allyxycarb, alpha-cypermethrin, alpha-ecdysone, alpha-endosulfan, amidithion, aminocarb, amiton, amiton oxalate, amitraz, anabasine, athidathion, azadirachtin, azamethiphos, azinphos-ethyl, azinphos-methyl, azothoate, barium hexafluorosilicate, barthrin, bendiocarb, benfuracarb, bensultap, beta-cyfluthrin, beta-cypermethrin, bifenthrin, bioallethrin, bioethanomethrin, biopermethrin, bistrifluron, borax, boric acid, bromfenvinfos, bromocyclen, bromo-DDT, bromophos, bromophos-ethyl, bufencarb, buprofezin, butacarb, butathiofos, butocarboxim, butonate, butoxycarboxim, cadusafos, calcium arsenate, calcium polysulfide, camphechlor, carbanolate, carbaryl, carbofuran, carbon disulfide, carbon tetrachloride, carbophenothion, carbosulfan, cartap, cartap hydrochloride, chlorantraniliprole, chlorbicyclen, chlordane, chlordecone, chlordimeform, chlordimeform hydrochloride, chlorethoxyfos, chlorfenapyr, chlorfenvinphos, chlorfluazuron, chlormephos, chloroform, chloropicrin, chlorphoxim, chlorprazophos, chlorpyrifos, chlorpyrifos-methyl, chlorthiophos, chromafenozide, cinerin I, cinerin II, cinerins, cismethrin, cloethocarb, closantel, clothianidin, copper acetoarsenite, copper arsenate, copper naphthenate, copper oleate, coumaphos, coumithoate, crotamiton, crotoxyphos, crufomate, cryolite, cyanofenphos, cyanophos, cyanthoate, cyantraniliprole, cyclethrin, cycloprothrin, cyfluthrin, cyhalothrin, cypermethrin, cyphenothrin, cyromazine, cythioate, DDT, decarbofuran, deltamethrin, demephion, demephion-O, demephion-S, demeton, demeton-methyl, demeton-O, demeton-O-methyl, demeton-S, demeton-S-methyl, demeton-S-methylsulphon, diafenthiuron, dialifos, diatomaceous earth, diazinon, dicapthon, dichlofenthion, dichlorvos, dicresyl, dicrotophos, dicyclanil, dieldrin, diflubenzuron, dilor, dimefluthrin, dimefox, dimetan, dimethoate, dimethrin, dimethylvinphos, dimetilan, dinex, dinex-diclexine, dinoprop, dinosam, dinotefuran, diofenolan, dioxabenzofos, dioxacarb, dioxathion, disulfoton, dithicrofos, d-limonene, DNOC, DNOC-ammonium, DNOC-potassium, DNOC-sodium, doramectin, ecdysterone, emamectin, emamectin benzoate, EMPC, empenthrin, endosulfan, endothion, endrin, EPN, epofenonane, eprinomectin, esdepallethrine, esfenvalerate, etaphos, ethiofencarb, ethion, ethiprole, ethoate-methyl, ethoprophos, ethyl formate, ethyl-DDD, ethylene dibromide, ethylene dichloride, ethylene oxide, etofenprox, etrimfos, EXD, famphur, fenamiphos, fenazaflor, fenchlorphos, fenethacarb, fenfluthrin, fenitrothion, fenobucarb, fenoxacrim, fenoxycarb, fenpirithrin, fenpropathrin, fensulfothion, fenthion, fenthion-ethyl, fenvalerate, fipronil, flonicamid, flubendiamide, flucofuron, flucycloxuron, flucythrinate, flufenerim, flufenoxuron, flufenprox, fluvalinate, fonofos, formetanate, formetanate hydrochloride, formothion, formparanate, formparanate hydrochloride, fosmethilan, fospirate, fosthietan, furathiocarb, furethrin, gamma-cyhalothrin, gamma-HCH, halfenprox, halofenozide, HCH, HEOD, heptachlor, heptenophos, heterophos, hexaflumuron, HHDN, hydramethylnon, hydrogen cyanide, hydroprene, hyquincarb, imidacloprid, imiprothrin, indoxacarb, iodomethane, IPSP, isazofos, isobenzan, isocarbophos, isodrin, isofenphos, isofenphos-methyl, isoprocarb, isoprothiolane, isothioate, isoxathion, ivermectin, jasmolin I, jasmolin II, jodfenphos, juvenile hormone I, juvenile hormone II, juvenile hormone III, kelevan, kinoprene, lambda-cyhalothrin, lead arsenate, lepimectin, leptophos, lindane, lirimfos, lufenuron, lythidathion, malathion, malonoben, mazidox, mecarbam, mecarphon, menazon, mephosfolan, mercurous chloride, mesulfenfos, metaflumizone, methacrifos, methamidophos, methidathion, methiocarb, methocrotophos, methomyl, methoprene, methoxychlor, methoxyfenozide, methyl bromide, methyl isothiocyanate, methylchloroform, methylene chloride, metofluthrin, metolcarb, metoxadiazone, mevinphos, mexacarbate, milbemectin, milbemycin oxime, mipafox, mirex, molosultap, monocrotophos, monomehypo, monosultap, morphothion, moxidectin, naftalofos, naled, naphthalene, nicotine, nifiuridide, nitenpyram, nithiazine, nitrilacarb, novaluron, noviflumuron, omethoate, oxamyl, oxydemeton-methyl, oxydeprofos, oxydisulfoton, para-dichlorobenzene, parathion, parathion-methyl, penfiuron, pentachlorophenol, permethrin, phenkapton, phenothrin, phenthoate, phorate, phosalone, phosfolan, phosmet, phosnichlor, phosphamidon, phosphine, phoxim, phoxim-methyl, pirimetaphos, pirimicarb, pirimiphos-ethyl, pirimiphos-methyl, potassium arsenite, potassium thiocyanate, pp′-DDT, prallethrin, precocene I, precocene II, precocene III, primidophos, profenofos, profluralin, promacyl, promecarb, propaphos, propetamphos, propoxur, prothidathion, prothiofos, prothoate, protrifenbute, pyraclofos, pyrafiuprole, pyrazophos, pyresmethrin, pyrethrin I, pyrethrin II, pyrethrins, pyridaben, pyridalyl, pyridaphenthion, pyrifiuquinazon, pyrimidifen, pyrimitate, pyriprole, pyriproxyfen, quassia, quinalphos, quinalphos-methyl, quinothion, rafoxanide, resmethrin, rotenone, ryania, sabadilla, schradan, selamectin, silafluofen, silica gel, sodium arsenite, sodium fluoride, sodium hexafluorosilicate, sodium thiocyanate, sophamide, spinetoram, spinosad, spiromesifen, spirotetramat, sulcofuron, sulcofuron-sodium, sulfluramid, sulfotep, sulfoxaflor, sulfuryl fluoride, sulprofos, tau-fluvalinate, tazimcarb, TDE, tebufenozide, tebufenpyrad, tebupirimfos, teflubenzuron, tefluthrin, temephos, TEPP, terallethrin, terbufos, tetrachloroethane, tetrachlorvinphos, tetramethrin, tetramethylfluthrin, theta-cypermethrin, thiacloprid, thiamethoxam, thicrofos, thiocarboxime, thiocyclam, thiocyclam oxalate, thiodicarb, thiofanox, thiometon, thiosultap, thiosultap-disodium, thiosultap-monosodium, thuringiensin, tolfenpyrad, tralomethrin, transfluthrin, transpermethrin, triarathene, triazamate, triazophos, trichlorfon, trichlormetaphos-3, trichloronat, trifenofos, triflumuron, trimethacarb, triprene, vamidothion, vaniliprole, XMC, xylylcarb, zeta-cypermethrin, zolaprofos, and any combinations thereof.

The term “biologically effective amount” refers to an amount of a biologically active compound (e.g., a compound of Formula I and I′) sufficient to produce the desired biological effect when applied to (i.e., contacted with) a pest insect to be controlled, or its environment, or a plant, a seed from which a plant has grown, or the location of a plant (e.g., growth medium), thereby protecting the plant from damages caused by the pest insect or achieving another desired effect (e.g., increasing activity of the plant). A compound of the present invention may also be applied prophylactically where a pest insect or a parasite is expected to appear.

The content of a compound as an active ingredient may be varied as required, and the compound as an active ingredient may be used in a proportion properly chosen in the range of 0.01 to 90 parts by weight per 100 parts of the agrohorticultural agents of the present invention. For example, in dusts, granules, emulsion or wettable powders, the suitable content of a compound as an active ingredient is from 0.01 to 50 parts by weight (0.01 to 50 wt % of the total weight of the agrohorticultural insecticide).

The applicable amount of the agrohorticultural insecticide of the present invention varies depending upon various factors such as a purpose, an insect pest to be controlled, a growth state of a plant, tendency of emergence of insect pests, weather, environmental conditions, a preparation form, an application method, an application site and application time. It may be properly chosen in the range of 0.001 g to 10 kg, preferably 0.01 g to 1 kg, (in terms of the compound as an active ingredient) per 10 acres depending upon purposes.

### DESCRIPTION OF FIGURES

FIG. 1 shows the diagram of single crystal X-ray structure analysis of Compound 117′ of the present invention.

### DETAILED EMBODIMENTS OF THE INVENTION

The following examples are used to illustrate the present invention and should not be construed as limiting the present invention in any way. The scope of rights claimed by the present invention is described in the Claims.

In view of the economy and diversity of the compounds, some compounds were preferably synthesized. Among the many synthesized compounds, selected ones are listed in Table 1 below. The specific compound structures and corresponding compound information are as set forth in Tables 1 and 2. The compounds in Table 1 are only to better illustrate the present invention but do not limit the present invention. For a person skilled in the art, it should not be understood that the scope of the above-mentioned subject matters of the present invention is limited to the following compounds.

TABLE 1
Compound structure

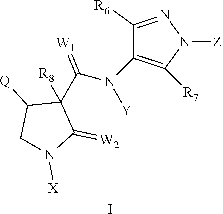

| No. | Q | R₆ | R₇ | R₈ | X | Y | Z | W₁ | W₂ |
|---|---|---|---|---|---|---|---|---|---|
| 1 | 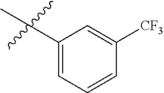 [SMILES: *c1cccc(C(F)(F)F)c1] | H | Cl | H | CH₃ | H | CH₃ | O | O |
| 2 | 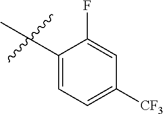 [SMILES: *c1ccc(C(F)(F)F)cc1F] | H | Cl | H | CH₃ | H | CH₃ | O | O |
| 3 | 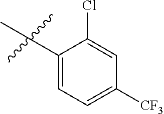 [SMILES: *c1ccc(C(F)(F)F)cc1Cl] | H | Cl | H | CH₃ | H | CH₃ | O | O |
| 4 | 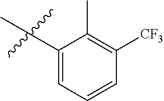 [SMILES: *c1cccc(C(F)(F)F)c1C] | H | Cl | H | CH₃ | H | CH₃ | O | O |
| 5 | 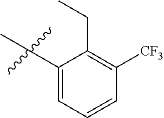 [SMILES: *c1cccc(C(F)(F)F)c1CC] | H | Cl | H | CH₃ | H | CH₃ | O | O |
| 6 | 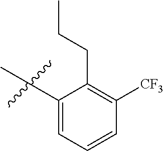 [SMILES: *c1cccc(C(F)(F)F)c1CCC] | H | Cl | H | CH₃ | H | CH₃ | O | O |
| 7 | 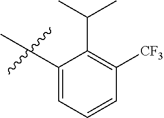 [SMILES: *c1cccc(C(F)(F)F)c1C(C)C] | H | Cl | H | CH₃ | H | CH₃ | O | O |
| 8 | 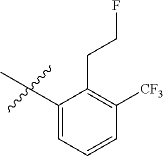 [SMILES: *c1cccc(C(F)(F)F)c1CCF] | H | Cl | H | CH₃ | H | CH₃ | O | O |
| 9 | 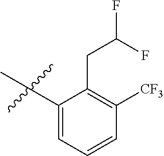 [SMILES: *c1cccc(C(F)(F)F)c1CC(F)F] | H | Cl | H | CH₃ | H | CH₃ | O | O |
| 10 | 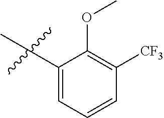 [SMILES: *c1cccc(C(F)(F)F)c1OC] | H | Cl | H | CH₃ | H | CH₃ | O | O |
| 11 | 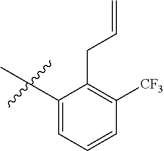 [SMILES: *c1cccc(C(F)(F)F)c1CC=C] | H | Cl | H | CH₃ | H | CH₃ | O | O |
| 12 | 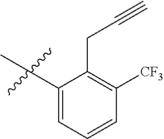 [SMILES: *c1cccc(C(F)(F)F)c1CC#C] | H | Cl | H | CH₃ | H | CH₃ | O | O |
| 13 | 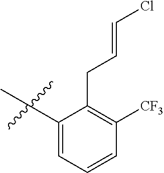 [SMILES: *c1cccc(C(F)(F)F)c1C/C=C/Cl] | H | Cl | H | CH₃ | H | CH₃ | O | O |
| 14 | 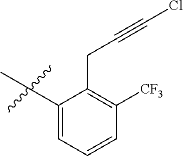 [SMILES: *c1cccc(C(F)(F)F)c1CC#CCl] | H | Cl | H | CH₃ | H | CH₃ | O | O |
| 15 | 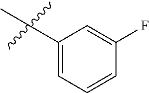 [SMILES: *c1cccc(F)c1] | H | Cl | H | CH₃ | H | CH₃ | O | O |
| 16 | 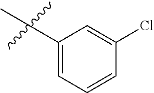 [SMILES: *c1cccc(Cl)c1] | H | Cl | H | CH₃ | H | CH₃ | O | O |
| 17 | 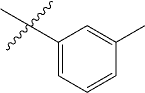 [SMILES: *c1cccc(C)c1] | H | Cl | H | CH₃ | H | CH₃ | O | O |
| 18 | 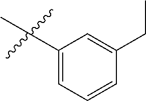 [SMILES: *c1cccc(CC)c1] | H | Cl | H | CH₃ | H | CH₃ | O | O |
| 19 | 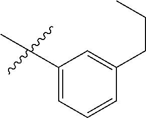 [SMILES: *c1cccc(CCC)c1] | H | Cl | H | CH₃ | H | CH₃ | O | O |
| 20 | 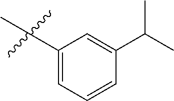 [SMILES: *c1cccc(C(C)C)c1] | H | Cl | H | CH₃ | H | CH₃ | O | O |
| 21 | 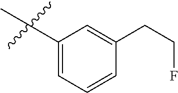 [SMILES: *c1cccc(CCF)c1] | H | Cl | H | CH₃ | H | CH₃ | O | O |
| 22 | 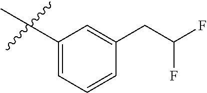 [SMILES: *c1cccc(CC(F)F)c1] | H | Cl | H | CH₃ | H | CH₃ | O | O |
| 23 | 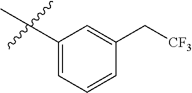 [SMILES: *c1cccc(CC(F)(F)F)c1] | H | Cl | H | CH₃ | H | CH₃ | O | O |
| 24 | 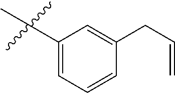 [SMILES: *c1cccc(CC=C)c1] | H | Cl | H | CH₃ | H | CH₃ | O | O |
| 25 | 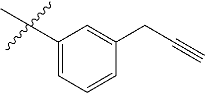 [SMILES: *c1cccc(CC#C)c1] | H | Cl | H | CH₃ | H | CH₃ | O | O |
| 26 | 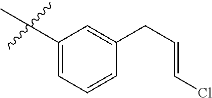 [SMILES: *c1cccc(C/C=C/Cl)c1] | H | Cl | H | CH₃ | H | CH₃ | O | O |
| 27 | 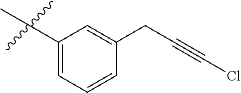 [SMILES: *c1cccc(CC#CCl)c1] | H | Cl | H | CH₃ | H | CH₃ | O | O |
| 28 | 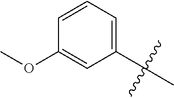 [SMILES: *c1cccc(OC)c1] | H | Cl | H | CH₃ | H | CH₃ | O | O |
| 29 | 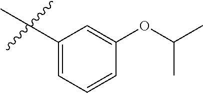 [SMILES: *c1cccc(OC(C)C)c1] | H | Cl | H | CH₃ | H | CH₃ | O | O |
| 30 | 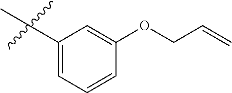 [SMILES: *c1cccc(OCC=C)c1] | H | Cl | H | CH₃ | H | CH₃ | O | O |
| 31 | 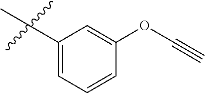 [SMILES: *c1cccc(OC#C)c1] | H | Cl | H | CH₃ | H | CH₃ | O | O |
| 32 | 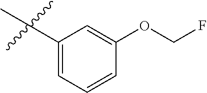 [SMILES: *c1cccc(OCF)c1] | H | Cl | H | CH₃ | H | CH₃ | O | O |
| 33 | 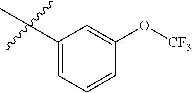 [SMILES: *c1cccc(OC(F)(F)F)c1] | H | Cl | H | CH₃ | H | CH₃ | O | O |
| 34 | 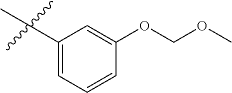 [SMILES: *c1cccc(OCOC)c1] | H | Cl | H | CH₃ | H | CH₃ | O | O |
| 35 | 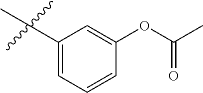 [SMILES: *c1cccc(OC(C)=O)c1] | H | Cl | H | CH₃ | H | CH₃ | O | O |
| 36 | 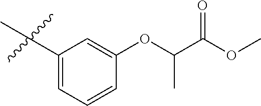 [SMILES: *c1cccc(OC(C)C(=O)OC)c1] | H | Cl | H | CH₃ | H | CH₃ | O | O |
| 37 | 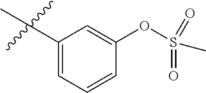 [SMILES: *c1cccc(OS(C)(=O)=O)c1] | H | Cl | H | CH₃ | H | CH₃ | O | O |
| 38 | 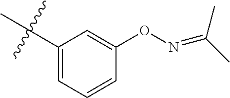 [SMILES: *c1cccc(ON=C(C)C)c1] | H | Cl | H | CH₃ | H | CH₃ | O | O |
| 39 | 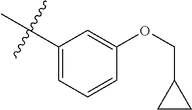 [SMILES: *c1cccc(OCC2CC2)c1] | H | Cl | H | CH₃ | H | CH₃ | O | O |
| 40 | 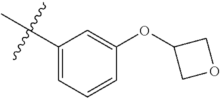 [SMILES: *c1cccc(OC2COC2)c1] | H | Cl | H | CH₃ | H | CH₃ | O | O |
| 41 | 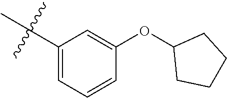 [SMILES: *c1cccc(OC2CCCC2)c1] | H | Cl | H | CH₃ | H | CH₃ | O | O |
| 42 | 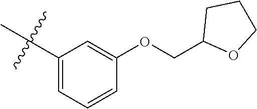 [SMILES: *c1cccc(OCC2CCCO2)c1] | H | Cl | H | CH₃ | H | CH₃ | O | O |
| 43 | 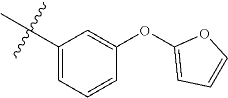 [SMILES: *c1cccc(Oc2ccco2)c1] | H | Cl | H | CH₃ | H | CH₃ | O | O |
| 44 | 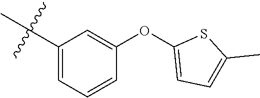 [SMILES: *c1cccc(Oc2ccc(C)s2)c1] | H | Cl | H | CH₃ | H | CH₃ | O | O |
| 45 | 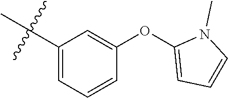 [SMILES: *c1cccc(Oc2cccn2C)c1] | H | Cl | H | CH₃ | H | CH₃ | O | O |
| 46 | 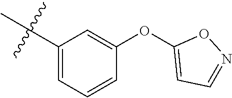 [SMILES: *c1cccc(Oc2ccno2)c1] | H | Cl | H | CH₃ | H | CH₃ | O | O |
| 47 | 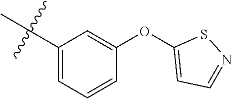 [SMILES: *c1cccc(Oc2ccns2)c1] | H | Cl | H | CH₃ | H | CH₃ | O | O |
| 48 | 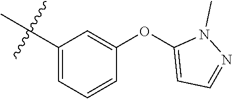 [SMILES: *c1cccc(Oc2ccnn2C)c1] | H | Cl | H | CH₃ | H | CH₃ | O | O |
| 49 | 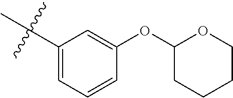 [SMILES: *c1cccc(OC2CCCCO2)c1] | H | Cl | H | CH₃ | H | CH₃ | O | O |
| 50 | 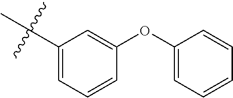 [SMILES: *c1cccc(Oc2ccccc2)c1] | H | Cl | H | CH₃ | H | CH₃ | O | O |
| 51 | 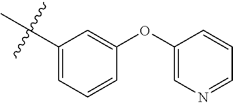 [SMILES: *c1cccc(Oc2cccnc2)c1] | H | Cl | H | CH₃ | H | CH₃ | O | O |
| 52 | 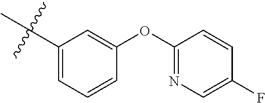 [SMILES: *c1cccc(Oc2ccc(F)cn2)c1] | H | Cl | H | CH₃ | H | CH₃ | O | O |
| 53 | 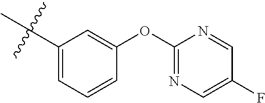 [SMILES: *c1cccc(Oc2ncc(F)cn2)c1] | H | Cl | H | CH₃ | H | CH₃ | O | O |
| 54 | 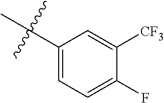 [SMILES: *c1ccc(F)c(C(F)(F)F)c1] | H | Cl | H | CH₃ | H | CH₃ | O | O |
| 55 | 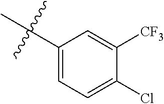 [SMILES: *c1ccc(Cl)c(C(F)(F)F)c1] | H | Cl | H | CH₃ | H | CH₃ | O | O |
| 56 | 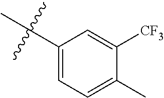 [SMILES: *c1ccc(C)c(C(F)(F)F)c1] | H | Cl | H | CH₃ | H | CH₃ | O | O |
| 57 | 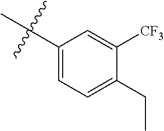 [SMILES: *c1ccc(CC)c(C(F)(F)F)c1] | H | Cl | H | CH₃ | H | CH₃ | O | O |
| 58 | 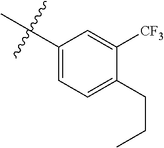 [SMILES: *c1ccc(CCC)c(C(F)(F)F)c1] | H | Cl | H | CH₃ | H | CH₃ | O | O |
| 59 | 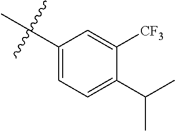 [SMILES: *c1ccc(C(C)C)c(C(F)(F)F)c1] | H | Cl | H | CH₃ | H | CH₃ | O | O |
| 60 | 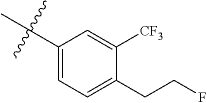 [SMILES: *c1ccc(CCF)c(C(F)(F)F)c1] | H | Cl | H | CH₃ | H | CH₃ | O | O |
| 61 | 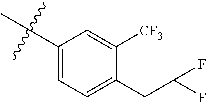 [SMILES: *c1ccc(CC(F)F)c(C(F)(F)F)c1] | H | Cl | H | CH₃ | H | CH₃ | O | O |
| 62 | 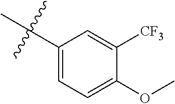 [SMILES: *c1ccc(OC)c(C(F)(F)F)c1] | H | Cl | H | CH₃ | H | CH₃ | O | O |
| 63 | 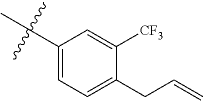 [SMILES: *c1ccc(CC=C)c(C(F)(F)F)c1] | H | Cl | H | CH₃ | H | CH₃ | O | O |
| 64 | 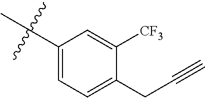 [SMILES: *c1ccc(CC#C)c(C(F)(F)F)c1] | H | Cl | H | CH₃ | H | CH₃ | O | O |
| 65 | 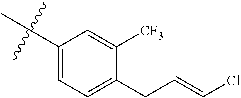 [SMILES: *c1ccc(C/C=C/Cl)c(C(F)(F)F)c1] | H | Cl | H | CH₃ | H | CH₃ | O | O |
| 66 | 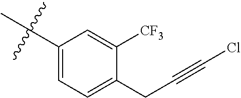 [SMILES: *c1ccc(CC#CCl)c(C(F)(F)F)c1] | H | Cl | H | CH₃ | H | CH₃ | O | O |
| 67 | 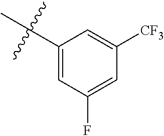 [SMILES: *c1cc(F)cc(C(F)(F)F)c1] | H | Cl | H | CH₃ | H | CH₃ | O | O |
| 68 | 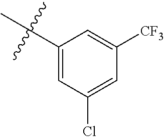 [SMILES: *c1cc(Cl)cc(C(F)(F)F)c1] | H | Cl | H | CH₃ | H | CH₃ | O | O |
| 69 | 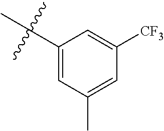 [SMILES: *c1cc(C)cc(C(F)(F)F)c1] | H | Cl | H | CH₃ | H | CH₃ | O | O |
| 70 | 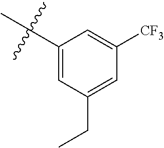 [SMILES: *c1cc(CC)cc(C(F)(F)F)c1] | H | Cl | H | CH₃ | H | CH₃ | O | O |
| 71 | 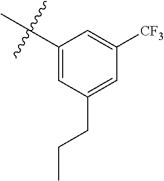 [SMILES: *c1cc(CCC)cc(C(F)(F)F)c1] | H | Cl | H | CH₃ | H | CH₃ | O | O |
| 72 | 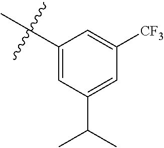 [SMILES: *c1cc(C(C)C)cc(C(F)(F)F)c1] | H | Cl | H | CH₃ | H | CH₃ | O | O |
| 73 | 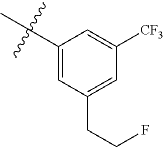 [SMILES: *c1cc(CCF)cc(C(F)(F)F)c1] | H | Cl | H | CH₃ | H | CH₃ | O | O |
| 74 | 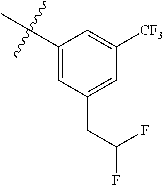 [SMILES: *c1cc(CC(F)F)cc(C(F)(F)F)c1] | H | Cl | H | CH₃ | H | CH₃ | O | O |
| 75 | 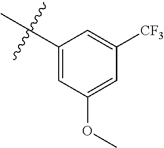 [SMILES: *c1cc(OC)cc(C(F)(F)F)c1] | H | Cl | H | CH₃ | H | CH₃ | O | O |
| 76 | 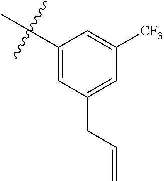 [SMILES: *c1cc(CC=C)cc(C(F)(F)F)c1] | H | Cl | H | CH₃ | H | CH₃ | O | O |
| 77 | 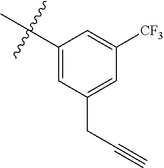 [SMILES: *c1cc(CC#C)cc(C(F)(F)F)c1] | H | Cl | H | CH₃ | H | CH₃ | O | O |
| 78 | 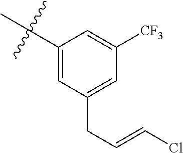 [SMILES: *c1cc(C/C=C/Cl)cc(C(F)(F)F)c1] | H | Cl | H | CH₃ | H | CH₃ | O | O |
| 79 | 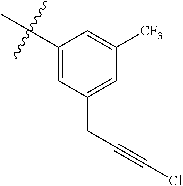 [SMILES: *c1cc(CC#CCl)cc(C(F)(F)F)c1] | H | Cl | H | CH₃ | H | CH₃ | O | O |
| 80 | 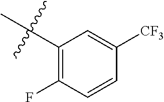 [SMILES: *c1cc(C(F)(F)F)ccc1F] | H | Cl | H | CH₃ | H | CH₃ | O | O |
| 81 | 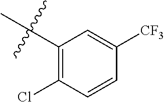 [SMILES: *c1cc(C(F)(F)F)ccc1Cl] | H | Cl | H | CH₃ | H | CH₃ | O | O |
| 82 | 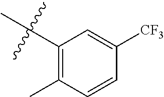 [SMILES: *c1cc(C(F)(F)F)ccc1C] | H | Cl | H | CH₃ | H | CH₃ | O | O |
| 83 | 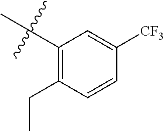 [SMILES: *c1cc(C(F)(F)F)ccc1CC] | H | Cl | H | CH₃ | H | CH₃ | O | O |
| 84 | 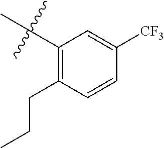 [SMILES: *c1cc(C(F)(F)F)ccc1CCC] | H | Cl | H | CH₃ | H | CH₃ | O | O |
| 85 | 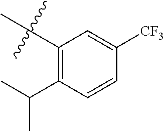 [SMILES: *c1cc(C(F)(F)F)ccc1C(C)C] | H | Cl | H | CH₃ | H | CH₃ | O | O |
| 86 | 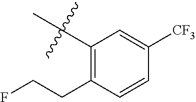 [SMILES: *c1cc(C(F)(F)F)ccc1CCF] | H | Cl | H | CH₃ | H | CH₃ | O | O |
| 87 | 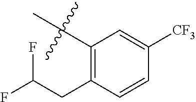 [SMILES: *c1cc(C(F)(F)F)ccc1CC(F)F] | H | Cl | H | CH₃ | H | CH₃ | O | O |
| 88 | 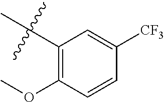 [SMILES: *c1cc(C(F)(F)F)ccc1OC] | H | Cl | H | CH₃ | H | CH₃ | O | O |
| 89 | 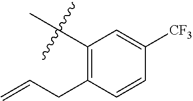 [SMILES: *c1cc(C(F)(F)F)ccc1CC=C] | H | Cl | H | CH₃ | H | CH₃ | O | O |
| 90 | 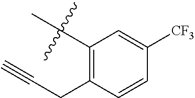 [SMILES: *c1cc(C(F)(F)F)ccc1CC#C] | H | Cl | H | CH₃ | H | CH₃ | O | O |
| 91 | 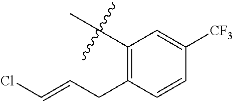 [SMILES: *c1cc(C(F)(F)F)ccc1C/C=C/Cl] | H | Cl | H | CH₃ | H | CH₃ | O | O |
| 92 | 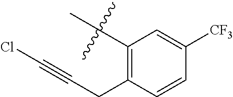 [SMILES: *c1cc(C(F)(F)F)ccc1CC#CCl] | H | Cl | H | CH₃ | H | CH₃ | O | O |
| 93 | 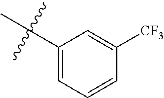 [SMILES: *c1cccc(C(F)(F)F)c1] | F | Cl | H | CH₃ | H | CH₃ | O | O |
| 94 | 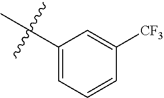 [SMILES: *c1cccc(C(F)(F)F)c1] | Cl | Cl | H | CH₃ | H | CH₃ | O | O |
| 95 | 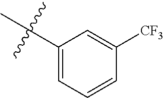 [SMILES: *c1cccc(C(F)(F)F)c1] | H | Cl | H | CH₃ | H | CH₂CH₃ | O | O |
| 96 | 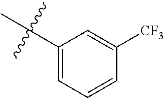 [SMILES: *c1cccc(C(F)(F)F)c1] | H | Cl | H | CH₃ | H | 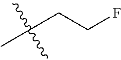 [SMILES: *CCF] | O | O |
| 97 | 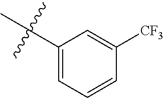 [SMILES: *c1cccc(C(F)(F)F)c1] | H | Cl | H | CH₃ | H | 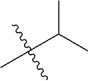 [SMILES: *C(C)C] | O | O |
| 98 | 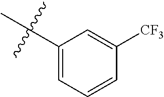 [SMILES: *c1cccc(C(F)(F)F)c1] | H | Cl | H | CH₃ | H | 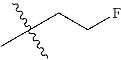 [SMILES: *CCF] | O | O |
| 99 | 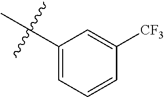 [SMILES: *c1cccc(C(F)(F)F)c1] | H | Cl | H | CH₃ | H | 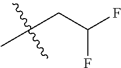 [SMILES: *CC(F)F] | O | O |
| 100 | 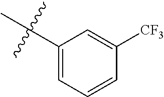 [SMILES: *c1cccc(C(F)(F)F)c1] | H | Cl | H | H | H | CH₃ | O | O |
| 101 | 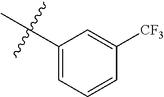 [SMILES: *c1cccc(C(F)(F)F)c1] | H | Cl | H | CH₂CH₃ | H | CH₃ | O | O |
| 102 | 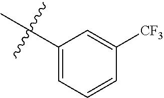 [SMILES: *c1cccc(C(F)(F)F)c1] | H | Cl | H | 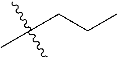 [SMILES: *CCC] | H | CH₃ | O | O |
| 103 | 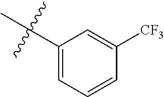 [SMILES: *c1cccc(C(F)(F)F)c1] | H | Cl | H | 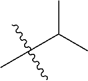 [SMILES: *C(C)C] | H | CH₃ | O | O |
| 104 | 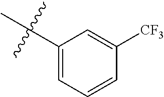 [SMILES: *c1cccc(C(F)(F)F)c1] | H | Cl | H | 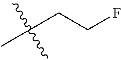 [SMILES: *CCF] | H | CH₃ | O | O |
| 105 | 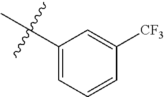 [SMILES: *c1cccc(C(F)(F)F)c1] | H | Cl | H | 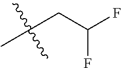 [SMILES: *CC(F)F] | H | CH₃ | O | O |
| 106 | 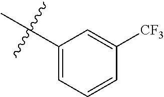 [SMILES: *c1cccc(C(F)(F)F)c1] | H | Cl | H | 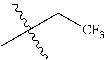 [SMILES: *CC(F)(F)F] | H | CH₃ | O | O |
| 107 | 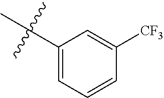 [SMILES: *c1cccc(C(F)(F)F)c1] | H | Cl | H | CH₃ | CH₃ | CH₃ | O | O |
| 108 | 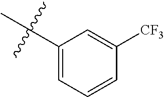 [SMILES: *c1cccc(C(F)(F)F)c1] | H | Cl | H | CH₃ | CH₂CH₃ | CH₃ | O | O |
| 109 | 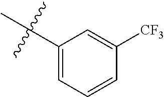 [SMILES: *c1cccc(C(F)(F)F)c1] | H | Cl | H | CH₃ | 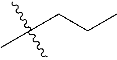 [SMILES: *CCC] | CH₃ | O | O |
| 110 | 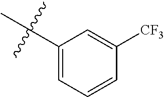 [SMILES: *c1cccc(C(F)(F)F)c1] | H | Cl | H | CH₃ | 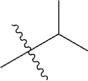 [SMILES: *C(C)C] | CH₃ | O | O |
| 111 | 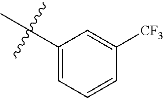 [SMILES: *c1cccc(C(F)(F)F)c1] | H | Cl | H | CH₃ | 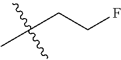 [SMILES: *CCF] | CH₃ | O | O |
| 112 | 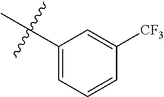 [SMILES: *c1cccc(C(F)(F)F)c1] | H | Cl | H | CH₃ | 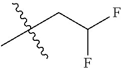 [SMILES: *CC(F)F] | CH₃ | O | O |
| 113 | 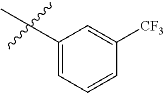 [SMILES: *c1cccc(C(F)(F)F)c1] | H | Cl | H | CH₃ | 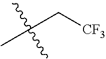 [SMILES: *CC(F)(F)F] | CH₃ | O | O |
| 114 | 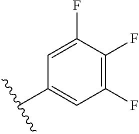 [SMILES: *c1cc(F)c(F)c(F)c1] | H | Cl | H | CH₃ | H | CH₃ | O | O |
| 115 | 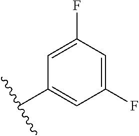 [SMILES: *c1cc(F)cc(F)c1] | H | Cl | H | CH₃ | H | CH₃ | O | O |
| 116 | 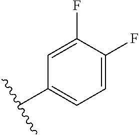 [SMILES: *c1ccc(F)c(F)c1] | H | Cl | H | CH₃ | H | CH₃ | O | O |
| 117 | 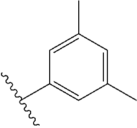 [SMILES: *c1cc(C)cc(C)c1] | H | Cl | H | CH₃ | H | CH₃ | O | O |
| 118 | 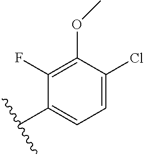 [SMILES: *c1ccc(Cl)c(OC)c1F] | H | Cl | H | CH₃ | H | CH₃ | O | O |
| 119 | 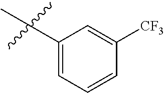 [SMILES: *c1cccc(C(F)(F)F)c1] | H | F | H | CH₃ | H | CH₃ | O | O |
| 120 | 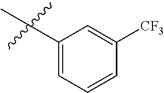 [SMILES: *c1cccc(C(F)(F)F)c1] | H | Br | H | CH₃ | H | CH₃ | O | O |
| 121 | 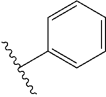 [SMILES: *c1ccccc1] | H | Cl | H | CH₃ | H | CH₃ | O | O |
| 122 | 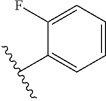 [SMILES: *c1ccccc1F] | H | Cl | H | CH₃ | H | CH₃ | O | O |
| 123 | 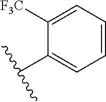 [SMILES: *c1ccccc1C(F)(F)F] | H | Cl | H | CH₃ | H | CH₃ | O | O |
| 124 | 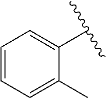 [SMILES: *c1ccccc1C] | H | Cl | H | CH₃ | H | CH₃ | O | O |
| 125 | 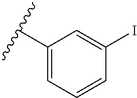 [SMILES: *c1cccc(I)c1] | H | Cl | H | CH₃ | H | CH₃ | O | O |
| 126 | 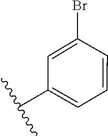 [SMILES: *c1cccc(Br)c1] | H | Cl | H | CH₃ | H | CH₃ | O | O |
| 127 | 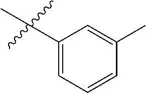 [SMILES: *c1cccc(C)c1] | H | Cl | H | CH₃ | H | 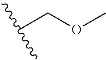 [SMILES: *COC] | O | O |
| 128 | 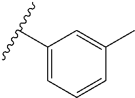 [SMILES: *c1cccc(C)c1] | H | Cl | H | CH₃ | H | 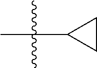 [SMILES: *C1CC1] | O | O |
| 129 | 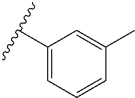 [SMILES: *c1cccc(C)c1] | H | Cl | H | CH₃ | H | CH₂CF₃ | O | O |
| 130 | 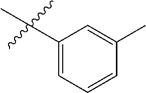 [SMILES: *c1cccc(C)c1] | H | Cl | H | CH₃ | H | 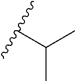 [SMILES: *C(C)C] | O | O |
| 131 | 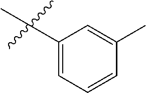 [SMILES: *c1cccc(C)c1] | H | Cl | H | CH₃ | H | CHF₂ | O | O |
| 132 | 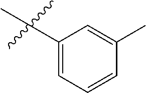 [SMILES: *c1cccc(C)c1] | H | Cl | H | CH₃ | H | 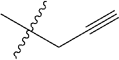 [SMILES: *CC#C] | O | O |
| 133 | 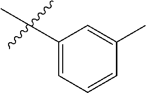 [SMILES: *c1cccc(C)c1] | H | H | H | CH₃ | H | CH₃ | O | O |
| 134 | 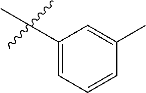 [SMILES: *c1cccc(C)c1] | H | CH₃ | H | CH₃ | H | CH₃ | O | O |
| 135 | 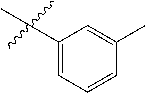 [SMILES: *c1cccc(C)c1] | H | CH₂CH₃ | H | CH₃ | H | CH₃ | O | O |
| 136 | 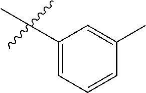 [SMILES: *c1cccc(C)c1] | H | CF₃ | H | CH₃ | H | CH₃ | O | O |
| 137 | 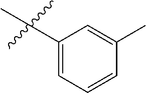 [SMILES: *c1cccc(C)c1] | H | 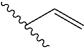 [SMILES: *C=C] | H | CH₃ | H | CH₃ | O | O |
| 138 | 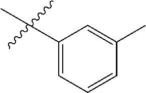 [SMILES: *c1cccc(C)c1] | H | 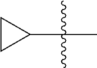 [SMILES: *C1CC1] | H | CH₃ | H | 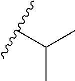 [SMILES: *C(C)C] | O | O |
| 139 | 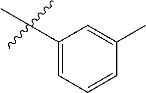 [SMILES: *c1cccc(C)c1] | H | 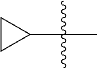 [SMILES: *C1CC1] | H | CH₃ | H | CH₃ | O | O |
| 140 | 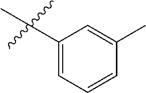 [SMILES: *c1cccc(C)c1] | H | 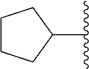 [SMILES: *C1CCCC1] | H | CH₃ | H | CH₃ | O | O |
| 141 | 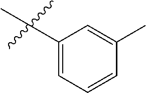 [SMILES: *c1cccc(C)c1] | H | 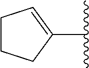 [SMILES: *C1=CCCC1] | H | CH₃ | H | CH₃ | O | O |
| 142 | 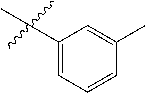 [SMILES: *c1cccc(C)c1] | H | 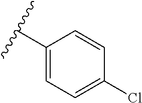 [SMILES: *c1ccc(Cl)cc1] | H | CH₃ | H | CH₃ | O | O |
| 143 | 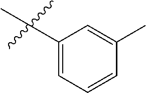 [SMILES: *c1cccc(C)c1] | Cl | H | H | CH₃ | H | CH₃ | O | O |
| 144 | 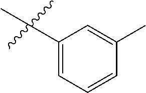 [SMILES: *c1cccc(C)c1] | Br | H | H | CH₃ | H | CH₃ | O | O |
| 145 | 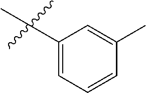 [SMILES: *c1cccc(C)c1] | CH₃ | Cl | H | CH₃ | H | CH₃ | O | O |
| 146 | 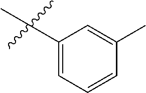 [SMILES: *c1cccc(C)c1] | CF₃ | H | H | CH₃ | H | CH₃ | O | O |
| 147 | 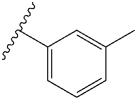 [SMILES: *c1cccc(C)c1] | OH | F | H | CH₃ | H | CH₃ | O | O |
| 148 | 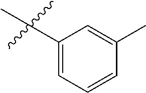 [SMILES: *c1cccc(C)c1] | OH | H | H | CH₃ | H | CH₃ | O | O |
| 149 | 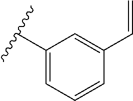 [SMILES: *c1cccc(C=C)c1] | H | Cl | H | CH₃ | H | CH₃ | O | O |
| 150 | 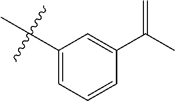 [SMILES: *c1cccc(C(=C)C)c1] | H | Cl | H | CH₃ | H | CH₃ | O | O |
| 151 | 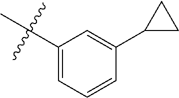 [SMILES: *c1cccc(C2CC2)c1] | H | Cl | H | CH₃ | H | CH₃ | O | O |
| 152 | 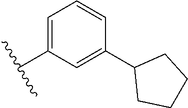 [SMILES: *c1cccc(C2CCCC2)c1] | H | Cl | H | CH₃ | H | CH₃ | O | O |
| 153 | 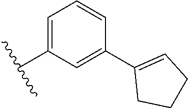 [SMILES: *c1cccc(C2=CCCC2)c1] | H | Cl | H | CH₃ | H | CH₃ | O | O |
| 154 | 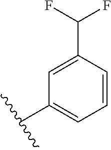 [SMILES: *c1cccc(C(F)F)c1] | H | Cl | H | CH₃ | H | CH₃ | O | O |
| 155 | 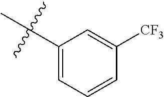 [SMILES: *c1cccc(C(F)(F)F)c1] | H | H | H | 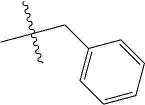 [SMILES: *Cc1ccccc1] | H | CH₃ | O | O |
| 156 | 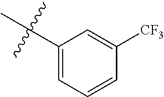 [SMILES: *c1cccc(C(F)(F)F)c1] | H | H | H | CH₃ | H | CH₃ | O | O |
| 157 | 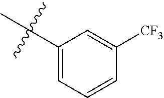 [SMILES: *c1cccc(C(F)(F)F)c1] | H | Cl | H | CH₃ | H | 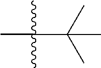 [SMILES: *C(C)(C)C] | O | O |
| 158 | 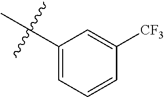 [SMILES: *c1cccc(C(F)(F)F)c1] | H | Cl | H | CH₃ | H | 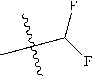 [SMILES: *C(F)F] | O | O |
| 159 | 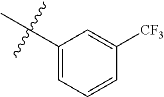 [SMILES: *c1cccc(C(F)(F)F)c1] | H | H | H | CH₃ | H | 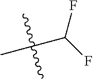 [SMILES: *C(F)F] | O | O |
| 160 | 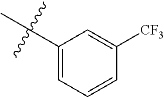 [SMILES: *c1cccc(C(F)(F)F)c1] | H | Cl | H | CH₃ | H | 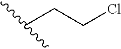 [SMILES: *CCCl] | O | O |
| 161 | 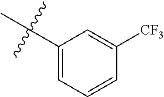 [SMILES: *c1cccc(C(F)(F)F)c1] | H | Cl | H | CH₃ | H | CH₂CF₃ | O | O |
| 162 | 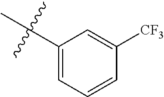 [SMILES: *c1cccc(C(F)(F)F)c1] | H | Cl | H | CH₃ | H | 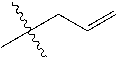 [SMILES: *CC=C] | O | O |
| 163 | 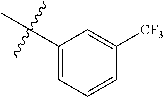 [SMILES: *c1cccc(C(F)(F)F)c1] | H | Cl | H | CH₃ | H | 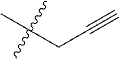 [SMILES: *CC#C] | O | O |
| 164 | 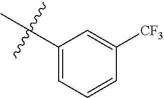 [SMILES: *c1cccc(C(F)(F)F)c1] | H | Cl | H | CH₃ | H | 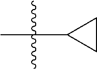 [SMILES: *C1CC1] | O | O |
| 165 | 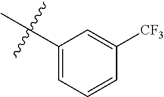 [SMILES: *c1cccc(C(F)(F)F)c1] | H | Cl | H | CH₃ | H | 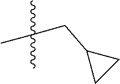 [SMILES: *CC1CC1] | O | O |
| 166 | 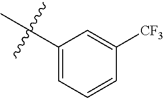 [SMILES: *c1cccc(C(F)(F)F)c1] | H | Cl | H | CH₃ | H | 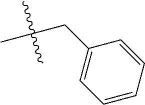 [SMILES: *Cc1ccccc1] | O | O |
| 167 | 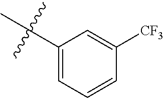 [SMILES: *c1cccc(C(F)(F)F)c1] | H | Cl | H | CH₃ | H | NH₂ | O | O |
| 168 | 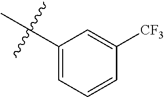 [SMILES: *c1cccc(C(F)(F)F)c1] | H | Cl | H | CH₃ | H | NO₂ | O | O |
| 169 | 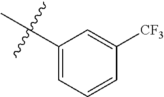 [SMILES: *c1cccc(C(F)(F)F)c1] | H | Cl | H | CH₃ | H | OH | O | O |
| 170 | 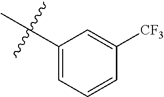 [SMILES: *c1cccc(C(F)(F)F)c1] | H | Cl | H | CH₃ | H | OCH₃ | O | O |
| 171 | 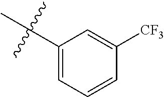 [SMILES: *c1cccc(C(F)(F)F)c1] | H | Cl | H | CH₃ | H | 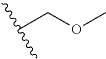 [SMILES: *COC] | O | O |
| 172 | 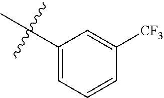 [SMILES: *c1cccc(C(F)(F)F)c1] | H | Cl | H | CH₃ | H | 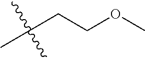 [SMILES: *CCOC] | O | O |
| 173 | 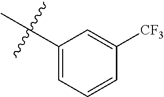 [SMILES: *c1cccc(C(F)(F)F)c1] | H | Cl | H | CH₃ | H | 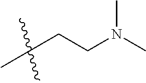 [SMILES: *CCN(C)C] | O | O |
| 174 | 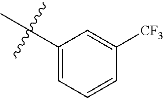 [SMILES: *c1cccc(C(F)(F)F)c1] | H | Cl | H | CH₃ | H | 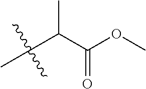 [SMILES: *C(C)C(=O)OC] | O | O |
| 175 | 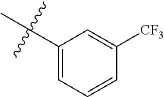 [SMILES: *c1cccc(C(F)(F)F)c1] | H | Cl | H | CH₃ | H | 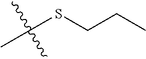 [SMILES: *SCCC] | O | O |
| 176 | 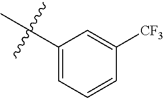 [SMILES: *c1cccc(C(F)(F)F)c1] | H | Cl | H | CH₃ | H | SO₂CH₃ | O | O |
| 177 | 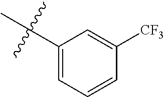 [SMILES: *c1cccc(C(F)(F)F)c1] | H | Cl | H | CH₃ | H | SO₂N(CH₃)₂ | O | O |
| 178 | 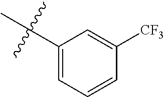 [SMILES: *c1cccc(C(F)(F)F)c1] | H | Cl | H | CH₃ | H | TMS | O | O |
| 179 | 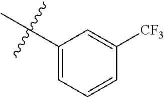 [SMILES: *c1cccc(C(F)(F)F)c1] | H | Cl | H | CH₃ | H | 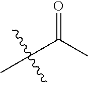 [SMILES: *C(C)=O] | O | O |
| 180 | 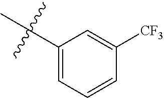 [SMILES: *c1cccc(C(F)(F)F)c1] | H | Cl | H | CH₃ | H | 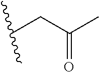 [SMILES: *CC(C)=O] | O | O |
| 181 | 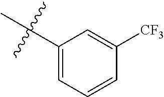 [SMILES: *c1cccc(C(F)(F)F)c1] | H | Cl | H | CH₃ | H | 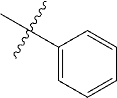 [SMILES: *c1ccccc1] | O | O |
| 182 | 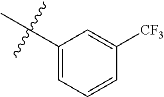 [SMILES: *c1cccc(C(F)(F)F)c1] | H | Cl | H | CH₃ | H | 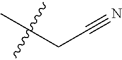 [SMILES: *CC#N] | O | O |
| 183 | 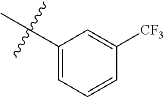 [SMILES: *c1cccc(C(F)(F)F)c1] | H | Cl | H | 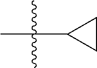 [SMILES: *C1CC1] | H | CH₃ | O | O |
| 184 | 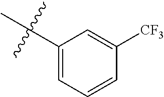 [SMILES: *c1cccc(C(F)(F)F)c1] | H | Cl | H | 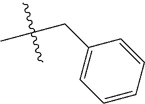 [SMILES: *Cc1ccccc1] | H | CH₃ | O | O |
| 185 | 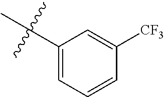 [SMILES: *c1cccc(C(F)(F)F)c1] | H | Cl | H | 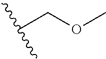 [SMILES: *COC] | H | CH₃ | O | O |
| 186 | 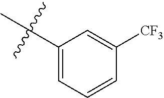 [SMILES: *c1cccc(C(F)(F)F)c1] | H | I | H | CH₃ | H | CH₃ | O | O |
| 187 | 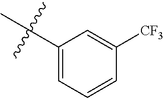 [SMILES: *c1cccc(C(F)(F)F)c1] | H | CN | H | CH₃ | H | CH₃ | O | O |
| 188 | 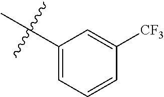 [SMILES: *c1cccc(C(F)(F)F)c1] | H | NO₂ | H | CH₃ | H | CH₃ | O | O |
| 189 | 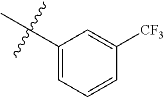 [SMILES: *c1cccc(C(F)(F)F)c1] | H | CH₃ | H | CH₃ | H | CH₃ | O | O |
| 190 | 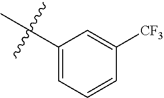 [SMILES: *c1cccc(C(F)(F)F)c1] | H | CH₂CH₃ | H | CH₃ | H | CH₃ | O | O |
| 191 | 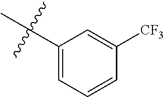 [SMILES: *c1cccc(C(F)(F)F)c1] | H | CF₃ | H | CH₃ | H | CH₃ | S | S |
| 192 | 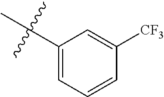 [SMILES: *c1cccc(C(F)(F)F)c1] | H | OCH₃ | H | CH₃ | H | CH₃ | O | O |
| 193 | 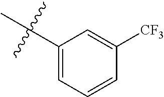 [SMILES: *c1cccc(C(F)(F)F)c1] | H | 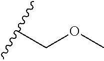 [SMILES: *COC] | H | CH₃ | H | CH₃ | O | O |
| 194 | 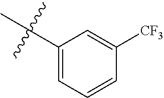 [SMILES: *c1cccc(C(F)(F)F)c1] | H | 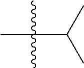 [SMILES: *C(C)C] | H | CH₃ | H | CH₃ | O | O |
| 195 | 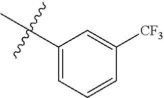 [SMILES: *c1cccc(C(F)(F)F)c1] | H | 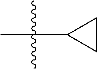 [SMILES: *C1CC1] | H | CH₃ | H | CH₃ | O | O |
| 196 | 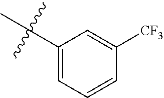 [SMILES: *c1cccc(C(F)(F)F)c1] | H | 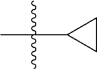 [SMILES: *C1CC1] | H | CH₃ | H | CHF₂ | O | O |
| 197 | 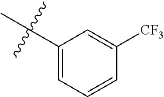 [SMILES: *c1cccc(C(F)(F)F)c1] | H | 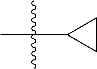 [SMILES: *C1CC1] | H | CH₃ | H | 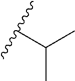 [SMILES: *C(C)C] | O | O |
| 198 | 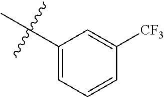 [SMILES: *c1cccc(C(F)(F)F)c1] | H | NH₂ | H | CH₃ | H | CH₃ | O | O |
| 199 | 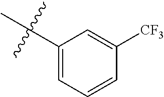 [SMILES: *c1cccc(C(F)(F)F)c1] | H | 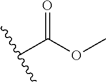 [SMILES: *C(=O)OC] | H | CH₃ | H | CH₃ | O | O |
| 200 | 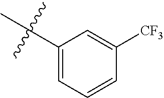 [SMILES: *c1cccc(C(F)(F)F)c1] | H | 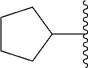 [SMILES: *C1CCCC1] | H | CH₃ | H | CH₃ | O | O |
| 201 | 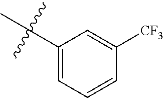 [SMILES: *c1cccc(C(F)(F)F)c1] | H | 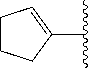 [SMILES: *C1=CCCC1] | H | CH₃ | H | CH₃ | O | O |
| 202 | 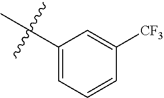 [SMILES: *c1cccc(C(F)(F)F)c1] | H | 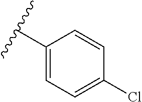 [SMILES: *c1ccc(Cl)cc1] | H | CH₃ | H | CH₃ | O | O |
| 203 | 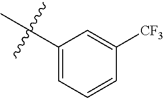 [SMILES: *c1cccc(C(F)(F)F)c1] | H | 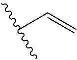 [SMILES: *C=C] | H | CH₃ | H | CH₃ | O | O |
| 204 | 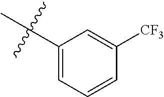 [SMILES: *c1cccc(C(F)(F)F)c1] | H | 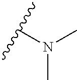 [SMILES: *N(C)C] | H | CH₃ | H | CH₃ | O | O |
| 205 | 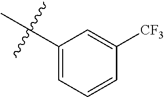 [SMILES: *c1cccc(C(F)(F)F)c1] | Cl | H | H | CH₃ | H | CH₃ | O | O |
| 206 | 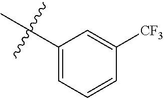 [SMILES: *c1cccc(C(F)(F)F)c1] | Br | H | H | CH₃ | H | CH₃ | O | O |
| 207 | 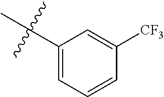 [SMILES: *c1cccc(C(F)(F)F)c1] | Br | Cl | H | CH₃ | H | CH₃ | O | O |
| 208 | 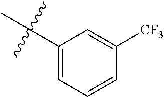 [SMILES: *c1cccc(C(F)(F)F)c1] | CH₃ | Cl | H | CH₃ | H | CH₃ | O | O |
| 209 | 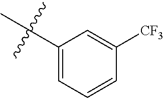 [SMILES: *c1cccc(C(F)(F)F)c1] | CH₃ | Cl | H | CH₃ | H | CHF₂ | O | O |
| 210 | 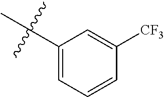 [SMILES: *c1cccc(C(F)(F)F)c1] | 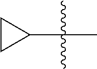 [SMILES: *C1CC1] | H | H | CH₃ | H | CH₃ | O | O |
| 211 | 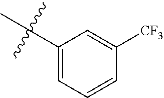 [SMILES: *c1cccc(C(F)(F)F)c1] | OH | Cl | H | CH₃ | H | CH₃ | O | O |
| 212 | 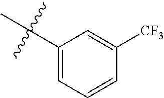 [SMILES: *c1cccc(C(F)(F)F)c1] | OH | F | H | CH₃ | H | CH₃ | O | O |
| 213 | 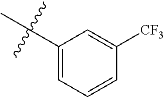 [SMILES: *c1cccc(C(F)(F)F)c1] | 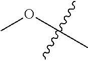 [SMILES: *OC] | H | H | CH₃ | H | CH₃ | O | O |
| 214 | 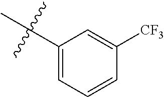 [SMILES: *c1cccc(C(F)(F)F)c1] | CF₃ | H | H | CH₃ | H | CH₃ | O | O |
| 215 | 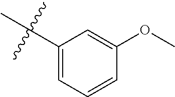 [SMILES: *c1cccc(OC)c1] | H | Cl | H | CH₃ | H | CHF₂ | O | O |
| 216 | 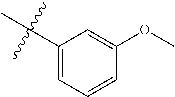 [SMILES: *c1cccc(OC)c1] | CH₃ | Cl | H | CH₃ | H | CH₃ | O | O |
| 217 | 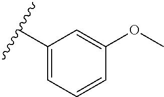 [SMILES: *c1cccc(OC)c1] | H | 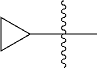 [SMILES: *C1CC1] | H | CH₃ | H | CH₃ | O | O |
| 218 | 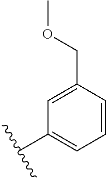 [SMILES: *c1cccc(COC)c1] | H | Cl | H | CH₃ | H | CH₃ | O | O |
| 219 | 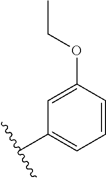 [SMILES: *c1cccc(OCC)c1] | H | Cl | H | CH₃ | H | CH₃ | O | O |
| 220 | 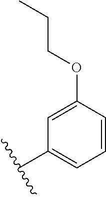 [SMILES: *c1cccc(OCCC)c1] | H | Cl | H | CH₃ | H | CH₃ | O | O |
| 221 | 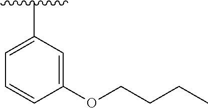 [SMILES: *c1cccc(OCCCC)c1] | H | Cl | H | CH₃ | H | CH₃ | O | O |
| 222 | 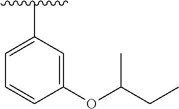 [SMILES: *c1cccc(OC(C)CC)c1] | H | Cl | H | CH₃ | H | CH₃ | O | O |
| 223 | 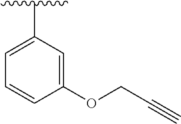 [SMILES: *c1cccc(OCC#C)c1] | H | Cl | H | CH₃ | H | CH₃ | O | O |
| 224 | 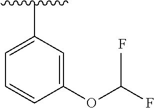 | H | Cl | H | CH₃ | H | CH₃ | O | O |
| 225 | 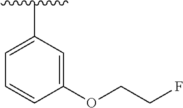 [SMILES: *c1cccc(OCCF)c1] | H | Cl | H | CH₃ | H | CH₃ | O | O |
| 226 | 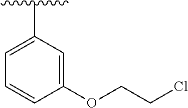 [SMILES: *c1cccc(OCCCl)c1] | H | Cl | H | CH₃ | H | CH₃ | O | O |
| 227 | 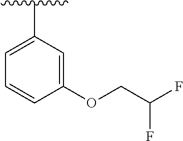 [SMILES: *c1cccc(OCC(F)F)c1] | H | Cl | H | CH₃ | H | CH₃ | O | O |
| 228 | 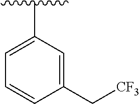 [SMILES: *c1cccc(CC(F)(F)F)c1] | CF₃ | H | H | CH₃ | H | CH₃ | O | O |
| 229 | 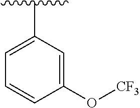 [SMILES: *c1cccc(OC(F)(F)F)c1] | OH | F | H | CH₃ | H | CH₃ | O | O |
| 230 | 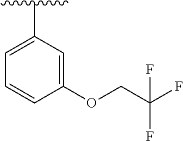 [SMILES: *c1cccc(OCC(F)(F)F)c1] | H | Cl | H | CH₃ | H | CH₃ | O | O |
| 231 | 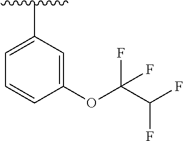 [SMILES: *c1cccc(OC(F)(F)C(F)F)c1] | H | Cl | H | CH₃ | H | CH₃ | O | O |
| 232 | 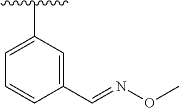 [SMILES: *c1cccc(/C=N/OC)c1] | H | Cl | H | CH₃ | H | CH₃ | O | O |
| 233 | 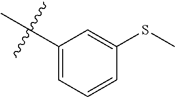 [SMILES: *c1cccc(SC)c1] | H | Cl | H | CH₃ | H | CH₃ | O | O |
| 234 | 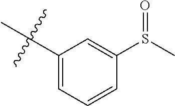 [SMILES: *c1cccc(S(C)=O)c1] | H | Cl | H | CH₃ | H | CH₃ | O | O |
| 235 | 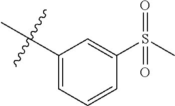 [SMILES: *c1cccc(S(C)(=O)=O)c1] | H | Cl | H | CH₃ | H | CH₃ | O | O |
| 236 | 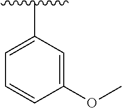 [SMILES: *c1cccc(OC)c1] | OH | F | H | CH₃ | H | CH₃ | O | O |
| 237 | 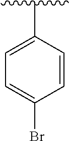 [SMILES: *c1ccc(Br)cc1] | OH | F | H | CH₃ | H | CH₃ | O | O |
| 238 | 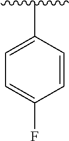 [SMILES: *c1ccc(F)cc1] | H | Cl | H | CH₃ | H | CH₃ | O | O |
| 239 | 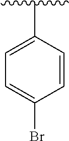 [SMILES: *c1ccc(Br)cc1] | H | Cl | H | CH₃ | H | CH₃ | O | O |
| 240 | 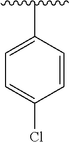 [SMILES: *c1ccc(Cl)cc1] | H | Cl | H | CH₃ | H | CH₃ | O | O |
| 241 | 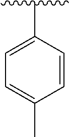 [SMILES: *c1ccc(C)cc1] | H | Cl | H | CH₃ | H | CH₃ | O | O |
| 242 | 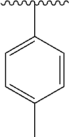 [SMILES: *c1ccc(C)cc1] | OH | F | H | CH₃ | H | CH₃ | O | O |
| 243 | 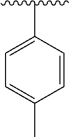 [SMILES: *c1ccc(C)cc1] | H | 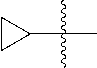 [SMILES: *C1CC1] | H | CH₃ | H | CH₃ | O | O |
| 244 | 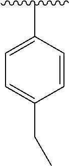 [SMILES: *c1ccc(CC)cc1] | H | Cl | H | CH₃ | H | CH₃ | O | O |
| 245 | 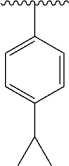 [SMILES: *c1ccc(C(C)C)cc1] | H | Cl | H | CH₃ | H | CH₃ | O | O |
| 246 | 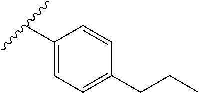 [SMILES: *c1ccc(CCC)cc1] | H | Cl | H | CH₃ | H | CH₃ | O | O |
| 247 | 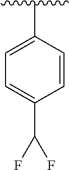 [SMILES: *c1ccc(C(F)F)cc1] | H | Cl | H | CH₃ | H | CH₃ | O | O |
| 248 | 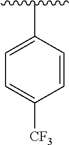 [SMILES: *c1ccc(C(F)(F)F)cc1] | CF₃ | H | H | CH₃ | H | CH₃ | O | O |
| 249 | 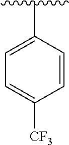 [SMILES: *c1ccc(C(F)(F)F)cc1] | H | Cl | H | CH₃ | H | CH₃ | O | O |
| 250 | 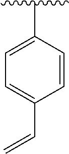 [SMILES: *c1ccc(C=C)cc1] | H | Cl | H | CH₃ | H | CH₃ | O | O |
| 251 | 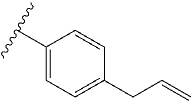 [SMILES: *c1ccc(CC=C)cc1] | H | Cl | H | CH₃ | H | CH₃ | O | O |
| 252 | 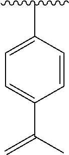 [SMILES: *c1ccc(C(=C)C)cc1] | H | Cl | H | CH₃ | H | CH₃ | O | O |
| 253 | 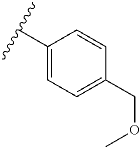 [SMILES: *c1ccc(COC)cc1] | H | Cl | H | CH₃ | H | CH₃ | O | O |
| 254 | 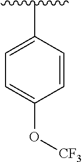 [SMILES: *c1ccc(OC(F)(F)F)cc1] | H | Cl | H | CH₃ | H | CH₃ | O | O |
| 255 | 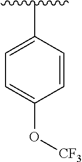 [SMILES: *c1ccc(OC(F)(F)F)cc1] | OH | F | H | CH₃ | H | CH₃ | O | O |
| 256 | 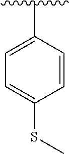 [SMILES: *c1ccc(SC)cc1] | H | Cl | H | CH₃ | H | CH₃ | O | O |
| 257 | 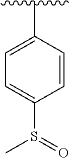 [SMILES: *c1ccc(S(C)=O)cc1] | H | Cl | H | CH₃ | H | CH₃ | O | O |
| 258 | 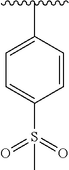 [SMILES: *c1ccc(S(C)(=O)=O)cc1] | H | Cl | H | CH₃ | H | CH₃ | O | O |
| 259 | 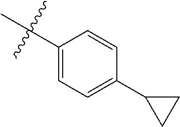 [SMILES: *c1ccc(C2CC2)cc1] | H | Cl | H | CH₃ | H | CH₃ | O | O |
| 260 | 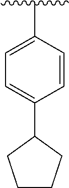 [SMILES: *c1ccc(C2CCCC2)cc1] | H | Cl | H | CH₃ | H | CH₃ | O | O |
| 261 | 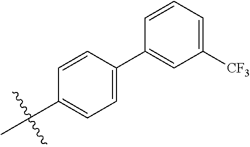 [SMILES: *c1ccc(-c2cccc(C(F)(F)F)c2)cc1] | H | Cl | H | CH₃ | H | CH₃ | O | O |
| 262 | 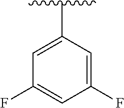 [SMILES: *c1cc(F)cc(F)c1] | H | F | H | CH₃ | H | CH₃ | O | O |
| 263 | 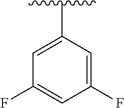 [SMILES: *c1cc(F)cc(F)c1] | OH | F | H | CH₃ | H | CH₃ | O | O |
| 264 | 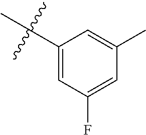 [SMILES: *c1cc(C)cc(F)c1] | H | Cl | H | CH₃ | H | CH₃ | O | O |
| 265 | 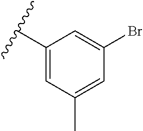 [SMILES: *c1cc(C)cc(Br)c1] | H | Cl | H | CH₃ | H | CH₃ | O | O |
| 266 | 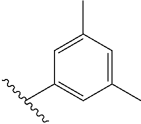 [SMILES: *c1cc(C)cc(C)c1] | H | 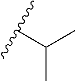 [SMILES: *C(C)C] | H | CH₃ | H | CH₃ | O | O |
| 267 | 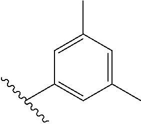 [SMILES: *c1cc(C)cc(C)c1] | H | Cl | H | CH₃ | H | 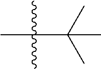 [SMILES: *C(C)(C)C] | O | O |
| 268 | 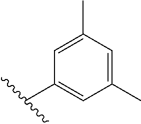 [SMILES: *c1cc(C)cc(C)c1] | H | Cl | H | CH₃ | H | CH₂CF₃ | O | O |
| 269 | 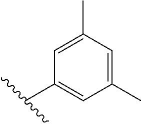 [SMILES: *c1cc(C)cc(C)c1] | H | Cl | H | CH₃ | H | CHF₂ | O | O |
| 270 | 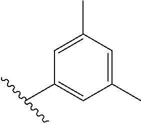 [SMILES: *c1cc(C)cc(C)c1] | H | Cl | H | CH₃ | H | 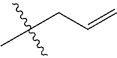 [SMILES: *CC=C] | O | O |
| 271 | 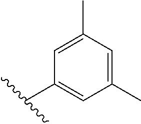 [SMILES: *c1cc(C)cc(C)c1] | H | F | H | CH₃ | H | CH₃ | O | O |
| 272 | 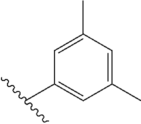 [SMILES: *c1cc(C)cc(C)c1] | H | CH₃ | H | CH₃ | H | CH₃ | O | O |
| 273 | 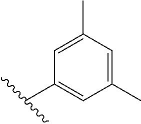 [SMILES: *c1cc(C)cc(C)c1] | H | CH₂CH₃ | H | CH₃ | H | CH₃ | O | O |
| 274 | 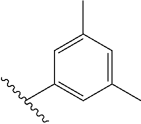 [SMILES: *c1cc(C)cc(C)c1] | H | H | H | CH₃ | H | CH₃ | O | O |
| 275 | 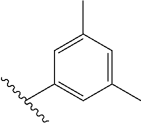 [SMILES: *c1cc(C)cc(C)c1] | H | 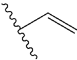 [SMILES: *C=C] | H | CH₃ | H | CH₃ | O | O |
| 276 | 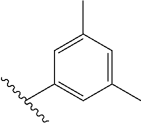 [SMILES: *c1cc(C)cc(C)c1] | H | 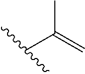 [SMILES: *C(=C)C] | H | CH₃ | H | CH₃ | O | O |
| 277 | 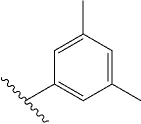 [SMILES: *c1cc(C)cc(C)c1] | H | 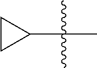 [SMILES: *C1CC1] | H | CH₃ | H | CH₃ | O | O |
| 278 | 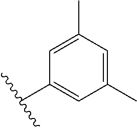 [SMILES: *c1cc(C)cc(C)c1] | H | 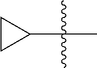 [SMILES: *C1CC1] | H | CH₃ | H | 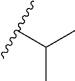 [SMILES: *C(C)C] | O | O |
| 279 | 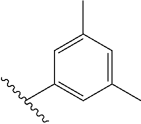 [SMILES: *c1cc(C)cc(C)c1] | H | 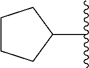 [SMILES: *C1CCCC1] | H | CH₃ | H | CH₃ | O | O |
| 280 | 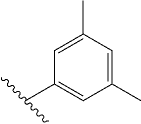 [SMILES: *c1cc(C)cc(C)c1] | H | 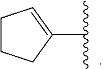 [SMILES: *C1=CCCC1] | H | CH₃ | H | CH₃ | O | O |
| 281 | 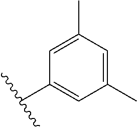 [SMILES: *c1cc(C)cc(C)c1] | H | 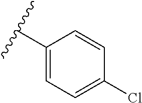 [SMILES: *c1ccc(Cl)cc1] | H | CH₃ | H | CH₃ | O | O |
| 282 | 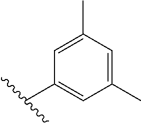 [SMILES: *c1cc(C)cc(C)c1] | CH₃ | Cl | H | CH₃ | H | CH₃ | O | O |
| 283 | 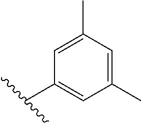 [SMILES: *c1cc(C)cc(C)c1] | CF₃ | H | H | CH₃ | H | CH₃ | O | O |
| 284 | 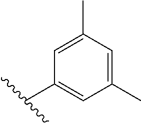 [SMILES: *c1cc(C)cc(C)c1] | Br | H | H | CH₃ | H | CH₃ | O | O |
| 285 | 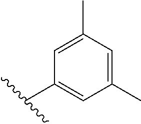 [SMILES: *c1cc(C)cc(C)c1] | OH | H | H | CH₃ | H | CH₃ | O | O |
| 286 | 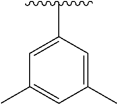 | OH | F | H | CH₃ | H | CH₃ | O | O |
| 287 | 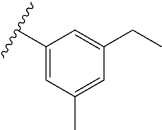 [SMILES: *c1cc(C)cc(CC)c1] | H | Cl | H | CH₃ | H | CH₃ | O | O |
| 288 | 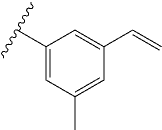 [SMILES: *c1cc(C)cc(C=C)c1] | H | Cl | H | CH₃ | H | CH₃ | O | O |
| 289 | 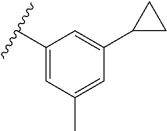 [SMILES: *c1cc(C)cc(C2CC2)c1] | H | Cl | H | CH₃ | H | CH₃ | O | O |
| 290 | 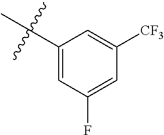 [SMILES: *c1cc(F)cc(C(F)(F)F)c1] | H | Cl | H | CH₃ | H | 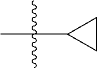 [SMILES: *C1CC1] | O | O |
| 291 | 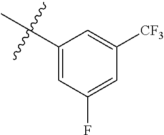 [SMILES: *c1cc(F)cc(C(F)(F)F)c1] | H | Cl | H | CH₃ | H | 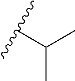 [SMILES: *C(C)C] | O | O |
| 292 | 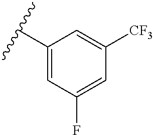 [SMILES: *c1cc(F)cc(C(F)(F)F)c1] | H | Cl | H | CH₃ | H | CHF₂ | O | O |
| 293 | 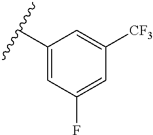 [SMILES: *c1cc(F)cc(C(F)(F)F)c1] | H | 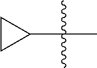 [SMILES: *C1CC1] | H | CH₃ | H | CH₃ | O | O |
| 294 | 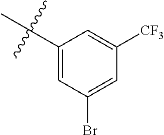 [SMILES: *c1cc(Br)cc(C(F)(F)F)c1] | H | Cl | H | CH₃ | H | CH₃ | O | O |
| 295 | 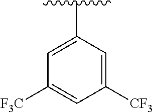 [SMILES: *c1cc(C(F)(F)F)cc(C(F)(F)F)c1] | H | Cl | H | CH₃ | H | CH₃ | O | O |
| 296 | 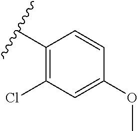 [SMILES: *c1ccc(OC)cc1Cl] | H | Cl | H | CH₃ | H | CH₃ | O | O |
| 297 | 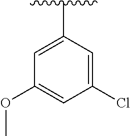 [SMILES: *c1cc(Cl)cc(OC)c1] | H | Cl | H | CH₃ | H | CH₃ | O | O |
| 298 | 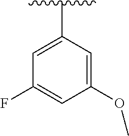 [SMILES: *c1cc(F)cc(OC)c1] | H | Cl | H | CH₃ | H | CH₃ | O | O |
| 299 | 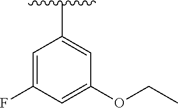 [SMILES: *c1cc(F)cc(OCC)c1] | H | Cl | H | CH₃ | H | CH₃ | O | O |
| 300 | 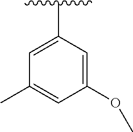 | H | Cl | H | CH₃ | H | CH₃ | O | O |
| 301 | 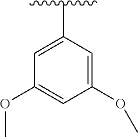 [SMILES: *c1cc(OC)cc(OC)c1] | H | 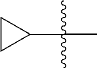 [SMILES: *C1CC1] | H | CH₃ | H | CH₃ | O | O |
| 302 | 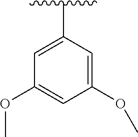 [SMILES: *c1cc(OC)cc(OC)c1] | H | Cl | H | CH₃ | H | CH₃ | O | O |
| 303 | 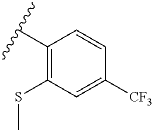 [SMILES: *c1ccc(C(F)(F)F)cc1SC] | H | Cl | H | CH₃ | H | CH₃ | O | O |
| 304 | 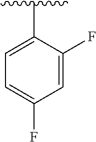 [SMILES: *c1ccc(F)cc1F] | H | Cl | H | CH₃ | H | CH₃ | O | O |
| 305 | 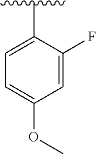 [SMILES: *c1ccc(OC)cc1F] | H | Cl | H | CH₃ | H | CH₃ | O | O |
| 306 | 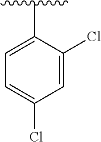 [SMILES: *c1ccc(Cl)cc1Cl] | H | Cl | H | CH₃ | H | CH₃ | O | O |
| 307 | 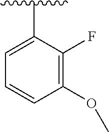 [SMILES: *c1cccc(OC)c1F] | H | Cl | H | CH₃ | H | CH₃ | O | O |
| 308 | 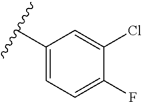 [SMILES: *c1ccc(F)c(Cl)c1] | H | 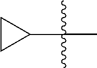 [SMILES: *C1CC1] | H | CH₃ | H | CH₃ | O | O |
| 309 | 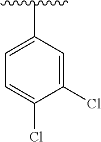 [SMILES: *c1ccc(Cl)c(Cl)c1] | H | Cl | H | CH₃ | H | CH₃ | O | O |
| 310 | 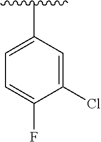 [SMILES: *c1ccc(F)c(Cl)c1] | H | Cl | H | CH₃ | H | CH₃ | O | O |
| 311 | 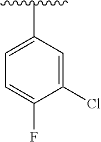 [SMILES: *c1ccc(F)c(Cl)c1] | OH | F | H | CH₃ | H | CH₃ | O | O |
| 312 | 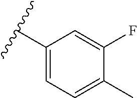 [SMILES: *c1ccc(C)c(F)c1] | H | 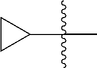 [SMILES: *C1CC1] | H | CH₃ | H | CH₃ | O | O |
| 313 | 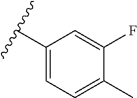 [SMILES: *c1ccc(C)c(F)c1] | H | Cl | H | CH₃ | H | CH₃ | O | O |
| 314 | 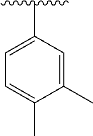 [SMILES: *c1ccc(C)c(C)c1] | H | Cl | H | CH₃ | H | CH₃ | O | O |
| 315 | 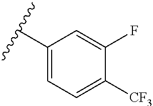 [SMILES: *c1ccc(C(F)(F)F)c(F)c1] | H | Cl | H | CH₃ | H | CH₃ | O | O |
| 316 | 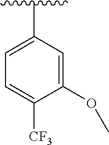 [SMILES: *c1ccc(C(F)(F)F)c(OC)c1] | H | Cl | H | CH₃ | H | CH₃ | O | O |
| 317 | 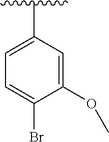 [SMILES: *c1ccc(Br)c(OC)c1] | H | Cl | H | CH₃ | H | CH₃ | O | O |
| 318 | 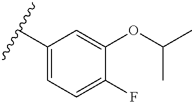 [SMILES: *c1ccc(F)c(OC(C)C)c1] | H | Cl | H | CH₃ | H | CH₃ | O | O |
| 319 | 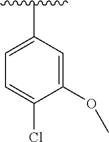 [SMILES: *c1ccc(Cl)c(OC)c1] | H | Cl | H | CH₃ | H | CH₃ | O | O |
| 320 | 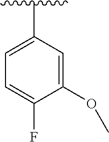 [SMILES: *c1ccc(F)c(OC)c1] | H | Cl | H | CH₃ | H | CH₃ | O | O |
| 321 | 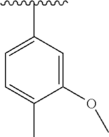 [SMILES: *c1ccc(C)c(OC)c1] | H | Cl | H | CH₃ | H | CH₃ | O | O |
| 322 | 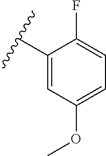 [SMILES: *c1cc(OC)ccc1F] | H | Cl | H | CH₃ | H | CH₃ | O | O |
| 323 | 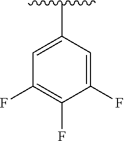 [SMILES: *c1cc(F)c(F)c(F)c1] | H | F | H | CH₃ | H | CH₃ | O | O |
| 324 | 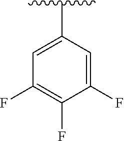 [SMILES: *c1cc(F)c(F)c(F)c1] | OH | F | H | CH₃ | H | CH₃ | O | O |
| 325 | 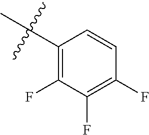 [SMILES: *c1ccc(F)c(F)c1F] | H | Cl | H | CH₃ | H | CH₃ | O | O |
| 326 | 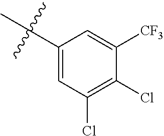 [SMILES: *c1cc(Cl)c(Cl)c(C(F)(F)F)c1] | H | Cl | H | CH₃ | H | CH₃ | O | O |
| 327 | 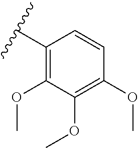 [SMILES: *c1ccc(OC)c(OC)c1OC] | H | Cl | H | CH₃ | H | CH₃ | O | O |
| 328 | 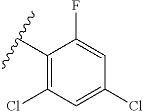 [SMILES: *c1c(F)cc(Cl)cc1Cl] | H | Cl | H | CH₃ | H | CH₃ | O | O |
| 329 | 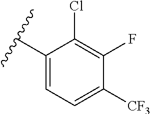 [SMILES: *c1ccc(C(F)(F)F)c(F)c1Cl] | H | Cl | H | CH₃ | H | CH₃ | O | O |
| 330 | 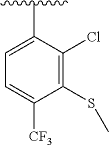 [SMILES: *c1ccc(C(F)(F)F)c(SC)c1Cl] | H | Cl | H | CH₃ | H | CH₃ | O | O |
| 331 | 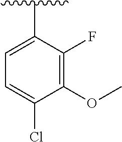 [SMILES: *c1ccc(Cl)c(OC)c1F] | OH | F | H | CH₃ | H | CH₃ | O | O |
| 332 | 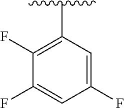 [SMILES: *c1cc(F)cc(F)c1F] | H | Cl | H | CH₃ | H | CH₃ | O | O |
| 333 | 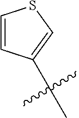 [SMILES: *c1ccsc1] | H | Cl | H | CH₃ | H | CH₃ | O | O |
| 334 | 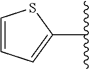 [SMILES: *c1cccs1] | H | Cl | H | CH₃ | H | CH₃ | O | O |
| 335 | 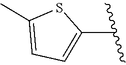 [SMILES: *c1ccc(C)s1] | H | Cl | H | CH₃ | H | CH₃ | O | O |
| 336 | 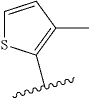 [SMILES: *c1sccc1C] | H | Cl | H | CH₃ | H | CH₃ | O | O |
| 337 | 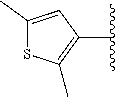 [SMILES: *c1cc(C)sc1C] | H | Cl | H | CH₃ | H | CH₃ | O | O |
| 338 | 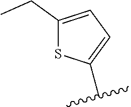 [SMILES: *c1ccc(CC)s1] | H | Cl | H | CH₃ | H | CH₃ | O | O |
| 339 | 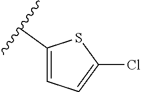 [SMILES: *c1ccc(Cl)s1] | H | Cl | H | CH₃ | H | CH₃ | O | O |
| 340 | 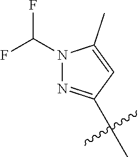 [SMILES: *c1cc(C)n(C(F)F)n1] | H | Cl | H | CH₃ | H | CH₃ | O | O |
| 341 | 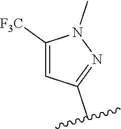 [SMILES: *c1cc(C(F)(F)F)n(C)n1] | H | Cl | H | CH₃ | H | CH₃ | O | O |
| 342 | 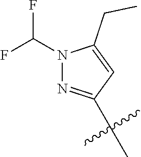 [SMILES: *c1cc(CC)n(C(F)F)n1] | H | Cl | H | CH₃ | H | CH₃ | O | O |
| 343 | 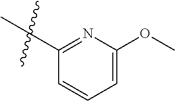 [SMILES: *c1cccc(OC)n1] | H | Cl | H | CH₃ | H | CH₃ | O | O |
| 344 | 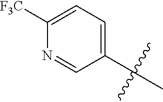 [SMILES: *c1ccc(C(F)(F)F)nc1] | H | Cl | H | CH₃ | H | CH₃ | O | O |
| 345 | 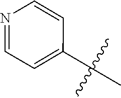 [SMILES: *c1ccncc1] | H | Cl | H | CH₃ | H | CH₃ | O | O |
| 346 | 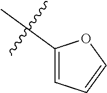 [SMILES: *c1ccco1] | H | Cl | H | CH₃ | H | CH₃ | O | O |
| 347 | 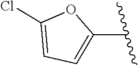 [SMILES: *c1ccc(Cl)o1] | H | Cl | H | CH₃ | H | CH₃ | O | O |
| 348 | 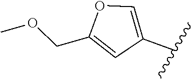 [SMILES: *c1coc(COC)c1] | H | Cl | H | CH₃ | H | CH₃ | O | O |
| 349 | 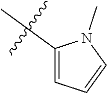 [SMILES: *c1cccn1C] | H | Cl | H | CH₃ | H | CH₃ | O | O |
| 350 | 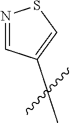 [SMILES: *c1cnsc1] | H | Cl | H | CH₃ | H | CH₃ | O | O |
| 351 | 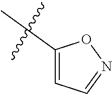 [SMILES: *c1ccno1] | H | Cl | H | CH₃ | H | CH₃ | O | O |
| 352 | 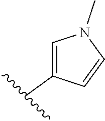 [SMILES: *c1ccn(C)c1] | H | Cl | H | CH₃ | H | CH₃ | O | O |
| 353 | 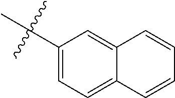 [SMILES: *c1ccc2ccccc2c1] | H | Cl | H | CH₃ | H | CH₃ | O | O |
| 354 | 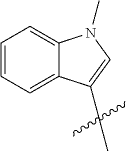 [SMILES: *c1cn(C)c2ccccc12] | H | Cl | H | CH₃ | H | CH₃ | O | O |
| 355 | 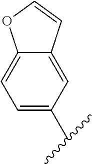 [SMILES: *c1ccc2occc2c1] | H | Cl | H | CH₃ | H | CH₃ | O | O |
| 356 | 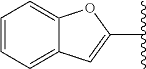 [SMILES: *c1cc2ccccc2o1] | H | Cl | H | CH₃ | H | CH₃ | O | O |
| 357 | 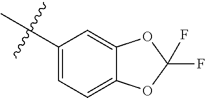 [SMILES: *c1ccc2c(c1)OC(F)(F)O2] | H | Cl | H | CH₃ | H | CH₃ | O | O |
| 358 | 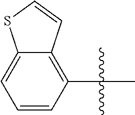 [SMILES: *c1cccc2sccc12] | H | Cl | H | CH₃ | H | CH₃ | O | O |
| 359 | 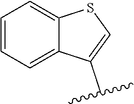 [SMILES: *c1csc2ccccc12] | H | Cl | H | CH₃ | H | CH₃ | O | O |
| 360 | 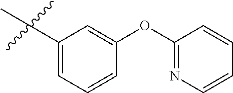 [SMILES: *c1cccc(Oc2ccccn2)c1] | H | Cl | H | CH₃ | H | CH₃ | O | O |
| 361 | 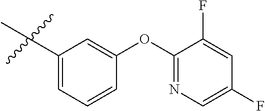 [SMILES: *c1cccc(Oc2ncc(F)cc2F)c1] | H | Cl | H | CH₃ | H | CH₃ | O | O |
| 362 | 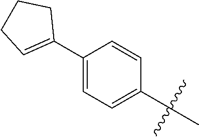 [SMILES: *c1ccc(C2=CCCC2)cc1] | H | Cl | H | CH₃ | H | CH₃ | O | O |
| 363 | 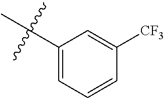 [SMILES: *c1cccc(C(F)(F)F)c1] | CH₃ | Cl | H | CH₃ | H | CH₂CH₃ | O | O |
| 364 | 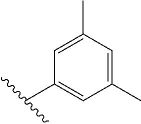 [SMILES: *c1cc(C)cc(C)c1] | H | Cl | H | CH₃ | CH₃ | CH₂CH₃ | O | O |
| 365 | 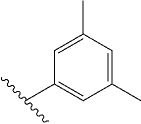 [SMILES: *c1cc(C)cc(C)c1] | H | Cl | H | CH₃ | 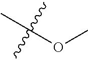 [SMILES: *OC] | CH₃ | O | O |
| 366 | 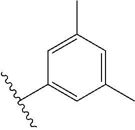 [SMILES: *c1cc(C)cc(C)c1] | H | Cl | H | CH₃ | 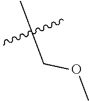 [SMILES: *COC] | CH₃ | O | O |
| 367 | 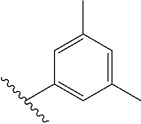 [SMILES: *c1cc(C)cc(C)c1] | H | Cl | H | CH₃ | 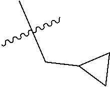 [SMILES: *CC1CC1] | CH₃ | O | O |
| 368 | 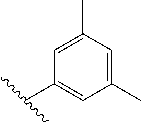 [SMILES: *c1cc(C)cc(C)c1] | H | Cl | H | CH₃ | 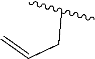 [SMILES: *CC=C] | CH₃ | O | O |
| 369 | 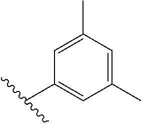 [SMILES: *c1cc(C)cc(C)c1] | H | Cl | H | CH₃ | 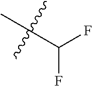 [SMILES: *C(F)F] | CH₃ | O | O |
| 370 | 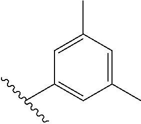 [SMILES: *c1cc(C)cc(C)c1] | H | Cl | H | H | H | CH₃ | S | O |
| 371 | 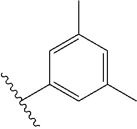 [SMILES: *c1cc(C)cc(C)c1] | H | Cl | H | 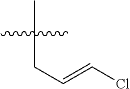 [SMILES: *C/C=C/Cl] | H | CH₃ | O | O |
| 372 | 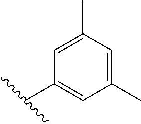 [SMILES: *c1cc(C)cc(C)c1] | H | Cl | H | 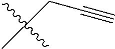 [SMILES: *CC#C] | H | CH₃ | O | O |
| 373 | 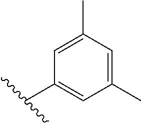 [SMILES: *c1cc(C)cc(C)c1] | H | Cl | H | 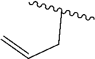 [SMILES: *CC=C] | H | CH₃ | O | O |
| 374 | 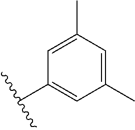 [SMILES: *c1cc(C)cc(C)c1] | H | Cl | H | 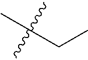 [SMILES: *CC] | H | CH₃ | O | O |
| 375 | 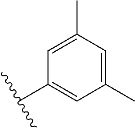 [SMILES: *c1cc(C)cc(C)c1] | H | Cl | H | 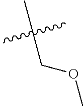 [SMILES: *COC] | H | CH₃ | O | O |
| 376 | 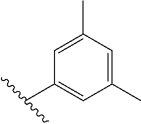 [SMILES: *c1cc(C)cc(C)c1] | H | Cl | H | 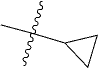 [SMILES: *C1CC1] | H | CH₃ | O | O |
| 377 | 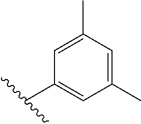 [SMILES: *c1cc(C)cc(C)c1] | H | Cl | H | 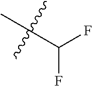 [SMILES: *C(F)F] | H | CH₃ | O | O |
| 378 | 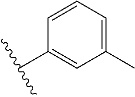 [SMILES: *c1cccc(C)c1] | H | 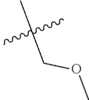 [SMILES: *COC] | H | CH₃ | H | CH₃ | O | O |
| 379 | 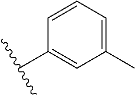 [SMILES: *c1cccc(C)c1] | H | 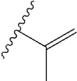 [SMILES: *C(=C)C] | H | CH₃ | H | CH₃ | O | O |
| 380 | 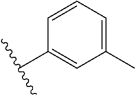 [SMILES: *c1cccc(C)c1] | H | 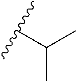 [SMILES: *C(C)C] | H | CH₃ | H | CH₃ | O | O |
| 381 | 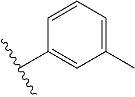 [SMILES: *c1cccc(C)c1] | H | Cl | H | CH₃ | H | 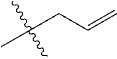 [SMILES: *CC=C] | O | O |
| 382 | 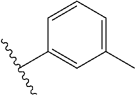 [SMILES: *c1cccc(C)c1] | H | Cl | H | CH₃ | H | 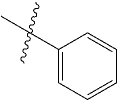 [SMILES: *c1ccccc1] | O | O |
| 383 | 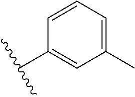 [SMILES: *c1cccc(C)c1] | H | Cl | H | CH₃ | H | CH₃ | S | S |
| 384 | 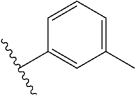 [SMILES: *c1cccc(C)c1] | H | Cl | H | CH₃ | H | 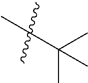 [SMILES: *C(C)(C)C] | O | O |
| 385 | 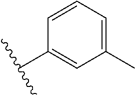 [SMILES: *c1cccc(C)c1] | H | Cl | H | CH₃ | H | CH₂CH₃ | O | O |
| 386 | 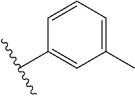 [SMILES: *c1cccc(C)c1] | H | Cl | H | CH₃ | H | 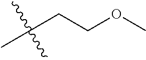 [SMILES: *CCOC] | O | O |
| 387 | 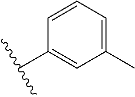 [SMILES: *c1cccc(C)c1] | H | Cl | H | CH₃ | H | 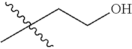 [SMILES: *CCO] | O | O |
| 388 | 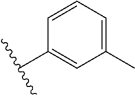 [SMILES: *c1cccc(C)c1] | H | F | H | CH₃ | H | CH₃ | O | O |
| 389 | 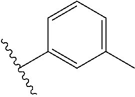 [SMILES: *c1cccc(C)c1] | H | Br | H | CH₃ | H | CH₃ | O | O |
| 390 | 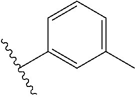 [SMILES: *c1cccc(C)c1] | H | CF₂CF₃ | H | CH₃ | H | CH₃ | O | O |
| 391 | 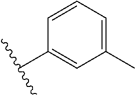 [SMILES: *c1cccc(C)c1] | H | 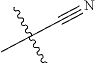 [SMILES: *C#N] | H | CH₃ | H | CH₃ | O | O |
| 392 | 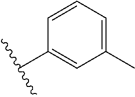 [SMILES: *c1cccc(C)c1] | H | 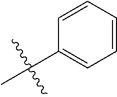 [SMILES: *c1ccccc1] | H | CH₃ | H | CH₃ | O | O |
| 393 | 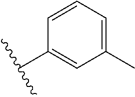 [SMILES: *c1cccc(C)c1] | H | 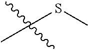 [SMILES: *SC] | H | CH₃ | H | CH₃ | O | O |
| 394 | 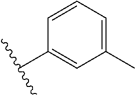 [SMILES: *c1cccc(C)c1] | H | 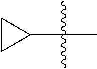 [SMILES: *C1CC1] | H | CH₃ | H | CH₂CH₃ | O | O |
| 395 | 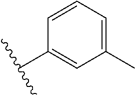 [SMILES: *c1cccc(C)c1] | H | 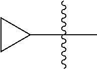 [SMILES: *C1CC1] | H | CH₃ | H | 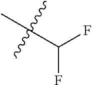 [SMILES: *C(F)F] | O | O |
| 396 | 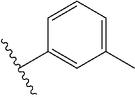 [SMILES: *c1cccc(C)c1] | H | 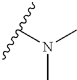 [SMILES: *N(C)C] | H | CH₃ | H | CH₃ | O | O |
| 397 | 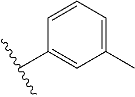 [SMILES: *c1cccc(C)c1] | CH₃ | H | H | CH₃ | H | CH₃ | O | O |
| 398 | 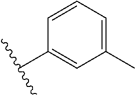 [SMILES: *c1cccc(C)c1] | 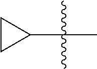 [SMILES: *C1CC1] | H | H | CH₃ | H | CH₃ | O | O |
| 399 | 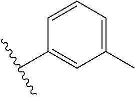 [SMILES: *c1cccc(C)c1] | I | H | H | CH₃ | H | CH₃ | O | O |
| 400 | 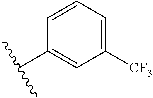 [SMILES: *c1cccc(C(F)(F)F)c1] | H | Cl | H | CH₃ | H | CH₂CH₃ | S | S |
| 401 | 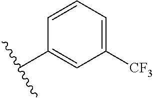 [SMILES: *c1cccc(C(F)(F)F)c1] | H | CH₃ | H | CH₃ | H | CH₃ | S | S |
| 402 | 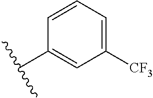 [SMILES: *c1cccc(C(F)(F)F)c1] | H | Cl | H | CH₃ | H | 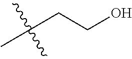 [SMILES: *CCO] | O | O |
| 403 | 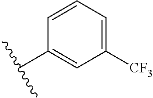 [SMILES: *c1cccc(C(F)(F)F)c1] | H | CH₂CH₃ | H | CH₃ | H | 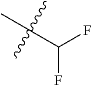 [SMILES: *C(F)F] | O | O |
| 404 | 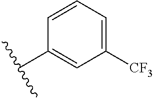 [SMILES: *c1cccc(C(F)(F)F)c1] | H | CF₃ | H | CH₃ | H | CH₃ | O | O |
| 405 | 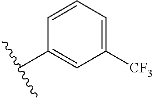 [SMILES: *c1cccc(C(F)(F)F)c1] | H | CF₂CF₃ | H | CH₃ | H | CH₃ | O | O |
| 406 | 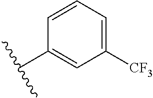 [SMILES: *c1cccc(C(F)(F)F)c1] | H | 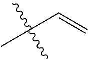 [SMILES: *C=C] | H | CH₃ | H | 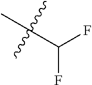 [SMILES: *C(F)F] | O | O |
| 407 | 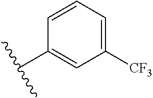 [SMILES: *c1cccc(C(F)(F)F)c1] | H | 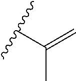 [SMILES: *C(=C)C] | H | CH₃ | H | CH₃ | O | O |
| 408 | 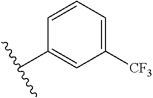 [SMILES: *c1cccc(C(F)(F)F)c1] | H | 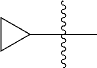 [SMILES: *C1CC1] | H | CH₃ | H | CH₂CH₃ | O | O |
| 409 | 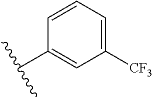 [SMILES: *c1cccc(C(F)(F)F)c1] | H | 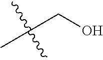 [SMILES: *CO] | H | CH₃ | H | CH₃ | O | O |
| 410 | 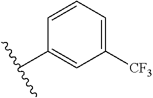 [SMILES: *c1cccc(C(F)(F)F)c1] | H | 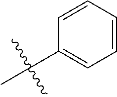 [SMILES: *c1ccccc1] | H | CH₃ | H | CH₃ | O | O |
| 411 | 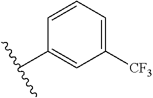 [SMILES: *c1cccc(C(F)(F)F)c1] | H | 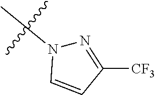 [SMILES: *n1ccc(C(F)(F)F)n1] | H | CH₃ | H | CH₃ | O | O |
| 412 | 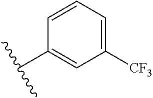 [SMILES: *c1cccc(C(F)(F)F)c1] | CH₃ | H | H | CH₃ | H | CH₃ | O | O |
| 413 | 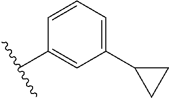 [SMILES: *c1cccc(C2CC2)c1] | H | Cl | H | CH₃ | H | 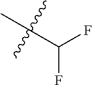 [SMILES: *C(F)F] | O | O |
| 414 | 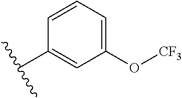 [SMILES: *c1cccc(OC(F)(F)F)c1] | H | Cl | H | CH₃ | H | 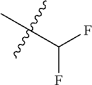 [SMILES: *C(F)F] | O | O |
| 415 | 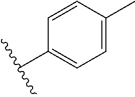 [SMILES: *c1ccc(C)cc1] | H | CH₃ | H | CH₃ | H | CH₃ | O | O |
| 416 | 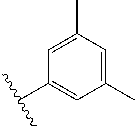 [SMILES: *c1cc(C)cc(C)c1] | H | Cl | H | H | H | CH₃ | O | O |
| 417 | 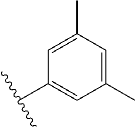 [SMILES: *c1cc(C)cc(C)c1] | H | Cl | H | CH₃ | H | CH₂CH₂CH₃ | O | O |
| 418 | 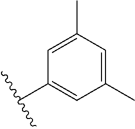 [SMILES: *c1cc(C)cc(C)c1] | H | Cl | H | CH₃ | H | CH₂CH₃ | O | O |
| 419 | 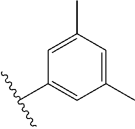 [SMILES: *c1cc(C)cc(C)c1] | H | Cl | H | CH₃ | H | 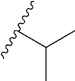 [SMILES: *C(C)C] | O | O |
| 420 | 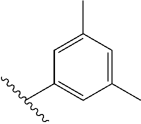 [SMILES: *c1cc(C)cc(C)c1] | H | Cl | H | CH₃ | H | 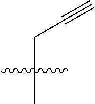 [SMILES: *CC#C] | O | O |
| 421 | 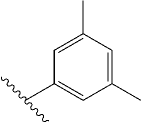 [SMILES: *c1cc(C)cc(C)c1] | H | Cl | H | CH₃ | H | 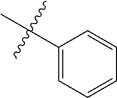 [SMILES: *c1ccccc1] | O | O |
| 422 | 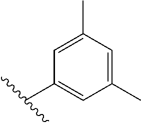 [SMILES: *c1cc(C)cc(C)c1] | H | Cl | H | CH₃ | H | 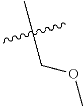 [SMILES: *COC] | O | O |
| 423 | 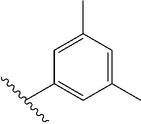 [SMILES: *c1cc(C)cc(C)c1] | H | Cl | H | CH₃ | H | 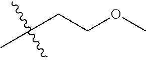 [SMILES: *CCOC] | O | O |
| 424 | 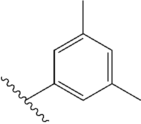 [SMILES: *c1cc(C)cc(C)c1] | H | Cl | H | CH₃ | H | 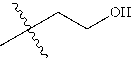 [SMILES: *CCO] | O | O |
| 425 | 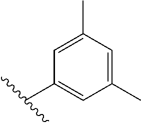 [SMILES: *c1cc(C)cc(C)c1] | H | Br | H | CH₃ | H | CH₃ | O | O |
| 426 | 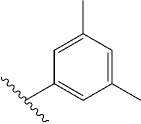 [SMILES: *c1cc(C)cc(C)c1] | H | I | H | CH₃ | H | CH₃ | O | O |
| 427 | 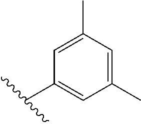 [SMILES: *c1cc(C)cc(C)c1] | H | CF₃ | H | CH₃ | H | CH₃ | O | O |
| 428 | 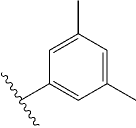 [SMILES: *c1cc(C)cc(C)c1] | H | CF₂CF₃ | H | CH₃ | H | CH₃ | O | O |
| 429 | 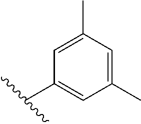 [SMILES: *c1cc(C)cc(C)c1] | H | 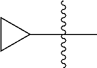 [SMILES: *C1CC1] | H | CH₃ | H | 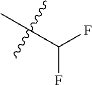 [SMILES: *C(F)F] | O | O |
| 430 | 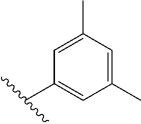 [SMILES: *c1cc(C)cc(C)c1] | H | 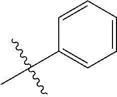 [SMILES: *c1ccccc1] | H | CH₃ | H | CH₃ | O | O |
| 431 | 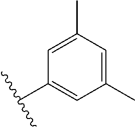 [SMILES: *c1cc(C)cc(C)c1] | H | 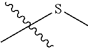 [SMILES: *SC] | H | CH₃ | H | CH₃ | O | O |
| 432 | 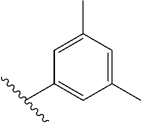 [SMILES: *c1cc(C)cc(C)c1] | H | 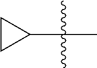 [SMILES: *C1CC1] | H | CH₃ | H | CH₂CH₃ | O | O |
| 433 | 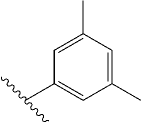 [SMILES: *c1cc(C)cc(C)c1] | H | 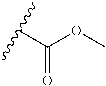 [SMILES: *C(=O)OC] | H | CH₃ | H | CH₃ | O | O |
| 434 | 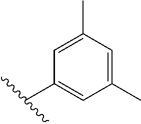 [SMILES: *c1cc(C)cc(C)c1] | H | 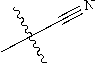 [SMILES: *C#N] | H | CH₃ | H | CH₃ | O | O |
| 435 | 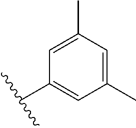 [SMILES: *c1cc(C)cc(C)c1] | CH₃ | H | H | CH₃ | H | CH₃ | O | O |
| 436 | 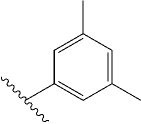 [SMILES: *c1cc(C)cc(C)c1] | Cl | H | H | CH₃ | H | CH₃ | O | O |
| 437 | 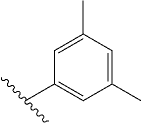 [SMILES: *c1cc(C)cc(C)c1] | 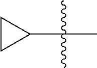 [SMILES: *C1CC1] | H | H | CH₃ | H | CH₃ | O | O |
| 438 | 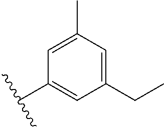 [SMILES: *c1cc(C)cc(CC)c1] | H | Cl | H | CH₃ | H | 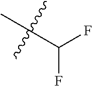 [SMILES: *C(F)F] | O | O |
| 439 | 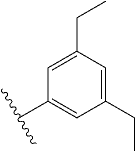 [SMILES: *c1cc(CC)cc(CC)c1] | H | Cl | H | CH₃ | H | CH₃ | O | O |
| 440 | 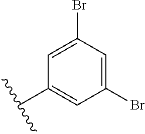 [SMILES: *c1cc(Br)cc(Br)c1] | H | Cl | H | CH₃ | H | CH₃ | O | O |
| 441 | 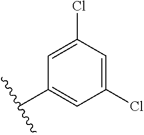 [SMILES: *c1cc(Cl)cc(Cl)c1] | H | Cl | H | CH₃ | H | CH₃ | O | O |
| 442 | 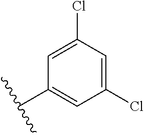 [SMILES: *c1cc(Cl)cc(Cl)c1] | H | Cl | H | CH₃ | H | 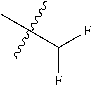 [SMILES: *C(F)F] | O | O |
| 443 | 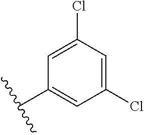 [SMILES: *c1cc(Cl)cc(Cl)c1] | H | 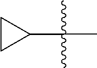 [SMILES: *C1CC1] | H | CH₃ | H | CH₃ | O | O |
| 444 | 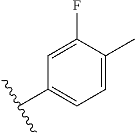 [SMILES: *c1ccc(C)c(F)c1] | H | Cl | H | CH₃ | H | 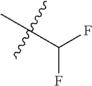 [SMILES: *C(F)F] | O | O |
| 445 | 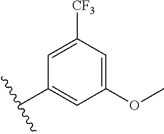 [SMILES: *c1cc(OC)cc(C(F)(F)F)c1] | H | Cl | H | CH₃ | H | 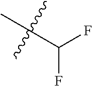 [SMILES: *C(F)F] | O | O |
| 446 | 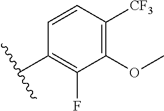 [SMILES: *c1ccc(C(F)(F)F)c(OC)c1F] | H | Cl | H | CH₃ | H | CH₃ | O | O |
| 447 | 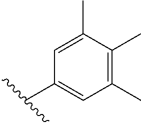 [SMILES: *c1cc(C)c(C)c(C)c1] | H | Cl | H | CH₃ | H | CH₃ | O | O |
| 448 | 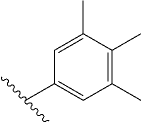 [SMILES: *c1cc(C)c(C)c(C)c1] | H | 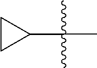 [SMILES: *C1CC1] | H | CH₃ | H | CH₃ | O | O |
| 449 | 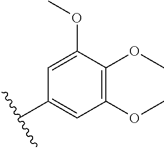 [SMILES: *c1cc(OC)c(OC)c(OC)c1] | H | Cl | H | CH₃ | H | CH₃ | O | O |
| 450 | 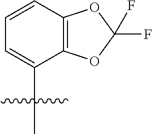 [SMILES: *c1cccc2c1OC(F)(F)O2] | H | Cl | H | CH₃ | H | CH₃ | O | O |
| 451 | 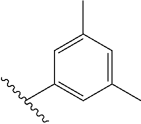 [SMILES: *c1cc(C)cc(C)c1] | H | Cl | H | CH₃ | H | CH₃ | S | S |
| 452 | 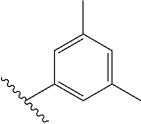 [SMILES: *c1cc(C)cc(C)c1] | H | Cl | H | CH₃ | H | CH₃ | O | S |
| 453 | 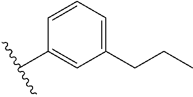 [SMILES: *c1cccc(CCC)c1] | H | Cl | H | CH₃ | H | CH₃ | O | S |
| 454 | 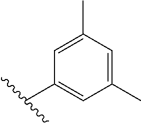 [SMILES: *c1cc(C)cc(C)c1] | H | Cl | CH₃ | CH₃ | H | CH₃ | O | O |
| 455 | 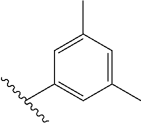 [SMILES: *c1cc(C)cc(C)c1] | H | Cl | 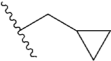 [SMILES: *CC1CC1] | CH₃ | H | CH₃ | O | O |
| 456 | 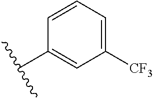 [SMILES: *c1cccc(C(F)(F)F)c1] | H | H | H | CH₃ | H | CH₂CH₃ | O | O |
| 457 | 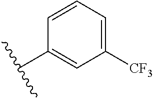 [SMILES: *c1cccc(C(F)(F)F)c1] | CHF₂ | H | H | CH₃ | H | CH₃ | O | O |
| 458 | 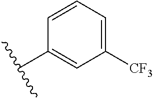 [SMILES: *c1cccc(C(F)(F)F)c1] | CF₃ | H | H | CH₃ | H | CH₂CH₃ | O | O |
| 459 | 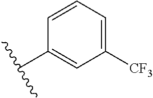 [SMILES: *c1cccc(C(F)(F)F)c1] | CF₃ | H | H | CH₃ | H | 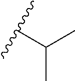 [SMILES: *C(C)C] | O | O |
| 460 | 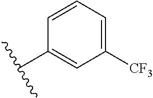 [SMILES: *c1cccc(C(F)(F)F)c1] | CF₃ | H | H | CH₃ | H | 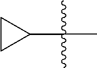 [SMILES: *C1CC1] | O | O |
| 461 | 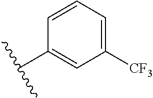 [SMILES: *c1cccc(C(F)(F)F)c1] | CH₃ | CH₃ | H | CH₃ | H | CH₃ | O | O |
| 462 | 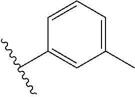 [SMILES: *c1cccc(C)c1] | H | Cl | H | CH₃ | H | CH₂CH₂CH₃ | O | O |
| 463 | 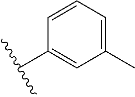 [SMILES: *c1cccc(C)c1] | H | Cl | H | CH₃ | H | Boc | O | O |
| 464 | 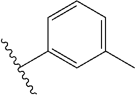 [SMILES: *c1cccc(C)c1] | H | Cl | H | CH₃ | H | 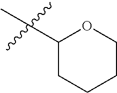 [SMILES: *C1CCCCO1] | O | O |
| 465 | 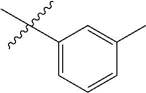 [SMILES: *c1cccc(C)c1] | H | CH₃ | H | CH₃ | H | 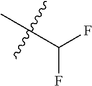 [SMILES: *C(F)F] | O | O |
| 466 | 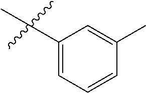 [SMILES: *c1cccc(C)c1] | H | CH₂CH₃ | H | CH₃ | H | 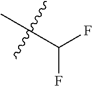 [SMILES: *C(F)F] | O | O |
| 467 | 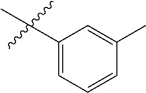 [SMILES: *c1cccc(C)c1] | H | CH₂F | H | CH₃ | H | CH₃ | O | O |
| 468 | 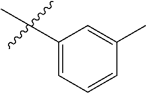 [SMILES: *c1cccc(C)c1] | H | 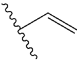 [SMILES: *C=C] | H | CH₃ | H | 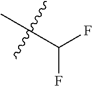 [SMILES: *C(F)F] | O | O |
| 469 | 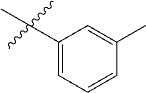 [SMILES: *c1cccc(C)c1] | H | 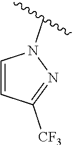 [SMILES: *n1ccc(C(F)(F)F)n1] | H | CH₃ | H | CH₃ | O | O |
| 470 | 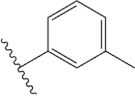 [SMILES: *c1cccc(C)c1] | H | 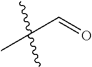 [SMILES: *C=O] | H | CH₃ | H | CH₃ | O | O |
| 471 | 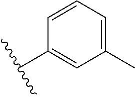 [SMILES: *c1cccc(C)c1] | H | 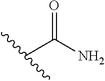 [SMILES: *C(N)=O] | H | CH₃ | H | CH₃ | O | O |
| 472 | 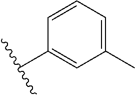 [SMILES: *c1cccc(C)c1] | H | 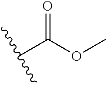 [SMILES: *C(=O)OC] | H | CH₃ | H | CH₃ | O | O |
| 473 | 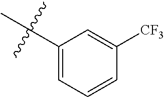 [SMILES: *c1cccc(C(F)(F)F)c1] | H | Cl | H | CH₃ | H | 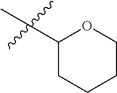 [SMILES: *C1CCCCO1] | O | O |
| 474 | 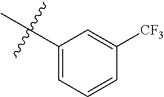 [SMILES: *c1cccc(C(F)(F)F)c1] | H | Cl | H | CH₃ | H | Boc | O | O |
| 475 | 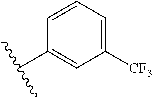 [SMILES: *c1cccc(C(F)(F)F)c1] | H | CH₃ | H | CH₃ | H | 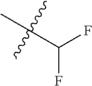 [SMILES: *C(F)F] | O | O |
| 476 | 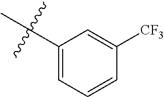 [SMILES: *c1cccc(C(F)(F)F)c1] | H | 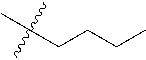 [SMILES: *CCCC] | H | CH₃ | H | CH₃ | O | O |
| 477 | 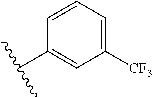 [SMILES: *c1cccc(C(F)(F)F)c1] | H | CH₂F | H | CH₃ | H | CH₃ | O | O |
| 478 | 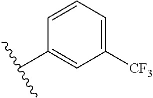 [SMILES: *c1cccc(C(F)(F)F)c1] | H | CHF₂ | H | CH₃ | H | CH₃ | O | O |
| 479 | 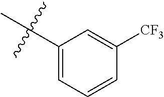 [SMILES: *c1cccc(C(F)(F)F)c1] | H | SCH₃ | H | CH₃ | H | CH₃ | O | O |
| 480 | 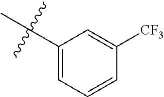 [SMILES: *c1cccc(C(F)(F)F)c1] | H | SO₂CH₃ | H | CH₃ | H | CH₃ | O | O |
| 481 | 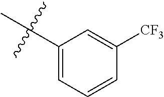 [SMILES: *c1cccc(C(F)(F)F)c1] | H | 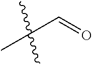 [SMILES: *C=O] | H | CH₃ | H | CH₃ | O | O |
| 482 | 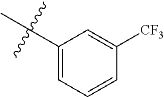 [SMILES: *c1cccc(C(F)(F)F)c1] | H | 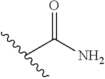 [SMILES: *C(N)=O] | H | CH₃ | H | CH₃ | O | O |
| 483 | 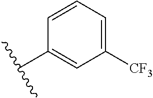 [SMILES: *c1cccc(C(F)(F)F)c1] | H | 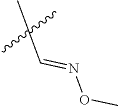 [SMILES: */C=N/OC] | H | CH₃ | H | CH₃ | O | O |
| 484 | 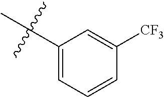 [SMILES: *c1cccc(C(F)(F)F)c1] | Cl | H | H | CH₃ | H | Boc | O | O |
| 485 | 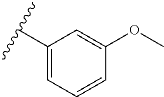 [SMILES: *c1cccc(OC)c1] | H | CF₃ | H | CH₃ | H | CH₃ | O | O |
| 486 | 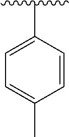 [SMILES: *c1ccc(C)cc1] | H | Cl | H | CH₃ | H | 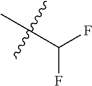 [SMILES: *C(F)F] | O | O |
| 487 | 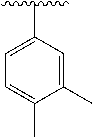 [SMILES: *c1ccc(C)c(C)c1] | H | Cl | H | CH₃ | H | 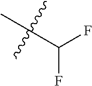 [SMILES: *C(F)F] | O | O |
| 488 | 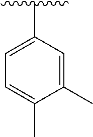 [SMILES: *c1ccc(C)c(C)c1] | H | CF₃ | H | CH₃ | H | CH₃ | O | O |
| 489 | 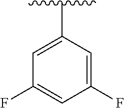 [SMILES: *c1cc(F)cc(F)c1] | H | CF₃ | H | CH₃ | H | CH₃ | O | O |
| 490 | 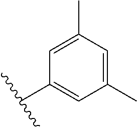 [SMILES: *c1cc(C)cc(C)c1] | H | Cl | H | CH₃ | CH₃ | CH₃ | O | O |
| 491 | 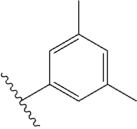 [SMILES: *c1cc(C)cc(C)c1] | H | Cl | H | CH₃ | H | Boc | O | O |
| 492 | 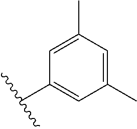 [SMILES: *c1cc(C)cc(C)c1] | H | Cl | H | CH₃ | H | 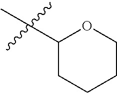 [SMILES: *C1CCCCO1] | O | O |
| 493 | 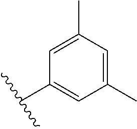 [SMILES: *c1cc(C)cc(C)c1] | H | Cl | H | CH₃ | 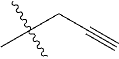 [SMILES: *CC#C] | CH₃ | O | O |
| 494 | 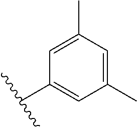 [SMILES: *c1cc(C)cc(C)c1] | H | CH₃ | H | CH₃ | H | 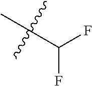 [SMILES: *C(F)F] | O | O |
| 495 | 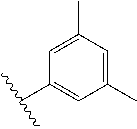 [SMILES: *c1cc(C)cc(C)c1] | H | CH₂F | H | CH₃ | H | CH₃ | O | O |
| 496 | 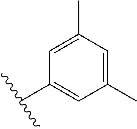 [SMILES: *c1cc(C)cc(C)c1] | H | 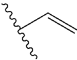 [SMILES: *C=C] | H | CH₃ | H | 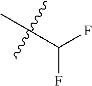 [SMILES: *C(F)F] | O | O |
| 497 | 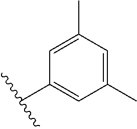 [SMILES: *c1cc(C)cc(C)c1] | H | 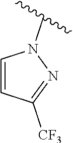 [SMILES: *n1ccc(C(F)(F)F)n1] | H | CH₃ | H | CH₃ | O | O |
| 498 | 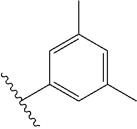 [SMILES: *c1cc(C)cc(C)c1] | H | SO₂Me | H | CH₃ | H | CH₃ | O | O |
| 499 | 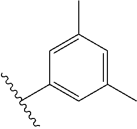 [SMILES: *c1cc(C)cc(C)c1] | H | N(CH₃)₂ | H | CH₃ | H | CH₃ | O | O |
| 500 | 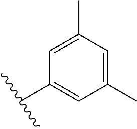 [SMILES: *c1cc(C)cc(C)c1] | H | 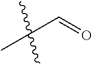 [SMILES: *C=O] | H | CH₃ | H | CH₃ | O | O |
| 501 | 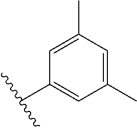 [SMILES: *c1cc(C)cc(C)c1] | H | 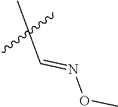 [SMILES: */C=N/OC] | H | CH₃ | H | CH₃ | O | O |
| 502 | 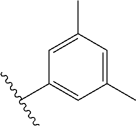 [SMILES: *c1cc(C)cc(C)c1] | H | 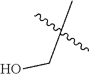 [SMILES: *CO] | H | CH₃ | H | CH₃ | O | O |
| 503 | 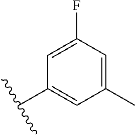 [SMILES: *c1cc(C)cc(F)c1] | H | 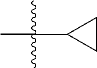 [SMILES: *C1CC1] | H | CH₃ | H | CH₃ | O | O |
| 504 | 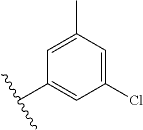 [SMILES: *c1cc(C)cc(Cl)c1] | H | Cl | H | CH₃ | H | CH₃ | O | O |
| 505 | 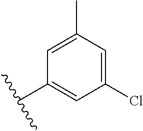 [SMILES: *c1cc(C)cc(Cl)c1] | H | Cl | H | CH₃ | H | 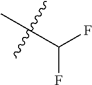 [SMILES: *C(F)F] | O | O |
| 506 | 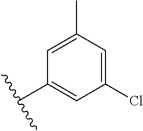 [SMILES: *c1cc(C)cc(Cl)c1] | H | 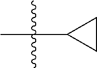 [SMILES: *C1CC1] | H | CH₃ | H | CH₃ | O | O |
| 507 | 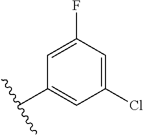 [SMILES: *c1cc(F)cc(Cl)c1] | H | Cl | H | CH₃ | H | CH₃ | O | O |
| 508 | 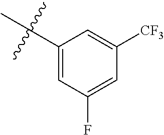 [SMILES: *c1cc(F)cc(C(F)(F)F)c1] | H | CF₃ | H | CH₃ | H | CH₃ | O | O |
| 509 | 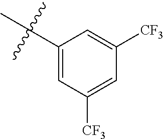 [SMILES: *c1cc(C(F)(F)F)cc(C(F)(F)F)c1] | H | Cl | H | CH₃ | H | 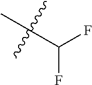 [SMILES: *C(F)F] | O | O |
| 510 | 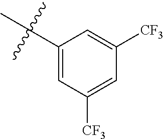 [SMILES: *c1cc(C(F)(F)F)cc(C(F)(F)F)c1] | H | 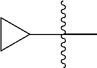 [SMILES: *C1CC1] | H | CH₃ | H | CH₃ | O | O |
| 511 | 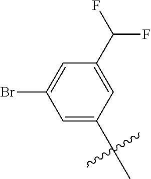 [SMILES: *c1cc(Br)cc(C(F)F)c1] | H | Cl | H | CH₃ | H | CH₃ | O | O |
| 512 | 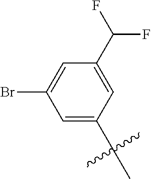 [SMILES: *c1cc(Br)cc(C(F)F)c1] | H | Cl | H | CH₃ | H | 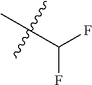 [SMILES: *C(F)F] | O | O |
| 513 | 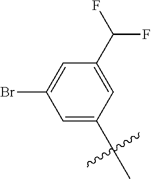 [SMILES: *c1cc(Br)cc(C(F)F)c1] | H | 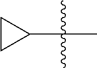 [SMILES: *C1CC1] | H | CH₃ | H | CH₃ | O | O |
| 514 | 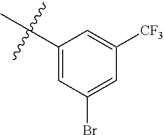 [SMILES: *c1cc(Br)cc(C(F)(F)F)c1] | H | 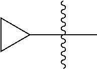 [SMILES: *C1CC1] | H | CH₃ | H | CH₃ | O | O |
| 515 | 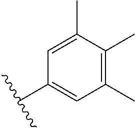 [SMILES: *c1cc(C)c(C)c(C)c1] | H | Cl | H | CH₃ | H | 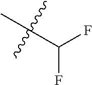 [SMILES: *C(F)F] | O | O |
| 516 | 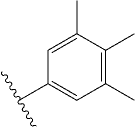 [SMILES: *c1cc(C)c(C)c(C)c1] | H | Br | H | CH₃ | H | CH₃ | O | O |
| 517 | 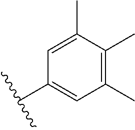 [SMILES: *c1cc(C)c(C)c(C)c1] | H | CF₃ | H | CH₃ | H | CH₃ | O | O |
| 518 | 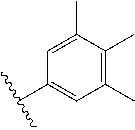 [SMILES: *c1cc(C)c(C)c(C)c1] | H | CN | H | CH₃ | H | CH₃ | O | O |
| 519 | 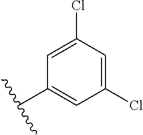 [SMILES: *c1cc(Cl)cc(Cl)c1] | H | Cl | H | CH₃ | H | CH₃ | O | O |
| 520 | 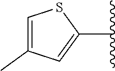 [SMILES: *c1cc(C)cs1] | H | Cl | H | CH₃ | H | CH₃ | O | O |
| 521 | 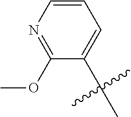 [SMILES: *c1cccnc1OC] | H | Cl | H | CH₃ | H | CH₃ | O | O |
| 522 | 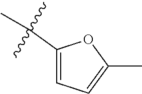 [SMILES: *c1ccc(C)o1] | H | Cl | H | CH₃ | H | CH₃ | O | O |
| 523 | 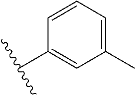 [SMILES: *c1cccc(C)c1] | H | F | H | CH₃ | H | CH₂CH₃ | O | O |
| 524 | 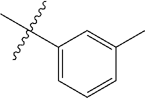 [SMILES: *c1cccc(C)c1] | H | CHF₂ | H | CH₃ | H | CH₃ | O | O |
| 525 | 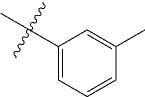 [SMILES: *c1cccc(C)c1] | H | CF₃ | H | CH₃ | H | CH₂CH₃ | O | O |
| 526 | 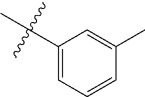 [SMILES: *c1cccc(C)c1] | H | CF₃ | H | CH₃ | H | 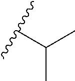 [SMILES: *C(C)C] | O | O |
| 527 | 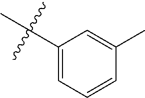 [SMILES: *c1cccc(C)c1] | H | CF₃ | H | CH₃ | H | 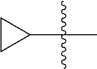 [SMILES: *C1CC1] | O | O |
| 528 | 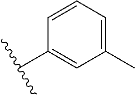 [SMILES: *c1cccc(C)c1] | H | 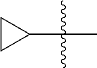 [SMILES: *C1CC1] | H | CH₃ | H | 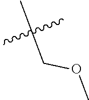 [SMILES: *COC] | O | O |
| 529 | 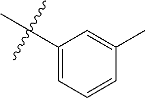 [SMILES: *c1cccc(C)c1] | H | 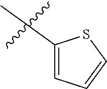 [SMILES: *c1cccs1] | H | CH₃ | H | CH₃ | O | O |
| 530 | 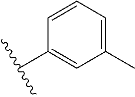 [SMILES: *c1cccc(C)c1] | H | SOCH₃ | H | CH₃ | H | CH₃ | O | O |
| 531 | 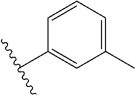 [SMILES: *c1cccc(C)c1] | H | SO₂CH₃ | H | CH₃ | H | CH₃ | O | O |
| 532 | 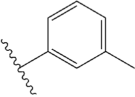 [SMILES: *c1cccc(C)c1] | H | 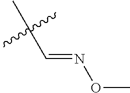 [SMILES: */C=N/OC] | H | CH₃ | H | CH₃ | O | O |
| 533 | 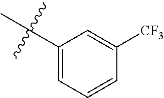 [SMILES: *c1cccc(C(F)(F)F)c1] | H | F | H | CH₃ | H | CH₂CH₃ | O | O |
| 534 | 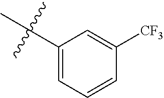 [SMILES: *c1cccc(C(F)(F)F)c1] | H | CH₂CH₃ | H | CH₃ | H | 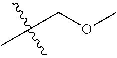 [SMILES: *COC] | O | O |
| 535 | 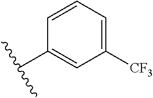 [SMILES: *c1cccc(C(F)(F)F)c1] | H | CF₃ | H | CH₃ | H | CH₂CH₃ | O | O |
| 536 | 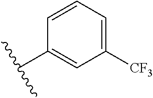 [SMILES: *c1cccc(C(F)(F)F)c1] | H | CF₃ | H | CH₃ | H | 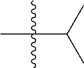 [SMILES: *C(C)C] | O | O |
| 537 | 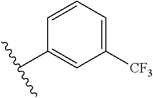 [SMILES: *c1cccc(C(F)(F)F)c1] | H | CF₃ | H | CH₃ | H | 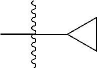 [SMILES: *C1CC1] | O | O |
| 538 | 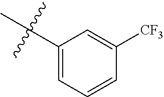 [SMILES: *c1cccc(C(F)(F)F)c1] | H | CF₃ | H | CH₃ | H | CH₃ | O | O |
| 539 | 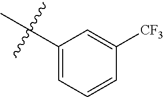 [SMILES: *c1cccc(C(F)(F)F)c1] | H | SOCH₃ | H | CH₃ | H | CH₃ | O | O |
| 540 | 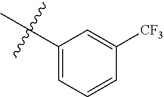 [SMILES: *c1cccc(C(F)(F)F)c1] | H | 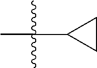 [SMILES: *C1CC1] | H | CH₃ | H | 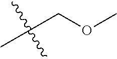 [SMILES: *COC] | O | O |
| 541 | 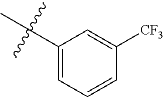 [SMILES: *c1cccc(C(F)(F)F)c1] | H | COOH | H | CH₃ | H | CH₃ | O | O |
| 542 | 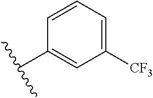 [SMILES: *c1cccc(C(F)(F)F)c1] | H | 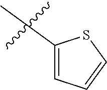 [SMILES: *c1cccs1] | H | CH₃ | H | CH₃ | O | O |
| 543 | 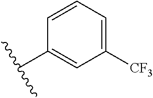 [SMILES: *c1cccc(C(F)(F)F)c1] | H | 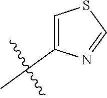 [SMILES: *c1cscn1] | H | CH₃ | H | CH₃ | O | O |
| 544 | 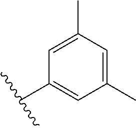 [SMILES: *c1cc(C)cc(C)c1] | H | CH₂CH₃ | H | CH₃ | H | 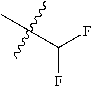 [SMILES: *C(F)F] | O | O |
| 545 | 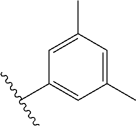 [SMILES: *c1cc(C)cc(C)c1] | H | CH₂CH₃ | H | CH₃ | H | 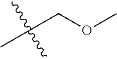 [SMILES: *COC] | O | O |
| 546 | 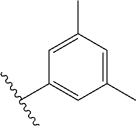 [SMILES: *c1cc(C)cc(C)c1] | H | CHF₂ | H | CH₃ | H | CH₃ | O | O |
| 547 | 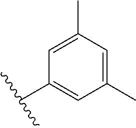 [SMILES: *c1cc(C)cc(C)c1] | H | CF₃ | H | CH₃ | H | 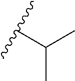 [SMILES: *C(C)C] | O | O |
| 548 | 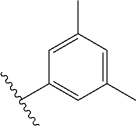 [SMILES: *c1cc(C)cc(C)c1] | H | CF₃ | H | CH₃ | H | 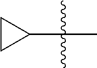 [SMILES: *C1CC1] | O | O |
| 549 | 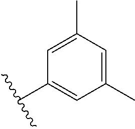 [SMILES: *c1cc(C)cc(C)c1] | H | 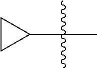 [SMILES: *C1CC1] | H | CH₃ | H | 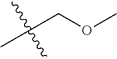 [SMILES: *COC] | O | O |
| 550 | 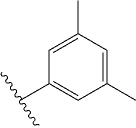 [SMILES: *c1cc(C)cc(C)c1] | H | 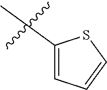 [SMILES: *c1cccs1] | H | CH₃ | H | CH₃ | O | O |
| 551 | 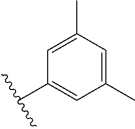 [SMILES: *c1cc(C)cc(C)c1] | H | 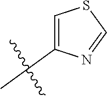 [SMILES: *c1cscn1] | H | CH₃ | H | CH₃ | O | O |
| 552 | 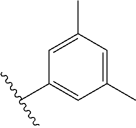 [SMILES: *c1cc(C)cc(C)c1] | H | SOMe | H | CH₃ | H | CH₃ | O | O |
| 553 | 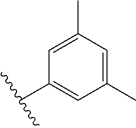 [SMILES: *c1cc(C)cc(C)c1] | H | 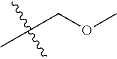 [SMILES: *COC] | H | CH₃ | H | CH₃ | O | O |
| 554 | 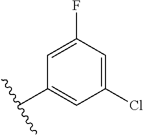 [SMILES: *c1cc(F)cc(Cl)c1] | H | Cl | H | CH₃ | H | 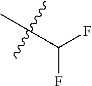 [SMILES: *C(F)F] | O | O |
| 555 | 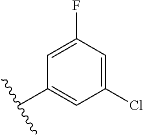 [SMILES: *c1cc(F)cc(Cl)c1] | H | 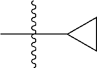 [SMILES: *C1CC1] | H | CH₃ | H | CH₃ | O | O |
| 556 | 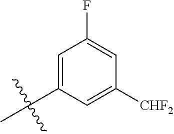 [SMILES: *c1cc(F)cc(C(F)F)c1] | H | Cl | H | CH₃ | H | CH₃ | O | O |
| 557 | 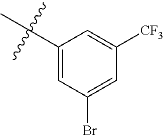 [SMILES: *c1cc(Br)cc(C(F)(F)F)c1] | H | Cl | H | CH₃ | H | 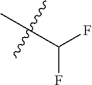 [SMILES: *C(F)F] | O | O |
| 558 | 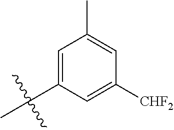 [SMILES: *c1cc(C)cc(C(F)F)c1] | H | Cl | H | CH₃ | H | CH₃ | O | O |
| 559 | 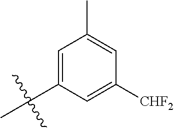 [SMILES: *c1cc(C)cc(C(F)F)c1] | H | Cl | H | CH₃ | H | 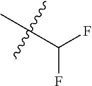 [SMILES: *C(F)F] | O | O |
| 560 | 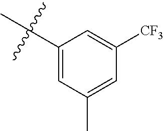 [SMILES: *c1cc(C)cc(C(F)(F)F)c1] | H | Cl | H | CH₃ | H | 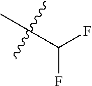 [SMILES: *C(F)F] | O | O |
| 561 | 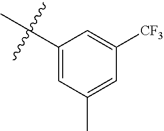 [SMILES: *c1cc(C)cc(C(F)(F)F)c1] | H | 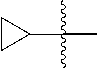 [SMILES: *C1CC1] | H | CH₃ | H | CH₃ | O | O |
| 562 | 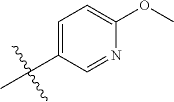 [SMILES: *c1ccc(OC)nc1] | H | Cl | H | CH₃ | H | CH₃ | O | O |

TABLE 2
¹H NMR of Compounds

| No. | ¹H NMR |
|---|---|
| 1 | ¹H NMR (300 MHz, Chloroform-d) δ 9.44 (s, 1H), 7.93 (s, 1H), 7.63-7.40 (m, 4H), 4.21-4.12 (m, 1H), 3.86-3.76 (m, 4H), |
| | 3.60 (d, J = 9.3 Hz, 1H), 3.49-3.41 (m, 1H), 3.00 (s, 3H). |
| 15 | ¹H NMR (300 MHz, Chloroform-d) δ 9.40 (s, 1H), 7.96 (s, 1H), 7.36-7.28 (m, 1H), 7.14 6.93 (m, 3H), 4.14-4.04 (m, 1H), |
| | 3.82-3.75 (m, 4H), 3.57 (d, J = 9.0 Hz, 1H), 3.40-3.37 (m, 1H), 2.99 (s, 3H). |
| 16 | ¹H NMR (300 MHz, Chloroform-d) δ 9.40 (s, 1H), 7.95 (s, 1H), 7.34-7.26 (m, 4H), 4.15-4.01 (m, 1H), 3.80-3.75 (m, 4H), |
| | 3.57 (d, J = 9.0 Hz, 1H), 3.48-3.38 (m, 1H), 2.99 (s, 3H). |
| 17 | ¹H NMR (300 MHz, Chloroform-d) δ 9.38 (s, 1H), 7.96 (s, 1H), 7.25-7.22 (m, 1H), 7.14-7.06 (m, 3H), 4.08-4.00 (m, 1H), |
| | 3.82-3.72 (m, 4H), 3.59 (d, J = 8.7 Hz, 1H), 3.46-3.40 (m, 1H), 2.98 (s, 3H), 2.34 (s, 3H). |
| 20 | ¹H NMR (300 MHz, Chloroform-d) δ 9.35 (s, 1H), 7.97 (s, 1H), 7.32-7.26 (m, 1H), 7.18-7.11 (m, 3H), 4.14-4.03 (m, 1H), |
| | 3.82-3.75 (m, 4H), 3.64-3.57 (m, 1H), 3.48-3.41 (m, 1H), 2.99 (s, 3H), 2.93-2.84 (m, 1H), 1.25 (d, J = 6.9 Hz, 6H). |
| 24 | ¹H NMR (300 MHz, Chloroform-d) δ 9.38 (s, 1H), 7.97 (s, 1H), 7.33-7.26 (m, 1H), 7.22-7.13 (m, 2H), 7.10 (d, J = 9.0 Hz, |
| | 1H), 6.03-5.87 (m, 1H), 5.14-5.05 (m, 2H), 4.09-4.01 (m, 1H), 3.80 (s, 3H), 3.78-3.73 (m, 1H), 3.60 (d, J = 9.0 Hz, 1H), |
| | 3.48-3.42 (m, 1H), 3.41-3.35 (m, 2H), 2.98 (s, 3H). |
| 28 | ¹H NMR (300 MHz, Chloroform-d) δ 9.36 (s, 1H), 7.97 (s, 1H), 7.32-7.26 (m, 1H), 6.96-6.78 (m, 3H), 4.15-4.01 (m, 1H), |
| | 3.83-3.79 (m, 6H), 3.77 (d, J = 9.6 Hz, 1H), 3.60 (d, J = 8.7 Hz, 1H), 3.49-4.01 (m, 1H), 2.98 (s, 3H). |
| 29 | ¹H NMR (300 MHz, Chloroform-d) δ 9.35 (s, 1H), 7.97 (s, 1H), 7.28-7.24 (m, 1H), 6.91-6.75 (m, 3H), 4.59-4.51 (m, 1H), |
| | 4.08-4.00 (m, 1H), 3.82-3.74 (m, 4H), 3.58 (d, J = 8.4 Hz, 1H), 3.50-3.40 (m, 1H), 2.97 (s, 3H), 1.33 (d, J = 6.0 Hz, 6H). |
| 30 | ¹H NMR (300 MHz, Chloroform-d) δ 9.36 (s, 1H), 7.97 (s, 1H), 7.33-7.19 (m, 1H), 7.00-6.77 (m, 3H), 6.13-5.94 (m, 1H), |
| | 5.46-5.38 (m, 1H), 5.34-5.23 (m, 1H), 4.58-4.49 (m, 2H), 4.11-4.02 (m, 1H), 3.85-3.69 (m, 4H), 3.59 (d, J = 8.7 Hz, 1H), |
| | 3.51-3.37 (m, 1H), 2.98 (s, 3H). |
| 33 | ¹H NMR (300 MHz, Chloroform-d) δ 9.41 (s, 1H), 7.95 (s, 1H), 7.45-7.30 (m, 2H), 7.20-7.10 (m, 2H), 4.17-4.08 (m, 1H), |
| | 3.87-3.73 (m, 4H), 3.56 (d, J = 9.0 Hz, 1H), 3.48-3.41 (m, 1H), 3.00 (s, 3H). |
| 39 | ¹H NMR (300 MHz, Chloroform-d) δ 9.43 (s, 1H), 7.98 (s, 1H), 7.28-7.22 (m, 1H), 7.92-6.88 (m, 2H), 6.81-6.77 (m, 1H), |
| | 4.10-4.00 (m, 1H), 3.81-3.75 (m, 6H), 3.63 (d, J = 9.0 Hz, 1H), 3.49-3.41 (m, 1H), 2.97 (s, 3H), 1.30-1.22 (m, 1H), 0.67- |
| | 0.61 (m, 2H), 0.37-0.32 (m, 2H). |
| 41 | ¹H NMR (300 MHz, Chloroform-d) δ 9.34 (s, 1H), 7.95 (s, 1H), 7.23-7.24 (m, 1H), 6.88 (d, J = 7.5 Hz, 1H), 6.83 (s, 1H), |
| | 6.77 (d, J = 8.4 Hz, 1H), 4.78-4.72 (m, 1H), 4.09-4.00 (m, 1H), 3.82-3.73 (m, 4H), 3.58 (d, J = 8.7 Hz, 1H), 3.46-3.40 (m, |
| | 1H), 2.97 (s, 3H), 2.01-1.70 (m, 8H). |
| 50 | ¹H NMR (300 MHz, Chloroform-d) δ 9.38 (s, 1H), 7.97 (s, 1H), 7.37-7.28 (m, 3H), 7.15-7.06 (m, 2H), 7.03-6.98 (m, 3H), |
| | 6.88 (d, J = 7.5 Hz, 1H), 4.12-4.03 (m, 1H), 3.83-3.75 (m, 4H), 3.58 (d, J = 9.6 Hz, 1H), 3.45-3.39 (m, 1H), 2.97 (s, 3H). |
| 52 | ¹H NMR (300 MHz, Chloroform-d) δ 9.33 (s, 1H), 8.02 (d, J = 6.0 Hz, 1H), 7.95 (d, J = 3.0 Hz, 1H), 7.48-7.41 (m, 1H), 7.37 |
| | (d, J = 6.0 Hz, 1H), 7.22-7.16 (m, 1H), 7.11-7.10 (m, 1H), 7.04-7.00 (m, 1H), 6.96-6.90 (m, 1H), 4.16-4.07 (m, 1H), 3.86- |
| | 3.74 (m, 4H), 3.58 (d, J = 9.0 Hz, 1H), 3.51-3.49 (m, 1H), 2.98 (s, 3H). |
| 53 | ¹H NMR (300 MHz, Chloroform-d) δ 9.31 (s, 1H), 8.41 (s, 2H), 7.95 (s, 1H), 7.45-7.40 (m, 1H), 7.27-7.24 (m, 1H), 7.17 (s, |
| | 1H), 7.13-7.03 (m, 1H), 4.19-4.10 (m, 1H), 3.84-3.77 (m, 4H), 3.58 (d, J = 8.4 Hz, 1H), 3.52-3.46 (m, 1H), 2.98 (s, 3H). |
| 54 | ¹H NMR (300 MHz, Chloroform-d) δ 9.48 (s, 1H), 7.93 (s, 1H), 7.59-7.53 (m, 2H), 7.24-7.12 (m, 1H), 4.11-4.06 (m, 1H), |
| | 3.76 (s, 3H), 3.65-3.52 (m, 2H), 3.45-3.39 (m, 1H), 2.99 (s, 3H). |
| 56 | ¹H NMR (300 MHz, Chloroform-d) δ 9.45 (s, 1H), 7.95 (s, 1H), 7.55 (s, 1H), 7.47-7.40 (m, 1H), 7.30-7.26 (m, 1H), 4.17- |
| | 4.02 (m, 1H), 3.84 (s, 3H), 3.57-3.52 (m, 1H), 3.46-3.36 (m, 2H), 3.00 (s, 3H), 2.46 (s, 3H). |
| 67 | ¹H NMR (300 MHz, Chloroform-d) δ 9.43 (s, 1H), 7.93 (s, 1H), 7.39-7.37 (m, 1H), 7.25-7.23 (m, 2H), 4.19-4.10 (m, 1H), |
| | 3.81-3.79 (m, 4H), 3.57-3.55 (m, 1H), 3.48-3.42 (m, 1H), 3.00 (s, 3H). |
| 69 | ¹H NMR (300 MHz, Chloroform-d) δ 9.42 (s, 1H), 7.94 (s, 1H), 7.36-7.34 (m, 3H), 4.16-4.07 (m, 1H), 3.80 (s, 3H), 3.79- |
| | 3.76 (m, 1H) 3.60 (d, J = 9.0 Hz, 1H), 3.46-3.40 (m, 1H), 3.00 (s, 3H), 2.41 (s, 3H). |
| 75 | ¹H NMR (300 MHz, Chloroform-d) δ 9.41 (s, 1H), 7.94 (s, 1H), 7.15-7.02 (m, 3H), 4.12-7.09 (m, 1H), 3.87-3.75 (m, 7H), |
| | 3.60-3.57 (m, 1H), 3.47-3.41 (m, 1H), 2.99 (s, 3H). |
| 80 | ¹H NMR (300 MHz, Chloroform-d) δ 9.53 (s, 1H), 7.94 (s, 1H), 7.68-7.54 (m, 2H), 7.25-7.17 (m, 1H), 4.24-4.20 (m, 1H), |
| | 3.82 (s, 3H), 3.78-3.75 (m, 2H), 3.54-3.38 (m, 1H), 3.01 (s, 3H). |
| 94 | ¹H NMR (300 MHz, Chloroform-d) δ 8.84 (s, 1H), 7.60-7.43 (m, 4H), 4.22-4.13 (m, 1H), 3.86-3.79 (m, 1H), 3.78 (s, 3H), |
| | 3.58 (d, J = 9.0 Hz, 1H), 3.52-3.44 (m, 1H), 3.01 (s, 3H). |
| 95 | ¹H NMR (300 MHz, Chloroform-d) δ 9.43 (s, 1H), 7.96 (s, 1H), 7.59-7.46 (m, 4H), 4.18-4.09 (m, 3H), 3.82 (t, J = 9.7 Hz, |
| | 1H), 3.60 (d, J = 9.3 Hz, 1H), 3.49-3.43 (m, 1H), 3.00 (s, 3H), 1.39 (t, J = 7.5 Hz, 3H). |
| 96 | ¹H NMR (300 MHZ, Chloroform-d) δ 9.45 (s, 1H), 7.97 (s, 1H), 7.60-7.46 (m, 4H), 4.21-4.02 (m, 3H), 3.85-3.78 (m, 1H), |
| | 3.63-3.57 (m, 1H), 3.49-3.42 (m, 1H), 3.00 (s, 3H), 1.79 (m, 2H), 0.89 (t, J = 7.5 Hz, 3H). |
| 97 | ¹H NMR (300 MHz, Chloroform-d) δ 9.42 (s, 1H), 7.98 (s, 1H), 7.58-7.47 (m, 4H), 4.70-4.50 (m, 1H), 4.25-4.10 (m, 1H), |
| | 3.85-3.80 (m, 1H), 3.65-3.55 (m, 1H), 3.49-3.41 (m, 1H), 3.00 (s, 3H), 1.50-1.40 (m, 6H). |
| 98 | ¹H NMR (300 MHz, Chloroform-d) δ 9.51 (s, 1H), 8.06 (s, 1H), 7.64-7.47 (m, 4H), 4.87-4.80 (m, 1H), 4.70-4.64 (m, 1H), |
| | 4.48-4.33 (m, 2H), 4.21-4.12 (m, 1H), 3.86-3.80 (m, 1H), 3.62 (d, J = 9.0 Hz, 1H), 3.51-3.43 (m, 1H), 3.02 (s, 3H). |
| 100 | ¹H NMR (300 MHz, Chloroform-d) δ 9.00 (s, 1H), 7.75 (s, 1H), 7.41-7.38 (m, 4H), 4.05-4.03 (m, 1H), 3.93-3.91 (m, 1H), |
| | 3.65 (s, 3H), 3.34-3.41 (m, 2H), |
| 114 | ¹H NMR (300 MHz, Chloroform-d) δ 9.43 (s, 1H), 7.92 (s, 1H), 7.01-6.96 (m, 2H), 4.05-3.95 (m, 1H), 3.79-3.73 (m, 4H), |
| | 3.48 (d, J = 9.0 Hz, 1H), 3.42-3.34 (m, 1H), 2.98 (s, 3H). |
| 115 | ¹H NMR (300 MHz, Chloroform-d) δ 9.41 (s, 1H), 7.94 (s, 1H), 6.90-6.87 (m, 2H), 6.76-6.68 (m, 1H), 4.15-4.03 (m, 1H), |
| | 3.81 (s, 3H), 3.77-3.75 (m, 1H), 3.54-3.52 (m, 1H), 3.44-3.38 (m, 1H), 2.99 (s, 3H). |
| 116 | ¹H NMR (300 MHz, Chloroform-d) δ 9.44 (s, 1H), 7.95 (s, 1H), 7.23-7.03 (m, 3H), 4.09-4.00 (m, 1H), 3.81 (s, 3H), 3.76- |
| | 3.75 (m, 1H), 3.51-3.49 (m, 1H), 3.44-3.38 (m, 1H), 2.99 (s, 3H). |
| 117 | ¹H NMR (300 MHz, Chloroform-d) δ 9.40 (s, 1H), 7.97 (s, 1H), 6.93 (s, 2H), 6.90 (s, 1H), 4.06-3.95 (m, 1H), 3.83-3.70 (m, |
| | 4H), 3.61 (d, J = 8.7 Hz, 1H), 3.47-3.36 (m, 1H), 2.97 (s, 3H), 2.30 (s, 6H). |
| 118 | ¹H NMR (300 MHz, Chloroform-d) δ 9.55 (s, 1H), 7.95 (s, 1H), 7.17-7.11 (m, 1H), 7.07-6.97 (m, 1H), 4.19 4.11 (m, 1H), |
| | 3.95 (s, 3H), 3.80 (s, 3H), 3.76-3.69 (m, 2H), 3.47-3.39 (m, 1H), 2.98 (s, 3H). |
| 119 | ¹H NMR (300 MHz, Chloroform-d) δ 9.07 (s, 1H), 7.58-7.46 (m, 5H), 4.20-4.11 (m, 1H), 3.84-3.78 (m, 1H), 3.69 (s, 3H), |
| | 3.55 (d, J = 9.0 Hz, 1H), 3.50-3.42 (m, 1H), 2.99 (s, 3H). |
| 120 | ¹H NMR (300 MHz, Chloroform-d) δ 9.48 (s, 1H), 7.98 (s, 1H), 7.60-7.46 (m, 4H), 4.20-4.11 (m, 1H), 3.87-3.78 (m, 4H), |
| | 3.80 (d, J = 9.6 Hz, 1H), 3.50-3.42 (m, 1H), 3.01 (d, J = 0.9 Hz, 3H). |
| 121 | ¹H NMR (300 MHz, Chloroform-d) δ 9.40 (s, 1H), 7.97 (s, 1H), 7.40-7.30 (m, 5H), 4.12-4.05 (q, J = 8.4 Hz, 1H), 3.85-3.75 |
| | (m, 4H), 3.59 (d, J = 8.7 Hz, 1H), 3.50-3.40 (m, 1H), 2.98 (s, 3H). |
| 122 | ¹H NMR (300 MHz, Chloroform-d) δ 9.53 (s, 1H), 7.97 (s, 1H), 7.43-7.27 (m, 2H), 7.21-7.00 (m, 2H), 4.23-4.07 (m, 1H), |
| | 3.87-3.70 (m, 5H), 3.54-3.45 (m, 1H), 2.99 (s, 3H). |
| 123 | ¹H NMR (300 MHz, Chloroform-d) δ 9.22 (s, 1H), 7.96 (s, 1H), 7.71 (d, J = 6.0 Hz, 1H), 7.58 (m, 1H), 7.41 (m, 2H), 4.52- |
| | 4.49 (m, 1H), 4.12-4.10 (m, 1H), 3.90-3.73 (m, 4H), 3.25-3.23 (m, 1H), 2.98 (s, 3H). |
| 124 | ¹H NMR (300 MHz, Chloroform-d) δ 9.27 (s, 1H), 7.94 (s, 1H), 7.23-7.18 (m, 4H), 4.42-4.43 (m, 1H), 3.88-3.75 (m, 4H), |
| | 3.66-3.63 (m, 1H), 3.38-3.32 (m, 1H), 2.99 (s, 3H), 2.50 (s, 3H). |
| 125 | ¹H NMR (300 MHz, Chloroform-d) δ 9.40 (s, 1H), 7.95 (s, 1H), 7.70-7.58 (m, 2H), 7.35-7.30 (m, 1H), 7.13-7.07 (m, 1H), |
| | 4.07-3.98 (m, 1H), 3.81 (s, 3H), 3.77-3.73 (m, 1H), 3.56 (d, J = 9.0 Hz, 1H), 3.45-3.39 (m, 1H), 2.99 (s, 3H). |
| 126 | ¹H NMR (300 MHz, Chloroform-d) δ 9.39 (s, 1H), 7.95 (s, 1H), 7.48 (s, 1H), 7.42-7.37 (m, 1H), 7.32-7.25 (m, 2H), 4.10- |
| | 4.01 (m, 1H), 3.80 (s, 3H), 3.76-3.73 (m, 1H), 3.56-3.53 (m, 1H), 3.46-3.40 (m, 1H), 2.99 (s, 3H). |
| 127 | ¹H NMR (300 MHz, Chloroform-d) δ 9.50 (s, 1H), 8.10 (s, 1H), 7.30-7.22 (m, 1H), 7.16-7.06 (m, 3H), 5.38 (s, 2H), 4.14 |
| | 3.99 (m, 1H), 3.83-3.73 (m, 1H), 3.62 (d, J = 9.0 Hz, 1H), 3.47-3.40 (m, 1H), 3.31 (s, 3H), 2.98 (s, 3H), 2.35 (s, 3H). |
| 128 | ¹H NMR (300 MHz, Chloroform-d) δ 9.40 (s, 1H), 7.94 (s, 1H), 7.25-7.07 (m, 4H), 4.12-3.99 (m, 1H), 3.82-3.70 (m, 1H), |
| | 3.60 (d, J = 8.7 Hz, 1H), 3.45-3.40 (m, 2H), 2.98 (s, 3H), 2.35 (s, 3H), 1.20-1.13 (m, 2H), 1.06-1.03 (m, 2H). |
| 129 | ¹H NMR (300 MHZ, Chloroform-d) δ 9.55 (s, 1H), 8.16 (s, 1H), 7.26-7.22 (m, 1H), 7.19-7.05 (m, 3H), 4.73-4.59 (m, 2H), |
| | 4.10-3.98 (m, 1H), 3.82-3.72 (m, 1H), 3.61 (d, J = 9.0 Hz, 1H), 3.49-3.40 (m, 1H), 2.99 (s, 3H), 2.36 (s, 3H). |
| 130 | ¹H NMR (300 MHz, Chloroform-d) δ 9.36 (s, 1H), 8.01 (s, 1H), 7.29-7.23 (m, 1H), 7.18-7.00 (m, 3H), 4.65-4.52 (m, 1H), |
| | 4.11-3.99 (m, 1H), 3.80-3.72 (m, 1H), 3.60 (d, J = 8.4 Hz, 1H), 3.49-3.37 (m, 1H), 2.98 (s, 3H), 2.35 (s, 3H), 1.48-1.42 (m, |
| | 6H). |
| 131 | ¹H NMR (300 MHz, Chloroform-d) δ 9.71 (s, 1H), 8.25 (s, 1H), 7.30-7.08 (m, 4H), 7.01-6.95 (m, 1H), 4.04-3.98 (m, 1H), |
| | 3.80-3.73 (m, 1H), 3.64-3.61 (m, 1H), 3.48-3.44 (m, 1H), 3.01 (s, 3H), 2.35 (s, 3H). |
| 132 | ¹H NMR (300 MHZ, Chloroform-d) δ 9.44 (s, 1H), 8.06 (s, 1H), 7.25-7.23 (m, 1H), 7.14-7.07 (m, 3H), 4.88 (d, J = 3.0 Hz, |
| | 2H), 4.09-4.11 (m, 1H), 3.80-3.74 (m, 1H), 3.60 (d, J = 9.0 Hz, 1H), 3.47-3.41 (m, 1H), 2.98 (s, 3H), 2.41-2.39 (m, 1H), |
| | 2.35 (s, 3H). |
| 133 | ¹H NMR (300 MHz, Chloroform-d) δ 9.47 (s, 1H), 7.79 (s, 1H), 7.41 (s, 1H), 7.28-7.18 (m, 1H), 7.13-7.04 (m, 3H), 4.11- |
| | 4.00 (m, 1H), 3.82-3.73 (m, 4H), 3.54 (d, J = 8.4 Hz, 1H), 3.42 (m, 1H), 2.95 (s, 3H), 2.34 (s, 3H). |
| 134 | ¹H NMR (300 MHz, Chloroform-d) δ 8.96 (s, 1H), 7.67 (s, 1H), 7.26-7.22 (m, 1H), 7.14-7.06 (m, 3H), 4.13-4.05 (m, 1H), |
| | 3.81-3.75 (m, 1H), 3.74 (s, 3H), 3.55 (d, J = 8.4 Hz, 1H), 3.47-3.41 (m, 1H), 2.97 (s, 3H), 2.35 (s, 3H), 2.19 (s, 3H). |
| 135 | ¹H NMR (300 MHz, Chloroform-d) δ 9.03 (s, 1H), 7.74 (s, 1H), 7.22-7.07 (m, 4H), 4.13-4.05 (m, 1H), 3.77-3.71 (m, 4H), |
| | 3.56 (d, J = 8.1 Hz, 1H), 3.47-3.41 (m, 1H), 2.98 (s, 3H), 2.64 (q, J = 7.5 Hz, 2H) 2.35 (s, 3H), 1.19 (t, J = 7.5 Hz, 3H). |
| 136 | ¹H NMR (300 MHz, Chloroform-d) δ 9.90 (s, 1H), 8.16 (s, 1H), 7.26-7.23 (m, 1H), 7.15-7.12 (m, 2H), 7.10-7.08 (m, 1H), |
| | 4.07-4.00 (m, 1H), 3.94 (s, 3H), 3.79-3.73 (m, 1H), 3.60 (d, J = 9.6 Hz, 1H), 3.45-3.40 (m, 1H), 2.97 (s, 3H), 2.36 (s, 3H). |
| 137 | ¹H NMR (300 MHz, Chloroform-d) δ 9.43 (s, 1H), 7.95 (s, 1H), 7.27-7.25 (m 1H), 7.15-7.08 (m, 3H), 6.63-6.53 (m, 1H), |
| | 5.74 (d, J = 18.0 Hz, 1H), 5.59 (d, J = 12.0 Hz, 1H), 4.12-4.07 (m, 1H), 3.82-3.60 (m, 4H), 3.58 (d, J = 6.0 Hz, 1H), 3.46- |
| | 3.40 (m, 1H), 2.97 (s, 3H), 2.35 (s, 3H). |
| 138 | ¹H NMR (300 MHz, Chloroform-d) δ 9.26 (s, 1H), 7.92 (s, 1H), 7.26-7.19 (m, 1H), 7.15-7.01 (m, 3H), 4.80-4.70 (m, 1H), |
| | 4.11-4.03 (m, 1H), 3.80-3.70 (m, 1H), 3.56 (d, J = 8.1 Hz, 1H), 3.45-3.50 (m, 1H), 2.96 (s, 3H), 2.33 (s, 3H), 1.70-1.55 (m, |
| | 1H), 1.50-1.35 (m, 6H), 1.12-1.00 (m, 2H), 0.80-0.60 (m, 2H). |
| 139 | ¹H NMR (300 MHz, Chloroform-d) δ 9.31 (s, 1H), 7.86 (s, 1H), 7.27-7.20 (m, 1H), 7.15-7.02 (m, 3H), 4.11-4.02 (m, 1H), |
| | 3.82-3.72 (m, 4H), 3.59-3.54 (m, 1H), 3.46-3.36 (m, 1H), 2.96 (s, 3H), 2.33 (s, 3H), 1.68-1.61 (m, 1H), 1.10-1.00 (m, 2H), |
| | 0.79-0.68 (m, 2H). |
| 140 | ¹H NMR (300 MHz, Chloroform-d) δ 8.94 (s, 1H), 7.75 (s, 1H), 7.23-7.21 (m, 1H), 7.13-7.05 (m, 3H), 4.13-4.05 (m, 1H), |
| | 3.82-3.72 (m, 4H), 3.53 (d, J = 8.1 Hz, 1H), 3.46-3.40 (m, 1H), 3.13-2.97 (m, 1H), 2.96 (s, 3H), 2.34 (s, 3H), 2.02-1.98 (m, |
| | 2H), 1.89-1.64 (m, 6H). |
| 141 | ¹H NMR (300 MHz, Chloroform-d) δ 9.15 (s, 1H), 7.96 (s, 1H), 7.23-7.21 (m, 1H), 7.12-7.05 (m, 3H), 6.04-6.03 (m, 1H), |
| | 4.10-4.05 (m, 1H), 3.80-3.74 (m, 4H), 3.53 (d, J = 8.4 Hz, 1H), 3.43-3.38 (m, 1H), 2.96 (s, 3H), 2.75-2.56 (m, 4H), 2.35 (s, |
| | 3H), 2.10-2.00 (m, 2H). |
| 142 | ¹H NMR (300 MHz, Chloroform-d) δ 9.10 (s, 1H), 8.07 (s, 1H), 7.53-7.50 (m, 2H), 7.34-7.31 (m, 2H), 7.23-7.18 (m, 1H), |
| | 7.10-7.06 (m, 3H), 4.11-4.02 (m, 1H), 3.78-3.71 (m, 4H), 3.47 (d, J = 8.7 Hz, 1H), 3.45-3.36 (m, 1H), 2.92 (s, 3H), 2.34 (s, |
| | 3H). |
| 143 | ¹H NMR (300 MHz, Chloroform-d) δ 9.61 (s, 1H), 7.93 (s, 1H), 7.25-7.23 (m, 1H), 7.15-7.08 (m, 3H), 4.05-3.97 (m, 1H), |
| | 3.79 (s, 3H), 3.76-3.71 (m, 1H), 3.61 (d, J = 9.3 Hz, 1H), 3.47-3.41 (m, 1H), 2.99 (s, 3H), 2.36 (s, 3H). |
| 144 | ¹H NMR (300 MHz, Chloroform-d) δ 9.63 (s, 1H), 7.92 (s, 1H), 7.28-7.25 (m, 1H), 7.17-7.06 (m, 3H), 4.05-3.96 (m, 1H), |
| | 3.82-3.73 (m, 4H), 3.61 (d, J = 9.3 Hz, 1H), 3.46-3.40 (m, 1H), 2.99 (s, 3H), 2.35 (s, 3H). |
| 145 | ¹H NMR (300 MHz, Chloroform-d) δ 8.54 (s, 1H), 7.25-7.15 (m, 1H), 7.13-7.05 (m, 3H), 4.13-4.05 (m, 1H), 3.85-3.70 (m, |
| | 4H), 3.55 (d, J = 8.4 Hz, 1H), 3.45 (m, 1H), 2.98 (s, 3H), 2.34 (s, 3H), 2.12 (s, 3H). |
| 146 | ¹H NMR (300 MHz, Chloroform-d) δ 9.98 (s, 1H), 8.14 (s, 1H), 7.28-7.23 (m, 1H), 7.15-7.08 (m, 3H), 4.04-3.95 (m, 1H), |
| | 3.87 (s, 3H), 3.78-3.71 (m, 1H), 3.61 (d, J = 9.3 Hz, 1H), 3.46-3.40 (m, 1H), 2.98 (s, 3H), 2.36 (s, 3H). |
| 147 | ¹H NMR (300 MHZ, Chloroform-d) δ 9.40 (s, 1H), 7.96 (s, 1H), 7.24-7.21 (m, 1H), 7.14-7.05 (m, 3H), 4.06-3.99 (m, 1H), |
| | 3.79 (s, 3H), 3.77-3.74 (m, 1H), 3.60 (d, J = 8.7 Hz, 1H), 3.46-3.40 (m, 1H), 2.98 (s, 3H), 2.34 (s, 3H). |
| 148 | ¹H NMR (300 MHz, Chloroform-d) δ 9.44 (s, 1H), 7.43 (s, 1H), 7.25-7.20 (m, 1H), 7.12-7.06 (m, 3H), 4.08-3.99 (m, 1H), |
| | 3.80-3.73 (m, 1H), 3.63-3.53 (m, 4H), 3.48-3.40 (m, 1H), 2.96 (s, 3H), 2.34 (s, 3H). |
| 149 | ¹H NMR (300 MHz, Chloroform-d) δ 9.37 (s, 1H), 7.96 (s, 1H), 7.38-7.29 (m, 3H), 7.25-7.17 (m, 1H), 6.77-6.64 (m, 1H), |
| | 5.76 (d, J = 18.0 Hz, 1H), 5.27 (d, J = 12.0 Hz, 1H), 4.15-4.02 (m, 1H), 3.84-3.74 (m, 4H), 3.61 (d, J = 9.0 Hz, 1H), 3.49- |
| | 3.40 (m, 1H), 3.00 (s, 3H). |
| 150 | ¹H NMR (300 MHZ, Chloroform-d) δ 9.36 (s, 1H), 7.96 (s, 1H), 7.41-7.30 (m, 3H), 7.23-7.21 (m, 1H), 5.36-5.34 (m, 1H), |
| | 5.12-5.10 (m, 1H), 4.12-4.10 (m, 1H), 3.81 (s, 3H), 3.77-3.74 (s, 1H), 3.62 (d, J = 9.0 Hz, 1H), 3.49-3.43 (m, 1H), 2.99 (s, |
| | 3H), 2.15 (s, 3H). |
| 151 | ¹H NMR (300 MHz, Chloroform-d) δ 9.34 (s, 1H), 7.96 (s, 1H), 7.25-7.21 (m, 1H), 7.14-7.05 (m, 2H), 6.95-6.90 (m, 1H), |
| | 4.10-4.02 (m, 1H), 3.80 (s, 3H), 3.77-3.74 (m, 2H), 3.59 (d, J = 8.4 Hz, 1H), 3.46-3.40 (m, 1H), 2.98 (s, 3H), 0.99-0.93 (m, |
| | 2H), 0.72-0.67 (m, 2H). |
| 152 | ¹H NMR (300 MHz, Chloroform-d) δ 9.35 (s, 1H), 7.96 (s, 1H), 7.30-7.25 (m, 1H), 7.22-7.10 (m, 3H), 4.14-4.02 (m, 1H), |
| | 3.85-3.71 (m, 4H), 3.60 (d, J = 9.0 Hz, 1H), 3.49-3.38 (m, 1H), 3.06-2.90 (m, 4H), 2.14-1.99 (m, 2H), 1.88-1.75 (m, 2H), |
| | 1.73-1.67 (m, 2H), 1.63-1.50 (m, 2H). |
| 153 | ¹H NMR (300 MHz, Chloroform-d) δ 9.35 (s, 1H), 7.96 (s, 1H), 7.40-7.38 (m, 1H), 7.36-7.27 (m, 2H), 7.21-7.15 (m, 1H), |
| | 6.23-6.17 (m, 1H), 4.14-4.02 (m, 1H), 3.84-3.73 (m, 4H), 3.61 (d, J = 9.0 Hz, 1H), 3.49-3.39 (m, 1H), 2.99 (s, 3H), 2.75- |
| | 2.66 (m, 2H), 2.57-2.48 (m, 2H), 2.07-1.94 (m, 2H). |
| 154 | ¹H NMR (300 MHz, Chloroform-d) δ 9.44 (s, 1H), 7.95 (s, 1H), 7.48-7.43 (m, 4H), 6.65 (t, J = 56.4 Hz, 1H), 4.18-4.10 (m, |
| | 1H), 3.84-3.77 (m, 4H), 3.61 (d, J = 10.5 Hz, 1H), 3.49-3.43 (m, 1H), 3.00 (s, 3H). |
| 155 | ¹H NMR (300 MHz, Chloroform-d) δ 8.89 (s, 1H), 6.96-6.84 (m, 5H), 6.78-6.75 (m, 2H), 6.71-6.65 (m, 4H), 3.98 (s, 2H), |
| | 3.51-3.49 (m, 1H), 3.26 (s, 3H), 3.19-3.16 (m, 1H), 3.05-3.03 (m, 1H), 2.72-2.69 (m, 1H). |
| 156 | ¹H NMR (300 MHz, Chloroform-d) δ 9.57 (s, 1H), 7.74 (s, 1H), 7.59-7.37 (m, 5H), 4.20-4.11 (m, 1H), 3.87-3.70 (m, 4H), |
| | 3.61-3.54 (m, 1H), 3.65-3.35 (m, 1H), 2.96 (s, 3H). |
| 157 | ¹H NMR (300 MHz, Chloroform-d) δ 9.45 (s, 1H), 7.93 (s, 1H), 7.65-7.45 (m, 4H), 4.22-4.12 (m, 1H), 3.88-3.78 (m, 1H), |
| | 3.62 (d, J = 9.0 Hz, 1H), 3.50-3.40 (m, 1H), 3.00 (s, 3H), 1.66 (s, 9H). |
| 158 | ¹H NMR (300 MHz, Chloroform-d) δ 9.74 (s, 1H), 8.23 (s, 1H), 7.55-7.51 (m, 4H), 7.15 (t, J = 57.0 Hz, 1H), 4.18-4.09 (m, |
| | 1H), 3.91-3.78 (m, 1H), 3.63 (d, J = 9.3 Hz, 1H), 3.52-3.43 (m, 1H), 3.02 (s, 3H). |
| 159 | ¹H NMR (300 MHz, Chloroform-d) δ 9.74 (s, 1H), 8.24 (s, 1H), 7.65 (s, 1H), 7.60-7.50 (m, 4H), 7.09 (t, J = 60.3 Hz, 1H), |
| | 4.15-4.05 (m, 1H), 3.90-3.78 (m, 1H), 3.67-3.39 (m, 2H), 3.00 (s, 3H). |
| 160 | ¹H NMR (300 MHz, Chloroform-d) δ 9.51 (s, 1H), 8.05 (s, 1H), 7.58-7.47 (m, 4H), 4.40 (t, J = 6.3 Hz, 2H), 4.20-4.11 (m, |
| | 1H), 3.86-3.78 (m, 3H), 3.61 (d, J = 9.3 Hz, 1H), 3.49-3.43 (m, 1H), 3.01 (s, 3H). |
| 161 | ¹H NMR (300 MHz, Chloroform-d) δ 9.59 (s, 1H), 8.13 (s, 1H), 7.59-7.50 (m, 4H), 4.72-4.64 (m, 2H), 4.20-4.11 (m, 1H), |
| | 3.86-3.79 (m, 1H), 3.62 (d, J = 9.3 Hz, 1H), 3.52-3.45 (m, 1H), 3.01 (s, 3H). |
| 162 | ¹H NMR (300 MHz, Chloroform-d) δ 9.30 (s, 1H), 7.83 (s, 1H), 7.44-7.26 (m, 4H), 5.81-5.62 (m, 1H), 5.15-4.86 (m, 2H), |
| | 4.57-4.47 (m, 2H), 4.00-3.97 (m, 1H), 3.64-3.61 (m, 1H), 3.45-3.28 (m, 2H), 2.83 (s, 3H). |
| 163 | ¹H NMR (300 MHz, Chloroform-d) δ 9.50 (s, 1H), 8.03 (s, 1H), 7.59-7.47 (m, 4H), 4.89-4.88 (m, 2H), 4.20-4.11 (m, 1H), |
| | 3.87-3.79 (m, 1H), 3.60 (d, J = 9.0 Hz, 1H), 3.49-3.43 (m, 1H), 3.03 (s, 3H), 2.43-2.39 (m, 1H). |
| 164 | ¹H NMR (300 MHz, Chloroform-d) δ 9.44 (s, 1H), 7.91 (s, 1H), 7.58-7.46 (m, 4H), 4.20-4.09 (m, 1H), 3.85-3.77 (m 1H), |
| | 3.60 (d, J = 9.0 Hz, 1H), 3.49-3.36 (m, 2H), 3.00 (s, 3H), 1.21-1.12 (m, 2H), 1.09-1.00 (m, 2H). |
| 165 | ¹H NMR (300 MHz, Chloroform-d) δ 9.44 (s, 1H), 7.98 (s, 1H), 7.61-7.45 (m, 4H), 4.21-4.13 (m, 1H), 3.96 (d, J = 6.9 Hz, |
| | 2H), 3.85-3.78 (m 1H), 3.60 (d, J = 9.0 Hz, 1H), 3.49-3.42 (m, 1H), 3.01 (s, 3H), 0.90-0.80 (m, 1H), 0.58-0.52 (m, 2H), |
| | 0.40-0.35 (m, 2H). |
| 166 | ¹H NMR (300 MHz, Chloroform-d) δ 9.48 (s, 1H), 8.04 (s, 1H), 7.59-7.47 (m, 4H), 7.32-7.25 (m, 3H), 7.20-7.15 (m, 2H), |
| | 5.29 (s, 2H), 4.19-4.11 (m, 1H), 3.84-3.77 (m, 1H), 3.60 (d, J = 9.3 Hz, 1H), 3.47-3.42 (m, 1H), 3.00 (s, 3H). |
| 171 | ¹H NMR (300 MHz, Chloroform-d) δ 9.54 (s, 1H), 8.08 (s, 1H), 7.60-7.49 (m, 4H), 5.39 (s, 2H), 4.20-4.10 (m, 1H), 3.86- |
| | 3.79 (m, 1H), 3.62 (d, J = 9.3 Hz, 1H), 3.50-3.44 (m, 1H), 3.33 (s, 3H), 3.01 (s, 3H). |
| 172 | ¹H NMR (300 MHz, Chloroform-d) δ 9.45 (s, 1H), 8.01 (s, 1H), 7.60-7.45 (m, 4H), 4.27 (t, J = 5.7 Hz, 2H), 4.20-4.09 (m, |
| | 1H), 3.88-3.67 (m, 3H), 3.60 (d, J = 8.7 Hz, 1H), 3.51-3.41 (m, 1H), 3.30 (s, 3H), 3.01 (s, 3H). |
| 173 | ¹H NMR (300 MHz, Chloroform-d) δ 9.47 (s, 1H), 7.99 (s, 1H), 7.58-7.46 (m, 4H), 4.32-4.05 (m, 3H), 3.88-3.76 (m, 1H), |
| | 3.61 (d, J = 9.3 Hz, 1H), 3.52-3.40 (m, 1H), 3.00 (s, 3H), 2.81-2.72 (m, 2H), 2.30 (s, 6H). |
| 174 | 1H NMR (300 MHz, Chloroform-d) δ 9.53 (s, 1H), 8.09 (s, 1H), 7.62-7.47 (m, 4H), 5.09 (q, J = 6.9 Hz, 1H), 4.20-4.10 (m, |
| | 1H), 3.87-3.80 (m, 1H), 3.74 (s, 3H), 3.63 (d, J = 6.9 Hz, 1H), 3.50-3.45 (m, 1H), 3.03 (s, 3H), 1.83 (d, J = 6.9 Hz, 3H). |
| 180 | ¹H NMR (300 MHz, Chloroform-d) δ 9.54 (s, 1H), 8.09 (s, 1H), 7.59-7.50 (m, 4H), 4.87 (s, 2H), 4.19-4.10 (m, 1H), 3.87- |
| | 3.77 (m, 1H), 3.61 (d, J = 9.6 Hz, 1H), 3.50-3.44 (m, 1H), 3.01 (s, 3H), 2.11 (s, 3H). |
| 181 | ¹H NMR (300 MHz, Chloroform-d) δ 9.62 (s, 1H), 8.22 (s, 1H), 7.61-7.40 (m, 9H), 4.23-4.15 (m, 1H), 3.87-3.80 (m, 1H), |
| | 3.65 (d, J = 9.3 Hz, 1H), 3.51-3.45 (m, 1H), 3.02 (s, 3H). |
| 182 | ¹H NMR (300 MHz, Chloroform-d) δ 9.62 (s, 1H), 8.11 (s, 1H), 7.59-7.50 (m, 4H), 5.01 (d, J = 10.8 Hz, 2H), 4.17-4.09 (m, |
| | 1H), 3.85-3.78 (m, 1H), 3.63-3.58 (m, 1H), 3.50-3.43 (m, 1H), 3.02 (s, 3H). |
| 184 | ¹H NMR (300 MHz, Chloroform-d) δ 9.45 (s, 1H), 7.98 (s, 1H), 7.53-7.45 (m, 3H), 7.41-7.33 (m, 3H), 7.30-7.25 (m, 3H), |
| | 4.59 (s, 2H), 4.20-4.04 (m, 1H), 3.83 (s, 3H), 3.75-3.61 (m, 2H), 3.36-3.23 (m, 1H). |
| 186 | ¹H NMR (300 MHz, Chloroform-d) δ 9.46 (s, 1H), 7.99 (s, 1H), 7.60-7.45 (m, 4H), 4.19-4.10 (m, 1H), 3.88 (s, 3H), 3.84- |
| | 3.77 (m, 1H), 3.62 (d, J = 9.3 Hz, 1H), 3.47-3.41 (m, 1H), 3.00 (s, 3H). |
| 187 | ¹H NMR (300 MHz, Chloroform-d) δ 10.00 (s, 1H), 7.91 (s, 1H), 7.57-7.49 (m, 4H), 4.23-4.12 (m, 1H), 3.97 (s, 3H), 3.87- |
| | 3.81 (m, 1H), 3.63 (d, J = 9.3 Hz, 1H), 3.51-3.45 (m, 1H), 3.00 (s, 3H). |
| 188 | ¹H NMR (300 MHz, Chloroform-d) δ 11.02 (s, 1H), 8.25 (s, 1H), 7.61-7.56 (m, 4H), 4.19-4.08 (m, 1H), 3.95 (s, 3H), 3.85- |
| | 3.76 (m, 1H), 3.67 (d, J = 9.3 Hz, 1H), 3.51-3.44 (m, 1H), 3.02 (s, 3H). |
| 189 | ¹H NMR (300 MHz, Chloroform-d) δ 9.03 (s, 1H), 7.65 (s, 1H), 7.58-7.48 (m, 4H), 4.24-4.14 (m, 1H), 3.85-3.79 (m, 1H), |
| | 3.74 (s, 3H), 3.56 (d, J = 9.0 Hz, 1H), 3.49-3.43 (m, 1H), 3.00 (s, 3H), 2.20 (s, 3H). |
| 190 | ¹H NMR (300 MHz, Chloroform-d) δ 9.12 (s, 1H), 7.71 (s, 1H), 7.57-7.47 (m, 4H), 4.23-4.14 (m, 1H), 3.85-3.78 (m, 1H), |
| | 3.77 (s, 3H), 3.56 (d, J = 9.0 Hz, 1H), 3.48-3.42 (m, 1H), 2.99 (s, 3H), 2.67-2.62 (q, J = 7.5 Hz, 2H), 1.18 (t, J = 7.5 Hz, 3H). |
| 192 | ¹H NMR (300 MHz, Chloroform-d) δ 8.89 (s, 1H), 7.57-7.45 (m, 5H), 4.24-4.14 (m 1H), 3.94 (s, 3H), 3.84-3.78 (m, 1H), |
| | 3.63 (s, 3H), 3.54-3.43 (m, 2H), 2.99 (s, 3H). |
| 193 | ¹H NMR (300 MHz, Chloroform-d) δ 9.46 (s, 1H), 7.83 (s, 1H), 7.57-7.45 (m, 4H), 4.46 (s, 2H), 4.23-4.12 (m, 1H), 3.85- |
| | 3.76 (m, 4H), 3.57 (d, J = 8.7 Hz, 1H), 3.50-3.40 (m, 1H), 3.36 (s, 3H), 2.98 (s, 3H). |
| 194 | ¹H NMR (300 MHz, Chloroform-d) δ 9.17 (s, 1H), 7.75 (s, 1H), 7.58-7.45 (m, 4H), 4.23-4.12 (m, 1H), 3.86-3.77 (m, 4H), |
| | 3.55 (d, J = 8.7 Hz, 1H), 3.48-3.42 (m, 1H), 3.18-3.03 (m, 1H), 3.00 (s, 3H), 1.38-1.33 (m, 6H). |
| 195 | ¹H NMR (300 MHz, Chloroform-d) δ 9.39 (s, 1H), 7.87 (s, 1H), 7.58-7.46 (m, 4H), 4.25-4.16 (m, 1H), 3.87-3.78 (m, 4H), |
| | 3.58 (d, J = 8.7 Hz, 1H), 3.48-3.42 (m, 1H), 3.01 (s, 3H), 1.67-1.62 (m, 1H), 1.09-1.06 (m, 2H), 0.78-0.66 (m, 2H). |
| 196 | ¹H NMR (300 MHz, Chloroform-d) δ 9.62 (s, 1H), 8.14 (s, 1H), 7.60-7.48 (m, 4H), 7.22 (t, J = 57.9 Hz, 1H), 4.23-4.13 (m, |
| | 1H), 3.88-3.79 (m, 1H), 3.60 (d, J = 9.0 Hz, 1H), 3.50-3.42 (m, 1H), 3.01 (s, 3H), 1.85-1.74 (m, 1H), 1.18-1.10 (m, 2H), |
| | 0.91-0.81 (m, 2H). |
| 197 | ¹H NMR (300 MHz, Chloroform-d) δ 9.34 (s, 1H), 7.92 (s, 1H), 7.61-7.44 (m, 4H), 4.82-4.71 (m, 1H), 4.26-4.15 (m, 1H), |
| | 3.85-3.78 (m, 1H), 3.58 (d, J = 8.4 Hz, 1H), 3.49-3.38 (m, 1H), 3.00 (s, 3H), 1.68-1.60 (m, 1H), 1.44 (d, J = 6.7 Hz, 6H), |
| | 1.10-1.04 (m, 2H), 0.77-0.64 (m, 2H). |
| 198 | ¹H NMR (300 MHz, Chloroform-d) δ 9.13 (s, 1H), 7.58-7.44 (m, 4H), 7.36 (s, 1H), 4.23-4.10 (m, 1H), 3.88-3.77 (m, 1H), |
| | 3.65 (s, 3H), 3.60-3.54 (m, 1H), 3.50-3.41 (m, 1H), 2.99 (s, 3H). |
| 199 | ¹H NMR (300 MHz, Chloroform-d) δ 10.62 (s, 1H), 8.22 (s, 1H), 7.59-7.47 (m, 4H), 4.24-4.15 (m, 1H), 4.11 (s, 3H), 4.04 (s, |
| | 3H), 3.85-3.79 (m, 1H) 3.61 (d, J = 9.0 Hz, 1H), 3.46-3.41 (m, 1H), 3.00 (s, 3H). |
| 200 | ¹H NMR (300 MHz, Chloroform-d) δ 9.02 (s, 1H), 7.73 (s, 1H), 7.57-7.45 (m, 4H), 4.24-4.16 (m, 1H), 3.85-3.72 (m, 4H), |
| | 3.53 (d, J = 8.7 Hz, 1H), 3.48-3.42 (m, 1H), 3.11-3.05 (m, 1H), 2.99 (s, 3H), 2.01-1.71 (m, 8H). |
| 201 | ¹H NMR (300 MHz, Chloroform-d) δ 9.24 (s, 1H), 7.94 (s, 1H), 7.60-7.41 (m, 4H), 6.06-6.01 (m, 1H), 4.23-4.15 (m, 1H), |
| | 3.84-3.75 (m, 4H), 3.54 (d, J = 8.4 Hz, 1H), 3.45-3.39 (m, 1H), 2.98 (s, 3H), 2.69-2.56 (m, 4H), 2.10-2.00 (m, 2H). |
| 202 | ¹H NMR (300 MHz, Chloroform-d) δ 9.19 (s, 1H), 8.04 (s, 1H), 7.56-7.47 (m, 6H), 7.34 (d, J = 8.4 Hz, 2H), 4.42-4.41 (m, |
| | 1H), 3.82-3.74 (m, 4H), 3.48-3.37 (m, 2H), 2.94 (s, 3H). |
| 203 | ¹H NMR (300 MHz, Chloroform-d) δ 9.48 (s, 1H), 7.90 (s, 1H), 7.62-7.40 (m, 4H), 6.65-6.48 (m, 1H), 5.71 (d, J = 18.0 Hz, |
| | 1H), 5.57 (d, J = 12.0 Hz, 1H), 4.17-4.07 (m, 1H), 3.79-3.75 (m, 4H), 3.55 (d, J = 8.4 Hz, 1H), 3.48-3.38 (m, 1H), 2.97 (s, |
| | 3H). |
| 204 | ¹H NMR (300 MHz, Chloroform-d) δ 9.18 (s, 1H), 7.75 (s, 1H), 7.60-7.47 (m, 4H), 4.21-4.12 (m, 1H), 3.88-3.78 (m, 1H), |
| | 3.71 (s, 3H), 3.61 (d, J = 9.0 Hz, 1H), 3.51-3.42 (m, 1H), 2.99 (s, 3H), 2.81 (s, 6H). |
| 205 | ¹H NMR (300 MHz, Chloroform-d) δ 9.68 (s, 1H), 7.91 (s, 1H), 7.63-7.40 (m, 4H), 4.16-4.06 (m, 1H), 3.84-3.73 (m, 4H), |
| | 3.61 (d, J = 9.6 Hz, 1H), 3.52-3.40 (m, 1H), 3.01 (s, 3H). |
| 206 | ¹H NMR (300 MHz, Chloroform-d) δ 9.70 (s, 1H), 7.90 (s, 1H), 7.60-7.49 (m, 4H), 4.16-4.07 (m, 1H), 3.85-3.77 (m, 4H), |
| | 3.62 (d, J = 9.3 Hz, 1H), 3.49-3.41 (m, 1H), 3.01 (s, 3H). |
| 207 | ¹H NMR (300 MHz, Chloroform-d) δ 8.83 (s, 1H), 7.57-7.45 (m, 4H), 4.22-4.14 (m, 1H), 3.88-3.76 (m, 4H), 3.59 (d, J = 9.0 |
| | Hz, 1H), 3.51-3.45 (m, 1H), 3.02 (s, 3H). |
| 208 | ¹H NMR (300 MHz, Chloroform-d) δ 8.63 (s, 1H), 7.60-7.45 (m, 4H), 4.27-4.15 (m, 1H), 3.89-3.82 (m, 1H), 3.75 (s, 3H), |
| | 3.57 (d, J = 7.7 Hz, 1H), 3.53-3.45 (m 1H), 3.02 (s, 3H), 2.14 (s, 3H). |
| 209 | ¹H NMR (300 MHz, Chloroform-d) δ 8.89 (s, 1H), 7.57-7.46 (m, 4H), 7.07 (d, J = 57.9 Hz, 1H), 4.22-4.13 (m, 1H), 3.88- |
| | 3.81 (m, 1H), 3.59 (d, J = 9.0 Hz, 1H), 3.53-3.47 (m, 1H), 3.03 (s, 3H), 2.19 (s, 3H). |
| 210 | ¹H NMR (300 MHz, Chloroform-d) δ 9.66 (s, 1H), 7.82 (s, 1H), 7.62-7.46 (m, 4H), 4.20-4.11 (m, 1H), 3.85-3.78 (m, 1H), |
| | 3.74 (s, 3H), 3.61 (d, J = 9.0 Hz, 1H), 3.49-3.43 (m, 1H), 3.01 (s, 3H), 1.78-1.71 (m, 1H), 0.97-0.93 (m, 2H), 0.86-0.79 (m, |
| | 2H). |
| 211 | ¹H NMR (300 MHz, Chloroform-d) δ 9.88 (s, 1H), 7.59-7.47 (m, 4H), 4.13-4.02 (m, 1H), 3.85-3.77 (m, 1H), 3.67-3.58 (m, |
| | 4H), 3.53-3.43 (m, 1H), 3.02 (s, 3H). |
| 212 | ¹H NMR (300 MHz, Chloroform-d) δ 9.46 (s, 1H), 7.94 (s, 1H), 7.66-7.42 (m, 4H), 4.21-4.09 (m, 1H), 3.85-3.75 (m, 4H), |
| | 3.61 (d, J = 9.3 Hz, 1H), 3.51-3.41 (m, 1H), 3.00 (s, 3H). |
| 213 | ¹H NMR (300 MHz, Chloroform-d) δ 9.26 (s, 1H), 7.71 (s, 1H), 7.56-7.46 (m, 4H), 4.22-4.06 (m, 1H), 3.93 (s, 3H), 3.83- |
| | 3.77 (m, 1H), 3.67 (s, 3H), 3.59-3.53 (m, 1H), 3.49-3.41 (m, 1H), 3.00 (s, 3H). |
| 214 | ¹H NMR (300 MHz, Chloroform-d) δ 10.03 (s, 1H), 8.12 (s, 1H), 7.60-7.47 (m, 4H), 4.15-4.06 (m, 1H), 3.88 (s, 3H), 3.83- |
| | 3.76 (m, 1H) 3.61 (d, J = 9.0 Hz, 1H), 3.49-3.42 (m, 1H), 3.01 (s, 3H). |
| 215 | ¹H NMR (300 MHz, Chloroform-d) δ 9.68 (s, 1H), 8.25 (s, 1H), 7.32-7.27 (m, 1H), 7.14 (t, J = 57.9 Hz, 1H), 6.95-6.91 (m, |
| | 1H), 6.89-6.88 (m, 1H), 6.84-6.80 (m, 1H), 4.08-4.00 (m, 1H), 3.82-3.77 (m, 4H), 3.64-3.60 (m, 1H), 3.49-3.43 (m, 1H), |
| | 2.99 (s, 3H). |
| 216 | ¹H NMR (300 MHz, Chloroform-d) δ 8.53 (s, 1H), 7.29-7.23 (m, 1H), 6.91-6.78 (m, 3H), 4.16-4.07 (m, 1H), 3.83-3.77 (m, |
| | 4H), 3.73 (s, 3H), 3.56 (d, J = 8.4 Hz, 1H), 3.49-3.39 (m, 1H), 2.98 (s, 3H), 2.12 (s, 3H). |
| 217 | ¹H NMR (300 MHz, Chloroform-d) δ 9.28 (s, 1H), 7.85 (s, 1H), 7.30-7.24 (m, 1H), 6.93-6.84 (m, 2H), 6.78 (d, J = 7.8 Hz, |
| | 1H), 4.12-4.02 (m, 1H), 3.82-3.69 (m, 7H), 3.56 (d, J = 8.4 Hz, 1H), 3.45-3.37 (m, 1H), 2.95 (s, 3H), 1.64-1.55 (m, 1H), |
| | 1.03 (d, J = 7.2 Hz, 2H), 0.75-0.62 (m, 2H). |
| 218 | ¹H NMR (300 MHz, Chloroform-d) δ 9.40 (s, 1H), 7.96 (s, 1H), 7.42-7.26 (m, 4H), 4.45 (s, 2H), 4.10 (m, 1H), 3.80-3.75 (m, |
| | 4H), 3.61 (d, J = 8.7 Hz, 1H), 3.52-3.36 (m, 4H), 2.99 (s, 3H). |
| 219 | ¹H NMR (300 MHz, Chloroform-d) δ 9.36 (s, 1H), 7.97 (s, 1H), 7.29-7.23 (m, 1H), 6.95-6.83 (m, 2H), 6.82-6.72 (m, 1H), |
| | 4.16-4.06 (m, 1H), 4.02 (d, J = 6.9 Hz, 2H), 3.80 (s, 3H), 3.77-3.74 (m, 1H), 3.58 (d, J = 8.7 Hz, 1H), 3.47-3.40 (m, 1H), |
| | 2.97 (s, 3H), 1.40 (t, J = 6.9 Hz, 3H). |
| 220 | ¹H NMR (300 MHz, Chloroform-d) δ 9.35 (s, 1H), 7.94 (s, 1H), 7.25-7.20 (m, 1H), 6.90-6.84 (m, 2H), 6.81-6.74 (m, 1H), |
| | 4.07-3.99 (m, 1H), 3.89 (t, J = 6.3 Hz, 2H), 3.79-3.74 (m, 4H), 3.58 (d, J = 8.7 Hz, 1H), 3.46-3.38 (m, 1H), 2.95 (s, 3H), |
| | 1.81-1.73 (m, 2H), 1.01 (t, J = 7.5 Hz, 3H). |
| 221 | ¹H NMR (300 MHz, Chloroform-d) δ 9.36 (s, 1H), 7.97 (s, 1H), 7.29-7.25 (m, 1H), 6.93-6.84 (m, 2H), 6.82-6.74 (m, 1H), |
| | 4.13-4.01 (m, 1H), 3.97 (t, J = 6.6 Hz, 2H), 3.80-3.73 (m, 1H), 3.59 (d, J = 8.7 Hz, 1H), 3.49-3.37 (m, 1H), 2.97 (s, 3H), 2.80 |
| | (s, 3H), 1.80-1.70 (m, 2H), 1.55-1.42 (m, 2H), 0.97 (t, J = 7.2 Hz, 3H). |
| 222 | ¹H NMR (300 MHz, Chloroform-d) δ 9.35 (s, 1H), 7.97 (s, 1H), 7.29-7.15 (m, 1H), 6.93-6.83 (m, 2H), 6.81-6.74 (m, 1H), |
| | 4.35-4.25 (m, 1H), 4.12-4.00 (m, 1H), 3.83-3.73 (m, 4H), 3.59 (d, J = 8.4 Hz, 1H), 3.49-3.40 (m, 1H), 2.97 (s, 3H), 1.80- |
| | 1.60 (m, 2H), 1.28 (d, J = 6.3 Hz, 3H), 0.97 (t, J = 7.2 Hz, 3H). |
| 223 | ¹H NMR (300 MHz, DMSO-d6) δ 9.84 (s, 1H), 7.73 (s, 1H), 7.30-7.22 (m, 1H), 6.95-6.83 (m, 3H), 4.76 (d, J = 2.4 Hz, 2H), |
| | 3.94-3.82 (m, 1H), 3.81-3.68 (m, 5H), 3.55 (t, J = 2.4 Hz, 1H), 3.37 (t, J = 9.0 Hz, 1H), 2.79 (s, 3H). |
| 224 | ¹H NMR (300 MHz, Chloroform-d) δ 9.43 (s, 1H), 7.94 (d, J = 10.5 Hz, 1H), 7.39-7.33 (m, 1H), 7.24-7.17 (m, 1H), 7.12- |
| | 7.02 (m, 1H), 7.04 (d, J = 8.1 Hz, 1H), 6.52 (t, J = 73.8 Hz, 1H), 4.15-4.03 (m, 1H), 3.81 (s, 3H), 3.80-3.76 (m, 1H), 3.58 (d, |
| | J = 9.0 Hz, 1H), 3.47-3.41 (m, 1H), 3.00 (s, 3H). |
| 225 | ¹H NMR (300 MHz, Chloroform-d) δ 9.37 (s, 1H), 7.95 (s, 1H), 7.30-7.25 (m, 1H), 6.96-6.91 (m, 2H), 6.83-6.80 (m, 1H), |
| | 4.83-4.81 (m, 1H), 4.68-4.65 (m, 1H), 4.27-4.24 (m, 1H), 4.18-4.15 (m, 1H), 4.10-4.01 (m, 1H), 3.80-3.74 (m, 4H), 3.58 (d, |
| | J = 8.7 Hz, 1H), 3.47-3.41 (m, 1H), 2.97 (s, 3H). |
| 226 | ¹H NMR (300 MHz, Chloroform-d) δ 9.40 (s, 1H), 7.96 (s, 1H), 7.31-7.26 (m, 1H), 7.00-6.78 (m, 3H), 4.31-4.21 (m, 2H), |
| | 4.10-4.00 (m, 1H), 3.86-3.70 (m, 6H), 3.59 (d, J = 9.0 Hz, 1H), 3.49-3.40 (m, 1H), 2.98 (s, 3H). |
| 227 | ¹H NMR (300 MHz, Chloroform-d) δ 9.40 (s, 1H), 7.96 (s, 1H), 7.34-7.27 (m, 1H), 7.00 (d, J = 7.2 Hz, 1H), 6.92 (s, 1H), |
| | 6.81 (d, J = 6.9 Hz, 1H), 6.08 (d, J = 54.0 Hz, 1H), 4.23-4.02 (m, 3H), 3.82-3.74 (m, 4H), 3.58 (d, J = 9.3 Hz, 1H), 3.49-3.39 |
| | (m, 1H), 2.98 (s, 3H). |
| 228 | ¹H NMR (300 MHz, Chloroform-d) δ 10.02 (s, 1H), 8.12 (s, 1H), 7.44-7.12 (m, 4H), 4.11-4.01 (m, 1H), 3.88 (s, 3H), 3.82- |
| | 3.75 (m, 1H), 3.58 (d, J = 9.3 Hz, 1H), 3.48-3.40 (m, 1H), 2.99 (s, 3H). |
| 229 | ¹H NMR (300 MHz, Chloroform-d) δ 9.41 (s, 1H), 7.95 (s, 1H), 7.45-7.30 (m, 2H), 7.20-7.10 (m, 2H), 4.18-4.08 (m, 1H), |
| | 3.90-3.70 (m, 4H), 3.57 (d, J = 9.0 Hz, 1H), 3.49-3.41 (m, 1H), 3.00 (s, 3H). |
| 230 | ¹H NMR (300 MHz, Chloroform-d) δ 9.41 (s, 1H), 7.95 (s, 1H), 7.34-7.27 (m, 1H), 7.03 (d, J = 8.1 Hz, 1H), 6.95 (s, 1H), |
| | 6.83 (d, J = 6.9 Hz, 1H), 4.40-4.30 (m, 2H), 4.10-4.03 (m, 1H), 3.82-3.72 (m, 4H), 3.57 (d, J = 9.6 Hz, 1H), 3.49-3.38 (m, |
| | 1H), 2.98 (s, 3H). |
| 231 | ¹H NMR (300 MHz, Chloroform-d) δ 9.44 (s, 1H), 7.95 (s, 1H), 7.42-7.32 (m, 1H), 7.30-7.26 (m, 1H), 7.17-7.12 (m, 2H), |
| | 5.90 (t, J = 52.8 Hz, 1H), 4.20-4.05 (m, 1H), 3.85-3.72 (m, 4H), 3.59 (d, J = 9.0 Hz, 1H), 3.50-3.35 (m, 1H), 2.98 (s, 3H). |
| 232 | ¹H NMR (300 MHz, Chloroform-d) δ 9.43 (s, 1H), 8.05 (s, 1H), 7.96 (s, 1H), 7.63-7.33 (m, 4H), 4.17-4.03 (m, 1H), 3.97 (s, |
| | 3H), 3.80-3.72 (m, 4H), 3.62-3.57 (m, 1H), 3.52-3.41 (m, 1H), 3.00 (s, 3H). |
| 233 | ¹H NMR (300 MHz, Chloroform-d) δ 9.36 (s, 1H), 7.96 (s, 1H), 7.35-7.28 (m, 1H), 7.22 (s, 1H), 7.17-7.09 (m, 2H), 4.11- |
| | 4.02 (m, 1H), 3.81 (s, 3H), 3.78-3.75 (m, 1H), 3.59 (d, J = 9.0 Hz, 1H), 3.46-3.41 (m, 1H), 2.99 (s, 3H), 2.49 (s, 3H). |
| 234 | ¹H NMR (300 MHz, Chloroform-d) δ 9.50 (d, J = 12.0 Hz, 1H), 7.93 (s, 1H), 7.71 (s, 1H), 7.62-7.40 (m, 3H), 4.22-4.11 (m, |
| | 1H), 3.85-3.75 (m, 4H), 3.69-3.59 (m, 1H), 3.55-3.45 (m, 1H), 3.00 (s, 3H), 2.74 (d, J = 1.5 Hz, 3H). |
| 235 | ¹H NMR (300 MHz, Chloroform-d) δ 9.54 (s, 1H), 7.93-7.92 (m, 2H), 7.97-7.84 (m, 1H), 7.72-7.68 (m, 1H), 7.61-7.55 (m, |
| | 1H), 4.22-4.13 (m, 1H), 3.85-3.75 (m, 4H), 3.63 (d, J = 9.6 Hz, 1H), 3.50-3.44 (m, 1H), 3.07 (s, 3H), 3.00 (s, 3H). |
| 236 | ¹H NMR (300 MHz, Chloroform-d) δ 9.39 (s, 1H), 7.96 (s, 1H), 7.24-7.30 (m, 1H), 6.94-6.85 (m, 2H), 6.82-6.76 (m, 1H), |
| | 4.10-4.00 (m, 1H), 3.79-3.73 (m, 7H), 3.60 (d, J = 8.4 Hz, 1H), 3.47-3.39 (m, 1H), 2.97 (s, 3H). |
| 237 | ¹H NMR (300 MHz, Chloroform-d) δ 9.42 (s, 1H), 7.94 (s, 1H), 7.48 (d, J = 8.1 Hz, 2H), 7.36-7.19 (m, 2H), 4.07-3.98 (m, |
| | 1H), 3.80 (s, 3H), 3.79-3.73 (m, 1H), 3.53 (d, J = 9.3 Hz, 1H), 3.44-3.37 (m, 1H), 2.98 (s, 3H). |
| 238 | ¹H NMR (300 MHz, Chloroform-d) δ 9.43 (s, 1H), 7.96 (s, 1H), 7.34-7.30 (m, 2H), 7.08-7.02 (m, 2H), 4.11-4.05 (m, 1H), |
| | 3.80 (s, 3H), 3.76-3.75 (m, 1H), 3.53-3.51 (m, 1H), 3.45-3.39 (m, 1H), 2.99 (s, 3H). |
| 239 | ¹H NMR (300 MHz, Chloroform-d) δ 9.43 (s, 1H), 7.94 (s, 1H), 7.47 (d, J = 8.4 Hz, 2H), 7.22 (d, J = 8.4 Hz, 2H), 4.07-3.97 |
| | (m, 1H), 3.83-3.72 (m, 4H), 3.53 (d, J = 9.0 Hz, 1H), 3.44-3.36 (m, 1H), 2.97 (s, 3H). |
| 240 | ¹H NMR (300 MHz, Chloroform-d) δ 9.44 (s, 1H), 7.94 (s, 1H), 7.36-7.27 (m, 4H), 4.08-3.99 (m, 1H), 3.79 (s, 3H), 3.77- |
| | 3.72 (m, 1H), 3.53 (d, J = 9.0 Hz, 1H), 3.45-3.36 (m, 1H), 2.97 (s, 3H). |
| 241 | ¹H NMR (300 MHz, DMSO-d6) δ 9.85 (s, 1H), 7.76 (s, 1H), 7.24-7.12 (m, 4H), 3.94-3.84 (m, 1H), 3.81-3.72 (m, 5H), 3.42 |
| | 3.34 (m, 1H), 2.82 (s, 3H), 2.27 (s, 3H). |
| 242 | ¹H NMR (300 MHz, Chloroform-d) δ 9.40 (s, 1H), 7.97 (s, 1H), 7.25-7.13 (m, 4H), 4.07-3.98 (m, 1H), 3.85-3.70 (m, 4H), |
| | 3.56 (d, J = 8.7 Hz, 1H), 3.48-3.38 (m, 1H), 2.98 (s, 3H), 2.33 (s, 3H). |
| 243 | ¹H NMR (300 MHz, Chloroform-d) δ 9.33 (s, 1H), 7.88 (s, 1H), 7.26-7.14 (m, 4H), 4.09-4.03 (m, 1H), 3.83-3.73 (m, 4H), |
| | 3.54 (d, J = 8.4 Hz, 1H), 3.41 (s, 1H), 2.97 (s, 3H), 2.32 (s, 3H), 1.65-1.55 (m, 1H), 1.09-1.01 (m, 2H), 0.75-0.68 (m, 2H). |
| 244 | ¹H NMR (300 MHz, Chloroform-d) δ 9.41 (s, 1H), 7.97 (s, 1H), 7.31-7.22 (m, 2H), 7.22-7.12 (m, 2H), 4.10-3.98 (m, 1H), |
| | 3.79 (s, 3H), 3.78-3.68 (m, 1H), 3.57 (d, J = 9.0 Hz, 1H), 3.48-3.39 (m, 1H), 2.97 (s, 3H), 2.62 (q, J = 7.5 Hz, 2H), 1.22 (t, |
| | J = 7.5 Hz, 3H). |
| 245 | ¹H NMR (300 MHz, Chloroform-d) δ 9.42 (s, 1H), 7.98 (s, 1H), 7.32-7.14 (m, 4H), 4.16-3.98 (m, 1H), 3.78 (s, 3H), 3.77- |
| | 3.70 (m, 1H), 3.58 (d, J = 9.0 Hz, 1H), 3.49-3.37 (m, 1H), 2.98 (s, 3H), 2.91-2.80 (m, 1H), 1.23 (d, J = 6.6 Hz, 6H). |
| 246 | ¹H NMR (300 MHz, Chloroform-d) δ 9.39 (s, 1H), 7.97 (s, 1H), 7.26-7.24 (m, 2H), 7.19-7.15 (m, 2H), 4.12-4.01 (m, 1H), |
| | 3.80 (s, 3H), 3.78-3.74 (m, 1H), 3.57 (d, J = 9.0 Hz, 1H), 3.48-3.41 (m, 1H), 2.98 (s, 3H), 2.59-2.53 (m, 2H), 1.68-1.62 (m, |
| | 2H), 0.93 (t, J = 7.3 Hz, 3H). |
| 247 | ¹H NMR (300 MHz, Chloroform-d) δ 9.44 (s, 1H), 7.95 (s, 1H), 7.56-7.39 (m, 4H), 6.63 (t, J = 56.4 Hz, 1H), 4.17-4.08 (m, |
| | 1H), 3.83-3.75 (m, 4H), 3.60 (d, J = 10.5 Hz, 1H), 3.50-3.40 (m, 1H), 3.00 (s, 3H). |
| 248 | ¹H NMR (300 MHz, Chloroform-d) δ 10.06 (s, 1H), 8.11 (s, 1H), 7.63 (d, J = 8.7 Hz, 2H), 7.48 (d, J = 8.7 Hz, 2H), 4.13-4.04 |
| | (m, 1H), 3.87 (s, 3H), 3.83-3.73 (m, 1H), 3.61 (d, J = 9.6 Hz, 1H), 3.49-3.41 (m, 1H), 3.00 (s, 3H). |
| 249 | ¹H NMR (300 MHz, Chloroform-d) δ 9.46 (s, 1H), 7.94 (s, 1H), 7.62 (d, J = 8.1 Hz, 2H), 7.48 (d, J = 8.1 Hz, 2H), 4.18-4.08 |
| | (m, 1H), 3.80 (s, 3H), 3.76-3.73 (m, 1H), 3.59-3.56 (m, 1H), 3.48-3.42 (m, 1H), 3.00 (s, 3H). |
| 250 | ¹H NMR (300 MHz, Chloroform-d) δ 9.41 (s, 1H), 7.96 (s, 1H), 7.53-7.44 (m, 1H), 7.43-7.37 (m, 1H), 7.33-7.28 (m, 1H), |
| | 7.25-7.20 (m, 1H), 6.78-6.59 (m, 1H), 5.81-5.65 (m, 1H), 5.30-5.18 (m, 1H), 4.19-3.99 (m, 1H), 3.80 (s, 3H), 3.75 (d, J = |
| | 9.3 Hz, 1H), 3.61-3.50 (m, 1H), 3.48-3.70 (m, 1H), 2.98 (s, 3H). |
| 251 | ¹H NMR (300 MHz, Chloroform-d) δ 9.40 (s, 1H), 7.97 (s, 1H), 7.29-7.26 (m, 2H), 7.20-7.17 (m, 2H), 6.01-5.87 (m, 1H), |
| | 5.11-5.06 (m, 2H), 4.10-4.02 (m, 1H), 3.79 (s, 3H), 3.77-3.73 (m, 1H), 3.57 (d, J = 9.0 Hz, 1H), 3.47-3.41 (m, 1H), 3.38- |
| | 3.36 (m, 2H), 2.98 (s, 3H). |
| 252 | ¹H NMR (300 MHZ, Chloroform-d) δ 9.44 (s, 1H), 7.97 (s, 1H), 7.49-7.42 (m, 2H), 7.37-7.28 (m, 2H), 5.43-5.27 (m, 1H), |
| | 5.14-5.00 (m, 1H), 4.11-4.00 (m, 1H), 3.80 (d, J = 0.9 Hz, 3 H), 3.78-3.71 (m, 1H), 3.60 (d, J = 9.0 Hz, 1H), 3.50-3.40 (m, |
| | 1H), 2.99 (s, 3H), 2.19-2.07 (m, 3H). |
| 253 | ¹H NMR (300 MHz, Chloroform-d) δ 9.40 (s, 1H), 7.96 (s, 1H), 7.38-7.29 (m, 4H), 4.43 (s, 2H), 4.16-4.00 (m, 1H), 3.81- |
| | 3.74 (m, 4H), 3.58 (d, J = 9.0 Hz, 1H), 3.47-3.37 (m, 4H), 2.98 (s, 3H). |
| 254 | ¹H NMR (300 MHz, Chloroform-d) δ 9.44 (s, 1H), 7.95 (s, 1H), 7.39 (d, J = 8.4 Hz, 2H), 7.21 (d, J = 8.4 Hz, 2H), 4.10 (m, |
| | 1H), 3.82-3.76 (m, 4H), 3.56-3.53 (m, 1H), 3.43-3.40 (m, 1H), 2.99 (s, 3H). |
| 255 | ¹H NMR (300 MHz, Chloroform-d) δ 9.44 (s, 1H), 7.94 (s, 1H), 7.37 (d, J = 8.7 Hz, 2H), 7.20 (d, J = 8.7 Hz, 2H), 4.12-4.04 |
| | (m, 1H), 3.79 (s, 3H), 3.56 (d, J = 9.3 Hz, 1H), 3.49-3.38 (m, 1H), 2.98 (s, 3H). |
| 256 | ¹H NMR (300 MHz, DMSO-d6) δ 9.83 (s, 1H), 7.73 (s, 1H), 7.25-7.18 (m, 4H), 3.89-3.81 (m, 1H), 3.77-3.70 (m, 5H), 3.37 |
| | (d, J = 9.0 Hz, 1H), 2.79 (s, 3H), 2.43 (s, 3H). |
| 257 | ¹H NMR (300 MHz, Chloroform-d) δ 9.49 (s, 1H), 7.91 (s, 1H), 7.61 (d, J = 8.1 Hz, 2H), 7.50 (d, J = 8.1 Hz, 2H), 4.19-4.06 |
| | (m, 1H), 3.85-3.75 (m, 4H), 3.63-3.57 (m, 1H), 3.47-3.39 (m, 1H), 2.97 (s, 3H), 2.69 (s, 3H). |
| 258 | ¹H NMR (300 MHz, Chloroform-d) δ 9.52 (s, 1H), 8.03-7.87 (m, 3H), 7.58 (d, J = 8.1 Hz, 2H), 4.22-4.11 (m, 1H), 3.80 (s, |
| | 3H), 3.61 (d, J = 9.3 Hz, 1H), 3.53-3.42 (m, 1H), 3.24-3.19 (m, 1H), 3.04 (s, 3H), 3.00 (s, 3H). |
| 259 | ¹H NMR (300 MHz, Chloroform-d) δ 9.40 (s, 1H), 7.96 (s, 1H), 7.25-7.19 (m, 2H), 7.08-7.04 (m, 2H), 4.08-3.99 (m, 1H), |
| | 3.80 (s, 3H), 3.76-3.72 (m, 1H), 3.55 (d, J = 9.0 Hz, 1H), 3.45-3.39 (m, 1H), 2.97 (s, 3H), 1.92-1.82 (m, 1H), 0.97-0.91 (m, |
| | 2H), 0.69-0.64 (m, 2H). |
| 260 | ¹H NMR (300 MHz, Chloroform-d) δ 9.42 (s, 1H), 7.96 (s, 1H), 7.23-7.18 (m, 4H), 4.14-3.97 (m, 1H), 3.80-3.70 (m, 4H), |
| | 3.57 (d, J = 8.7 Hz, 1H), 3.46-3.38 (m, 1H), 3.02-2.87 (m, 4H), 2.12-1.97 (m, 2H), 1.87-1.43 (m, 6H). |
| 261 | ¹H NMR (300 MHz, DMSO-d6) δ 9.89 (s, 1H), 7.99-7.89 (m, 2H), 7.80-7.62 (m, 5H), 7.49-7.37 (m, 2H), 4.05-3.92 (m, 1H), |
| | 3.91-3.65 (m, 5H), 3.49-3.39 (m, 1H), 2.82 (s, 3H). |
| 262 | ¹H NMR (300 MHz, Chloroform-d) δ 9.01 (s, 1H), 7.58 (s, 1H), 6.89-6.67 (m, 3H), 4.11-4.02 (m, 1H), 3.84-3.75 (m, 1H), |
| | 3.71 (s, 3H), 3.52-3.33 (m, 2H), 3.00 (s, 3H). |
| 263 | ¹H NMR (300 MHZ, Chloroform-d) δ 9.42 (s, 1H), 7.95 (s, 1H), 6.93-6.84 (m, 2H), 6.77-6.64 (m, 1H), 4.13-4.02 (m, 1H), |
| | 3.85-3.70 (m, 4H), 3.54 (d, J = 9.0 Hz, 1H), 3.47-3.35 (m, 1H), 2.99 (s, 3H). |
| 264 | ¹H NMR (300 MHz, Chloroform-d) δ 9.40 (s, 1H), 7.96 (s, 1H), 6.94 (s, 1H), 6.85-6.77 (m, 2H), 4.08-4.00 (m, 1H), 3.81 (s, |
| | 3H), 3.77-3.73 (m, 1H), 3.57 (d, J = 9.0 Hz, 1H), 3.44-3.38 (m, 1H), 2.98 (s, 3H), 2.34 (s, 3H). |
| 265 | ¹H NMR (300 MHz, Chloroform-d) δ 9.39 (s, 1H), 7.95 (s, 1H), 7.28-7.22 (m, 2H), 7.10-7.07 (m, 1H), 4.06-3.97 (m, 1H), |
| | 3.81-3.72 (m, 4H), 3.57 (d, J = 8.7 Hz, 1H), 3.43-3.37 (m, 1H), 2.98 (s, 3H), 2.33 (s, 3H). |
| 266 | ¹H NMR (300 MHz, Chloroform-d) δ 9.09 (s, 1H), 7.78 (s, 1H), 6.94-6.88 (m, 3H), 4.10-4.00 (m, 1H), 3.81-3.75 (m, 4H), |
| | 3.55 (d, J = 7.8 Hz, 1H), 3.45-3.35 (m, 1H), 3.13-3.05 (m, 1H), 2.97 (s, 3H), 2.30 (s, 6H), 1.38-1.33 (m, 6H). |
| 267 | ¹H NMR (300 MHz, Chloroform-d) δ 9.39 (s, 1H), 7.94 (s, 1H), 6.92-6.87 (m, 3H), 4.05-3.96 (m, 1H), 3.80-3.70 (m, 1H), |
| | 3.61 (d, J = 8.4 Hz, 1H), 3.39 (m, 1H), 2.96 (s, 3H), 2.29 (s, 6H), 1.65 (s, 9H). |
| 268 | ¹H NMR (300 MHz, Chloroform-d) δ 9.52 (s, 1H), 8.16 (s, 1H), 6.95-6.89 (m, 3H), 4.71-4.63 (m, 2H), 4.04-3.96 (m, 1H), |
| | 3.82-3.72 (m, 1H), 3.61 (d, J = 9.0 Hz, 1H), 3.45-3.39 (m, 1H), 2.98 (s, 3H), 2.31 (s, 6H). |
| 269 | ¹H NMR (300 MHz, Chloroform-d) δ 9.69 (s, 1H), 8.26 (s, 1H), 7.30-6.89 (m, 4H), 4.00-3.88 (m, 1H), 3.80-3.70 (m, 1H), |
| | 3.65-3.60 (m, 1H), 3.47-3.39 (m, 1H), 2.99 (s, 3H), 2.32 (s, 6H). |
| 270 | ¹H NMR (300 MHz, Chloroform-d) δ 9.40 (s, 1H), 8.04 (s, 1H), 6.95-6.89 (m, 3H), 5.98-5.85 (m, 1H), 5.25-5.17 (m, 1H), |
| | 5.09 (d, J = 17.1 Hz, 1H), 4.73-4.70 (m, 2H), 4.06-3.97 (m, 1H), 3.78-3.72 (m, 1H), 3.60 (d, J = 8.7 Hz, 1H), 3.45-3.39 (m, |
| | 1H), 2.98 (s, 3H), 2.31 (s, 6H). |
| 271 | ¹H NMR (300 MHz, Chloroform-d) δ 8.99 (s, 1H), 7.59 (d, J = 2.7 Hz, 1H), 6.92-6.80 (m, 3H), 4.06-3.97 (m, 1H), 3.79-3.69 |
| | (m, 4H), 3.54 (d, J = 8.7 Hz, 1H), 3.45-3.37 (m, 1H), 2.97 (s, 3H), 2.30 (s, 6H). |
| 272 | ¹H NMR (300 MHz, Chloroform-d) δ 8.99 (s, 1H), 7.70 (s, 1H), 6.93-6.87 (m, 3H), 4.08-4.01 (m, 1H), 3.76 (s, 3H), 3.73- |
| | 3.71 (m, 1H), 3.56 (d, J = 9.0 Hz, 1H), 3.46-3.40 (m, 1H), 2.98 (s, 3H), 2.30 (s, 6H), 2.30 (s, 3H). |
| 273 | ¹H NMR (300 MHz, Chloroform-d) δ 9.01 (s, 1H), 7.74 (s, 1H), 6.94-6.90 (m, 3H), 4.09-4.01 (m, 1H), 3.77 (s, 3H), 3.76- |
| | 3.71 (m, 1H), 3.55 (d, J = 9.0 Hz, 1H), 3.45-3.39 (m, 1H), 2.97 (s, 3H), 2.67-2.60 (m, 2H), 2.30 (s, 6H), 1.19 (t, J = 9.0 Hz, |
| | 3H). |
| 274 | ¹H NMR (300 MHz, Chloroform-d) δ 9.48 (s, 1H), 7.78 (s, 1H), 7.42 (s, 1H), 6.91-6.88 (m, 3H), 4.06-3.98 (m, 1H), 3.80 (s, |
| | 3H), 3.78-3.71 (m, 1H), 3.61-3.53 (m, 1H), 3.43-3.37 (m, 1H), 2.95 (s, 3H), 2.31 (s, 6H). |
| 275 | ¹H NMR (300 MHz, Chloroform-d) δ 9.43 (s, 1H), 7.95 (s, 1H), 6.93-6.88 (m, 3H), 6.64-6.52 (m, 1H), 5.75 (d, J = 18.0 Hz, |
| | 1H), 5.59 (d, J = 12.0 Hz, 1H), 4.08-4.00 (m, 1H), 3.82-3.72 (m, 4H), 3.59 (d, J = 8.4 Hz, 1H), 3.45-3.36 (m, 1H), 2.97 (s, |
| | 3H), 2.31 (s, 6H). |
| 276 | ¹H NMR (300 MHZ, Chloroform-d) δ 9.20 (s, 1H), 7.98 (s, 1H), 6.94-6.88 (m, 3H), 5.60-5.52 (m, 1H), 5.19-5.16 (m, 1H), |
| | 4.00-4.10 (m, 1H), 3.79-3.69 (m, 4H), 3.54 (d, J = 8.7 Hz, 1H), 3.42-3.35 (m, 1H), 2.95 (s, 3H), 2.30 (s, 6H), 2.08 (s, 3H). |
| 277 | ¹H NMR (300 MHz, Chloroform-d) δ 9.30 (s, 1H), 7.89 (s, 1H), 6.93-6.88 (m, 3H), 4.11-3.98 (m, 1H), 3.87-3.76 (m, 4H), |
| | 3.58-3.55 (m, 1H), 3.46-3.36 (m, 1H), 2.97 (s, 3H), 2.31 (s, 6H), 1.68-1.61 (m, 1H), 1.10-1.00 (m, 2H), 0.79-0.68 (m, 2H). |
| 278 | ¹H NMR (300 MHz, Chloroform-d) δ 9.25 (s, 1H), 7.93 (s, 1H), 6.95-6.87 (m, 3H), 4.80-4.60 (m, 1H), 4.10-4.00 (m, 1H), |
| | 3.82-3.72 (m, 1H), 3.61-3.52 (m, 1H), 3.50-3.30 (m, 1H), 2.96 (s, 3H), 2.30 (s, 6H), 1.65-1.55 (m, 1H), 1.50-1.40 (m, 6H), |
| | 1.10-1.03 (m, 2H), 0.77-0.63 (m, 2H). |
| 279 | ¹H NMR (300 MHz, Chloroform-d) δ 9.20 (s, 1H), 7.75 (s, 1H), 6.92-6.89 (m, 3H), 4.10-3.97 (m, 1H), 3.78 (s, 3H), 3.76- |
| | 3.73 (m, 1H), 3.53-3.46 (m, 1H), 3.44-3.33 (m, 1H), 3.13-2.99 (m, 1H), 2.94 (s, 3H), 2.60 (s, 6H), 2.05-1.95 (m, 2H), 1.88- |
| | 1.66 (m, 6H). |
| 280 | ¹H NMR (300 MHz, Chloroform-d) δ 9.13 (s, 1H), 7.96 (s, 1H), 6.92-6.89 (m, 3H), 6.04-6.02 (m, 1H), 4.11-4.00 (m, 1H), |
| | 3.81 (s, 3H), 3.78-3.68 (m, 1H), 3.55-3.50 (m, 1H), 3.42-3.35 (m, 1H), 2.95 (s, 3H), 2.74-2.65 (m, 2H), 2.62-2.55 (m, 2H), |
| | 2.30 (s, 6H), 2.10-2.05 (m, 2H). |
| 281 | ¹H NMR (300 MHz, Chloroform-d) δ 9.09 (s, 1H), 8.07 (s, 1H), 7.56-7.46 (m, 2H), 7.37-7.30 (m, 2H), 6.92-6.88 (m, 3H), |
| | 4.07-3.97 (m, 1H), 3.81-3.69 (m, 4H), 3.47 (d, J = 8.4 Hz, 1H), 3.40-3.32 (m, 1H), 2.92 (s, 3H), 2.29 (s, 6H). |
| 282 | ¹H NMR (300 MHz, Chloroform-d) δ 8.52 (s, 1H), 6.92-6.88 (m, 3H), 4.15-4.00 (m, 1H), 3.85-3.70 (m, 4H), 3.55 (d, J = 8.1 |
| | Hz, 1H), 3.50-3.40 (m, 1H), 2.98 (s, 3H), 2.30 (s, 6H), 2.12 (s, 3H). |
| 283 | ¹H NMR (300 MHz, Chloroform-d) δ 9.96 (s, 1H), 8.14 (s, 1H), 6.94-6.88 (m, 3H), 3.99-3.94 (m, 1H), 3.87 (s, 3H), 3.77- |
| | 3.69 (m, 1H), 3.61 (d, J = 9.3 Hz, 1H), 3.44-3.38 (m, 1H), 2.98 (s, 3H), 2.31 (s, 6H). |
| 284 | ¹H NMR (300 MHz, Chloroform-d) δ 9.63 (s, 1H), 7.92 (s, 1H), 6.94-6.91 (m, 3H), 4.01-3.92 (m, 1H), 3.81 (s, 3H), 3.77- |
| | 3.70 (m, 1H), 3.62 (d, J = 9.0 Hz, 1H), 3.44-3.38 (m, 1H), 2.98 (s, 3H), 2.31 (s, 6H). |
| 285 | ¹H NMR (300 MHz, Chloroform-d) δ 9.46 (s, 1H), 7.34 (s, 1H), 6.93-6.88 (m, 3H), 4.09-3.94 (m, 1H), 3.78-3.71 (m, 1H), |
| | 3.64-3.55 (m, 4H), 3.45-3.39 (m, 1H), 2.97 (s, 3H), 2.31 (s, 6H). |
| 286 | ¹H NMR (300 MHz, Chloroform-d) δ 9.39 (s, 1H), 7.98 (s, 1H), 6.95 (s, 2H), 6.91 (s, 1H), 4.06-3.97 (m, 1H), 3.81 (s, 3H), |
| | 3.76-3.69 (m, 1H), 3.60 (d, J = 8.7 Hz, 1H), 3.45-3.39 (m, 1H), 2.99 (s, 3H), 2.31 (s, 6H). |
| 287 | ¹H NMR (300 MHz, Chloroform-d) δ 9.36 (s, 1H), 7.97 (s, 1H), 6.99-6.93 (m, 2H), 6.95-6.92 (m, 1H), 4.08-3.99 (m, 1H), |
| | 3.80 (s, 3H), 3.77-3.73 (m, 1H), 3.60 (d, J = 8.7 Hz, 1H), 3.46-3.40 (m, 1H), 2.98 (s, 3H), 2.60 (q, J = 7.5 Hz, 2H), 2.32 (s, |
| | 3H), 1.22 (t, J = 7.5 Hz, 3H). |
| 288 | ¹H NMR (300 MHz, Chloroform-d) δ 9.37 (s, 1H), 7.96 (s, 1H), 7.16-7.13 (m, 2H), 7.07-7.03 (m, 1H), 6.72-6.62 (m, 1H), |
| | 5.74 (d, J = 17.4 Hz, 1H), 5.24 (d, J = 10.8 Hz, 1H), 4.10-4.01 (m, 1H), 3.80-3.74 (m, 4H), 3.61 (d, J = 8.7 Hz, 1H), 3.46- |
| | 3.40 (m, 1H), 2.99 (s, 3H), 2.34 (s, 3H). |
| 289 | ¹H NMR (300 MHz, Chloroform-d) δ 9.34 (s, 1H), 7.97 (s, 1H), 6.93-6.90 (m, 1H), 6.88-6.85 (m, 1H), 6.76-6.73 (m, 1H), |
| | 4.06-3.97 (m, 1H), 3.80 (s, 3H), 3.80-3.72 (m, 1H), 3.59 (d, J = 8.7 Hz, 1H), 3.44-3.38 (m, 1H), 2.98 (s, 3H), 2.30 (s, 3H), |
| | 1.89-1.80 (m, 1H), 0.97-0.91 (m, 2H), 0.70-0.65 (m, 2H). |
| 290 | ¹H NMR (300 MHz, Chloroform-d) δ 9.43 (s, 1H), 7.92 (s, 1H), 7.40-7.38 (m, 1H), 7.32-7.26 (m, 2H), 4.19-4.09 (m, 1H), |
| | 3.91-3.74 (m, 1H), 3.57 (d, J = 9.0 Hz, 1H), 3.51-3.34 (m, 2H), 3.01 (s, 3H), 1.23-0.97 (m, 4H). |
| 291 | ¹H NMR (300 MHz, Chloroform-d) δ 9.41 (s, 1H), 7.96 (s, 1H), 7.41-7.38 (m, 1H), 7.34-7.20 (m, 2H), 4.66-4.55 (m, 1H), |
| | 4.20-4.11 (m, 1H), 3.89-3.75 (m, 1H), 3.58 (d, J = 9.0 Hz, 1H), 3.48-3.41 (m, 1H), 3.01 (s, 3H), 1.48-1.43 (m, 6H). |
| 292 | ¹H NMR (300 MHz, Chloroform-d) δ 9.73 (s, 1H), 8.21 (s, 1H), 7.41 (s, 1H), 7.36-6.95 (m, 3H), 4.17-4.08 (m, 1H), 3.86- |
| | 3.79 (m, 1H), 3.62-3.58 (m, 1H), 3.50-3.43 (m, 1H), 3.02 (s, 3H). |
| 293 | ¹H NMR (300 MHz, Chloroform-d) δ 9.40 (s, 1H), 7.85 (s, 1H), 7.42-7.37 (m, 1H), 7.32-7.26 (m, 1H), 7.25-7.20 (m, 1H), |
| | 4.24-4.12 (m, 1H), 3.93-3.77 (m, 4H), 3.60-3.52 (m, 1H), 3.47-3.36 (m, 1H), 3.00 (s, 3H), 1.68-1.61 (m, 1H), 1.10-1.00 (m, |
| | 2H), 0.79-0.68 (m, 2H) |
| 294 | ¹H NMR (300 MHz, Chloroform-d) δ 9.41 (s, 1H), 7.92 (s, 1H), 7.74-7.68 (m, 2H), 7.53 (s, 1H), 4.17-4.09 (m, 1H), 3.84- |
| | 3.77 (m, 4H), 3.58 (d, J = 9.0 Hz, 1H), 3.47-3.41 (m, 1H), 3.01 (s, 3H). |
| 295 | ¹H NMR (300 MHz, Chloroform-d) δ 9.45 (s, 1H), 7.92 (s, 1H), 7.82 (s, 2H), 7.18 (s, 1H), 4.27-4.18 (m, 1H), 3.86-3.84 (m, |
| | 1H), 3.81 (s, 3H), 3.61-3.58 (m, 1H), 3.50-3.44 (m, 1H), 3.02 (s, 3H). |
| 296 | ¹H NMR (300 MHz, Chloroform-d) δ 9.44 (s, 1H), 7.97 (s, 1H), 7.27-7.25 (m, 1H), 6.95 (d, J = 2.7 Hz, 1H), 6.83-6.79 (m, |
| | 1H), 4.39-4.30 (m, 1H), 3.87-3.74 (m, 8H), 3.46-3.40 (m, 1H), 2.97 (s, 3H). |
| 297 | ¹H NMR (300 MHz, Chloroform-d) δ 9.38 (s, 1H), 7.95 (s, 1H), 6.91 (s, 1H), 6.84-6.76 (m, 2H), 4.07-3.99 (m, 1H), 3.83- |
| | 3.73 (m, 7H), 3.57 (d, J = 8.4 Hz, 1H), 3.44-3.38 (m, 1H), 2.98 (s, 3H). |
| 298 | ¹H NMR (300 MHz, Chloroform-d) δ 9.38 (s, 1H), 7.97 (s, 1H), 6.69 (s, 1H), 6.64 (d, J = 9.0 Hz, 1H), 6.52 (d, J = 9.0 Hz, |
| | 1H), 4.09-4.00 (m, 1H), 3.81 (s, 3H), 3.80 (s, 3H), 3.76-3.73 (m, 1H), 3.56-3.53 (m, 1H), 3.44-3.38 (m, 1H), 2.99 (s, 3H). |
| 299 | ¹H NMR (300 MHz, Chloroform-d) δ 9.37 (s, 1H), 7.96 (s, 1H), 6.71-6.59 (m, 2H), 6.54-6.47 (m, 1H), 4.08-3.96 (m, 3H), |
| | 3.81 (s, 3H), 3.78-3.72 (m, 1H), 3.56 (d, J = 8.7 Hz, 1H), 3.46-3.39 (m, 1H), 2.98 (s, 3H), 1.40 (t, J = 7.5 Hz, 3H). |
| 300 | ¹H NMR (300 MHZ, Chloroform-d) δ 9.36 (s, 1H), 7.96 (s, 1H), 7.64-7.52 (m, 1H), 6.78-6.58 (m, 2H), 4.06-3.96 (m, 1H), |
| | 3.81-3.67 (m, 7H), 3.60 (d, J = 9.0 Hz, 1H), 3.46-3.37 (m, 1H), 2.99 (s, 3H), 2.18 (s, 3H). |
| 301 | ¹H NMR (300 MHz, Chloroform-d) δ 9.24 (s, 1H), 7.88 (s, 1H), 6.50-6.32 (m, 3H), 4.10-4.02 (m, 1H), 3.84-3.70 (m, 10H), |
| | 3.57-3.64 (m, 1H), 3.47-3.38 (m, 1H), 2.97 (s, 3H), 1.68-1.61 (m, 1H), 1.10-1.00 (m, 2H), 0.79-0.68 (m, 2H). |
| 302 | ¹H NMR (300 MHz, Chloroform-d) δ 9.33 (s, 1H), 7.96 (s, 1H), 6.47 (d, J = 2.4 Hz, 2H), 6.36-6.34 (m, 1H), 4.15-4.08 (m, |
| | 1H), 3.80 (s, 3H), 3.78 (s, 6H), 3.74-3.71 (m, 1H), 3.59-3.56 (m, 1H), 3.45-3.39 (m, 1H), 2.97 (s, 3H). |
| 303 | ¹H NMR (300 MHz, Chloroform-d) δ 9.85 (s, 1H), 7.71 (s, 1H), 7.60-7.45 (m, 3H), 4.31-4.21 (m, 1H), 3.97-3.74 (m, 3H), |
| | 3.72 (s, 3H), 2.80 (s, 3H), 2.52 (s, 3H). |
| 304 | ¹H NMR (300 MHz, DMSO-d6) δ 9.85 (s, 1H), 7.73 (s, 1H), 7.55-7.44 (m, 1H), 7.30-7.20 (m, 1H), 7.13-7.04 (m, 1H), 4.10- |
| | 3.97 (m, 1H), 3.90-3.83 (m, 1H), 3.76-3.70 (m, 4H), 3.42-3.35 (m, 1H), 2.80 (s, 3H). |
| 305 | ¹H NMR (300 MHz, Chloroform-d) δ 9.54 (s, 1H), 7.97 (s, 1H), 7.30-7.26 (m, 1H), 6.72-6.60 (m, 2H), 4.12-4.05 (m, 1H), |
| | 3.84-3.68 (m, 8H), 3.50-3.42 (m, 1H), 2.98 (s, 3H). |
| 306 | ¹H NMR (300 MHz, Chloroform-d) δ 9.40 (s, 1H), 7.93 (s, 1H), 7.40 (s, 1H), 7.30-7.23 (m, 2H), 4.41-4.31 (m, 1H), 3.82- |
| | 3.75 (m, 5H), 3.46-3.47 (m, 1H), 2.96 (s, 3H). |
| 307 | ¹H NMR (300 MHz, Chloroform-d) δ 9.53 (s, 1H), 7.98 (s, 1H), 7.12-7.03 (m, 1H), 6.96-6.89 (m, 2H), 4.21-4.12 (m, 1H), |
| | 3.95-3.747 (m, 8H), 3.52-3.44 (m, 1H), 2.98 (s, 3H). |
| 308 | ¹H NMR (300 MHz, Chloroform-d) δ 9.38 (s, 1H), 7.84 (s, 1H), 7.40-7.05 (m, 3H), 4.15-3.99 (m, 1H), 3.84-3.72 (m, 4H), |
| | 3.53-3.45 (m, 1H), 3.44-3.32 (m, 1H), 2.97 (s, 3H), 1.68-1.61 (m, 1H), 1.10-1.00 (m, 2H), 0.79-0.68 (m, 2H). |
| 309 | ¹H NMR (300 MHz, Chloroform-d) δ 9.44 (s, 1H), 7.95 (s, 1H), 7.49-7.42 (m, 2H), 7.24-7.20 (m, 1H), 4.09-4.00 (m, 1H), |
| | 3.82 (s, 3H), 3.76-3.75 (m, 1H), 3.54-3.51 (m, 1H), 3.46-3.39 (m, 1H), 3.01 (s, 3H). |
| 310 | ¹H NMR (300 MHz, Chloroform-d) δ 9.43 (s, 1H), 7.93 (s, 1H), 7.40-7.37 (m, 1H), 7.25-7.21 (m, 1H), 7.16-7.09 (m, 1H), |
| | 4.10-3.97 (m, 1H), 3.84-3.71 (m, 4H), 3.51 (d, J = 9.3 Hz, 1H), 3.44-3.36 (m, 1H), 2.98 (s, 3H). |
| 311 | ¹H NMR (300 MHz, Chloroform-d) δ 9.43 (s, 1H), 7.94 (s, 1H), 7.41-7.37 (m, 1H), 7.25-7.18 (m, 1H), 7.17-7.09 (m, 1H), |
| | 4.08-3.97 (m, 1H), 3.85-3.73 (m, 4H), 3.53 (d, J = 8.7 Hz, 1H), 3.46-3.37 (m, 1H), 2.99 (s, 3H). |
| 312 | ¹H NMR (300 MHz, Chloroform-d) δ 9.33 (s, 1H), 7.87 (s, 1H), 7.18-6.94 (m, 3H), 4.12-4.01 (m, 1H), 3.86-3.75 (m, 4H), |
| | 3.55-3.48 (m, 1H), 3.43-3.34 (m, 1H), 2.97 (s, 3H), 2.24 (s, 3H), 1.68-1.61 (m, 1H), 1.10-1.00 (m, 2H), 0.79-0.68 (m, 2H). |
| 313 | ¹H NMR (300 MHz, Chloroform-d) δ 9.42 (s, 1H), 7.97 (s, 1H), 7.20-7.13 (m, 1H), 7.06-6.97 (m, 2H), 4.09-3.99 (m, 1H), |
| | 3.81 (s, 3H), 3.76-3.73 (m, 1H), 3.56-3.52 (m, 1H), 3.46-3.38 (m, 1H), 2.98 (s, 3H), 2.25 (s, 3H). |
| 314 | ¹H NMR (300 MHz, Chloroform-d) δ 9.42 (s, 1H), 7.94 (s, 1H), 7.17-6.97 (m, 3H), 4.04-3.91 (m, 1H), 3.81-3.67 (m, 4H), |
| | 3.58 (d, J = 9.0 Hz, 1H), 3.48-3.34 (m, 1H), 2.95 (s, 3H), 2.23 (s, 3H), 2.21 (s, 3H). |
| 315 | ¹H NMR (300 MHz, Chloroform-d) δ 9.44 (s, 1H), 7.94 (s, 1H), 7.63-7.57 (m, 1H), 7.25-7.20 (m, 2H), 4.18-4.09 (m, 1H), |
| | 3.84-3.77 (m, 4H), 3.57 (d, J = 9.0 Hz, 1H), 3.52-4.45 (m, 1H), 3.01 (s, 3H). |
| 316 | ¹H NMR (300 MHz, Chloroform-d) δ 9.42 (s, 1H), 7.94 (s, 1H), 7.55 (d, J = 7.8 Hz, 1H), 7.07-6.87 (m, 2H), 4.16-4.07 (m, |
| | 1H), 3.93 (s, 3H), 3.83-3.76 (m, 4H), 3.60 (d, J = 9.0 Hz, 1H), 3.51-3.38 (m, 1H), 3.00 (s, 3H). |
| 317 | ¹H NMR (300 MHz, Chloroform-d) δ 9.40 (s, 1H), 7.95 (s, 1H), 7.52 (d, J = 8.1 Hz, 1H), 6.91 (s, 1H), 6.82 (d, J = 8.1 Hz, |
| | 1H), 4.10-4.01 (m, 1H), 3.92 (s, 3H), 3.81 (s, 3H), 3.79-3.75 (m, 1H), 3.57 (d, J = 9.3 Hz, 1H), 3.46-3.40 (m, 1H), 3.01 (s, |
| | 3H). |
| 318 | ¹H NMR (300 MHz, Chloroform-d) δ 9.36 (s, 1H), 7.95 (s, 1H), 7.08-7.01 (m, 1H), 6.99-6.95 (m, 1H), 6.90-6.82 (m, 1H), |
| | 4.60-4.52 (m, 1H), 4.08-3.99 (m, 1H), 3.81 (s, 3H), 3.78-3.73 (m, 1H), 3.52 (d, J = 9.0 Hz, 1H), 3.45-3.39 (m, 1H), 2.99 (s, |
| | 3H), 1.36 (d, J = 6.3 Hz, 6H). |
| 319 | ¹H NMR (300 MHz, Chloroform-d) δ 9.40 (s, 1H), 7.95 (s, 1H), 7.34 (d, J = 8.1 Hz 1H), 6.96-6.82 (m, 2H), 4.11-4.01 (m, |
| | 1H), 3.92 (s, 3H), 3.82-3.75 (m, 4H), 3.56 (d, J = 9.0 Hz, 1H), 3.48-3.38 (m, 1H), 2.99 (s, 3H). |
| 320 | ¹H NMR (300 MHz, Chloroform-d) δ 9.41 (s, 1H), 7.95 (s, 1H), 7.12-7.01 (m, 1H), 6.99-6.93 (m, 1H), 6.88-6.82 (m, 1H), |
| | 4.08-3.99 (m, 1H), 3.90 (s, 3H), 3.85-3.72 (m, 4H), 3.55 (d, J = 9.0 Hz, 1H), 3.47-3.38 (m, 1H), 2.99 (s, 3H). |
| 321 | ¹H NMR (300 MHz, Chloroform-d) δ 9.39 (s, 1H), 7.97 (s, 1H), 7.15-7.07 (m, 1H), 6.85-6.79 (m, 2H), 4.10-4.00 (m, 1H), |
| | 3.90-3.70 (m, 7H), 3.60 (d, J = 9.0 Hz, 1H), 3.48-3.41 (m, 1H), 3.00 (s, 3H), 2.19 (s, 3H). |
| 322 | ¹H NMR (300 MHz, Chloroform-d) δ 9.49 (s, 1H), 7.96 (s, 1H), 7.03-6.95 (m, 1H), 6.89-6.85 (m, 1H), 6.79-6.73 (m, 1H), |
| | 4.13-4.06 (m, 1H), 3.80 (s, 3H), 3.78 (s, 3H), 3.77-3.74 (m, 2H), 3.50-3.43 (m, 1H), 2.98 (s, 3H). |
| 323 | ¹H NMR (300 MHz, Chloroform-d) δ 9.05 (s, 1H), 7.56 (s, 1H), 7.05-6.94 (m, 2H), 4.08-3.98 (m, 1H), 3.81-3.67 (m, 4H), |
| | 3.48-3.36 (m, 2H), 2.98 (s, 3H). |
| 324 | ¹H NMR (300 MHz, Chloroform-d) δ 9.44 (s, 1H), 7.91 (s, 1H), 7.06-6.93 (m, 2H), 4.05-3.96 (m, 1H), 3.79-3.73 (m, 4H), |
| | 3.48 (d, J = 9.3 Hz, 1H), 3.42-3.34 (m, 1H), 2.98 (s, 3H). |
| 325 | ¹H NMR (300 MHz, Chloroform-d) δ 9.54 (s, 1H), 7.94 (s, 1H), 7.13-6.89 (m, 2H), 4.19-4.10 (m, 1H), 3.81 (s, 3H), 3.76- |
| | 3.70 (m, 2H), 3.46 (t, J = 9.3 Hz, 1H), 2.99 (s, 3H). |
| 326 | ¹H NMR (300 MHz, Chloroform-d) δ 9.46 (s, 1H), 7.91 (s, 1H), 7.70-7.60 (m, 2H), 4.14-4.04 (m, 1H), 3.81 (s, 3H), 3.79- |
| | 3.75 (m, 1H), 3.56 (d, J = 9.0 Hz, 1H), 3.46-3.39 (m, 1H), 3.01 (s, 3H). |
| 327 | ¹H NMR (300 MHz, Chloroform-d) δ 9.49 (s, 1H), 7.98 (s, 1H), 6.96 (d, J = 8.7 Hz, 1H), 6.63 (d, J = 8.7 Hz, 1H), 4.16-4.08 |
| | (m, 1H), 3.97 (s, 3H), 3.85 (s, 3H), 3.84 (s, 3H), 3.80 (s, 3H), 3.76-3.69 (m, 2H), 3.36-3.30 (m, 1H), 2.97 (s, 3H). |
| 328 | ¹H NMR (300 MHz, Chloroform-d) δ 9.54 (s, 1H), 7.95 (s, 1H), 7.44 (s, 1H), 7.15-7.01 (m, 1H), 4.71-4.60 (m, 1H), 3.97- |
| | 3.93 (m, 1H), 3.80 (s, 3H), 3.73-3.62 (m, 1H), 3.55-3.46 (m, 1H), 2.99 (s, 3H). |
| 329 | ¹H NMR (300 MHz, Chloroform-d) δ 9.49 (s, 1H), 7.92 (s, 1H), 7.54-7.49 (m, 1H), 7.30-7.25 (m, 1H), 4.53-4.45 (m, 1H), |
| | 3.93-3.83 (m, 2H), 3.79 (s, 3H), 3.45-3.36 (m, 1H), 2.99 (s, 3H). |
| 330 | ¹H NMR (300 MHz, Chloroform-d) δ 9.48 (s, 1H), 7.94 (s, 1H), 7.66 (d, J = 8.4 Hz, 1H), 7.49 (d, J = 8.4 Hz, 1H), 4.59-4.47 |
| | (m, 1H), 3.95-3.85 (m, 2H), 3.81 (s, 3H), 3.48-3.35 (m, 1H), 3.00 (s, 3H), 2.42 (s, 3H). |
| 331 | ¹H NMR (300 MHz, Chloroform-d) δ 9.56 (s, 1H), 7.96 (s, 1H), 7.20-7.12 (m, 1H), 7.09-6.97 (m, 1H), 4.19-4.07 (m, 1H), |
| | 3.96 (s, 3H), 3.85-3.68 (m, 5H), 3.52-3.39 (m, 1H), 2.99 (s, 3H). |
| 332 | ¹H NMR (300 MHz, Chloroform-d) δ 9.52 (s, 1H), 7.92 (s, 1H), 6.95-6.83 (m, 2H), 4.21-4.10 (m, 1H), 3.80 (s, 3H), 3.77- |
| | 3.70 (m, 2H), 3.51-3.37 (m, 1H), 2.98 (s, 3H). |
| 333 | ¹H NMR (300 MHz, Chloroform-d) δ 9.41 (s, 1H), 7.99 (s, 1H), 7.37-7.31 (m, 1H), 7.21 (s, 1H), 7.13-7.07 (m, 1H), 4.37- |
| | 3.97 (m, 1H), 3.82-3.76 (m, 4H), 3.54-3.46 (m, 2H), 2.99 (s, 3H). |
| 334 | ¹H NMR (300 MHz, Chloroform-d) δ 9.36 (s, 1H), 7.99 (s, 1H), 7.21 (d, J = 4.5 Hz, 1H), 7.05 (d, J = 3.6 Hz, 1H), 6.99-6.95 |
| | (m, 1H), 4.43-4.34 (m, 1H), 3.88-3.82 (m, 1H), 3.80 (s, 3H), 3.58-3.52 (m, 2H), 2.98 (s, 3H). |
| 335 | ¹H NMR (300 MHz, Chloroform-d) δ 9.36 (s, 1H), 7.99 (s, 1H), 6.80 (d, J = 3.3 Hz, 1H), 6.60 (d, J = 3.3 Hz, 1H), 4.31-4.22 |
| | (m, 1H), 3.82-3.74 (m, 4H), 3.54-3.45 (m, 2H), 2.96 (s, 3H), 2.43 (s, 3H). |
| 336 | ¹H NMR (300 MHz, Chloroform-d) δ 9.89 (s, 1H), 7.76 (s, 1H), 7.31 (d, J = 5.1 Hz, 1H), 6.82 (d, J = 5.1 Hz, 1H) 4.30-4.21 |
| | (m, 1H), 3.83-3.64 (m, 6H), 2.80 (s, 3H), 2.10 (s, 3H). |
| 337 | ¹H NMR (300 MHz, Chloroform-d) δ 9.77 (s, 1H), 7.72 (s, 1H), 6.72 (s, 1H), 3.95-3.87 (m, 1H), 3.74 (s, 3H), 3.65-3.58 (m, |
| | 2H), 3.31-3.27 (m, 1H), 2.78 (s, 3H), 2.32 (s, 3H), 2.23 (s, 3H). |
| 338 | ¹H NMR (300 MHz, Chloroform-d) δ 9.35 (s, 1H), 8.00 (s, 1H), 6.83 (d, J = 3.0 Hz, 1H), 6.64 (d, J = 3.0 Hz, 1H), 4.27-4.25 |
| | (m, 1H), 3.81 (s, 3H), 3.78-3.75 (m, 1H), 3.55-3.48 (m, 2H), 2.97 (d, J = 0.9 Hz, 3H), 2.83-2.71 (q, J = 9.0 Hz, 2H), 1.28 (t, |
| | J = 9.0 Hz, 3H). |
| 339 | ¹H NMR (300 MHz, Chloroform-d) δ 9.36 (s, 1H), 7.98 (s, 1H), 6.84-6.75 (m, 2H), 4.31-4.18 (m, 1H), 3.88-3.72 (m, 4H), |
| | 3.60-3.43 (m, 2H), 2.97 (s, 3H). |
| 340 | ¹H NMR (300 MHz, Chloroform-d) δ 9.57 (s, 1H), 8.01 (s, 1H), 7.12 (t, J = 59.4 Hz, 1H), 6.27 (s, 1H), 4.07-4.04 (m, 1H), |
| | 3.82 (s, 3H), 3.73-3.70 (m, 3H), 2.97 (s, 3H), 2.44 (s, 3H). |
| 341 | ¹H NMR (300 MHz, DMSO-d6) δ 9.92 (s, 1H), 7.75 (s, 1H), 6.81 (s, 1H), 4.17-4.05 (m, 1H), 3.84 (s, 3H), 3.83-3.77 (m, 2H), |
| | 3.77-3.70 (m, 4H), 2.80 (s, 3H). |
| 342 | ¹H NMR (300 MHz, Chloroform-d) δ 9.54 (s, 1H), 8.00 (s, 1H), 7.11 (t, J = 59.4 Hz, 1H), 6.28 (s, 1H), 4.13-4.02 (m, 1H), |
| | 3.82 (s, 3H), 3.72-3.69 (m, 3H), 2.96 (s, 3H), 2.85-2.78 (m, 2H), 1.29 (t, J = 7.8 Hz, 3H). |
| 343 | ¹H NMR (300 MHz, Chloroform-d) δ 9.65 (s, 1H), 7.97 (s, 1H), 7.56-7.52 (m, 1H), 6.99 (d, J = 7.8 Hz, 1H), 6.63 (d, J = 7.8 |
| | Hz, 1H), 4.12-4.06 (m, 1H), 3.89 (s, 3H), 3.80 (s, 3H), 3.79-3.75 (m, 1H), 3.67-3.62 (m, 2H), 2.97 (s, 3H). |
| 344 | ¹H NMR (300 MHz, Chloroform-d) δ 9.47 (s, 1H), 8.76 (s, 1H), 7.91-7.88 (m, 2H), 7.69 (d, J = 6.5 Hz, 1H), 4.22-4.13 (m, |
| | 1H), 3.88-3.8 (m, 4H), 3.60 (d, J = 9.6 Hz, 1H), 3.53-3.46 (m, 1H), 3.03 (s, 3H). |
| 347 | ¹H NMR (300 MHz, Chloroform-d) δ 9.44 (s, 1H), 7.98 (s, 1H), 6.32 (d, J = 3.0 Hz, 1H), 6.11 (d, J = 3.0 Hz, 1H), 4.16-4.08 |
| | (m, 1H), 3.82 (s, 3H), 3.68-3.65 (m, 2H), 3.59-3.53 (m, 1H), 2.97 (s, 3H). |
| 348 | ¹H NMR (300 MHz, Chloroform-d) δ 9.44 (s, 1H), 7.97 (s, 1H), 6.27-6.24 (m, 2H), 4.34 (s, 2H), 4.20-4.11 (m, 1H), 3.80 (s, |
| | 3H), 3.72-3.54 (m, 3H), 3.35 (s, 3H), 2.95 (s, 3H). |
| 352 | ¹H NMR (300 MHz, Chloroform-d) δ 9.44 (s, 1H), 8.00 (s, 1H), 6.64-6.63 (m, 1H), 6.56-6.54 (m, 1H), 6.06-6.04 (m, 1H), |
| | 4.00-3.92 (m, 1H), 3.80 (s, 3H), 3.72-3.66 (m, 1H), 3.61 (s, 3H), 3.44-3.38 (m, 2H), 2.95 (s, 3H). |
| 353 | ¹H NMR (300 MHz, Chloroform-d) δ 9.41 (s, 1H), 7.97 (s, 1H), 7.88-7.78 (m, 4H), 7.51-7.41 (m, 3H), 4.35-4.21 (m, 1H), |
| | 3.92-3.83 (m, 1H), 3.80 (s, 3H), 3.72 (d, J = 9.0 Hz, 1H), 3.59-3.51 (m, 1H), 3.02 (s, 3H). |
| 354 | ¹H NMR (300 MHz, Chloroform-d) δ 9.37 (s, 1H), 7.98 (s, 1H), 7.52 (d, J = 8.1 Hz, 1H), 7.33 (d, J = 8.1 Hz, 1H), 7.27-7.21 |
| | (m, 1H), 7.15-7.01 (m, 2H), 4.41-4.29 (m, 1H), 3.88-3.72 (m, 8H), 3.70-3.56 (m, 1H), 3.02 (s, 3H). |
| 355 | ¹H NMR (300 MHz, Chloroform-d) δ 9.41 (s, 1H), 7.96 (s, 1H), 7.66-7.55 (m, 2H), 7.49 (d, J = 8.4 Hz, 1H), 7.29-7.23 (m, |
| | 1H), 6.77-6.67 (m, 1H), 4.24-4.11 (m, 1H), 3.88-3.71 (m, 4H), 3.63 (d, J = 8.7 Hz, 1H), 3.55-3.45 (m, 1H), 3.00 (s, 3H). |
| 356 | ¹H NMR (300 MHz, Chloroform-d) δ 9.45 (s, 1H), 7.98 (s, 1H), 7.56-7.48 (m, 1H), 7.46-7.38 (m, 1H), 7.26-7.14 (m, 2H), |
| | 6.70 (s, 1H), 4.39-4.26 (m, 1H), 3.90-3.83 (m, 1H), 3.80 (s, 3H), 3.77-3.64 (m, 2H), 2.99 (s, 3H). |
| 357 | ¹H NMR (300 MHz, Chloroform-d) δ 9.45 (s, 1H), 7.94 (s, 1H), 7.15-6.99 (m, 3H), 4.11-4.01 (m, 1H), 3.83-3.75 (m, 4H), |
| | 3.58-3.36 (m, 2H), 2.99 (s, 3H). |
| 359 | ¹H NMR (300 MHz, DMSO-d6) δ 9.89 (s, 1H), 8.01-7.97 (m, 1H), 7.86-7.83 (m, 1H), 7.75-7.71 (m, 2H), 7.44-7.36 (m, 2H), |
| | 4.38-4.30 (m, 1H), 3.98-3.90 (m, 2H), 3.75 (s, 3H), 3.55-3.48 (m, 1H), 2.85 (s, 3H). |
| 360 | ¹H NMR (300 MHZ, Chloroform-d) δ 9.35 (s, 1H), 8.18-8.13 (m, 1H), 7.97-7.91 (m, 1H), 7.77-7.63 (m, 1H), 7.40-7.34 (m, |
| | 1H), 7.22-7.08 (m, 2H), 7.18-7.10 (m, 2H), 6.93-6.90 (m, 1H), 4.19-3.93 (m, 1H), 3.88-3.76 (m, 4H), 3.85-3.78 (m, 1H), |
| | 3.53-3.37 (m, 1H), 2.96 (s, 3H). |
| 361 | ¹H NMR (300 MHz, Chloroform-d) δ 9.43-9.31 (m, 1H), 7.95 (s, 1H), 7.84-7.78 (m, 1H), 7.42-7.31 (m, 2H), 7.23-7.03 (m, |
| | 3H), 4.18-4.05 (m, 1H), 3.88-3.84 (m, 1H), 3.81 (d, J = 3.0 Hz, 3H), 3.59-3.43 (m, 2H), 2.99-2.98 (m, 3H). |
| 362 | ¹H NMR (300 MHz, Chloroform-d) δ 9.39 (s, 1H), 7.97 (s, 1H), 7.43 (d, J = 7.8 Hz, 2H), 7.29 (d, J = 7.8 Hz, 2H), 6.20-6.14 |
| | (m, 1H), 4.08-4.06 (m, 1H), 3.80 (s, 3H), 3.77-3.74 (m, 1H), 3.58-3.56 (m, 1H), 3.47-3.44 (m, 1H), 2.99 (s, 3H), 3.68-3.66 |
| | (m, 2H), 3.55-3.48 (m, 2H), 2.09-1.96 (m, 2H). |
| 363 | ¹H NMR (300 MHz, Chloroform-d) δ 8.67 (s, 1H), 7.55-7.42 (m, 4H), 4.23-4.15 (m, 1H), 4.05 (q, J = 7.5 Hz, 2H), 3.85-3.79 |
| | (m, 1H), 3.57-3.53 (m, 1H), 3.49-3.40 (m, 1H), 2.98 (s, 3H), 2.11 (s, 3H), 1.35 (t, J = 7.5 Hz, 3H). |
| 367 | ¹H NMR (300 MHz, Chloroform-d) δ 7.00-6.84 (m, 4H), 4.13-4.00 (m, 1H), 3.84 (s, 3H), 3.63-3.38 (m, 5H), 2.97 (s, 3H), |
| | 2.31 (s, 6H), 0.94-0.55 (m, 1H), 0.33-0.05 (m, 4H). |
| 378 | ¹H NMR (300 MHz, Chloroform-d) δ 9.35 (s, 1H), 7.85 (s, 1H), 7.25-7.21 (m, 1H), 7.15-7.05 (m, 3H), 4.48 (s, 2H), 4.13- |
| | 4.03 (m, 1H), 3.84-3.73 (m, 4H), 3.56 (d, J = 8.4 Hz, 1H), 3.46-3.39 (m, 1H), 3.37 (s, 3H), 2.97 (s, 3H), 2.35 (s, 3H). |
| 379 | ¹H NMR (300 MHz, Chloroform-d) δ 9.21 (s, 1H), 7.97 (s, 1H), 7.26-7.18 (m, 1H), 7.15-7.05 (m, 3H), 5.58-5.54 (m, 1H), |
| | 5.20-5.16 (m, 1H), 4.13-4.03 (m, 1H), 3.80-3.73 (m, 4H), 3.54 (d, J = 8.4 Hz, 1H), 3.45-3.37 (m, 1H), 2.95 (s, 3H), 2.34 (s, |
| | 3H), 2.08 (s, 3H). |
| 380 | ¹H NMR (300 MHz, Chloroform-d) δ 9.10 (s, 1H), 7.77 (s, 1H), 7.26-7.19 (m, 1H), 7.16-7.03 (m, 3H), 4.15-4.03 (m, 1H), |
| | 3.81-3.74 (m, 4H), 3.55 (d, J = 8.4 Hz, 1H), 3.47-3.40 (m, 1H), 3.15-3.02 (m, 1H), 2.97 (s, 3H), 2.35 (s, 3H), 1.38-1.33 (m, |
| | 6H). |
| 381 | ¹H NMR (300 MHz, Chloroform-d) δ 9.41 (s, 1H), 8.04 (s, 1H), 7.26-7.22 (m, 1H), 7.16-7.06 (m, 3H), 5.99-5.86 (m, 1H), |
| | 5.25-5.19 (m, 1H), 5.13-5.05 (m, 1H), 4.73-4.67 (m, 2H), 4.09-4.01 (m, 1H), 3.80-3.73 (m, 1H), 3.60 (d, J = 8.7 Hz, 1H), |
| | 3.47-3.40 (m, 1H), 2.99 (s, 3H), 2.35 (s, 3H). |
| 382 | ¹H NMR (300 MHz, Chloroform-d) δ 9.58 (s, 1H), 8.25 (s, 1H), 7.57-7.54 (m, 2H), 7.50-7.45 (m, 2H), 7.42-7.36 (m, 1H), |
| | 7.29-7.27 (m, 1H), 7.17-7.14 (m, 2H), 7.11-7.09 (m, 1H), 4.11-4.04 (m, 1H), 3.84-3.75 (m, 1H), 3.65 (d, J = 9.0 Hz, 1H), |
| | 3.49-3.43 (m, 1H), 3.00 (s, 3H), 2.37 (s, 3H). |
| 384 | ¹H NMR (300 MHz, Chloroform-d) δ 9.38 (s, 1H), 7.95 (s, 1H), 7.25-7.21 (m, 1H), 7.17-7.05 (m, 3H), 4.11-4.02 (m, 1H), |
| | 3.82-3.74 (m, 1H), 3.64-3.58 (m, 1H), 3.45-3.39 (m, 7.4 Hz, 1H), 2.98 (s, 3H), 2.35 (s, 3H), 1.66 (s, 9H). |
| 385 | ¹H NMR (300 MHz, Chloroform-d) δ 9.37 (s, 1H), 7.99 (s, 1H), 7.25-7.23 (m, 1H), 7.15-7.07 (m, 3H), 4.18-4.01 (m, 3H), |
| | 3.80-3.74 (m, 1H), 3.61-3.58 (m, 1H), 3.46-3.41 (m, 1H), 2.98 (s, 3H), 2.35 (s, 3H), 1.40 (t, J = 7.2 Hz, 3H). |
| 386 | ¹H NMR (300 MHz, Chloroform-d) δ 9.41 (s, 1H), 8.06 (s, 1H), 7.27-7.22 (m, 1H), 7.14-7.07 (m, 3H), 4.28 (t, J = 5.4 Hz, |
| | 2H), 4.09-3.99 (m, 1H), 3.80-3.71 (m, 3H), 3.61 (d, J = 8.7 Hz, 1H), 3.47-3.41 (m, 1H), 3.30 (s, 3H), 2.98 (s, 3H), 2.35 (s, |
| | 3H). |
| 387 | ¹H NMR (300 MHz, Chloroform-d) δ 9.44 (s, 1H), 8.02 (s, 1H), 7.27-7.23 (m, 1H), 7.14-7.07 (m, 3H), 4.22-4.17 (m, 2H), |
| | 4.08-3.97 (m, 3H), 3.80-3.74 (m, 1H), 3.61 (d, J = 8.7 Hz, 1H), 3.47-3.41 (m, 1H), 3.17-3.13 (m, 1H), 2.98 (s, 3H), 2.36 (s, |
| | 3H). |
| 388 | ¹H NMR (300 MHz, Chloroform-d) δ 8.98 (s, 1H), 7.60 (d, J = 2.4 Hz, 1H), 7.26-7.21 (m, 1H), 7.16-7.05 (m, 3H), 4.12-4.02 |
| | (m, 1H), 3.80-3.74 (m, 1H), 3.70 (s, 3H), 3.54 (d, J = 8.7 Hz, 1H), 3.47-3.41 (m, 1H), 2.98 (s, 3H), 2.34 (s, 3H). |
| 389 | ¹H NMR (300 MHz, Chloroform-d) δ 9.42 (s, 1H), 7.94 (s, 1H), 7.26-7.15 (m, 2H), 7.15-7.06 (m, 2H), 4.10-4.00 (m, 1H), |
| | 3.82 (s, 3H), 3.78-3.71 (m, 1H), 3.60 (d, J = 9.0 Hz, 1H), 3.50-3.38 (m, 1H), 2.99 (s, 3H), 2.35 (s, 3H). |
| 390 | ¹H NMR (300 MHz, Chloroform-d) δ 9.98 (s, 1H), 8.20 (s, 1H), 7.26-7.23 (m, 1H), 7.15-7.08 (m, 3H), 4.04-3.95 (m, 1H), |
| | 3.88 (s, 3H), 3.78-3.70 (m, 1H), 3.62-3.59 (m, 1H), 3.45-3.39 (m, 1H), 2.98 (s, 3H), 2.36 (s, 3H). |
| 391 | ¹H NMR (300 MHz, Chloroform-d) δ 9.92 (s, 1H), 7.95 (s, 1H), 7.26-7.22 (m, 1H), 7.15-7.06 (m, 3H), 4.10-74.02 (m, 1H), |
| | 3.97 (s, 3H), 3.82-3.76 (m, 1H), 3.63-3.60 (m, 1H), 3.48-3.42 (m, 1H), 2.99 (s, 3H), 2.36 (s, 3H). |
| 393 | ¹H NMR (300 MHZ, Chloroform-d) δ 9.61 (s, 1H), 8.09 (s, 1H), 7.26-7.23 (m, 1H), 7.15-7.07 (m, 3H), 4.13-4.04 (m, 1H), |
| | 3.92 (s, 3H), 3.81-3.74 (m, 1H), 3.63-3.61 (m, 1H), 3.46-3.40 (m, 1H), 2.98 (s, 3H), 2.35 (s, 3H), 2.28 (s, 3H). |
| 394 | ¹H NMR (300 MHz, Chloroform-d) δ 9.28 (s, 1H), 7.90 (s, 1H), 7.26-7.21 (m, 1H), 7.16-7.05 (m, 3H), 4.20 (q, J = 7.2 Hz, |
| | 2H), 4.13-4.05 (m, 1H), 3.82-3.74 (m, 1H), 3.58 (d, J = 8.4 Hz, 1H), 3.44-3.39 (m, 1H), 2.97 (s, 3H), 2.35 (s, 3H), 1.85-1.75 |
| | (m, 1H), 1.41 (t, J = 7.2 Hz, 3H), 1.09-1.02 (m, 2H), 0.79-0.65 (m, 2H). |
| 395 | ¹H NMR (300 MHz, Chloroform-d) δ 9.56 (s, 1H), 8.16 (s, 1H), 7.28-7.25 (m, 1H), 7.21 (t, J = 59.1 Hz, 1H), 7.16-7.07 (m, |
| | 3H), 4.11-4.01 (m, 1H), 3.82-3.73 (m, 1H), 3.60 (d, J = 8.7 Hz, 1H), 3.47-3.39 (m, 1H), 2.99 (s, 3H), 2.36 (s, 3H), 1.84-1.75 |
| | (m, 1H), 1.17-1.10 (m, 2H), 0.92-0.78 (m, 2H). |
| 396 | ¹H NMR (300 MHz, Chloroform-d) δ 9.03 (s, 1H), 7.71 (s, 1H), 7.26-7.21 (m, 1H), 7.15-7.04 (m, 3H), 4.14-4.04 (m, 1H), |
| | 3.82-3.74 (m, 1H), 3.68 (s, 3H), 3.54 (d, J = 8.4 Hz, 1H), 3.45-3.39 (m, 1H), 2.97 (s, 3H), 2.82-2.79 (s, 6H), 2.35 (s, 3H). |
| 397 | ¹H NMR (300 MHz, DMSO-d6) δ 9.57 (s, 1H), 7.82 (s, 1H), 7.24-7.17 (m, 1H), 7.12-7.03 (m, 3H), 3.92-3.81 (m, 1H), 3.76- |
| | 3.65 (m, 5H), 3.41-3.34 (m, 1H), 2.80 (s, 3H), 2.27 (s, 3H), 2.04 (s, 3H). |
| 398 | ¹H NMR (300 MHz, Chloroform-d) δ 9.58 (s, 1H), 7.84 (s, 1H), 7.28-7.22 (m, 1H), 7.16-7.07 (m, 3H), 4.08-4.00 (m, 1H), |
| | 3.81-3.76 (m, 1H), 3.74 (s, 3H), 3.61 (d, J = 8.4 Hz, 1H), 3.46-3.41 (m, 1H), 2.98 (d, J = 0.8 Hz, 3H), 2.35 (s, 3H), 1.78-1.71 |
| | (m, 1H), 0.97-0.91 (m, 2H), 0.87-0.78 (m, 2H). |
| 399 | ¹H NMR (300 MHz, Chloroform-d) δ 9.40 (s, 1H), 8.03 (s, 1H), 7.26-7.21 (m, 1H), 7.18-7.05 (m, 3H), 4.09-4.01 (m, 1H), |
| | 3.92-3.85 (s, 3H), 3.82-3.72 (m, 1H), 3.61 (d, J = 8.7 Hz, 1H), 3.46-3.40 (m, 1H), 2.99 (s, 3H), 2.35 (s, 3H). |
| 402 | ¹H NMR (300 MHZ, Chloroform-d) δ 9.57 (s, 1H), 7.96 (s, 1H), 7.60-7.44 (m, 4H), 7.42-7.05 (m, 3H), 3.98-3.94 (m, 2H), |
| | 3.86-3.79 (m, 1H), 3.68-3.62 (m, 1H), 3.48-3.42 (m, 1H), 2.98 (s, 3H). |
| 403 | ¹H NMR (300 MHz, Chloroform-d) δ 9.45 (s, 1H), 8.01 (s, 1H), 7.60-7.50 (m, 4H), 7.16 (t, J = 59.1 Hz, 1H), 4.21-4.11 (m, |
| | 1H), 3.87-3.77 (m, 1H), 3.60 (d, J = 9.0 Hz, 1H), 3.51-3.42 (m, 1H), 3.00 (s, 3H), 2.91-2.81 (m, 2H), 1.28 (d, J = 7.5 Hz, |
| | 3H). |
| 404 | ¹H NMR (300 MHz, Chloroform-d) δ 9.94 (s, 1H), 8.13 (s, 1H), 7.60-7.50 (m, 4H), 4.19-4.10 (m, 1H), 3.94 (s, 3H), 3.84- |
| | 3.77 (m, 1H), 3.60 (d, J = 9.3 Hz, 1H), 3.48-3.40 (m, 1H), 3.00 (s, 3H). |
| 405 | ¹H NMR (300 MHz, Chloroform-d) δ 10.05 (s, 1H), 8.18 (s, 1H), 7.60-7.47 (m, 4H), 4.15-4.06 (m, 1H), 3.89 (s, 3H), 3.83- |
| | 3.76 (m, 1H), 3.62-3.59 (m, 1H), 3.48-3.42 (m, 1H), 3.00 (s, 3H). |
| 406 | ¹H NMR (300 MHz, Chloroform-d) δ 9.77 (s, 1H), 8.20 (s, 1H), 7.60-7.50 (m, 4H), 7.17 (t, J = 57.0 Hz, 1H), 6.88-6.78 (m, |
| | 1H), 5.86-5.92 (m, 1H), 5.75-5.71 (m, 1H), 4.21-4.13 (m, 1H), 3.85-3.79 (m, 1H), 3.62-3.59 (m, 1H), 3.49-3.43 (m, 1H), |
| | 3.00 (s, 3H). |
| 407 | ¹H NMR (300 MHz, Chloroform-d) δ 9.28 (s, 1H), 7.94 (s, 1H), 7.59-7.47 (m, 4H), 5.59-5.55 (m, 1H), 5.20-5.16 (m, 1H), |
| | 4.23-4.13 (m, 1H), 3.85-3.75 (m, 4H), 3.54 (d, J = 8.7 Hz, 1H), 3.46-3.38 (m, 1H), 2.98 (s, 3H), 2.08 (s, 3H). |
| 408 | ¹H NMR (300 MHz, Chloroform-d) δ 9.36 (s, 1H), 7.89 (s, 1H), 7.61-7.46 (m, 4H), 4.25-4.14 (m, 3H), 3.87-3.78 (m, 1H), |
| | 3.58 (d, J = 9.0 Hz, 1H), 3.48-3.40 (m, 1H), 3.00 (s, 3H), 1.45-1.36 (m, 4H), 1.12-1.02 (m, 2H), 0.80-0.66 (m, 2H). |
| 409 | ¹H NMR (300 MHz, Chloroform-d) δ 9.18 (s, 1H), 7.58-7.47 (m, 4H), 7.40 (s, 1H), 4.48 (s, 2H), 4.22-4.13 (m, 1H), 3.90- |
| | 3.79 (m, 4H), 3.57 (d, J = 8.7 Hz, 1H), 3.50-3.44 (m, 1H), 3.00 (s, 3H). |
| 411 | ¹H NMR (300 MHz, Chloroform-d) δ 9.21 (s, 1H), 7.93 (s, 1H), 7.79-7.78 (m, 1H), 7.55-7.47 (m, 4H), 6.80 (d, J = 2.7 Hz, |
| | 1H), 4.17-4.09 (m, 1H), 3.83-3.76 (m, 4H), 3.50-3.40 (m, 2H), 2.95 (s, 3H). |
| 412 | ¹H NMR (300 MHz, DMSO-d6) δ 9.61 (s, 1H), 7.81 (s, 1H), 7.69-7.59 (m, 4H), 4.11-3.94 (m, 1H), 3.84-3.72 (m, 2H), 3.67 |
| | (s, 3H), 3.49-3.41 (m, 1H), 2.81 (s, 3H), 2.03 (s, 3H). |
| 413 | ¹H NMR (300 MHz, Chloroform-d) δ 9.67 (s, 1H), 8.25 (s, 1H), 7.28 (t, J = 57.9 Hz, 1H), 7.28-7.24 (m, 1H), 7.14-7.12 (m, |
| | 1H), 7.10-7.06 (m, 1H), 6.94-6.92 (m, 1H), 4.07-3.98 (m, 1H), 3.80-3.73 (m, 1H), 3.62 (d, J = 9.0 Hz, 1H), 3.48-3.41 (m, |
| | 1H), 3.00 (s, 3H), 1.93-1.85 (m, 1H), 1.02-0.94 (m, 2H), 0.70-0.67 (m, 2H). |
| 414 | ¹H NMR (300 MHz, Chloroform-d) δ 9.71 (s, 1H), 8.23 (s, 1H), 7.44-7.38 (m, 1H), 7.34 6.95 (m, 4H), 4.17-4.02 (m, 1H), |
| | 3.86-3.75 (m, 1H), 3.59 (d, J = 9.3 Hz, 1H), 3.52-3.41 (m, 1H), 3.01 (s, 3H). |
| 415 | ¹H NMR (300 MHz, Chloroform-d) δ 9.91 (s, 1H), 8.16 (s, 1H), 7.25-7.23 (m, 2H), 7.19-7.16 (m, 2H), 4.06-3.90 (m, 4H), |
| | 3.78-3.72 (m, 1H), 3.58-3.55 (m, 1H), 3.45-3.39 (m, 1H), 2.98 (s, 3H), 2.33 (s, 3H). |
| 416 | ¹H NMR (300 MHz, Chloroform-d) δ 9.15 (s, 1H), 7.96 (s, 1H), 6.99-6.89 (m, 3H), 6.05 (s, 1H), 4.15-4.07 (m, 1H), 3.80- |
| | 3.74 (m, 4H), 3.57 (d, J = 9.3 Hz, 1H), 3.45-3.39 (m, 1H), 2.31 (s, 6H). |
| 418 | ¹H NMR (300 MHz, Chloroform-d) δ 9.36 (s, 1H), 7.99 (s, 1H), 7.27-7.25 (m, 1H), 6.94-6.90 (m, 2H), 4.18-3.97 (m, 3H), |
| | 3.79-3.72 (m, 1H), 3.61-3.58 (m, 1H), 3.44-3.39 (m, 1H), 2.98 (s, 3H), 2.31 (s, 6H), 1.40 (t, J = 7.2 Hz, 3H). |
| 419 | ¹H NMR (300 MHz, Chloroform-d) δ 9.36 (s, 1H), 8.01 (s, 1H), 6.97-6.89 (m, 3H), 4.64 4.55 (m, 1H), 4.08-3.97 (m, 1H), |
| | 3.79-3.71 (m, 1H), 3.60 (d, J = 8.7 Hz, 1H), 3.46-3.36 (m, 1H), 2.98 (s, 3H), 2.31 (s, 6H), 1.47-1.43 (m, 6H). |
| 420 | ¹H NMR (300 MHz, Chloroform-d) δ 9.44 (s, 1H), 8.07 (s, 1H), 6.99-6.88 (m, 3H), 4.95-4.83 (m, 2H), 4.07-3.97 (m, 1H), |
| | 3.84-3.70 (m, 1H), 3.60 (d, J = 8.7 Hz, 1H), 3.47-3.36 (m, 1H), 2.98 (s, 3H), 2.77 (s, 1H), 2.31 (s, 6H). |
| 421 | ¹H NMR (300 MHz, Chloroform-d) δ 9.56 (s, 1H), 8.25 (s, 1H), 7.57-7.54 (m, 2H), 7.50-7.47 (m, 2H), 7.41-7.39 (m, 1H), |
| | 6.96 (s, 2H), 6.92 (s, 1H), 4.08-3.99 (m, 1H), 3.81-3.77 (m, 1H), 3.65 (d, J = 9.0 Hz, 1H), 3.46-3.43 (m, 1H), 3.00 (s, 3H), |
| | 2.32 (s, 6H). |
| 422 | ¹H NMR (300 MHZ, Chloroform-d) δ 9.47 (s, 1H), 8.11 (s, 1H), 6.96-6.88 (m, 3H), 5.39 (s, 2H), 4.06-3.96 (m, 1H), 3.79- |
| | 3.71 (m, 1H), 3.61 (d, J = 8.7 Hz, 1H), 3.46-3.38 (m, 1H), 3.32 (s, 3H), 2.98 (s, 3H), 2.31 (s, 6H). |
| 423 | ¹H NMR (300 MHz, Chloroform-d) δ 9.38 (s, 1H), 8.04 (s, 1H), 6.93-6.90 (m, 3H), 4.26 (t, J = 5.7 Hz, 2H), 4.06-3.96 (m, |
| | 1H), 3.79-3.70 (m, 3H), 3.61-3.58 (m, 1H), 3.44-3.38 (m, 1H), 3.30 (s, 3H), 2.98 (s, 3H), 2.31 (s, 6H). |
| 424 | ¹H NMR (300 MHz, Chloroform-d) δ 9.43 (s, 1H), 8.01 (s, 1H), 6.92 (m, 3H), 4.21-4.19 (m, 2H), 4.01-3.96 (m, 3H), 3.78- |
| | 3.72 (m, 1H), 3.62-3.59 (m, 1H), 3.44-3.39 (m, 1H), 2.98 (s, 3H), 2.31 (s, 6H). |
| 425 | ¹H NMR (300 MHz, Chloroform-d) δ 9.38 (s, 1H), 7.99 (s, 1H), 7.00-6.85 (m, 3H), 4.08-3.94 (m, 1H), 3.87-3.71 (m, 4H), |
| | 3.65-3.53 (m, 1H), 3.53-3.35 (m, 1H), 2.98 (s, 3H), 2.32 (s, 6H). |
| 426 | ¹H NMR (300 MHz, Chloroform-d) δ 9.39 (s, 1H), 8.03 (s, 1H), 6.96-6.88 (m, 3H), 4.06-3.96 (m, 1H), 3.89 (s, 3H), 3.79- |
| | 3.71 (m, 1H), 3.61 (d, J = 9.0 Hz, 1H), 3.45-3.38 (m, 1H), 2.99 (s, 3H), 2.31 (s, 6H). |
| 427 | ¹H NMR (300 MHz, Chloroform-d) δ 9.89 (s, 1H), 8.16 (s, 1H), 6.95-6.88 (m, 3H), 4.04-3.96 (m, 1H), 3.94 (s, 3H), 3.78- |
| | 3.70 (m, 1H), 3.61 (d, J = 9.0 Hz, 1H), 3.44-3.37 (m, 1H), 2.97 (s, 3H), 2.31 (s, 6H). |
| 428 | ¹H NMR (300 MHz, Chloroform-d) δ 9.98 (s, 1H), 8.20 (s, 1H), 6.94-6.91 (m, 3H), 3.99-3.93 (m, 1H), 3.88 (s, 3H), 3.75- |
| | 3.69 (m, 1H), 3.62-3.59 (m, 1H), 3.43-3.37 (m, 1H), 2.98 (s, 3H), 2.31 (s, 6H). |
| 429 | ¹H NMR (300 MHz, Chloroform-d) δ 9.55 (s, 1H), 8.16 (s, 1H), 7.21 t, J = 59.1 Hz, 1H), 6.96-6.89 (m, 3H), 4.07-3.97 (m, |
| | 1H), 3.82-3.73 (m, 1H), 3.60 (d, J = 8.4 Hz, 1H), 3.45-3.37 (m, 1H), 2.98 (s, 3H), 2.31 (s, 6H), 1.82-1.75 (m, 1H), 1.17-1.10 |
| | (m, 2H), 0.93-0.80 (m, 2H). |
| 431 | ¹H NMR (300 MHz, Chloroform-d) δ 9.60 (s, 1H), 8.10 (s, 1H), 6.93-6.90 (m, 3H), 4.09-4.00 (m, 1H), 3.92 (s, 3H), 3.79 |
| | 3.72 (m, 1H), 3.64-3.61 (m, 1H), 3.44-3.38 (m, 1H), 2.98 (s, 3H), 2.32-2.28 (m, 9H). |
| 432 | ¹H NMR (300 MHz, Chloroform-d) δ 9.27 (s, 1H), 7.91 (s, 1H), 6.93-6.89 (m, 3H), 4.20 (q, J = 7.5 Hz, 2H), 4.12-4.01 (m, |
| | 1H), 3.80-3.72 (m, 1H), 3.57 (d, J = 7.8 Hz, 1H), 3.42-3.37 (m, 1H), 2.97 (s, 3H), 2.30 (s, 6H), 1.66-1.60 (m, 1H), 1.41 (t, |
| | J = 7.2 Hz, 3H), 1.08-1.03 (m, 2H), 0.77-0.67 (m, 2H). |
| 433 | ¹H NMR (300 MHz, Chloroform-d) δ 10.54 (s, 1H), 8.25 (s, 1H), 6.94-6.89 (m, 3H), 4.11 (s, 3H), 4.04 (s, 3H), 4.03-3.99 (m, |
| | 1H), 3.81-3.71 (m, 1H), 3.61 (d, J = 8.1 Hz, 1H), 3.43-3.34 (m, 1H), 2.97 (s, 3H), 2.31 (s, 6H). |
| 434 | ¹H NMR (300 MHz, Chloroform-d) δ 9.87 (s, 1H), 7.96 (s, 1H), 6.93-6.91 (m, 3H), 4.05-3.99 (m, 1H), 3.98 (s, 3H), 3.79- |
| | 3.72 (m, 1H), 3.63-3.60 (m, 1H), 3.47-3.40 (m, 1H), 2.98 (s, 3H), 2.31 (s, 6H). |
| 435 | ¹H NMR (300 MHz, DMSO-d6) δ 9.55 (s, 1H), 7.81 (s, 1H), 6.89-6.85 (m, 3H), 3.88-3.78 (m, 1H), 3.74-3.65 (m, 5H), 3.40- |
| | 3.34 (m, 1H), 2.79 (s, 3H), 2.22 (s, 6H), 2.04 (s, 3H). |
| 436 | ¹H NMR (300 MHz, Chloroform-d) δ 9.59 (s, 1H), 7.93 (s, 1H), 6.95-6.88 (m, 3H), 4.02-3.91 (m, 1H), 3.79 (s, 3H), 3.74- |
| | 3.70 (m, 1H), 3.61 (d, J = 9.0 Hz, 1H), 3.45-3.38 (m, 1H), 2.98 (s, 3H), 2.31 (s, 6H). |
| 437 | ¹H NMR (300 MHz, Chloroform-d) δ 9.56 (s, 1H), 7.84 (s, 1H), 6.96-6.87 (m, 3H), 4.06-3.96 (m, 1H), 3.79-3.71 (m, 4H), |
| | 3.60 (d, J = 8.7 Hz, 1H), 3.45-3.37 (m, 1H), 2.98 (s, 3H), 2.31 (s, 6H), 1.77-1.72 (m, 1H), 0.97-0.90 (m, 2H), 0.86-0.76 (m, |
| | 2H). |
| 438 | ¹H NMR (300 MHz, Chloroform-d) δ 9.67 (s, 1H), 8.25 (s, 1H), 7.33-6.93 (m, 4H), 4.06-3.96 (m, 1H), 3.82-3.72 (m, 1H), |
| | 3.63 (d, J = 9.3 Hz, 1H), 3.48-3.40 (m, 1H), 2.99 (s, 3H), 2.60 (q, J = 7.8 Hz, 2H), 2.33 (s, 3H), 1.24 (t, J = 7.8 Hz, 3H). |
| 439 | ¹H NMR (300 MHz, Chloroform-d) δ 9.31 (s, 1H), 7.96 (s, 1H), 6.96-6.94 (m, 3H), 4.10-4.01 (m, 1H), 3.82-3.73 (m, 4H), |
| | 3.61-3.59 (m, 1H), 3.46-3.40 (m, 1H), 2.98 (s, 3H), 2.62 (q, J = 7.2 Hz, 4H), 1.22 (t, J = 7.2 Hz, 6H). |
| 440 | ¹H NMR (300 MHz, Chloroform-d) δ 9.38 (s, 1H), 7.94 (s, 1H), 7.59-7.57 (m, 1H), 7.44-7.41 (m, 2H), 4.09-3.98 (m, 1H), |
| | 3.83-3.73 (m, 4H), 3.55 (d, J = 9.0 Hz, 1H), 3.44-3.37 (m, 1H), 2.99 (s, 3H). |
| 441 | ¹H NMR (300 MHz, Chloroform-d) δ 9.39 (s, 1H), 7.94 (s, 1H), 7.28-7.27 (m, 1H), 7.24-7.23 (m, 2H), 4.09-4.00 (m, 1H), |
| | 3.83-3.74 (m, 4H), 3.57-3.54 (m, 1H), 3.44-3.38 (m, 1H), 2.99 (s, 3H). |
| 442 | ¹H NMR (300 MHz, Chloroform-d) δ 9.69 (s, 1H), 8.22 (s, 1H), 7.30-7.29 (m, 1H), 7.25-7.24 (m, 2H), 7.15 (t, J = 57.9 Hz, |
| | 1H), 4.07-3.98 (m, 1H), 3.81-3.75 (m, 1H), 3.60-3.55 (m, 1H), 3.45-3.39 (m, 1H), 3.00 (s, 3H). |
| 443 | ¹H NMR (300 MHz, Chloroform-d) δ 9.35 (s, 1H), 7.85 (s, 1H), 7.28-7.26 (m, 1H), 7.24-7.23 (m, 2H), 4.12-4.04 (m, 1H), |
| | 3.85 (s, 3H), 3.82-3.75 (m, 1H), 3.54-3.51 (m, 1H), 3.42-3.37 (m, 1H), 2.98 (s, 3H), 1.82-1.80 (m, 1H), 1.09-1.04 (m, 2H), |
| | 0.87-0.83 (m, 2H). |
| 444 | ¹H NMR (300 MHz, Chloroform-d) δ 9.72 (s, 1H), 8.24 (s, 1H), 7.34-6.95 (s, 4H), 4.05-3.96 (m, 1H), 3.81-3.73 (m, 1H), |
| | 3.57 (d, J = 9.3 Hz, 1H), 3.48-3.38 (m, 1H), 2.99 (s, 3H), 2.25 (s, 3H). |
| 445 | ¹H NMR (300 MHz, Chloroform-d) δ 9.70 (s, 1H), 8.23 (s, 1H), 7.17-7.15 (m, 1H), 7.14 (t, J = 58.5 Hz, 1H), 7.10-7.03 (m, |
| | 2H), 4.11-4.04 (m, 1H), 3.87-3.76 (m, 4H), 3.62 (d, J = 9.3 Hz, 1H), 3.49-3.42 (m, 1H), 3.01 (s, 3H). |
| 446 | ¹H NMR (300 MHz, Chloroform-d) δ 9.56 (s, 1H), 7.94 (s, 1H), 7.38-7.32 (m, 1H), 7.19-7.11 (m, 1H), 4.31-4.12 (m, 1H), |
| | 4.00 (s, 3H), 3.83-3.75 (m, 5H), 3.51-3.41 (m, 1H), 3.00 (s, 3H). |
| 447 | ¹H NMR (300 MHZ, Chloroform-d) δ 9.39 (s, 1H), 7.97 (s, 1H), 6.97 (s, 2H), 4.00-3.91 (m, 1H), 3.80 (s, 3H), 3.76-3.70 (m, |
| | 1H), 3.59 (d, J = 9.0 Hz, 1H), 3.45-3.37 (m, 1H), 2.98 (s, 3H), 2.28 (s, 6H), 2.14 (s, 3H). |
| 448 | ¹H NMR (300 MHz, Chloroform-d) δ 9.31 (s, 1H), 7.89 (s, 1H), 6.97 (s, 2H), 4.05-3.96 (m, 1H), 3.84 (s, 3H), 3.81-3.71 (m, |
| | 2H), 3.58-3.55 (m, 1H), 3.42-3.36 (m, 1H), 2.97 (s, 3H), 2.27 (s, 6H), 2.14 (s, 3H), 1.08-1.01 (m, 2H), 0.75-0.69 (m, 2H). |
| 449 | ¹H NMR (500 MHZ, Chloroform-d) δ 9.62 (s, 1H), 7.52 (s, 1H), 6.47 (d, J = 0.6 Hz, 2H), 3.98-3.96 (m, 4H), 3.87-3.84 (m, |
| | 7H), 3.80-3.77 (m, 4H), 3.70-3.67 (m, 1H), 2.94 (s, 3H). |
| 450 | ¹H NMR (300 MHZ, Chloroform-d) δ 9.56 (s, 1H), 7.95 (s, 1H), 7.19-6.97 (m, 3H), 4.16-4.05 (m, 1H), 3.83-3.71 (m, 5H), |
| | 3.57-3.47 (m, 1H), 3.00 (s, 3H). |
| 451 | ¹H NMR (300 MHz, Chloroform-d) δ 10.55 (s, 1H), 8.44 (s, 1H), 6.91 (s, 1H), 6.81 (s, 2H), 4.87-4.78 (m, 1H), 4.39-4.31 (m, |
| | 1H), 4.07-4.04 (m, 1H), 3.85 (s, 3H), 3.74-3.67 (m, 1H), 3.34 (s, 3H), 2.30 (s, 6H). |
| 452 | ¹H NMR (300 MHz, Chloroform-d) δ 9.51 (s, 1H), 7.95 (s, 1H), 6.90-6.83 (m, 3H), 4.33-4.26 (m, 1H), 4.23-4.16 (m, 1H), |
| | 4.01-4.99 (m, 1H), 3.80 (s, 3H), 3.76-3.69 (m, 1H), 3.34 (s, 3H), 2.29 (s, 6H). |
| 453 | ¹H NMR (300 MHz, Chloroform-d) δ 9.35 (s, 1H), 7.97 (s, 1H), 7.29-7.24 (m, 1H), 7.16-7.07 (m, 3H), 4.11-4.03 (m, 1H), |
| | 3.83-3.74 (m, 4H), 3.60-3.57 (m, 1H), 3.47-3.41 (m, 1H), 2.99 (s, 3H), 2.58 (t, J = 7.2 Hz, 2H), 1.68-1.61 (m, 2H), 0.95 (t, |
| | J = 7.2 Hz, 3H). |
| 454 | ¹H NMR (300 MHz, Chloroform-d) δ 9.67 (s, 1H), 7.62 (s, 1H), 6.81-6.71 (m, 3H), 3.90-3.84 (m, 1H), 3.75 (s, 3H), 3.93- |
| | 3.37 (m, 1H), 3.30-3.26 (m, 1H), 3.00 (s, 3H), 2.21 (s, 6H), 1.66 (s, 3H) |
| 455 | ¹H NMR (300 MHz, Chloroform-d) δ 10.04 (s, 1H), 7.60 (s, 1H), 6.83-6.80 (m, 1H), 6.71-6.70 (m, 2H), 3.95-3.90 (m, 1H), |
| | 3.75 (s, 3H), 3.42-3.40 (m, 1H), 3.22-3.19 (m, 1H), 3.01 (s, 3H), 2.31-2.29 (m, 1H), 2.21 (s, 6H), 2.17-2.13 (m, 1H), 1.81- |
| | 1.73 (m, 1H), 0.48-0.45 (m, 2H), 0.17-0.05 (m, 2H). |
| 456 | ¹H NMR (300 MHz, Chloroform-d) δ 9.49 (s, 1H), 7.88 (s, 1H), 7.59-7.45 (m, 5H), 4.19 4.07 (m, 3H), 3.85-3.79 (m, 1H), |
| | 3.57 (d, J = 9.3 Hz, 1H), 3.55-3.43 (m, 1H), 3.01 (s, 3H), 1.45 (t, J = 7.2 Hz, 3H). |
| 457 | ¹H NMR (300 MHz, Chloroform-d) δ 10.06 (s, 1H), 8.13 (s, 1H), 7.72-7.50 (m, 4H), 6.80 (t, J = 54.0 Hz, 1H), 4.24-4.11 (m, |
| | 1H), 3.89 (s, 3H), 3.87-3.79 (m, 1H), 3.68 (d, J = 9.3 Hz, 1H), 3.55-3.43 (m, 1H), 3.06 (s, 3H). |
| 458 | ¹H NMR (300 MHz, Chloroform-d) δ 10.05 (s, 1H), 8.16 (s, 1H), 7.61-7.47 (m, 4H), 4.18-4.05 (m, 3H), 3.84-3.75 (m, 1H), |
| | 3.62 (d, J = 9.6 Hz, 1H), 3.47-3.41 (m, 1H), 3.01 (s, 3H), 1.46 (t, J = 7.2 Hz, 3H). |
| 459 | ¹H NMR (300 MHz, Chloroform-d) δ 10.06 (s, 1H), 8.17 (s, 1H), 7.62-7.46 (m, 4H), 4.51-4.42 (m, 1H), 4.15-4.05 (m, 1H), |
| | 3.84-3.76 (m, 1H), 3.63 (d, J = 9.3 Hz, 1H), 3.48-3.40 (m, 1H), 3.01 (s, 3H), 1.48 (d, J = 6.9 Hz, 6H). |
| 460 | ¹H NMR (300 MHz, Chloroform-d) δ 10.05 (s, 1H), 8.19 (s, 1H), 7.67-7.42 (m, 4H), 4.40-4.31 (m, 1H), 4.16-4.03 (m, 1H), |
| | 3.84-3.75 (m, 1H), 3.65-3.57 (m, 1H), 3.50-3.39 (m, 1H), 3.00 (s, 3H), 1.25-0.72 (m, 4H). |
| 461 | ¹H NMR (300 MHz, Chloroform-d) δ 8.90 (s, 1H), 7.59-7.47 (m, 4H), 4.24-4.09 (m, 1H), 3.94 (s, 3H), 3.85-3.70 (m, 1H), |
| | 3.75-3.65 (m, 1H), 3.56-3.48 (m, 1H), 3.00 (s, 3H), 2.28 (s, 3H), 2.22 (s, 3H). |
| 462 | ¹H NMR (300 MHz, Chloroform-d) δ 9.39 (s, 1H), 7.99 (s, 1H), 7.27-7.22 (m, 1H), 7.14-7.06 (m, 3H), 4.09-4.01 (m, 3H), |
| | 3.80-3.74 (m, 1H), 3.61-3.59 (m, 1H), 3.46-3.40 (m, 1H), 2.98 (s, 3H), 2.35 (s, 3H), 1.88-1.76 (m, 2H), 0.91-0.86 (m, 3H). |
| 463 | ¹H NMR (300 MHz, Chloroform-d) δ 9.91 (s, 1H), 8.53 (s, 1H), 7.31-7.27 (m, 1H), 7.17-7.08 (m, 3H), 4.04-3.93 (m, 1H), |
| | 3.82-3.72 (m, 1H), 3.69-3.63 (m, 1H), 3.48-3.39 (m, 1H), 3.00 (s, 3H), 2.37 (s, 3H), 1.60 (s, 9H). |
| 464 | ¹H NMR (300 MHz, Chloroform-d) δ 9.63 (s, 1H), 8.18 (s, 1H), 7.29-7.22 (m, 1H), 7.18-7.07 (m, 3H), 5.25-5.20 (m, 1H), |
| | 4.06-3.92 (m, 2H), 3.81-3.72 (m, 1H), 3.68-3.59 (m, 2H), 3.48-3.39 (m, 1H), 2.99 (s, 3H), 2.36 (s, 3H), 2.04-1.92 (m, 2H), |
| | 1.67-1.62 (m, 4H). |
| 465 | ¹H NMR (300 MHz, Chloroform-d) δ 9.32 (s, 1H), 7.95 (s, 1H), 7.34-6.93 (m, 5H), 4.13-4.02 (m, 1H), 3.81-3.75 (m, 1H), |
| | 3.61-3.58 (m, 1H), 3.48-3.42 (m, 1H), 2.99 (s, 3H), 2.40 (s, 3H), 2.36 (s, 3H). |
| 466 | ¹H NMR (300 MHz, Chloroform-d) δ 9.40 (s, 1H), 8.03 (s, 1H), 7.35-6.95 (m, 5H), 4.10-4.01 (m, 1H), 3.81-3.75 (m, 1H), |
| | 3.61-3.58 (m, 1H), 3.45 (m, 1H), 2.99 (s, 3H), 2.86 (q, J = 7.5 Hz, 2H), 2.36 (s, 3H), 1.27 (t, J = 7.5 Hz, 3H). |
| 467 | ¹H NMR (300 MHz, Chloroform-d) δ 9.33 (s, 1H), 7.75 (s, 1H), 7.16-7.02 (m, 4H), 5.45-5.29 (m, 2H), 4.10-4.02 (m, 1H), |
| | 3.89 (s, 3H), 3.81-3.75 (m, 1H), 3.58 (d, J = 8.7 Hz, 1H), 3.47-3.42 (m, 1H), 2.98 (s, 3H), 2.35 (s, 3H). |
| 468 | ¹H NMR (300 MHz, Chloroform-d) δ 9.73 (s, 1H), 8.23 (s, 1H), 7.26-7.24 (m, 1H), 7.17 (t, J = 58.8 Hz, 1H), 7.16-7.06 (m, |
| | 3H), 6.90-6.77 (m, 1H), 5.90 (d, J = 18.0 Hz, 1H), 5.72 (d, J = 11.7 Hz, 1H), 4.13-3.99 (m, 1H), 3.84-3.73 (m, 1H), 3.65- |
| | 3.57 (m, 1H), 3.49-3.40 (m, 1H), 2.98 (s, 3H), 2.36 (s, 3H). |
| 469 | ¹H NMR (300 MHz, Chloroform-d) δ 9.13 (s, 1H), 7.96 (s, 1H), 7.80 (d, J = 2.4 Hz, 1H), 7.25-7.21 (m, 1H), 7.11-7.04 (m, |
| | 3H), 6.80 (d, J = 2.4 Hz, 1H), 4.07-3.98 (m, 1H), 3.77-3.71 (m, 4H), 3.51-3.48 (m, 1H), 3.43-3.37 (m, 1H), 2.93 (s, 3H), |
| | 2.34 (s, 3H). |
| 470 | ¹H NMR (300 MHz, Chloroform-d) δ 10.66 (s, 1H), 10.07 (s, 1H), 8.19 (s, 1H), 7.26-7.22 (m, 1H), 7.15-7.07 (m, 3H), 4.12 |
| | (s, 3H), 4.09-4.03 (m, 1H), 3.81-3.73 (m, 1H), 3.63 (d, J = 8.8 Hz, 1H), 3.47-3.39 (m, 1H), 2.98 (s, 3H), 2.36 (s, 3H). |
| 471 | ¹H NMR (300 MHz, Chloroform-d) δ 9.07 (s, 1H), 7.72 (s, 1H), 7.26-7.24 (m, 1H), 7.15-7.11 (m, 3H), 6.38 (s, 2H), 4.16- |
| | 4.05 (m, 4H), 3.82-3.75 (m, 1H), 3.62-3.58 (m, 1H), 3.51-3.44 (m, 1H), 2.96 (s, 3H), 2.36 (s, 3H). |
| 472 | ¹H NMR (300 MHz, Chloroform-d) δ 10.55 (s, 1H), 8.24 (s, 1H), 7.17-7.06 (m, 4H), 4.11 (s, 3H), 4.10-4.06 (m, 1H), 4.04 (s, |
| | 3H), 3.82-3.74 (m, 1H), 3.61 (d, J = 8.4 Hz, 1H), 3.44-3.37 (m, 1H), 2.98 (s, 3H), 2.35 (s, 3H). |
| 473 | ¹H NMR (300 MHz, Chloroform-d) δ 9.69 (s, 1H), 8.15 (s, 1H), 7.65-7.43 (m, 4H), 5.25-5.19 (m, 1H), 4.17-4.05 (m, 1H), |
| | 4.02-3.93 (m, 1H), 3.84-3.75 (m, 1H), 3.67-3.55 (m, 2H), 3.49-3.40 (m, 1H), 3.00 (s, 3H), 2.02-1.93 (m, 2H), 1.68-1.59 (m, |
| | 2H), 1.57-1.52 (m, 2H). |
| 474 | ¹H NMR (300 MHz, Chloroform-d) δ 10.20 (s, 1H), 7.80 (s, 1H), 7.47-7.43 (m, 2H), 7.35-7.30 (m, 2H), 4.31-4.29 (m, 1H), |
| | 4.06-4.01 (m, 1H), 3.51-3.41 (m, 1H), 3.12-3.00 (m, 4H), 1.49 (s, 9H). |
| 475 | ¹H NMR (300 MHz, Chloroform-d) δ 9.36 (s, 1H), 7.94 (s, 1H), 7.60-7.48 (m, 4H), 7.15 (t, J = 58.8 Hz, 1H), 4.22-4.13 (m, |
| | 1H), 3.87-3.80 (m, 1H), 3.61-3.58 (m, 1H), 3.51-3.45 (m, 1H), 3.02 (s, 3H), 2.42 (s, 3H). |
| 476 | ¹H NMR (300 MHz, Chloroform-d) δ 9.15 (s, 1H), 7.75 (s, 1H), 7.58-7.45 (m, 4H), 4.23-4.13 (m, 1H), 3.85-3.76 (m, 4H), |
| | 3.56 (d, J = 8.7 Hz, 1H), 3.48-3.42 (m, 1H), 3.00 (s, 3H), 2.61 (t, J = 7.5 Hz, 2H), 1.59-1.49 (m, 2H), 1.41-1.31 (m, 2H), 0.92 |
| | (t, J = 7.2 Hz, 3H). |
| 477 | ¹H NMR (300 MHz, Chloroform-d) δ 9.39 (s, 1H), 7.74 (s, 1H), 7.58-7.49 (m, 4H), 5.45-5.29 (m, 2H), 4.22-4.13 (m, 1H), |
| | 3.89 (s, 3H), 3.84-3.81 (m, 1H), 3.58 (d, J = 8.7 Hz, 1H), 3.49-345 (m, 1H), 3.00 (s, 3H). |
| 478 | ¹H NMR (300 MHz, Chloroform-d) δ 9.62 (s, 1H), 7.80 (s, 1H), 7.60-7.47 (m, 4H), 6.85 (t, J = 60.0 Hz, 1H), 4.21-4.11 (m, |
| | 1H), 3.93 (s, 3H), 3.86-3.77 (m, 1H), 3.62-3.53 (m, 1H), 3.51-3.40 (m, 1H), 3.00 (s, 3H). |
| 479 | ¹H NMR (300 MHz, Chloroform-d) δ 9.68 (s, 1H), 8.00 (s, 1H), 7.61-7.46 (m, 4H), 4.24-4.15 (m, 1H), 3.92 (s, 3H), 3.87- |
| | 3.80 (m, 1H), 3.62 (d, J = 9.0 Hz, 1H), 3.48-3.42 (m, 1H), 3.00 (s, 3H), 2.28 (s, 3H). |
| 480 | ¹H NMR (300 MHz, Chloroform-d) δ 10.13 (s, 1H), 8.26 (s, 1H), 7.59-7.52 (m, 4H), 4.21-4.14 (m, 1H), 4.10 (s, 3H), 3.86- |
| | 3.77 (m, 1H), 3.61 (d, J = 9.3 Hz, 1H), 3.48-3.41 (m, 1H), 3.19 (s, 3H), 2.99 (s, 3H). |
| 481 | ¹H NMR (300 MHz, Chloroform-d) δ 10.73 (s, 1H), 10.08 (s, 1H), 8.17 (s, 1H), 7.58-7.49 (m, 4H), 4.24-4.11 (m, 4H), 3.85- |
| | 3.78 (m, 1H), 3.63 (d, J = 9.0 Hz, 1H), 3.48-3.42 (m, 1H), 3.01 (s, 3H). |
| 482 | ¹H NMR (300 MHz, Chloroform-d) δ 9.25 (s, 1H), 7.76 (s, 1H), 7.60-7.50 (m, 4H), 6.34 (s, 2H), 4.28-4.19 (m, 1H), 4.08 (s, |
| | 3H), 3.87-3.79 (m, 1H), 3.61 (d, J = 9.3 Hz, 1H), 3.53-3.46 (m, 1H), 2.98 (s, 3H). |
| 483 | ¹H NMR (300 MHz, Chloroform-d) δ 10.21 (s, 1H), 8.17-8.14 (m, 2H), 7.59-7.47 (m, 4H), 4.30-4.20 (m, 1H), 4.13 (s, 3H), |
| | 3.86 (s, 3H), 3.85-3.79 (m, 1H), 3.59-3.53 (m, 1H), 3.46-3.40 (m, 1H), 2.98 (s, 3H). |
| 484 | ¹H NMR (300 MHz, Chloroform-d) δ 9.94 (s, 1H), 8.51 (s, 1H), 7.56-7.48 (m, 4H), 4.16-4.07 (m, 1H), 3.85-3.79 (m, 1H), |
| | 3.65 (d, J = 9.3 Hz, 1H), 3.50-3.44 (m, 1H), 3.02 (s, 3H), 1.61 (s, 9H). |
| 485 | ¹H NMR (300 MHz, Chloroform-d) δ 9.88 (s, 1H), 8.16 (s, 1H), 7.32-7.28 (m, 1H), 6.95-6.78 (m, 3H), 4.09-4.00 (m, 1H), |
| | 3.94 (s, 3H), 3.84-3.72 (m, 4H), 3.62-3.57 (m, 1H), 3.46-3.40 (m, 1H), 2.98 (s, 3H). |
| 486 | ¹H NMR (300 MHz, Chloroform-d) δ 9.71 (s, 1H), 8.25 (s, 1H), 7.33-6.95 (m, 5H), 4.03-3.97 (m, 1H), 3.80-3.73 (m, 1H), |
| | 3.61-3.57 (m, 1H), 3.47-3.41 (m, 1H), 2.99 (s, 3H), 2.34 (s, 3H). |
| 487 | ¹H NMR (300 MHz, Chloroform-d) δ 9.72 (s, 1H), 8.25 (s, 1H), 7.33-6.95 (m, 4H), 4.02-3.93 (m, 1H), 3.78-3.71 (m, 1H), |
| | 3.62-3.59 (m, 1H), 3.46-3.40 (m, 1H), 2.99 (s, 3H), 2.27 (s, 3H), 2.24 (s, 3H). |
| 488 | ¹H NMR (300 MHz, Chloroform-d) δ 9.92 (s, 1H), 8.16 (s, 1H), 7.15-7.10 (m, 2H), 7.09-7.05 (m, 1H), 4.01-3.96 (m, 1H), |
| | 3.94 (s, 3H), 3.77-3.69 (m, 1H), 3.61-3.56 (m, 1H), 3.45-3.37 (m, 1H), 2.97 (s, 3H), 2.26 (s, 3H), 2.24 (s, 3H). |
| 489 | ¹H NMR (300 MHz, Chloroform-d) δ 9.91 (s, 1H), 8.14 (s, 1H), 6.91-6.73 (m, 3H), 4.08-4.01 (m, 1H), 3.95 (s, 3H), 3.82- |
| | 3.74 (m, 1H), 3.56-3.52 (m, 1H), 3.44-3.37 (m, 1H), 2.99 (s, 3H). |
| 490 | ¹H NMR (300 MHz, Chloroform-d) δ 6.92-6.78 (m, 3H), 6.68 (s, 1H), 4.13-3.93 (m, 1H), 3.81-3.74 (m, 3H), 3.73-3.63 (m, |
| | 1H), 3.59 3.41 (m, 1H), 3.35-3.26 (m, 1H), 3.18 (s, 2H), 2.96-2.92 (s, 3H), 2.86 (s, 1H), 2.29 (s, 6H). |
| 491 | ¹H NMR (300 MHz, Chloroform-d) δ 9.90 (s, 1H), 8.53 (s, 1H), 6.98-6.90 (m, 3H), 4.00-3.90 (m, 1H), 3.79-3.64 (m, 2H), |
| | 3.46-3.37 (m, 1H), 2.99 (s, 3H), 2.32 (s, 6H), 1.60 (s, 9H). |
| 492 | ¹H NMR (300 MHz, Chloroform-d) δ 9.63 (s, 1H), 8.18 (s, 1H), 6.96-6.89 (m, 3H), 5.25-5.20 (m, 1H), 4.02-3.94 (m, 2H), |
| | 3.78-3.70 (m, 1H), 3.65-3.58 (m, 2H), 3.45-3.37 (m, 1H), 2.98 (s, 3H), 2.31 (s, 6H), 2.00-1.94 (m, 2H), 1.72-1.59 (m, 4H). |
| 493 | ¹H NMR (300 MHz, Chloroform-d) δ 7.25 (s, 1H), 6.96-6.92 (m, 1H), 6.88-6.82 (m, 2H), 4.20-4.03 (m, 2H), 3.84 (s, 3H), |
| | 3.65-3.40 (m, 4H), 2.98 (s, 3H), 2.32 (s, 6H), 2.04-2.01 (m, 1H). |
| 494 | ¹H NMR (300 MHz, Chloroform-d) δ 9.30 (s, 1H), 7.95 (s, 1H), 7.13 (t, J = 58.8 Hz, 1H), 6.94-6.91 (m, 3H), 4.06-3.97 (m, |
| | 1H), 3.79-3.72 (m, 1H), 3.60-3.58 (m, 1H), 3.46-3.40 (m, 1H), 2.98 (s, 3H), 2.41 (s, 3H), 2.32 (s, 6H). |
| 495 | ¹H NMR (300 MHz, Chloroform-d) δ 9.32 (s, 1H), 7.75 (s, 1H), 6.93-6.91 (m, 3H), 5.45-5.28 (m, 2H), 4.06-3.98 (m, 1H), |
| | 3.89 (s, 3H), 3.79-3.72 (m, 1H), 3.59-3.56 (m, 1H), 3.46-3.38 (m, 1H), 2.98 (s, 3H), 2.31 (s, 6H). |
| 496 | ¹H NMR (300 MHz, Chloroform-d) δ 9.74 (s, 1H), 8.24 (s, 1H), 7.36-6.92 (m, 5H), 5.93-5.71 (m, 2H), 4.03-4.00 (m, 1H), |
| | 3.78-3.72 (m, 1H), 3.63-3.60 (m, 1H), 3.45-3.39 (m, 1H), 2.98 (s, 3H), 2.32 (s, 6H). |
| 497 | ¹H NMR (300 MHz, Chloroform-d) δ 9.11 (s, 1H), 7.96 (s, 1H), 7.80-7.79 (m, 1H), 6.89 (m, 3H), 6.81-6.80 (m, 1H), 4.03- |
| | 3.94 (m, 1H), 3.76 (s, 3H), 3.72-3.69 (m, 1H), 3.51-3.48 (m, 1H), 3.41-3.36 (m, 1H), 2.93 (s, 3H), 2.31 (s, 6H). |
| 498 | ¹H NMR (300 MHz, Chloroform-d) δ 10.02 (s, 1H), 8.29 (s, 1H), 6.94-6.91 (m, 3H), 4.09 (s, 3H), 4.08-4.00 (m, 1H), 3.77- |
| | 3.71 (m, 1H), 3.63-3.60 (m, 1H), 3.43-3.37 (m, 1H), 3.19 (s, 3H), 2.96 (s, 3H), 2.33 (s, 6H). |
| 499 | ¹H NMR (300 MHz, Chloroform-d) δ 9.05 (s, 1H), 7.74 (s, 1H), 6.93-6.90 (m, 3H), 4.13-4.01 (m, 1H), 3.83-3.73 (m, 1H), |
| | 3.69 (s, 3H), 3.55-3.53 (m, 1H), 3.44-3.38 (m, 1H), 2.97 (s, 3H), 2.81-2.79 (m, 6H), 2.30 (s, 6H). |
| 500 | ¹H NMR (300 MHz, Chloroform-d) δ 10.64 (s, 1H), 10.08 (s, 1H), 8.19 (s, 1H), 6.94-6.89 (m, 3H), 4.12 (s, 3H), 4.07-3.99 |
| | (m, 1H), 3.79-3.71 (m, 1H), 3.63 (d, J = 9.0 Hz, 1H), 3.45-3.37 (m, 1H), 2.98 (s, 3H), 2.31 (s, 6H). |
| 501 | ¹H NMR (300 MHz, Chloroform-d) δ 10.13 (s, 1H), 8.19-8.15 (m, 2H), 6.92-6.89 (m, 3H), 4.13-4.05 (m, 4H), 3.87 (s, 3H), |
| | 3.80-3.74 (m, 1H), 3.56 (d, J = 7.5 Hz, 1H), 3.41-3.36 (m, 1H), 2.96 (s, 3H), 2.31 (s, 6H). |
| 502 | ¹H NMR (300 MHz, Chloroform-d) δ 9.04 (s, 1H), 7.38 (s, 1H), 6.94-6.90 (m, 3H), 4.48 (s, 2H), 4.07-3.99 (m, 1H), 3.88 (s, |
| | 3H), 3.81-3.75 (m, 1H), 3.59-3.54 (m, 1H), 3.46-3.40 (m, 1H), 2.98 (s, 3H), 2.31 (s, 6H). |
| 503 | ¹H NMR (300 MHz, Chloroform-d) δ 9.34 (s, 1H), 7.88 (s, 1H), 6.94-6.75 (m, 3H), 4.12-4.04 (m, 1H), 3.85 (s, 3H), 3.82- |
| | 3.78 (m, 1H), 3.57-3.52 (m, 1H), 3.43-3.36 (m, 1H), 2.98 (s, 3H), 2.34 (s, 3H), 1.68-1.60 (m, 1H), 1.08-1.04 (m, 2H), 0.74- |
| | 0.65 (m, 2H). |
| 504 | ¹H NMR (300 MHz, Chloroform-d) δ 9.37 (s, 1H), 7.96 (s, 1H), 7.13-7.03 (m, 3H), 4.07-3.98 (m, 1H), 3.81 (s, 3H), 3.77- |
| | 3.72 (m, 1H), 3.59-3.54 (m, 1H), 3.44-3.37 (m, 1H), 2.99 (s, 3H), 2.33 (s, 3H). |
| 505 | ¹H NMR (300 MHz, Chloroform-d) δ 9.69 (s, 1H), 8.24 (s, 1H), 7.14 (t, J = 57.9 Hz, 1H), 7.13-7.08 (m, 2H), 7.05-7.03 (m, |
| | 1H), 4.04-3.96 (m, 1H), 3.80-3.73 (m, 1H), 3.60 (d, J = 9.0 Hz, 1H), 3.45-3.39 (m, 1H), 3.00 (s, 3H) 2.34 (s, 3H). |
| 506 | ¹H NMR (300 MHz, Chloroform-d) δ 9.31 (s, 1H), 7.87 (s, 1H), 7.15-7.01 (m, 3H), 4.11-4.02 (m, 1H), 3.84 (s, 3H), 3.78- |
| | 3.74 (m, 1H), 3.54 (d, J = 8.7 Hz, 1H), 3.44-3.36 (m, 1H), 2.96 (s, 3H), 2.32 (s, 3H), 1.62-1.57 (m, 1H), 1.09-1.04 (m, 2H), |
| | 0.77-0.65 (m, 2H). |
| 507 | ¹H NMR (300 MHz, Chloroform-d) δ 9.39 (s, 1H), 7.94 (s, 1H), 7.14-6.97 (m, 3H), 4.09-4.04 (m, 1H), 3.83-3.76 (m, 4H), |
| | 3.54 (d, J = 9.0 Hz, 1H), 3.43-3.40 (m, 1H), 2.99 (s, 3H). |
| 508 | ¹H NMR (300 MHz, Chloroform-d) δ 9.93 (s, 1H), 8.12 (s, 1H), 7.40-7.26 (m, 3H), 4.19-4.09 (m, 1H), 3.95 (s, 3H), 3.87- |
| | 3.76 (m, 1H), 3.57 (d, J = 9.0 Hz, 1H), 3.48-3.38 (m, 1H), 3.00 (s, 3H). |
| 509 | ¹H NMR (300 MHz, Chloroform-d) δ 9.71 (s, 1H), 8.22 (s, 1H), 7.57-7.54 (m, 2H), 7.51-7.48 (m, 1H), 7.15 (t, J = 57.9 Hz, |
| | 1H), 4.16-4.06 (m, 1H), 3.86-3.78 (m, 1H), 3.60 (d, J = 9.3 Hz, 1H), 3.50-3.42 (m, 1H), 3.02 (s, 3H). |
| 510 | ¹H NMR (300 MHz, Chloroform-d) δ 9.38 (s, 1H), 7.85 (s, 1H), 7.57-7.51 (m, 2H), 7.50-7.47 (m, 1H), 4.22-4.12 (m, 1H), |
| | 3.85 (s, 3H), 3.84-3.77 (m, 1H), 3.56 (d, J = 9.3 Hz, 1H), 3.47-3.39 (m, 1H), 3.00 (s, 3H), 1.44-1.37 (m, 1H), 1.10-1.04 (m, |
| | 2H), 0.79-0.67 (m, 2H). |
| 511 | ¹H NMR (300 MHz, Chloroform-d) δ 9.44 (s, 1H), 7.94-7.93 (m, 1H), 7.61-7.57 (m, 2H), 7.43 (s, 1H), 6.61 (t, J = 56.1 Hz, |
| | 1H), 4.19-3.96 (m, 1H), 3.82-3.76 (m, 4H), 3.58 (d, J = 8.7 Hz, 1H), 3.53-3.40 (m, 1H), 3.00 (s, 3H). |
| 512 | ¹H NMR (300 MHz, Chloroform-d) δ 9.70-9.25 (m, 1H), 8.23-8.20 (m, 1H), 7.62-7.59 (m, 2H), 7.43-7.35 (m, 1H), 7.15 (t, |
| | J = 57.9 Hz, 1H), 6.62 (t, J = 56.1 Hz, 1H), 4.10-4.04 (m, 1H), 3.92-3.90 (m, 1H), 3.82-3.79 (m, 1H), 3.62-3.59 (m, 1H), |
| | 3.49-3.43 (m, 1H), 3.01 (s, 2H). |
| 513 | ¹H NMR (300 MHz, Chloroform-d) δ 9.39 (s, 1H), 7.89-7.81 (m, 1H), 7.61-7.56 (m, 2H), 7.44-7.40 (m, 1H), 6.60 (t, J = 56.1 |
| | Hz,, 1H), 4.17-4.09 (m, 1H), 3.87-3.76 (m, 4H), 3.57 (d, J = 8.7 Hz,, 1H), 3.50-3.38 (m, 1H), 2.99 (s, 3H), 1.65-1.59 (m, |
| | 1H), 1.09-1.04 (m, 2H), 0.80-0.64 (m, 2H). |
| 514 | ¹H NMR (300 MHz, Chloroform-d) δ 9.36 (s, 1H), 7.84 (s, 1H), 7.73-7.65 (m, 2H), 7.53 (s, 1H), 4.20-4.09 (m, 1H), 3.85 (s, |
| | 3H), 3.81-3.79 (m, 1H), 3.55 (d, J = 8.9 Hz, 1H), 3.47-3.37 (m, 1H), 3.00 (s, 3H), 1.72-1.55 (m, 1H), 0.88-0.83 (m, 2H), |
| | 0.77-0.64 (m, 2H). |
| 515 | ¹H NMR (300 MHz, Chloroform-d) δ 9.69 (s, 1H), 8.25 (s, 1H), 7.13 (t, J = 57.9 Hz, 1H), 6.97 (s, 2H), 3.97-3.89 (m, 1H), |
| | 3.74-3.70 (m, 1H), 3.62 (d, J = 9.3 Hz, 1H), 3.44-3.40 (m, 1H), 2.98 (s, 3H), 2.28 (s, 6H), 2.14 (s, 3H). |
| 516 | ¹H NMR (300 MHz, Chloroform-d) δ 9.44 (s, 1H), 8.01 (s, 1H), 6.98 (s, 2H), 4.00-3.91 (m, 1H), 3.85-3.70 (m, 4H), 3.61 (d, |
| | J = 9.3 Hz, 1H), 3.43-3.37 (m, 1H), 2.98 (s, 3H), 2.28 (s, 6H), 2.14 (s, 3H). |
| 517 | ¹H NMR (300 MHz, Chloroform-d) δ 9.91 (s, 1H), 8.16 (s, 1H), 6.97 (s, 2H), 3.98-3.90 (m, 4H), 3.75-3.69 (m, 1H), 3.61- |
| | 3.58 (m, 1H), 3.42-3.36 (m, 1H), 2.97 (s, 3H), 2.28 (s, 6H), 2.14 (s, 3H). |
| 518 | ¹H NMR (300 MHz, Chloroform-d) δ 9.94 (s, 1H), 7.96 (s, 1H), 6.97 (s, 2H), 3.99-3.93 (m, 4H), 3.72-3.70 (m, 1H), 3.62 (d, |
| | J = 9.3 Hz, 1H), 3.44-3.41 (m, 1H), 3.28 (s, 3H), 2.28 (s, 6H), 2.15 (s, 3H). |
| 519 | ¹H NMR (300 MHz, Chloroform-d) δ 9.41 (s, 1H), 7.94 (s, 1H), 7.27 (s, 2H), 4.03-3.95 (m, 1H), 3.81 (s, 3H), 3.76-3.72 (m, |
| | 1H), 3.55-3.52 (m, 1H), 3.42-3.36 (m, 1H), 2.98 (s, 3H), 2.43 (s, 3H). |
| 520 | ¹H NMR (300 MHz, Chloroform-d) δ 9.33 (s, 1H), 7.99 (s, 1H), 6.87 (s, 1H), 6.78 (s 1H), 4.36-4.27 (m, 1H), 3.82-3.76 (m, |
| | 4H), 3.55-3.48 (m, 2H), 2.97 (s, 3H), 2.22 (s, 3H). |
| 521 | ¹H NMR (300 MHz, Chloroform-d) δ 9.56 (s, 1H), 8.12-8.09 (m, 1H), 7.95 (s, 1H), 7.64-7.60 (m, 1H), 6.92-6.88 (m, 1H), |
| | 4.04-3.99 (m, 1H), 3.97 (s, 3H), 3.93-3.88 (m, 1H), 3.80 (s, 3H), 3.66-3.60 (m, 1H), 3.54-3.47 (m, 1H), 2.98 (s, 3H). |
| 522 | ¹H NMR (300 MHz, Chloroform-d) δ 9.39 (s, 1H), 7.99 (s, 1H), 6.14 (d, J = 3.0 Hz, 1H), 5.90-5.88 (m, 1H), 4.16-4.07 (m, |
| | 1H), 3.80 (s, 3H), 3.82-3.78 (m, 2H), 3.56-3.50 (m, 1H), 2.95 (s, 3H), 2.25 (s, 3H). |
| 523 | ¹H NMR (300 MHz, Chloroform-d) δ 8.99 (s, 1H), 7.62 (d, J = 2.7 Hz, 1H), 7.25-7.20 (m, 1H), 7.15-7.04 (m, 3H), 4.24-4.04 |
| | (m, 3H), 3.81-3.73 (m, 1H), 3.54 (d, J = 8.7 Hz, 1H), 3.50-3.40 (m, 1H), 2.97 (s, 3H), 2.34 (s, 3H), 1.40 (t, J = 7.5 Hz, 3H). |
| 524 | ¹H NMR (300 MHz, Chloroform-d) δ 9.53 (s, 1H), 7.81 (s, 1H), 7.23-7.08 (m, 4H), 6.86 (t, J = 53.4 Hz, 1H), 4.10-4.01 (m, |
| | 1H), 3.94 (s, 3H), 3.81-3.74 (m, 1H), 3.58-3.56 (m, 1H), 3.47-3.41 (m, 1H), 2.98 (s, 3H), 2.36 (s, 3H). |
| 525 | ¹H NMR (300 MHz, Chloroform-d) δ 9.85 (s, 1H), 8.19 (s, 1H), 7.26-7.22 (m, 1H), 7.18-7.03 (m, 3H), 4.22 (q, J = 7.2 Hz, |
| | 2H), 4.10-3.99 (m, 1H), 3.80-3.75 (m, 1H), 3.60 (d, J = 8.4 Hz, 1H), 3.37-3.46 (m, 1H), 2.98 (s 3H), 2.35 (s, 3H), 1.44 (t, J = |
| | 7.2 Hz, 3H). |
| 526 | ¹H NMR (300 MHz, Chloroform-d) δ 9.85 (s, 1H), 8.22 (s, 1H), 7.28-7.23 (m, 1H), 7.15-7.08 (m, 3H), 4.60-4.52 (m, 1H), |
| | 4.08-4.00 (m, 1H), 3.80-3.71 (m, 1H), 3.60 (d, J = 8.7 Hz, 1H), 3.45-3.39 (m, 1H), 2.98 (s, 3H), 2.36 (s, 3H), 1.50-1.47 (m, |
| | 6H). |
| 527 | ¹H NMR (300 MHZ, Chloroform-d) δ 9.91 (s, 1H), 8.12 (s, 1H), 7.26-7.23 (m, 1H), 7.14-7.08 (m, 3H), 4.04-3.98 (m, 1H), |
| | 3.79-3.72 (m, 1H), 3.61-3.52 (m, 2H), 3.45-3.39 (m, 1H), 2.98 (s, 3H), 2.36 (s, 3H), 1.29-1.24 (m, 2H), 1.14-1.01 (m, 2H). |
| 528 | ¹H NMR (300 MHz, Chloroform-d) δ 9.43 (s, 1H), 8.02 (s, 1H), 7.26-7.22 (m, 1H), 7.16-7.06 (m, 3H), 5.45 (s, 2H), 4.13-4.03 |
| | (m, 1H), 3.82-3.73 (m, 1H), 3.62-3.57 (m, 1H), 3.47-3.38 (m, 1H), 3.30 (s, 3H), 2.98 (s, 3H), 2.35 (s, 3H), 1.75-1.68 (m, 1H), |
| | 1.11-1.05(m, 2H), 0.82-0.74(m, 2H). |
| 529 | ¹H NMR (300 MHz, Chloroform-d) δ 9.18 (s, 1H), 8.09 (s, 1H), 7.54-7.52 (m, 1H), 7.24-7.20 (m, 3H), 7.11-7.05 (m, 3H), |
| | 4.13-4.05 (m, 1H), 3.86 (s, 3H), 3.79-3.72 (m, 1H), 3.51 (d, J = 8.4 Hz, 1H), 3.43-3.35 (m, 1H), 2.93 (s, 3H), 2.34 (s, 3H). |
| 530 | ¹H NMR (300 MHz, Chloroform-d) δ 9.80 (s, 1H), 8.05 (s, 1H), 7.27-7.07 (m, 4H), 4.15-3.94 (m, 4H), 3.80-3.73 (m, 1H), |
| | 3.59-3.56 (m, 1H), 3.48-3.39 (m, 1H), 3.10-2.93 (m, 6H), 2.35 (s, 3H). |
| 531 | ¹H NMR (300 MHz, Chloroform-d) δ 10.03 (s, 1H), 8.28 (s, 1H), 7.28-7.07 (m, 4H), 4.12-4.03 (m, 4H), 3.79-3.73 (m, 1H), |
| | 3.61 (d, J = 9.0 Hz, 1H), 3.45-3.39 (m, 1H), 3.18 (s, 3H), 2.97 (s, 3H), 2.35 (s, 3H). |
| 532 | ¹H NMR (300 MHz, Chloroform-d) δ 10.14 (s, 1H), 8.18-8.15 (m, 2H), 7.24-7.20 (m, 1H), 7.14-7.04 (m, 3H), 4.18-4.08 (m, |
| | 4H), 3.86 (s, 3H), 3.83-3.76 (m, 1H), 3.57-3.53 (m, 1H), 3.44-3.37 (m, 1H), 2.96 (s, 3H), 2.34 (s, 3H). |
| 533 | ¹H NMR (300 MHz, Chloroform-d) δ 9.05 (s, 1H), 7.63-7.45 (m, 5H), 4.23-4.03 (m, 3H), 3.86-3.75 (m, 1H), 3.55 (d, J = 9.0 |
| | Hz, 1H), 3.51-3.42 (m, 1H), 2.99 (s, 3H), 1.40 (t, J = 7.2 Hz, 3H) |
| 534 | ¹H NMR (300 MHz, Chloroform-d) δ 9.25 (s, 1H), 7.85 (s, 1H), 7.60-7.47 (m, 4H), 5.35 (s, 2H), 4.24-4.13 (m, 1H), 3.88-3.77 |
| | (m, 1H), 3.62-3.55 (m, 1H), 3.50-3.40 (m, 1H), 3.26 (s, 3H), 3.00 (s, 3H), 2.73 (q, J = 7.5 Hz, 2H), 1.23 (t, J = 7.5 Hz, 3H). |
| 535 | ¹H NMR (300 MHz, Chloroform-d) δ 9.92 (s, 1H), 8.16 (s, 1H), 7.61-7.46 (m, 4H), 4.28-4.08 (m, 3H), 3.86-3.76 (m, 1H), |
| | 3.63-3.55 (m, 1H), 3.48-3.40 (m, 1H), 3.00 (s, 3H), 1.44 (t, J = 7.2 Hz, 3H). |
| 536 | ¹H NMR (300 MHz, Chloroform-d) δ 9.90 (s, 1H), 8.19 (s, 1H), 7.58-7.50 (m, 4H), 4.61-4.52 (m, 1H), 4.20-4.11 (m, 1H), |
| | 3.86-3.76 (m, 1H), 3.60 (d, J = 9.3 Hz, 1H), 3.47-3.41 (m, 1H), 3.00 (s, 3H), 1.49 (d, J = 6.6 Hz, 6H). |
| 537 | ¹H NMR (300 MHz, Chloroform-d) δ 9.94 (s, 1H), 8.09 (s, 1H), 7.58-7.47 (m, 4H), 4.18-4.09 (m, 1H), 3.84-3.53 (m, 1H), |
| | 3.61-3.53 (m, 2H), 3.47-3.41 (m, 1H), 3.00 (s, 3H), 1.29-1.24 (m, 2H), 1.12-1.02 (m, 2H). |
| 538 | ¹H NMR (300 MHz, Chloroform-d) δ 9.84 (s, 1H), 8.12 (s, 1H), 7.57-7.45 (m, 4H), 4.50-4.43 (m, 1H), 4.29-4.22 (m, 1H), |
| | 4.00-3.93 (m, 4H), 3.77-3.71 (m, 1H), 3.36 (s, 3H). |
| 539 | ¹H NMR (300 MHz, Chloroform-d) δ 9.91 (s, 1H), 8.03 (s, 1H), 7.57-7.46 (m, 4H), 4.26-4.14 (m, 1H), 3.96-3.93 (m, 3H), |
| | 3.85-3.79 (m, 1H), 3.58 (d, J = 9.0 Hz, 1H), 3.48-3.41 (m, 1H), 3.10-2.98 (m, 6H). |
| 540 | ¹H NMR (300 MHz, Chloroform-d) δ 9.50 (s, 1H), 8.00 (s, 1H), 7.61-7.47 (m, 4H), 5.45 (s, 2H), 4.25-4.13 (m, 1H), 3.88-3.76 |
| | (m, 1H), 3.64-3.56 (m, 1H), 3.51-3.40 (m, 1H), 3.30 (s, 3H), 3.01 (s, 3H), 1.78-1.70 (m, 1H), 1.14-1.07 (m, 2H), 0.85-0.72 |
| | (m, 2H). |
| 541 | ¹H NMR (300 MHz, DMSO-d6) δ 10.87 (s, 1H), 7.80 (s, 1H), 7.77-7.50 (m, 4H), 4.09-4.02 (m, 1H), 4.00 (s, 3H), 3.77-3.70 |
| | (m, 2H), 3.42-3.38 (m, 1H), 2.80 (s, 3H). |
| 542 | ¹H NMR (300 MHz, Chloroform-d) δ 9.31 (s, 1H), 8.09 (s, 1H), 7.56-7.52 (m, 4H), 7.49-7.47 (m, 1H), 7.23-7.21 (m, 2H), |
| | 4.23-4.13 (m, 1H), 3.88 (s, 3H), 3.83-3.77 (m, 1H), 3.52 (d, J = 9.0 Hz, 1H), 3.44-3.38 (m, 1H), 2.95 (s, 3H). |
| 543 | ¹H NMR (300 MHz, Chloroform-d) δ 10.27 (s, 1H), 9.04 (d, J = 2.1 Hz, 1H), 8.20 (s, 1H), 7.57-7.53 (m, 3H), 7.52-7.47 (m, |
| | 2H), 4.32-4.23 (m, 1H), 4.07 (s, 3H), 3.89-3.82 (m, 1H), 3.57 (d, J = 8.1 Hz, 1H), 3.46-3.40 (m, 1H), 2.97 (s, 3H). |
| 544 | ¹H NMR (300 MHz, Chloroform-d) δ 9.40 (s, 1H), 8.04 (s, 1H), 7.15 (t, J = 57.0 Hz, 1H), 6.95-6.91 (m, 3H), 4.06-3.97 (m, |
| | 1H), 3.79-3.72 (m, 1H), 3.63-3.58 (m, 1H), 3.46-3.39 (m, 1H), 2.98 (s, 3H), 2.85 (q, J = 7.2 Hz, 2H), 2.31 (s, 6H), 1.28 (t, J = |
| | 7.2 Hz, 3H). |
| 545 | ¹H NMR (300 MHz, Chloroform-d) δ 9.21 (s, 1H), 7.86 (s, 1H), 6.94-6.87 (m, 3H), 5.33 (s, 2H), 4.08-3.96 (m, 1H), 3.80-3.71 |
| | (m, 1H), 3.61-3.55 (m, 1H), 3.45-3.37 (m, 1H), 3.24 (s, 3H), 2.96 (s, 3H), 2.70 (q, J = 7.5 Hz, 2H), 2.27 (s, 6H), 1.20 (t, J = 7.5 |
| | Hz, 3H). |
| 546 | ¹H NMR (300 MHz, Chloroform-d) δ 9.54 (s, 1H), 7.81 (s, 1H), 6.95-6.89 (m, 3H), 6.86 (t, J = 39.9 Hz, 1H), 4.05-3.96 (m, |
| | 1H), 3.94 (s, 3H), 3.78-3.72 (m, 1H), 3.60-3.55 (m, 1H), 3.46-3.37 (m, 1H), 2.98 (s, 3H), 2.31 (s, 6H). |
| 547 | ¹H NMR (300 MHz, Chloroform-d) δ 9.84 (s, 1H), 8.21 (s, 1H), 6.95-6.88 (m, 3H), 4.60-4.51 (m, 1H), 4.04-3.95 (m, 1H), |
| | 3.78-3.69 (m, 1H), 3.59 (d, J = 8.7 Hz, 1H), 3.42-3.36 (m, 1H), 2.97 (s, 3H), 2.31 (s, 6H), 1.49-1.46 (m, 6H). |
| 548 | ¹H NMR (300 MHZ, Chloroform-d) δ 9.91 (s, 1H), 8.13 (s, 1H), 6.94-6.91 (m, 3H), 4.03-3.94 (m, 1H), 3.77-3.70 (m, 1H), |
| | 3.63-3.50 (m, 2H), 3.42-3.37 (m, 1H), 2.97 (s, 3H), 2.31 (s, 6H), 1.29-1.24 (m, 2H), 1.08-1.01 (m, 2H). |
| 549 | ¹H NMR (300 MHz, Chloroform-d) δ 9.39 (s, 1H), 8.01 (s, 1H), 6.96-6.89 (m, 3H), 5.45 (s, 2H), 4.11-3.99 (m, 1H), 3.81-3.71 |
| | (m, 1H), 3.64-3.54 (m, 1H), 3.45-3.37 (m, 1H), 3.30 (s, 3H), 2.98 (s, 3H), 2.31 (s, 6H), 1.76-1.71 (m, 1H), 1.11-1.05 (m, 2H), |
| | 0.87-0.77 (m, 2H). |
| 550 | ¹H NMR (300 MHz, Chloroform-d) δ 9.37 (s, 1H), 8.23 (s, 1H), 7.62-7.57 (m, 1H), 7.27-7.23 (m, 2H), 6.91-6.68 (m, 3H), |
| | 4.07-3.98 (m, 1H), 3.93 (s, 3H), 3.77-3.70 (m, 1H), 3.52 (d, J = 8.4 Hz, 1H), 3.41-3.35 (m, 1H), 2.93 (s, 3H), 2.29 (s, 6H). |
| 551 | ¹H NMR (300 MHz, Chloroform-d) δ 10.14 (s, 1H), 9.02 (d, J = 1.8 Hz, 1H), 8.22 (s, 1H), 7.55-7.53 (m, 1H), 6.93-6.87 (m, |
| | 3H), 4.16-4.09 (m, 1H), 4.07 (s, 3H), 3.83-3.75 (m, 1H), 3.56 (d, J = 6.9 Hz, 1H), 3.43-3.36 (m, 1H), 2.95 (s, 3H), 2.29 (s, 6H). |
| 552 | ¹H NMR (300 MHz, Chloroform-d) δ 9.79 (s, 1H), 8.05 (s, 1H), 6.92-6.91 (m, 3H), 4.11-3.94 (m, 4H), 3.76-3.73 (m, 1H), 3.58 |
| | (d, J = 8.7 Hz, 1H), 3.43-3.37 (m, 1H), 3.11-2.95 (m, 6H), 2.31 (s, 6H). |
| 553 | ¹H NMR (300 MHz, Chloroform-d) δ 9.35 (s, 1H), 7.86 (s, 1H), 6.95-6.87 (m, 3H), 4.48 (s, 2H), 4.07-3.99 (m, 1H), 3.90-3.70 |
| | (m, 4H), 3.56 (d, J = 8.4 Hz, 1H), 3.45-3.35 (m, 4H), 2.97 (s, 3H), 2.30 (s, 6H). |
| 554 | ¹H NMR (300 MHz, Chloroform-d) δ 9.67 (s, 1H), 8.22 (s, 1H), 7.33-6.95 (m, 4H), 4.08-3.99 (m, 1H), 3.81-3.75 (m, 1H), 3.56 |
| | (d, J = 9.3 Hz, 1H), 3.46-3.39 (m, 1H), 2.99 (s, 3H). |
| 555 | ¹H NMR (300 MHz, Chloroform-d) δ 9.35 (s, 1H), 7.86 (s, 1H), 7.14-6.97 (m, 3H), 4.14-4.06 (m, 1H), 3.90-3.75 (m, 5H), 3.52 |
| | (d, J = 8.7 Hz, 1H), 3.43-3.37 (m, 1H), 2.99 (s, 3H), 1.08-1.05 (m, 2H), 0.75-0.65 (m, 2H). |
| 556 | ¹H NMR (300 MHz, Chloroform-d) δ 9.44 (s, 1H), 7.95-7.91 (m, 1H), 7.29-7.26 (m, 1H), 7.23-7.11 (m, 2H), 6.62 (t, J = 56.1 |
| | Hz, 1H), 4.19-4.08 (m, 1H), 3.88-3.76 (m, 5H), 3.58 (d, J = 9.2 Hz, 1H), 3.49-3.41 (m, 1H), 3.01-2.99 (m, 2H). |
| 557 | ¹H NMR (300 MHz, Chloroform-d) δ 9.70 (s, 1H), 8.21 (s, 1H), 7.71-7.53 (m, 3H), 7.15 (t, J = 57.9 Hz, 1H), 4.19-4.04 (m, |
| | 1H), 3.91-3.77 (m, 1H), 3.60 (d, J = 9.4 Hz, 1H), 3.51-3.40 (m, 1H), 3.02 (s, 3H). |
| 558 | ¹H NMR (300 MHz, Chloroform-d) δ 9.43 (s, 1H), 7.95 (s, 1H), 7.30-7.22 (m, 3H), 6.61 (t, J = 56.4 Hz, 1H), 4.14-4.05 (m, |
| | 1H), 3.82-3.75 (m, 4H), 3.61 (d, J = 9.0 Hz, 1H), 3.47-3.41 (m, 1H), 3.01-3.00 (m, 3H), 2.40 (s, 3H). |
| 559 | ¹H NMR (300 MHz, Chloroform-d) δ 9.75 (s, 1H), 8.24 (s, 1H), 7.29-7.23 (m, 3H), 7.14 (t, J = 57.9 Hz, 1H), 6.62 (t, J = 56.4 |
| | Hz, 1H), 5.31-5.29 (m, 1H), 4.16-4.00 (m, 1H), 3.82-3.76 (m, 1H), 3.64 (d, J = 9.3 Hz, 1H), 3.48-3.42 (m, 1H), 3.01 (s, 3H), |
| | 2.41 (s, 2H). |
| 560 | ¹H NMR (300 MHz, Chloroform-d) δ 9.73 (s, 1H), 8.23 (s, 1H), 7.40-7.34(m, 3H),7.15 (t, J = 57.9Hz, 1H), 4.12-4.03 (m, 1H), |
| | 3.83-3.76 (m, 1H), 3.63 (d, J = 9.3 Hz, 1H), 3.48-3.42 (m, 1H), 3.01 (s, 3H), 2.43 (s, 3H). |
| 561 | ¹H NMR (300 MHz, Chloroform-d) δ 9.38 (s, 1H), 7.87 (s, 1H), 7.38-7.33 (m, 3H), 4.21-4.08 (m, 1H), 3.85 (s, 3H), 3.83-3.76 |
| | (m, 1H), 3.58 (d, J = 8.7 Hz, 1H), 3.46-3.36 (m, 1H), 3.00 (s, 3H), 2.41 (s, 3H), 2.07-1.97 (m, 1H), 1.25-1.02 (m, 2H), 0.88- |
| | 0.59 (m, 2H). |
| 562 | ¹H NMR (300 MHz, Chloroform-d) δ 9.41 (s, 1H), 8.18 (s, 1H), 7.95 (s, 1H), 7.59-7.53 (m, 1H), 6.75 (d, J = 8.4 Hz, 1H), 4.05- |
| | 3.98 (m, 1H), 3.92 (s, 3H), 3.81 (s, 3H), 3.78-3.71 (m, 1H), 3.50 (d, J = 9.2 Hz, 1H), 3.45-3.37 (m, 1H), 2.99 (s, 3H). |

Table A has the same structure as the above Table 1 does, except that the general formula I is replaced with the general formula I′

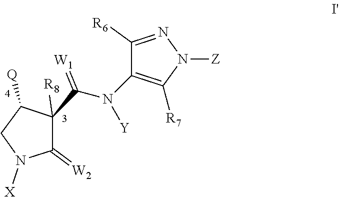

which has chiral centers, and in Table A, the items below the column heading “No.” are sequentially listed as 1′-562′. For example, 1′ corresponds to a compound that is Compound 1 in Table 1 with S-configuration at both the 3- and 4-positions

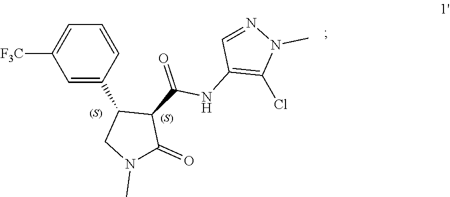

30′ corresponds to a compound that is Compound 30 in Table 1 with S-configuration at both the 3- and 4-positions

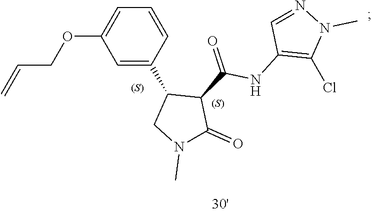

340′ corresponds to a compound that is Compound 340 in Table 1 with S- and R-configurations at the 3- and 4-positions, respectively

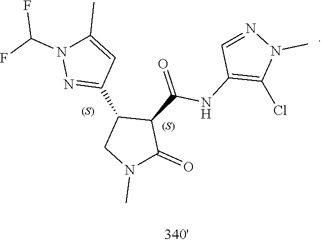

Several methods for preparing a compound of the present invention are illustrated in detail in the following schemes and examples. Raw materials can be purchased on the market or can be prepared by methods known in literature or as shown in the detailed description. A person skilled in the art should understand that other synthetic routes may also be used to synthesize a compound of the present invention. Although the specific raw materials and conditions in the synthetic routes have been illustrated below, they may be easily replaced with other similar raw materials and conditions. Various isomers and the like of the compounds resulted from these modifications or variants of the preparation methods of the present invention are all included in the scope of the present invention. In addition, the preparation methods as described below can be further modified according to the disclosure of the present invention using conventional chemical methods well known to a person skilled in the art. For example, the protection for an appropriate group during the reaction, etc.

Examples of methods provided below are used to enhance further understanding of the preparation methods of the present invention. The specific substances, varieties and conditions employed are determined to be further illustrations of the present invention and not to limit its reasonable scope. Reagents used in the synthesis of the compounds illustrated below are either commercially available or can be easily prepared by a person of ordinary skill in the art.

Examples of representative compounds are as follows. The synthesis methods of other compounds are similar and will not be described in detail here.

### 1. Synthesis of Compound 1

Py-BOP (6.80 g, 13.1 mmol), Compound 1-1 (1.72 g, 13.1 mmol) and triethylamine (2.41 g, 17 mmol) were added into the DCM solution (100 mL) of Compound 1-2 (2.50 g, 8.71 mmol) and stirred at room temperature. Water (100 mL*3) was added for extraction. The organic phase was dried over anhydrous sodium sulfate, added with silica gel powder, concentrated by decompression, and rotary-evaporated to completely remove the solvent followed by normal phase purification (mobile phase was petroleum ether:ethyl acetate=1:1) to obtain Compound 1 (2 g, yellow solid).

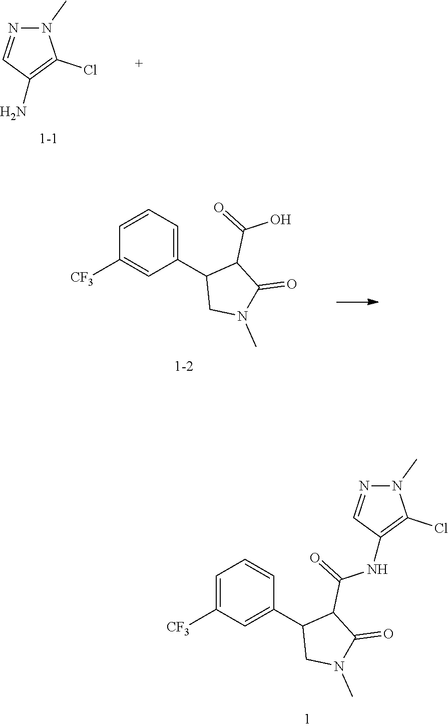

### 2. Synthesis of Compound 17′

(1) Compound 17-1 (5 g) was added into a flask, added with THF, and stirred evenly. At 0° C., the system was added with 3 eq of potassium tert-butoxide in batches, then slowly added with 2 eq of methyl iodide in drops using a pressure-equalized dropping funnel, and reacted at room temperature afterwards. After the reaction, the system was treated by adjusting pH to acidity, extraction, filtration and drying to obtain 3 g of Compound 17-2.

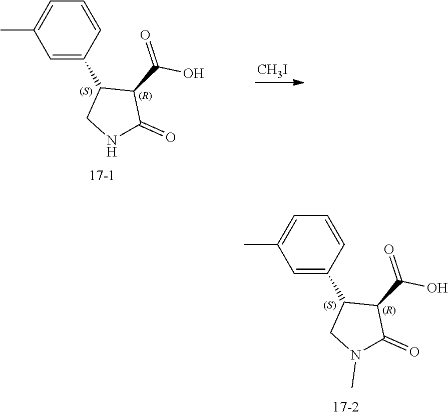

(2) Compound 17-2 (300 mg, 1 eq), the pyrazole compound 1-1 (1.2 eq), EDCI (1.5 eq), HOBT (1.5 eq), triethylamine (3 eq) and dichloromethane (10 mL) were added into a 50 mL single-necked flask and reacted at room temperature for 4 h. Compound 17′ (60 mg, 92% yield) was obtained by column chromatography.

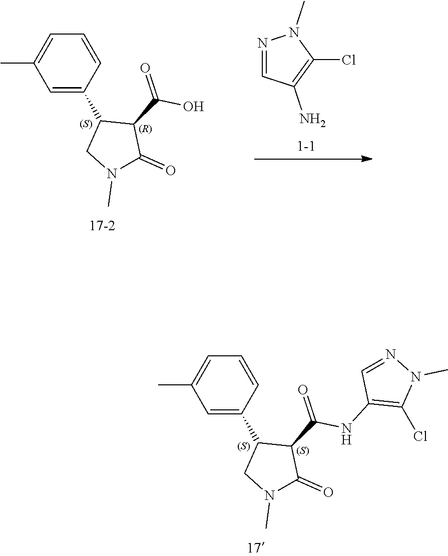

### 3. Synthesis of Compound 30

(1) Compound 30-1 was prepared with reference to Compound 17-2. Compound 30-1 (2 g) was dissolved in 20 mL of DCM, and in ice bath, added with boron tribromide (5 eq) and reacted for 30 min. After the In-Process Control was completed, the reaction solution was added with methanol to quench the reaction and spin-dried to obtain the product Compound 30-2 (1.9 g, white solid).

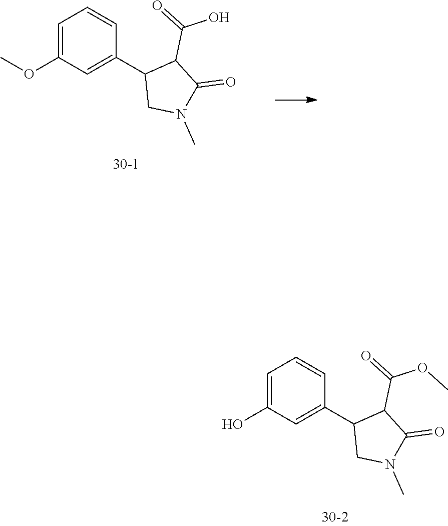 [SMILES: COc1cccc(C2CN(C)C(=O)C2C(=O)O)c1 ; COC(=O)C1C(=O)N(C)CC1c1cccc(O)c1]

(2) 100 mg of Compound 30-2 was dissolved in 5 mL of acetonitrile, added with potassium carbonate (2 eq), added with 30-3 (1.2 eq), and reacted overnight at 30° C. After the In-Process Control was completed, the reaction solution was immediately added with silica gel, stirred, and separated by silica gel column chromatography to obtain Compound 30-4 (90 mg, white solid).

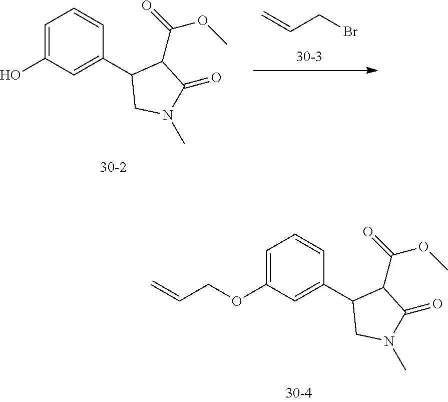

(3) 90 mg of Compound 30-4 was dissolved in 3 mL of dioxane, added with 2 mL of water, added with sodium hydroxide (1.5 eq), and reacted for 30 min. After the In-Process Control was completed, the reaction solution was concentrated by decompression to remove dioxane, pH of which was adjusted with HCl to acidity, and extracted twice with DCM. The organic phase was dried and spin-dried to obtain Compound 30-5 (70 mg, pale yellow solid).

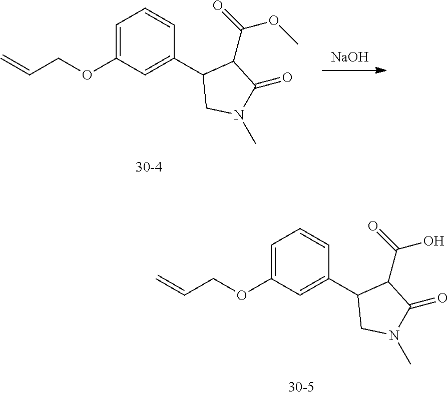

(4) 70 mg of Compound 30-5 was dissolved in 3 mL of DCM, added with triethylamine (1.5 eq), added with Compound 1-1 (1.2 eq), added with Py-BOP (1.5 eq), and reacted for 30 min. After the In-Process Control was completed, the reaction solution was added with silica gel, stirred, spin-dried, and separated and purified through a silica gel column to obtain Compound 30 (60 mg, white solid).

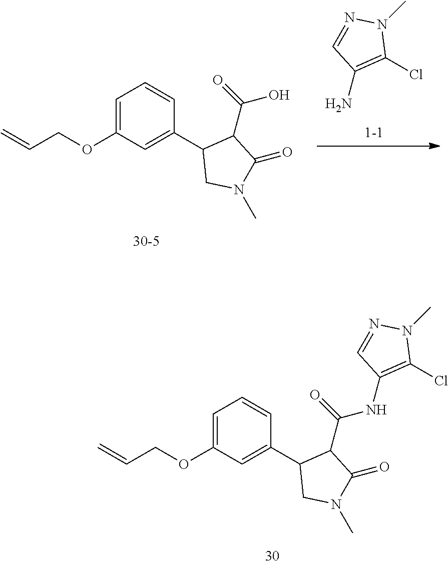

### 4. Synthesis of Compound 53

(1) 50 mL of dichloromethane, Compound 30-1 (300 mg, 1 eq), the pyrazole compound 1-1 (240 mg, 1.5 eq), triethylamine (240 mg, 2 eq) and Py-BOP (915 mg, 1.5 eq) were added into a 100 mL three-necked flask and reacted at room temperature for 4 h. After the reaction was completed, the reaction solution was extracted with water and dichloromethane. The organic phase was dried over anhydrous sodium sulfate to remove the water, concentrated by decompression to remove the solvent, and purified by silica gel column chromatography to obtain the white solid product Compound 53-1 (200 mg, 95% purity).

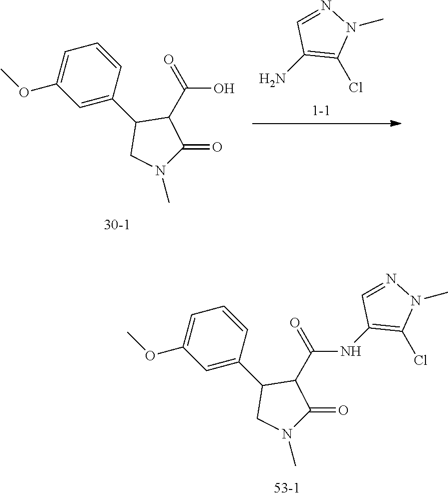

(2) 30 mL of dichloromethane and Compound 53-1 (200 mg, 1 eq) were added into a 100 mL three-necked flask. The system was added with boron tribromide (414 mg, 3 eq) in ice bath and naturally warmed to room temperature to continue the reaction for 4 h. After the reaction was completed, the reaction was quenched by methanol, and water and dichloromethane were employed for extraction. The organic phase was dried over anhydrous sodium sulfate to remove the water, concentrated by decompression to remove the solvent, and spin-dried to obtain the solid Compound 53-2 for use in the next step (176 mg, 95% purity, crude product).

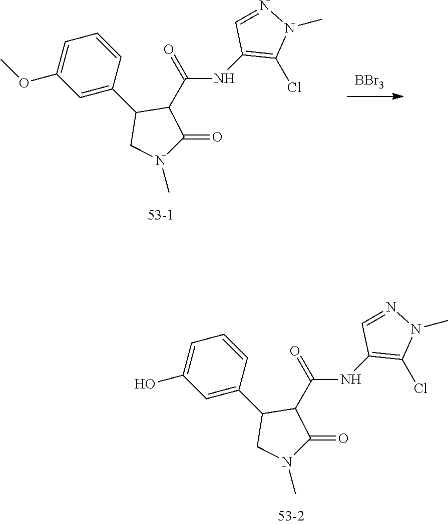

(3) 30 mL of DMF, Compound 53-2 (100 mg, 1 eq), Compound 53-3 (45 mg, 1.2 eq) and anhydrous potassium carbonate (120 mg, 3 eq) were added into a 100 mL three-necked flask and reacted at 50° C. for 4 h. After the reaction was completed, the reaction solution was extracted with water and ethyl acetate. The organic phase was washed twice with saturated saline solution, dried over anhydrous sodium sulfate to remove the water, concentrated by decompression to remove the solvent, and purified by silica gel column chromatography to obtain the white solid product Compound 53 (30 mg, 89% purity).

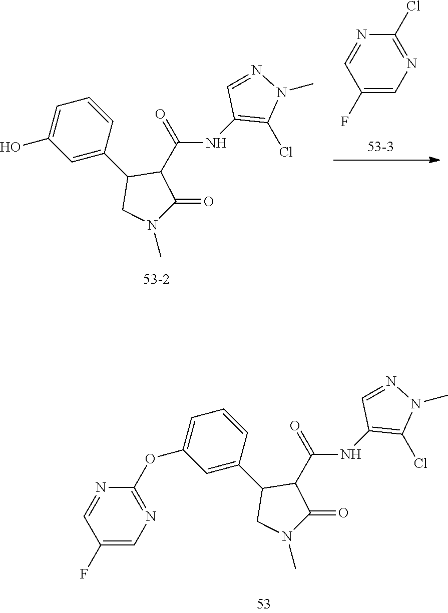

### 5. Synthesis of Compound 97

15 mL of DCM, Compound 1-2 (0.100 g, 1.0 eq), Compound 97-1 (0.836 g, 1.5 eq), TEA (0.710 g, 2.0 eq) and Py-BOP (0.270 g, 1.5 eq) were added into a 50 mL round-bottom flask and reacted at room temperature for 2 h. After the reaction was completed, the reaction solution was extracted with water and ethyl acetate, dried over anhydrous sodium sulfate, concentrated by decompression to remove the solvent, and purified by silica gel column chromatography to obtain the yellow oily matter Compound 97 (30 mg, 92% purity, 19% yield).

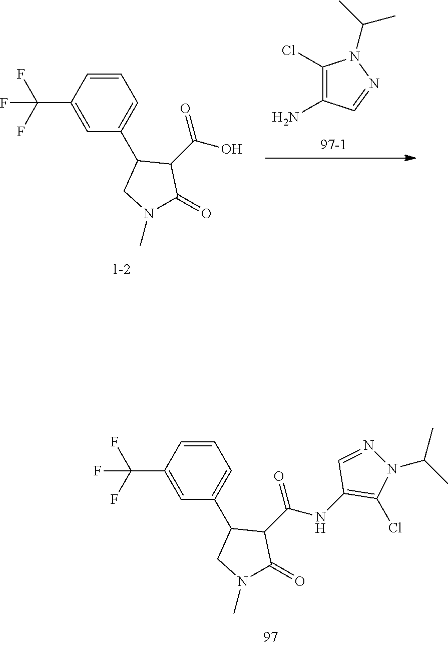

### 6. Synthesis of Compound 117′

(1) 100 g of Compound 117-1 was dissolved in 200 mL of glacial acetic acid, added with nitromethane (3.0 eq) and ammonium acetate (0.5 eq), displaced with nitrogen gas, and reacted at 100-105° C. for 2-5 h. The HPLC detected the completion of the reaction. The reaction system was cooled down to 20-25° C., added with 200 mL of purified water and filtered. The filter cake was soaked and washed with 200 mL×2 of isopropanol for twice and dried to obtain 90 g of the yellow solid Compound 117-2, with a yield of 68%.

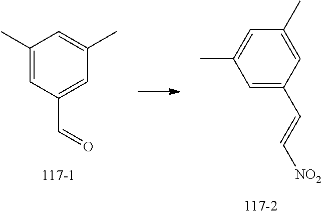

(2) A total of 100 g of Compound 117-2 was dissolved in 500 mL of toluene, added with 5% w/w catalyst (CAS No.: 862910-64-3) and diethyl malonate (1.25 eq), displaced with nitrogen gas, and reacted at 55-60° C. for 3-5 h. The HPLC detected the completion of the reaction. The reaction system was cooled down to 20-25. The organic phase was sequentially washed with 200 mL of purified water and 200 mL of 15% saline solution once each. The organic phase was concentrated by decompression until no fraction generated, added with 300 mL of n-heptane, slurried at 20-25° C. for 1 h, and filtered. The filter cake was dried to obtain 170 g of the pale yellow solid Compound 117-3, with (S):(R)=97:3 and a yield of 80%.

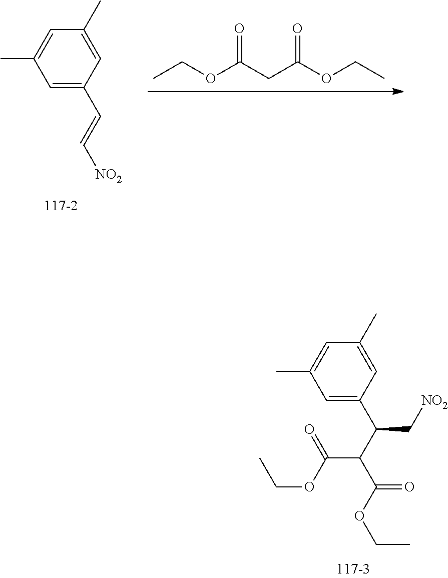 [SMILES: Cc1cc(C)cc(/C=C/[N+](=O)[O-])c1 ; CCOC(=O)CC(=O)OCC ; CCOC(=O)C(C(=O)OCC)[C@H](C[N+](=O)[O-])c1cc(C)cc(C)c1]

(3) 100 g of Compound 117-3 was dissolved in 800 mL of MeOH, added with NiCl_{2·6}H₂O (0.5 eq), kept at 0-20° C., added with NaBH₄ (3.0 eq) in batches, and reacted for 3 h at room temperature. The completion of the reaction was detected by HPLC In-Process Control. The methanol reaction solution of Compound 117-4 was obtained, and the next reaction was carried out directly without post-treatment.

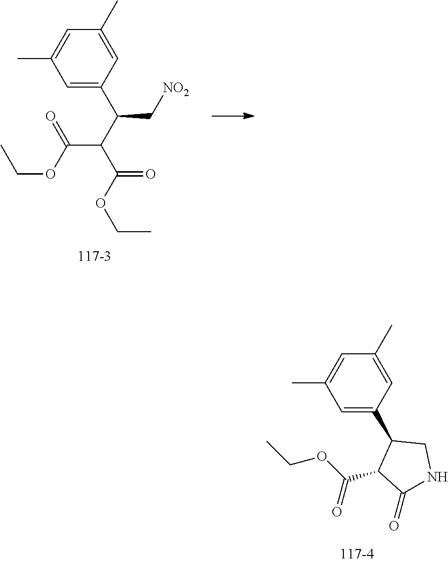 [SMILES: CCOC(=O)C(C(=O)OCC)[C@H](C[N+](=O)[O-])c1cc(C)cc(C)c1 ; CCOC(=O)[C@H]1C(=O)NC[C@@H]1c1cc(C)cc(C)c1]

(4) The methanol reaction solution of Compound 117-4 obtained in the previous step was added with 200 mL of purified water and NaOH (3.0 eq) and reacted at 20-25° C. for 24 h. The completion of the reaction was detected by HPLC In-Process Control. The reaction solution was adjusted to pH=2 using 4M HCl, extracted with 300 mL of DCM, and concentrated to obtain 70 g of the crude Compound 117-5 for direct use in the next step.

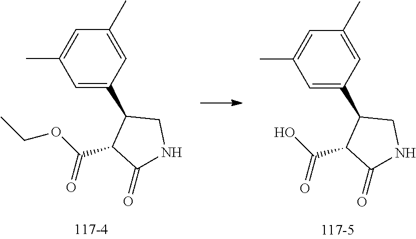 [SMILES: CCOC(=O)[C@H]1C(=O)NC[C@@H]1c1cc(C)cc(C)c1 ; Cc1cc(C)cc([C@H]2CNC(=O)[C@@H]2C(=O)O)c1]

(5) 70 g of the crude Compound 117-5 was dissolved in 560 mL of THF, added with tBuOK (3.0 eq) and dimethyl sulfate (1.5 eq) at 0° C., and stirred at room temperature for 2 h. The completion of the reaction was detected by HPLC In-Process Control. The reaction solution was added with 280 mL of purified water and concentrated. The concentrate was adjusted to pH=2 using 4M HCl and filtered. The filter cake was recrystallized using 350 mL of ethyl acetate and dried to obtain 45 g of the white solid Compound 117-6, with (3R,4S):(3S,4R)=97:3 and a yield of 65% over three steps (from Compound 117-3 to Compound 117-6).

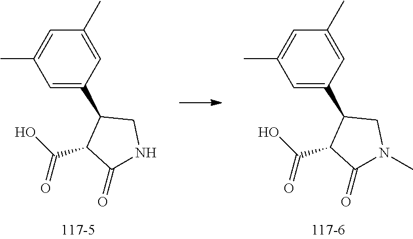

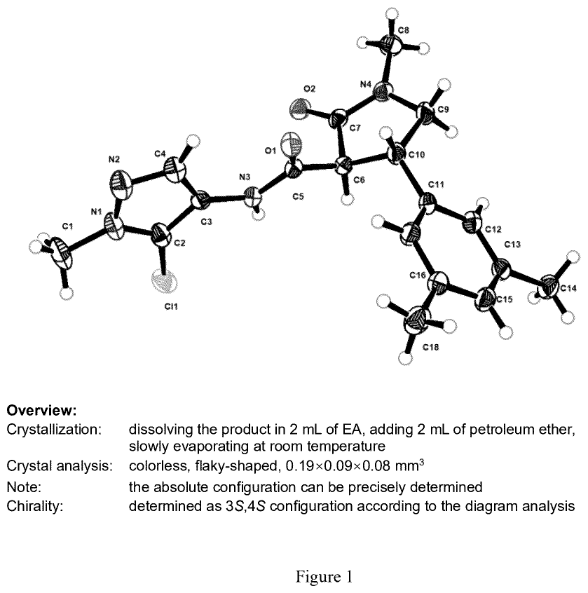

(6) A total of 100 g of Compound 117-6 was dissolved in 300 mL of DCE, added with 1 mL of DMF, added with oxalyl chloride (1.1 eq) in drops at 40-42° C. The completion of the reaction was detected by HPLC In-Process Control. The DCE was concentrated by decompression and added with 300 mL of DCM to obtain acyl chloride solution of Compound 117-5. The foregoing solution was added dropwise into DCM solution of Compound 1-1 (1.0 eq) at 40-42° C. Upon the completion of addition, the system was reacted for 0.5 h. The completion of the reaction was detected by HPLC In-Process Control. The temperature was cooled down to 20-25° C. The DCM organic phase was sequentially washed with 200 mL×3 of purified water for three times, with 200 mL of saturated sodium carbonate solution for once, and with 200 mL×2 of purified water for twice. The DCM organic phase was concentrated by decompression and added with 120 mL of anhydrous ethanol for recrystallization. The solution was filtered. The filter cake was washed three times with 0-5° C. anhydrous ethanol and dried to obtain 120 g of the pale yellow solid product Compound 117′, with (3S,4S):(3R,4R)>99:1 and a yield of 83%. Its absolute configuration was determined by single crystal X-ray structure analysis, see FIG. 1.

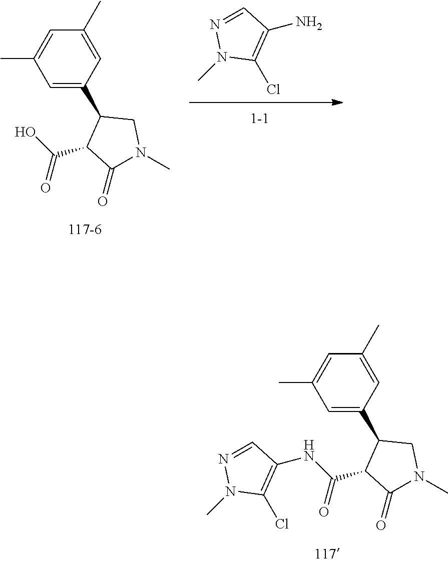

### 7. Synthesis of Compound 122

(1) 2.2 g of raw material Compound 122-1 was dissolved in tetrahydrofuran, and in ice bath, added with 2 eq of potassium tert-butoxide, added with 1.2 eq of methyl iodide in drops, and reacted at room temperature for 1 h. After the In-Process Control was completed, water was added to quench the reaction. The aqueous phase was adjusted to pH in acidity, and the solution was extracted with EA and dried to obtain 2 g of the white solid Compound 122-2.

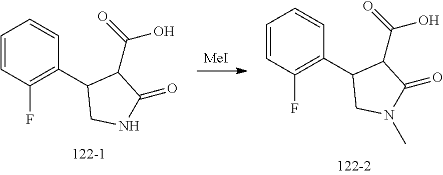 [SMILES: "MeI" ; O=C(O)C1C(=O)NCC1c1ccccc1F ; CN1CC(c2ccccc2F)C(C(=O)O)C1=O]

(2) 150 mg of Compound 122-2 was dissolved in dichloromethane, added with 1.1 eq of the pyrazole compound 1-1, added with 1.5 eq of EDCI, added with 1.5 eq of HATU, and reacted at room temperature for 1 h. After the In-Process Control was completed, the reaction solution was added with water to quench the reaction, washed with dilute hydrochloric acid, extracted with EA, and dried to obtain 134 mg of the white solid Compound 122 by normal phase column chromatography.

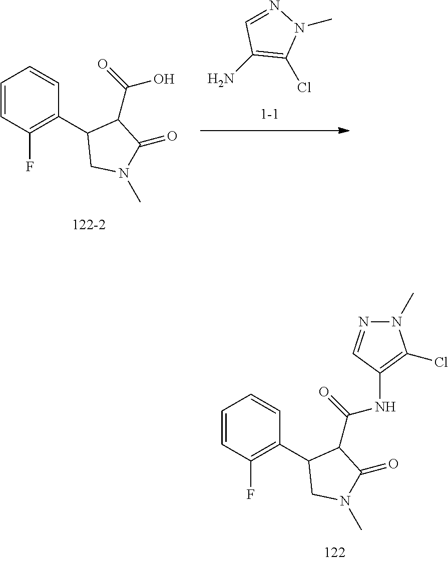

### 8. Synthesis of Compound 155

(1) Compound 155-1 (15 g, 0.0508 mol, 1 eq) was dissolved in DMF (150 mL). At room temperature, the reaction solution was added with benzyl bromide (8.69 g, 0.0508 mol, 1 eq) and reacted overnight at normal temperature. When liquid chromatography detected the raw materials had been completely consumed, the aqueous phase was extracted with water and ethyl acetate until no product present therein. The ethyl acetate phase was dried and concentrated to obtain 19 g of Compound 155-2 (97% yield).

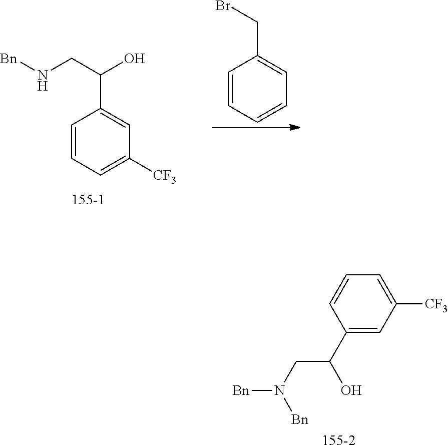

(2) Compound 155-2 (19 g, 0.049 mol, 1 eq) was dissolved in DCE (150 mL), added with a catalytic amount of DMF and POCl₃ (22.65 g, 0.147 mol, 3 eq), and reacted at 60° C. overnight. When liquid chromatography detected that the raw materials had been completely consumed, the reaction solution was added with water to quench the reaction (temperature control) and added with NaHCO₃ solution to adjust the pH to 7, and the solution was separated. The organic phase was dried and concentrated to obtain the crude product which was then purified into Compound 155-3 (5.2 g, 25% yield).

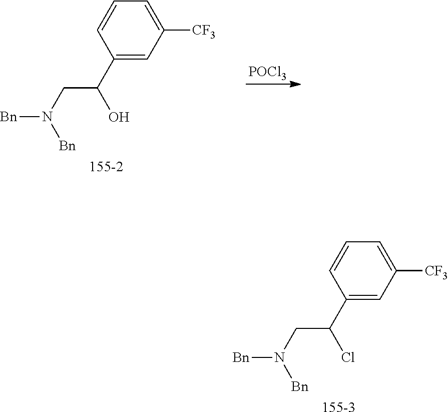

(3) Compound 155-3 (5.2 g, 0.012 mol, 1 eq) was dissolved in MeCN (50 mL), added with 20% (wt:wt) molecular sieves, added with K₂CO₃ (4.0 eq) and diethyl malonate (1.5 eq), and reacted at 60° C. overnight. When liquid chromatography detected that the raw materials had been completely consumed, K₂CO₃ was filtered out. The organic phase was concentrated, stirred and purified through a silica gel column to obtain the product Compound 155-4 (5 g, 73% yield).

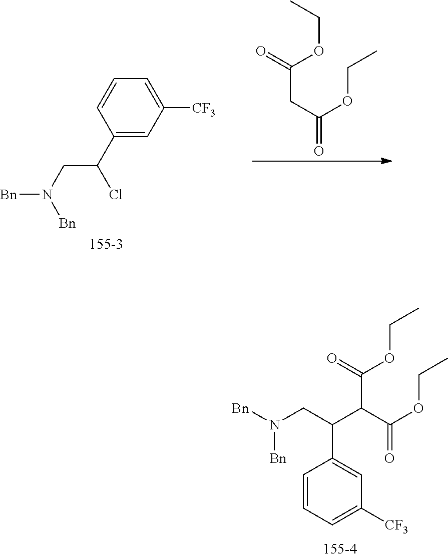

(4) Compound 155-4 was dissolved in MeOH, added with 0.05 eq of wet Pd/C (10%), and reacted under a double-layered hydrogen balloon (approximately 15 psi) at 50° C. for 24 h. When liquid chromatography detected the completion of the reaction, the wet Pd/C was filtered out. The mother liquor was concentrated, stirred, and purified to obtain the product Compound 155-5.

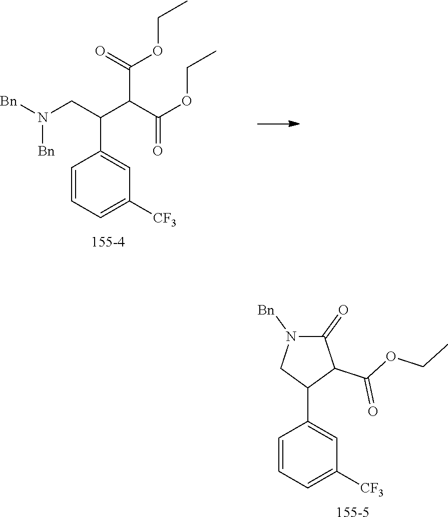

(5) Compound 155-5 (1 eq) and NaOH (3.5 eq) were dissolved in a mixed solvent (dioxane:H₂O=3:1) and reacted at normal temperature for 30 min. When the completion of the hydrolysis was detected, the reaction solution was concentrated, added with water, and extracted three times with DCM. The aqueous phase was adjusted to pH=4 and extracted with DCM. The organic phase was dried and concentrated to obtain the product, white solid Compound 155-6.

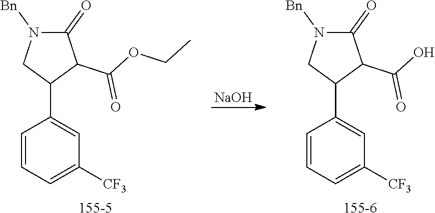

(6) Compound 155-6 (150 mg, 0.0004 mol, 1 eq), Compound 1-1 (1 eq) and triethylamine (2 eq) were dissolved in dichloromethane, added with HATU (2 eq) under stirring, and reacted at normal temperature for 60 min. When the completion of the reaction was detected, the reaction solution was concentrated, stirred, and purified to obtain the product, white solid Compound 155-7 (30 mg, 15% yield).

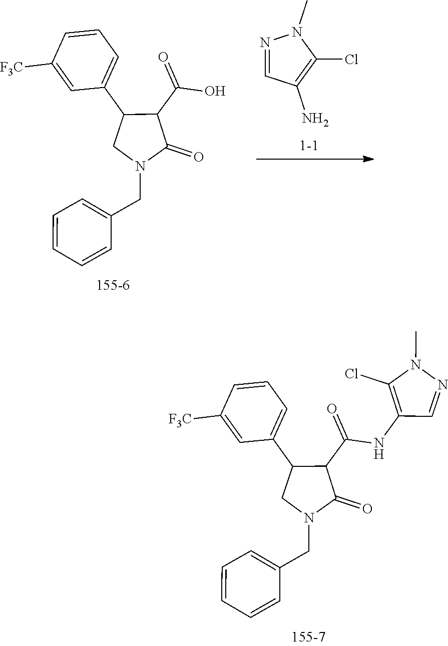

(7) Compound 155-7 (100 mg, 0.00021 mol, 1 eq) was dissolved in MeOH, added with 0.05 eq of wet Pd/C (10%), and reacted under a double-layered hydrogen balloon (approximately 15 psi) at 50° C. for 24 h. When the liquid chromatography detected the completion of the reaction, wet Pd/C was filtered out. The mother liquor was concentrated, stirred, and purified to obtain Compound 155 (80 mg, 86% yield).

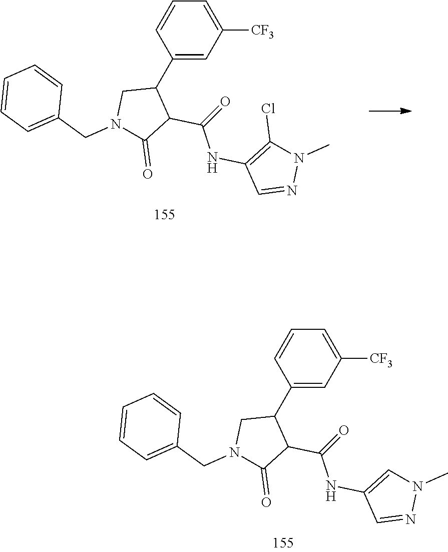

### 9. Synthesis of Compound 160′

(1) 20 mL of EtOH, 2 mL of H₂O, Compound 160-1 (0.500 g, 1.0 eq), iron powder (0.880 g, 5.5 eq) and ammonium chloride (0.460 g, 3.0 eq) were added into a 50 mL round-bottom flask and reacted at 50° C. overnight. Upon the completion of the reaction, the reaction solution was filtered with diatomaceous earth and extracted with water and dichloromethane. The organic phase was washed twice with saturated saline solution and concentrated by decompression to remove the solvent to obtain the yellow oily matter Compound 160-2 (400 mg, 60% purity, 80% yield).

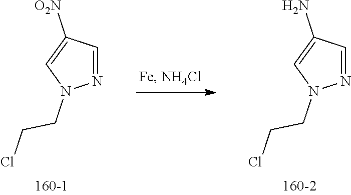 [SMILES: O=[N+]([O-])c1cnn(CCCl)c1 ; Nc1cnn(CCCl)c1]

(2) 20 mL of DCM, Compound 160-2 (0.400 g, 1.0 eq) and triethylamine (0.900 g, 3.0 eq) were added into a 50 mL round-bottom flask, added with Boc-acid anhydride (0.720 g, 1.2 eq) under stirring, and reacted at room temperature overnight. Upon the completion of the reaction, the reaction solution was extracted with water and dichloromethane. The organic phase was washed twice with saturated saline solution and concentrated by decompression to remove the solvent to obtain the yellow oily matter Compound 160-3 (240 mg, 80% purity, 70% yield).

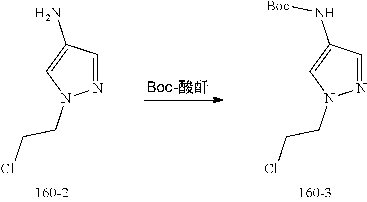 [SMILES: Nc1cnn(CCCl)c1 ; CC(C)(C)OC(=O)Nc1cnn(CCCl)c1]

(3) 20 mL of DMF, Compound 160-3 (0.240 g, 1.0 eq) and N-chlorosuccinimide (0.105 g, 0.8 eq) were added into a 50 mL round-bottom flask and reacted at 30° C. overnight. Upon the completion of the reaction, the reaction solution was extracted with water and dichloromethane. The organic phase was washed twice with saturated saline solution and concentrated by decompression to remove the solvent to obtain the yellow oily matter Compound 160-4 (330 mg, 80% purity, 70% yield).

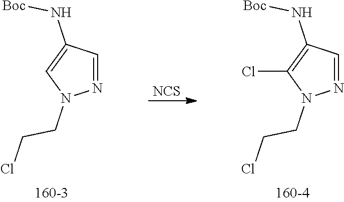 [SMILES: CC(C)(C)OC(=O)Nc1cnn(CCCl)c1 ; "NCS" ; CC(C)(C)OC(=O)Nc1cnn(CCCl)c1Cl]

(4) 20 mL of EA/HCl and Compound 160-4 (0.330 g, 1.0 eq) were added into a 50 mL round-bottom flask and reacted under stirring at room temperature overnight. Upon the completion of the reaction, the produ

$$\begin{matrix}Boc - acid \\ anhydride \\ \begin{matrix}⊥ & ⊥\end{matrix}\end{matrix}$$

ted, and then was filtered and concentrated by decompression to remove the solvent to obtain the white solid Compound 160-5 (130 mg, 80% purity, 50% yield).

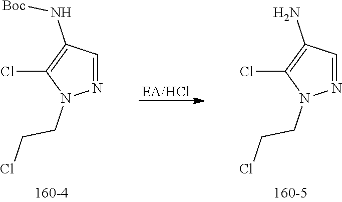 [SMILES: CC(C)(C)OC(=O)Nc1cnn(CCCl)c1Cl ; "EA/HCl" ; Nc1cnn(CCCl)c1Cl]

(5) 15 mL of DCM, Compound 160-5 (0.130 g, 1.0 eq), Compound 160-6 (0.145 g, 0.7 eq), triethylamine (0.220 g, 3.0 eq) and 1-hydroxybenzotriazole (0.150 g, 1.5 eq) were added into a 50 mL round-bottom flask, added with 1-ethyl-(3-dimethylaminopropyl)carbodiimide hydrochloride (EDCI) (0.210 g, 1.5 eq) under stirring, and reacted at room temperature for 1 h. Upon the completion of the reaction, the reaction solution was extracted with water and dichloromethane. The organic phase was washed twice with saturated saline solution, concentrated by decompression to remove the solvent, and purified by silica gel column chromatography to obtain the white solid Compound 160′ (80 mg, 96% purity, 20% yield).

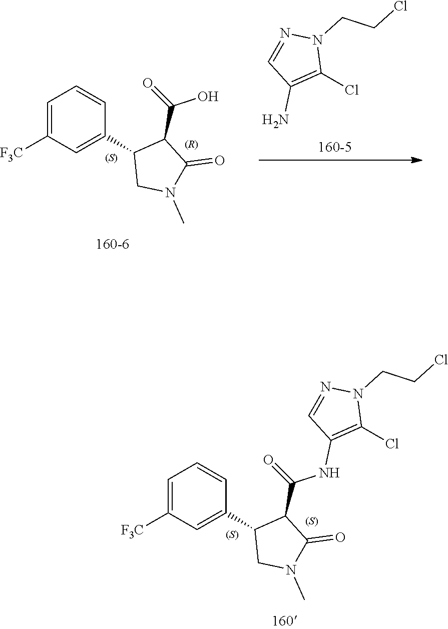

### 10. Synthesis of Compound 221

(1) 750 mg of Compound 30-2 was dissolved in 10 mL of acetonitrile, added with 3 eq of potassium carbonate, added with 1.2 eq of n-butyl iodide, and reacted at room temperature overnight. After the In-Process Control was completed, the reaction solution was immediately added with silica gel, stirred, and purified through a silica gel column to obtain 690 mg of the white solid product Compound 221-1.

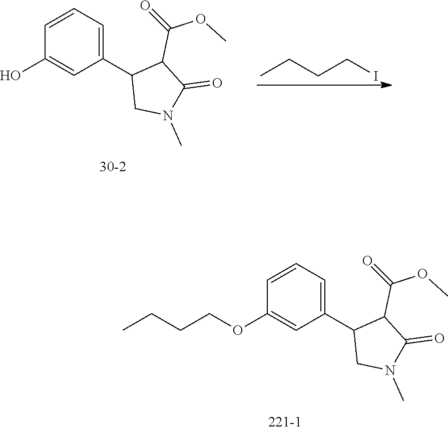

(2) 690 mg of Compound 221-1 was dissolved in 10 mL of tetrahydrofuran, added with 5 mL of water, added with 2 eq of sodium hydroxide, and reacted for 30 min. After the In-Process Control was completed, the tetrahydrofuran was spun out. The reaction solution was adjusted to acidity with dilute HCl and extracted twice with DCM. The organic phase was dried and spin-dried to obtain 600 mg of the white solid Compound 221-2.

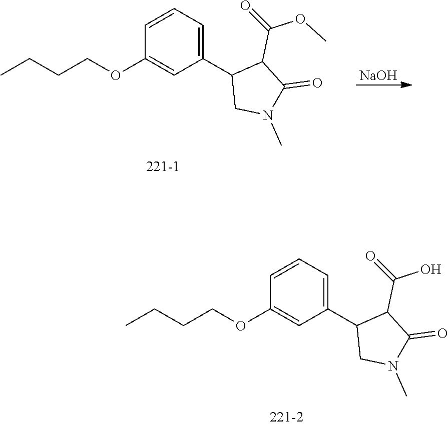

(3) 90 mg of Compound 221-2 was dissolved in 10 mL of DCM, added with 1.5 eq of triethylamine, added with 1.2 eq of the pyrazole compound 1-1, then added with 1.5 eq of EDCI and HATU respectively, and reacted for 30 min. After the In-Process Control was completed, the organic phase was washed with dilute hydrochloric acid, stirred and spin-dried, and purified through a silica gel column to obtain 107 mg of the white solid product Compound 221.

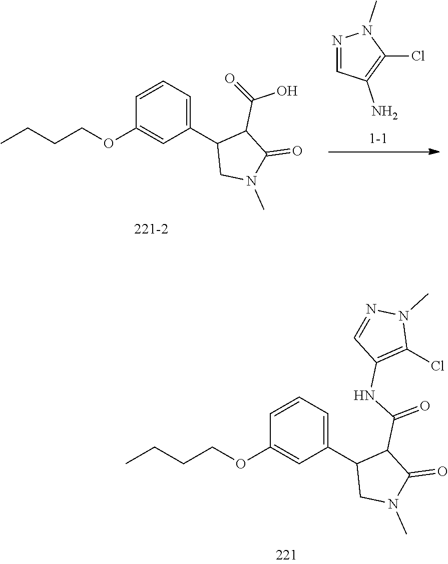

### 11. Synthesis of Compound 261

(1) Compound 261-1 (12.1 g, 43 mmol, 1 eq) was dissolved in 100 mL of anhydrous tetrahydrofuran, and in ice bath, slowly added with potassium tert-butoxide (130 mL, 0.13 mol, 3 eq, 1 M in THF). After being stirred for 1 h, the reaction solution was added with methyl iodide (18.3 g, 0.13 mol, 3 eq) and reacted at room temperature for 12 h. The reaction was monitored until the starting materials had disappeared. The reaction solution was concentrated. The residue was adjusted to acidity, added with water, extracted three times with ethyl acetate, washed three times with saturated saline solution, dried over anhydrous sodium sulfate, and spin-dried to obtain 9.1 g of the crude product Compound 261-2 (71% yield).

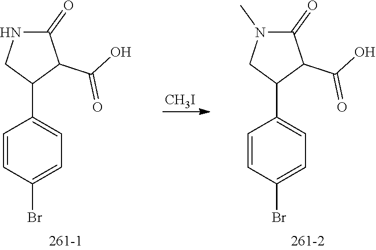 [SMILES: O=C(O)C1C(=O)NCC1c1ccc(Br)cc1 ; "CH3I" ; CN1CC(c2ccc(Br)cc2)C(C(=O)O)C1=O]

(2) Compound 261-2 (297 mg, 1 mmol, 1 eq), Compound 261-3 (285 mg, 1.5 mmol, 1.5 eq) and cesium fluoride (302 mg, 2 mmol, 2 eq) were dissolved in 16 mL of dioxane and 2 mL of water, displaced with nitrogen gas, added with a catalytic amount of Pd(dppf)Cl₂, displaced three times with nitrogen gas, and reacted at 100° C. for 12 h. The reaction was monitored until the starting materials had disappeared. The reaction solution was concentrated, added with water, extracted three times with ethyl acetate, washed three times with saturated saline solution, dried over anhydrous sodium sulfate, purified through a silica gel column, and spin-dried to obtain 299 mg of Compound 261-4 (82.8% yield).

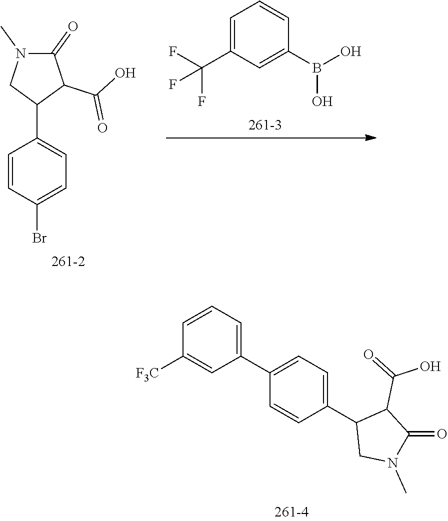 [SMILES: CN1CC(c2ccc(Br)cc2)C(C(=O)O)C1=O ; OB(O)c1cccc(C(F)(F)F)c1 ; CN1CC(c2ccc(-c3cccc(C(F)(F)F)c3)cc2)C(C(=O)O)C1=O]

(3) Compound 261-4 (100 mg, 0.28 mmol, 1 eq), the pyrazole compound 1-1 (44.5 mg, 0.34 mmol, 1.2 eq) and triethylamine (85 mg, 0.84 mmol, 3 eq) were dissolved in 10 mL of DCM, then added with Py-BOP (177 mg, 0.34 mmol, 1.2 eq), and reacted at room temperature for 1 h. The reaction was monitored until the starting materials had disappeared. The reaction solution was added with water, extracted three times with DCM, washed three times with saturated saline solution, dried over anhydrous sodium sulfate, purified through a silica gel column, and spin-dried to obtain 89 mg of Compound 261 (66.9% yield).

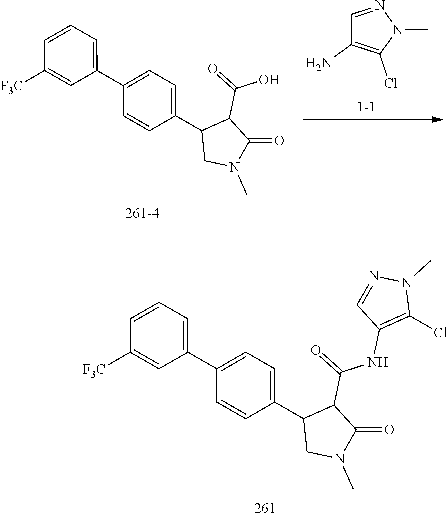

### 12. Synthesis of Compound 340′

(1) Compound 340-1 (4.6 g) was added into 50 mL of DCM, added with PCC (2 eq) in batches, and reacted at room temperature for 2 h. The solution was extracted with water and DCM. The organic phase was dried, concentrated, and purified by normal phase chromatography to obtain Compound 340-2 (2.8 g).

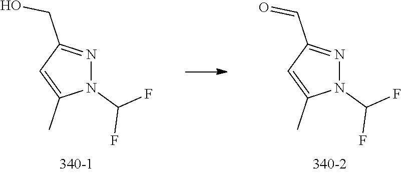

(2) Compound 340-2 (1 eq) was dissolved with 1000 mL of acetic acid, added into a 2000 mL single-necked flask, added with ammonium acetate (45 g, 0.05 mol, 0.5 eq), slowly added with nitromethane (200 g, 0.25 mol, 2.5 eq), stirred at 100° C., and reacted overnight. The reaction was monitored until the starting materials had disappeared. After the reaction, the system was treated by rotary-evaporation to remove the solvent, purification through a silica gel column, and spin-drying the fraction to obtain Compound 340-3 (54.9% yield).

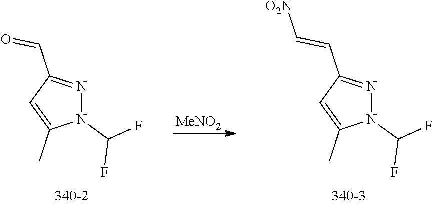

(3) Compound 340-3 (1 eq) was dissolved in 500 mL of toluene, added with a catalyst (CAS: 941321-23-9) of 10% (wt:wt) and dimethyl malonate (1.2 eq), heated up to 40° C., and reacted for 12 h. The reaction was monitored until the starting materials had disappeared. The reaction solution was added with water, extracted three times, washed three times with saturated saline solution, dried over anhydrous sodium sulfate, and spin-dried to obtain Compound 340-4 (90.9% yield).

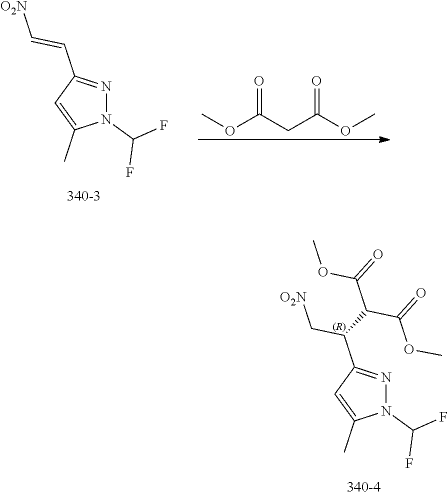

(4) Compound 340-4 (1 eq) was dissolved in 600 mL of anhydrous methanol, added with nickel chloride hexahydrate (204 g, 72 mmol, 1 eq), and in ice bath, added with sodium borohydride (97.7 g, 0.22 mol, 3 eq) in batches, and reacted at room temperature for 12 h. The reaction was monitored until the starting materials had disappeared. The reaction solution was concentrated. The residue was added with water, adjusted to acidity using hydrochloric acid, extracted three times with dichloromethane, washed three times with saturated saline solution, dried over anhydrous sodium sulfate, then stirred and purified through a silica gel column. The fraction was spin-dried to obtain Compound 340-5 (39% yield).

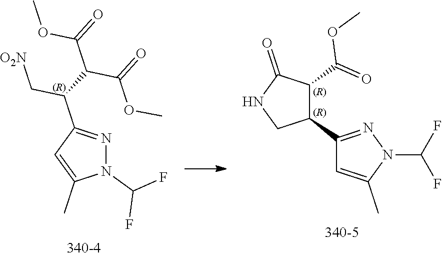

(5) Compound 340-5 (1 eq) was dissolved in 300 mL of tetrahydrofuran and 100 mL of water, and in ice bath, added with sodium hydroxide (40.1 g, 0.16 mol, 3 eq), and reacted at room temperature for 12 h. The reaction was monitored until the starting materials had disappeared. The reaction solution was concentrated. The residue was adjusted to acidity, added with water, extracted three times with ethyl acetate, washed three times with saturated saline solution, dried over anhydrous sodium sulfate, and spin-dried to obtain Compound 340-6 (85.4% yield).

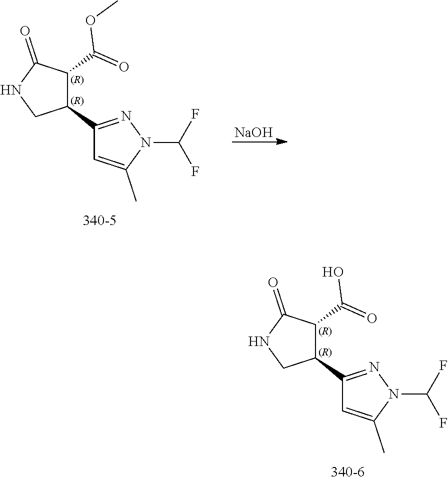

(6) Py-BOP (1.5 eq), Compound 1-1 (1.5 eq) and triethylamine (2 eq) were added into the DCM solution (100 mL) of Compound 340-6 (1 eq), and reacted at room temperature for 1 h. The reaction was monitored until the starting materials had disappeared. The reaction solution was added with water, extracted three times, and washed three times with saturated saline solution. The organic phase was dried over anhydrous sodium sulfate, purified through a silica gel column, and spin-dried to obtain Compound 340′ (50% yield).

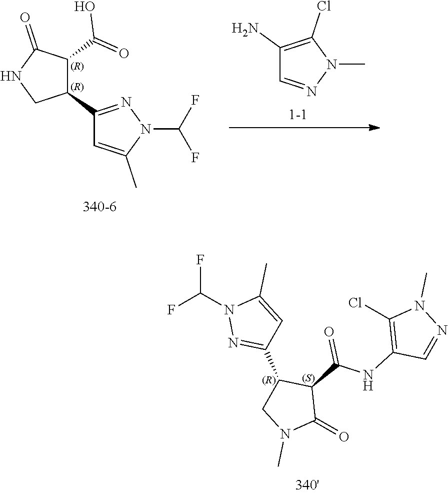

### 13. Synthesis of Compound 411′

(1) Compound 411-1 (1 eq), Compound 411-2 (1.2 eq), K₂CO₃ (3 eq) and 4,5-bis(diphenylphosphino)-9,9-dimethylxanthene (0.1 eq) were dissolved in toluene, displaced with nitrogen gas, added with tris(dibenzylideneacetone)dipalladium (0.1 eq), displaced with nitrogen gas again, and reacted for 12 h. The reaction solution was filtered by diatomaceous earth. The organic phase was extracted with water and ethyl acetate. The organic phases were collected and spin-dried to remove the solvent to obtain the crude product Compound 411-3.

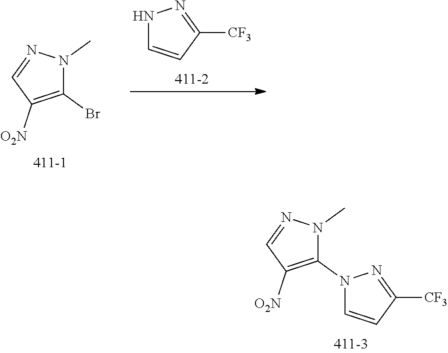

(2) Iron powder (3 eq) and ammonium chloride (3 eq) were added into a solution (EtOH:H₂O=3:1) of Compound 411-3 (1 eq), and reacted at 80° C. The reaction solution was filtered by diatomaceous earth to remove the iron powder and extracted with EA. The organic phase was dried over anhydrous sodium sulfate and concentrated by decompression to obtain Compound 411-4.

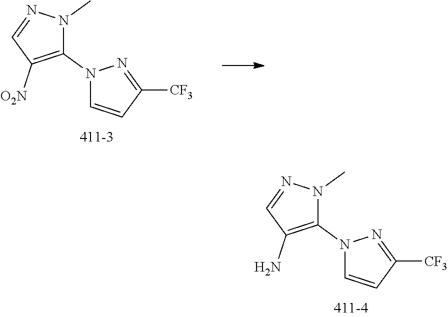 [SMILES: Cn1ncc([N+](=O)[O-])c1-n1ccc(C(F)(F)F)n1 ; Cn1ncc(N)c1-n1ccc(C(F)(F)F)n1]

(3) Py-BOP (1.5 eq), Compound 411-4 (1.5 eq) and triethylamine (2 eq) were added into the DCM solution of Compound 160-6 (1 eq) and stirred at room temperature. Water was added for extraction. The organic phase was dried over anhydrous sodium sulfate, added with silica gel powder, concentrated by decompression, and rotary-evaporated to dryness, followed by normal phase purification (mobile phase was petroleum ether/ethyl acetate=2/3) to obtain Compound 411′ (30% yield).

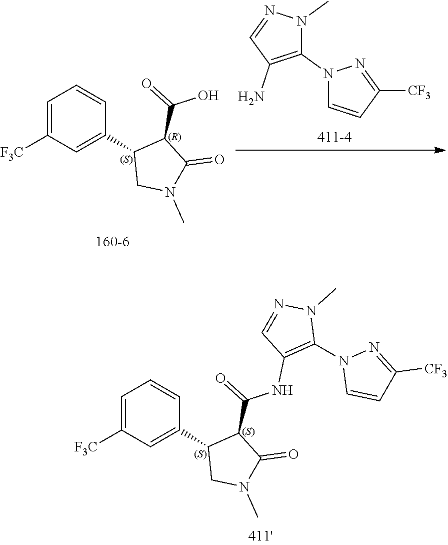

### 14. Synthesis of Compound 451′

Compound 117′ (0.10 g, 1 eq) and Lawesson's reagent (0.36 g, 3 eq) were added into 15 mL of toluene solvent in a 50 mL round-bottom flask and reacted at 100° C. for 30 min. Upon the completion of the reaction, the reaction solution was concentrated by decompression to remove the solvent and purified by silica gel column chromatography to obtain the yellow solid product Compound 451′ (40 mg, 95% purity, 36% yield).

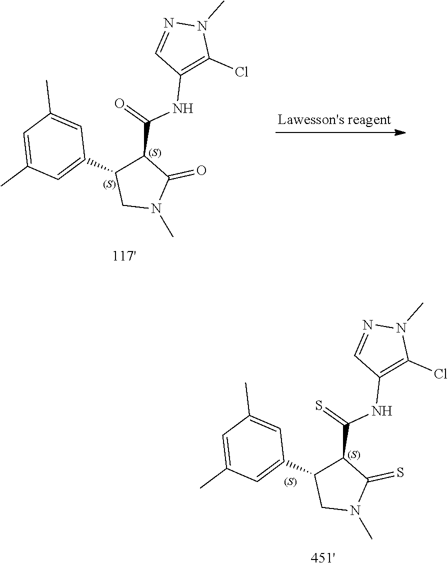

### Evaluation of Biological Activity

### (1) Seedling Stage Sealing Experiment for Water-Seeding:

The activity level criteria for plant damage (i.e., growth control rate) are as follows:
Level 9: completely dead;
Level 8: growth control rate greater than or equal to 90% and less than 100%;
Level 7: growth control rate greater than or equal to 80% and less than 90%;
Level 6: growth control rate greater than or equal to 70% and less than 80%;
Level 5: growth control rate greater than or equal to 50% and less than 70%;
Level 4: growth control rate greater than or equal to 30% and less than 50%;
Level 3: growth control rate greater than or equal to 20% and less than 30%;
Level 2: growth control rate greater than or equal to 10% and less than 20%;
Level 1: growth control rate less than 10%;
Level 0: no effect.

The above growth control rates are fresh weight control rates.

The seeds of Echinochloa

$$\begin{matrix}Lawesson ’ s \\ reagent\end{matrix}$$

Echinochloa phyllopogon, Digitaria sanguinalis, Huaidao (rice cultivar), etc. were broadcasted on the surface of nutrition bowls filled with soil and fully covered by soil. The foregoing well-prepared nutrition bowls were then placed in a cultivation box with water filled to a suitable height to keep the soil in the nutrition bowls moist. A test compound of the present invention was individually dissolved with acetone, then diluted into solution of a certain concentration using a certain amount of water, and sprayed using a spray tower. The plants were placed on a greenhouse seedbed and cultured for 15 days, and the experimental results were then collected. The compound was applied at 120, 60, 30 and 15 g a.i./ha in triplicate, and the results were averaged. Representative data are listed in Table 3.

TABLE 3
Results of seedling stage sealing experiment for water-seeding

| | Echinochloa | Echinochloa | Digitaria | Leptochloa | | Dosage |
|---|---|---|---|---|---|---|
| No. | crusgalli | phyllopogon | sanguinalis | chinensis | Huaidao | (g a.i./ha) |
| 1 | 9 | 9 | 9 | N | 0 | 120 |
| 1′ | 9 | 6 | N | N | 0 | 120 |
| 15 | 9 | 6 | 9 | N | N | 120 |
| 16 | 6 | 5 | N | 9 | N | 120 |
| 17′ | 9 | 6 | N | 9 | 0 | 120 |
| 20′ | 9 | 8 | N | 9 | N | 120 |
| 24 | 9 | 8 | N | 9 | N | 120 |
| 28 | 9 | 9 | 9 | N | 0 | 120 |
| 28′ | 8 | 6 | N | 9 | N | 120 |
| 29 | 9 | 9 | 9 | N | 0 | 120 |
| 30 | 9 | 9 | 9 | N | 0 | 120 |
| 33 | 9 | 5 | N | 9 | N | 120 |
| 33′ | 9 | 8 | N | 9 | N | 120 |
| 39 | 6 | N | N | 9 | N | 120 |
| 41 | 5 | N | N | 6 | N | 120 |
| 50′ | N | N | N | 6 | N | 120 |
| 52′ | 9 | N | N | N | 0 | 120 |
| 54 | 9 | 9 | N | 9 | 0 | 120 |
| 56 | 9 | 6 | 9 | 9 | N | 120 |
| 67 | 9 | 6 | N | 9 | N | 120 |
| 67′ | 9 | 8 | N | N | 1 | 120 |
| 69′ | 9 | 9 | N | N | 1 | 120 |
| 75′ | 9 | 6 | N | 9 | N | 120 |
| 80 | 6 | 6 | N | 6 | N | 120 |
| 94 | 5 | 5 | N | N | 0 | 120 |
| 95 | 6 | 6 | N | 9 | N | 120 |
| 95′ | 9 | 8 | N | N | N | 120 |
| 96 | 9 | 9 | N | 9 | 7 | 120 |
| 97 | 9 | 7 | N | 9 | 0 | 120 |
| 97′ | 9 | 9 | N | N | 0 | 120 |
| 98′ | 9 | 9 | N | 9 | N | 120 |
| 100 | 6 | N | N | 6 | N | 120 |
| 114 | 9 | 6 | 9 | 9 | N | 120 |
| 115 | 9 | 6 | N | 6 | N | 120 |
| 115′ | 9 | N | 8 | 8 | N | 120 |
| 116 | 7 | 6 | 9 | N | N | 120 |
| 117 | 9 | 6 | 9 | 9 | 0 | 120 |
| 117′ | 9 | 7 | N | 9 | N | 120 |
| 118 | 9 | 6 | 9 | 6 | N | 120 |
| 119 | 9 | 9 | 9 | N | 0 | 120 |
| 119′ | 9 | 9 | N | N | 0 | 120 |
| 120 | 9 | 9 | 9 | N | 0 | 120 |
| 120′ | 9 | 8 | N | 9 | N | 120 |
| 121 | 9 | 9 | N | 6 | 0 | 120 |
| 124′ | 6 | N | N | N | N | 120 |
| 125′ | 9 | 9 | N | 9 | N | 120 |
| 126 | 7 | 7 | N | 9 | N | 120 |
| 126′ | 9 | 9 | N | 9 | N | 120 |
| 127′ | 9 | 8 | N | N | 0 | 120 |
| 128′ | 9 | 8 | N | 9 | N | 120 |
| 129′ | 9 | 8 | N | 9 | N | 120 |
| 130′ | 9 | 9 | N | 9 | N | 120 |
| 131′ | 9 | 9 | 9 | 9 | N | 120 |
| 132′ | 7 | 7 | N | N | 0 | 120 |
| 134′ | 7 | 7 | N | N | 0 | 120 |
| 135′ | 9 | 7 | N | 9 | N | 120 |
| 136′ | 9 | 9 | N | N | 0 | 120 |
| 137′ | 6 | 5 | N | N | 0 | 120 |
| 138′ | 7 | 6 | N | N | 0 | 120 |
| 139′ | 9 | N | 7 | 9 | N | 120 |
| 147′ | 9 | 8 | N | 9 | N | 120 |
| 149′ | 5 | N | N | 5 | N | 120 |
| 150′ | 6 | N | N | N | 0 | 120 |
| 151′ | N | 9 | N | N | 0 | 120 |
| 152′ | 9 | 8 | N | 9 | N | 120 |
| 154′ | 9 | 8 | N | 9 | N | 120 |
| 155 | 5 | N | N | N | N | 120 |
| 156 | 6 | 6 | 7 | N | 0 | 120 |
| 156′ | 9 | 7 | N | N | 0 | 120 |
| 157′ | 5 | N | N | 7 | N | 120 |
| 158 | 9 | 8 | 9 | 9 | 0 | 120 |
| 158′ | 9 | 8 | N | 9 | N | 120 |
| 159 | 7 | 6 | 9 | N | N | 120 |
| 160′ | 6 | 5 | N | 9 | N | 120 |
| 161′ | 9 | 8 | N | 9 | N | 120 |
| 162′ | 9 | N | 8 | 9 | N | 120 |
| 163′ | 9 | 8 | N | N | 0 | 120 |
| 164 | 7 | 6 | N | 9 | N | 120 |
| 165′ | N | N | N | 6 | N | 120 |
| 171′ | 9 | 8 | N | N | 0 | 120 |
| 172 | 7 | 5 | N | 9 | N | 120 |
| 186′ | 9 | 7 | N | 9 | N | 120 |
| 187′ | 9 | 9 | N | N | 0 | 120 |
| 189 | 7 | 6 | 9 | 9 | N | 120 |
| 189′ | 9 | 9 | N | N | 0 | 120 |
| 190′ | 9 | 8 | N | 9 | N | 120 |
| 192 | 6 | N | N | 6 | N | 120 |
| 193′ | 9 | 7 | N | 9 | N | 120 |
| 194′ | 5 | 5 | N | 6 | N | 120 |
| 195′ | 9 | 7 | N | 9 | N | 120 |
| 196′ | 9 | 9 | N | 9 | N | 120 |
| 197′ | 8 | 6 | N | 9 | N | 120 |
| 198′ | 5 | N | N | N | N | 120 |
| 199′ | 6 | 6 | N | N | 0 | 120 |
| 200 | N | N | N | 6 | N | 120 |
| 202 | 7 | 6 | N | 8 | N | 120 |
| 203′ | 9 | 9 | N | 9 | N | 120 |
| 205 | 9 | 9 | N | N | 0 | 120 |
| 209′ | 7 | 6 | N | N | 0 | 120 |
| 212 | 9 | 6 | 9 | 9 | N | 120 |
| 212′ | 9 | 9 | N | 9 | N | 120 |
| 214 | N | N | 9 | N | 0 | 120 |
| 215′ | 9 | 8 | N | N | 0 | 120 |
| 217′ | 9 | 6 | N | 6 | N | 120 |
| 218′ | 6 | 6 | N | N | N | 120 |
| 219 | 7 | N | N | N | N | 120 |
| 220 | 9 | 6 | 9 | 6 | N | 120 |
| 221 | N | N | 9 | 9 | N | 120 |
| 222 | 6 | N | N | 6 | N | 120 |
| 223 | N | N | 9 | N | N | 120 |
| 224 | 6 | 6 | N | 9 | N | 120 |
| 225 | 7 | N | 9 | N | N | 120 |
| 226 | 5 | N | N | 6 | N | 120 |
| 227 | 7 | 6 | N | 9 | N | 120 |
| 228 | N | N | 7 | N | N | 120 |
| 229′ | 9 | 9 | N | 9 | N | 120 |
| 230 | 6 | 6 | N | 9 | N | 120 |
| 231′ | 9 | 6 | N | 9 | N | 120 |
| 232′ | 6 | N | N | N | N | 120 |
| 238 | 5 | N | 9 | N | N | 120 |
| 239 | 7 | 6 | N | 6 | N | 120 |
| 239′ | 9 | 8 | N | 9 | N | 120 |
| 240 | 7 | 7 | N | N | 0 | 120 |
| 241 | 9 | 7 | N | 9 | 0 | 120 |
| 241′ | 9 | 8 | N | 9 | N | 120 |
| 242 | 7 | 6 | N | 9 | N | 120 |
| 244 | 6 | N | N | N | N | 120 |
| 244′ | 9 | 7 | N | N | 0 | 120 |
| 245 | 9 | 9 | N | N | N | 120 |
| 245′ | N | 5 | N | N | N | 120 |
| 247 | 6 | N | N | N | N | 120 |
| 248 | 7 | 9 | N | N | 0 | 120 |
| 249 | 8 | 8 | 9 | N | N | 120 |
| 252 | 7 | 9 | N | N | N | 120 |
| 253′ | 6 | N | N | N | N | 120 |
| 254 | 5 | 5 | N | N | N | 120 |
| 259′ | 5 | N | N | N | 0 | 120 |
| 261 | 6 | 5 | N | N | N | 120 |
| 263′ | 9 | 8 | N | 9 | N | 120 |
| 264′ | 9 | 9 | N | N | 0 | 120 |
| 265′ | 9 | 9 | N | 9 | N | 120 |
| 267′ | 6 | N | N | N | 0 | 120 |
| 268′ | 7 | 6 | N | N | 0 | 120 |
| 269′ | 9 | 9 | N | 9 | N | 120 |
| 270′ | N | 6 | N | N | 0 | 120 |
| 271′ | 9 | 9 | N | N | 0 | 120 |
| 272′ | 9 | 7 | N | N | 0 | 120 |
| 273′ | 9 | 6 | N | N | 0 | 120 |
| 274′ | 7 | N | N | N | 0 | 120 |
| 275′ | 9 | 9 | N | 9 | N | 120 |
| 276′ | 9 | 6 | N | N | 0 | 120 |
| 277′ | 9 | N | 6 | 9 | N | 120 |
| 278′ | 6 | N | N | N | 0 | 120 |
| 280′ | 6 | N | N | N | 0 | 120 |
| 286 | 9 | 7 | N | 9 | N | 120 |
| 287′ | N | 9 | N | N | 0 | 120 |
| 289′ | N | 9 | N | N | 0 | 120 |
| 290′ | 9 | 8 | N | 9 | N | 120 |
| 291′ | 9 | 9 | N | 9 | N | 120 |
| 293′ | 9 | 9 | N | 9 | N | 120 |
| 294′ | 9 | 7 | N | N | 0 | 120 |
| 295′ | 7 | 6 | 9 | 9 | N | 120 |
| 297′ | 9 | 6 | N | 9 | N | 120 |
| 298′ | 7 | 6 | 9 | 7 | N | 120 |
| 299′ | 9 | 9 | N | 9 | N | 120 |
| 300′ | 9 | 7 | N | 9 | N | 120 |
| 301′ | N | N | N | 6 | N | 120 |
| 302 | 6 | 5 | N | N | N | 120 |
| 302′ | 9 | 6 | N | N | 0 | 120 |
| 304 | 7 | 6 | N | 5 | N | 120 |
| 307 | 6 | 6 | N | 5 | N | 120 |
| 308′ | 9 | 9 | N | 9 | N | 120 |
| 309 | 6 | 5 | N | 9 | N | 120 |
| 310 | 9 | 8 | N | 9 | N | 120 |
| 311′ | 9 | 8 | N | 9 | N | 120 |
| 313′ | 9 | N | 9 | 9 | N | 120 |
| 314 | 7 | 6 | N | 9 | N | 120 |
| 314′ | 9 | 9 | N | N | 0 | 120 |
| 315′ | 9 | 8 | N | 9 | N | 120 |
| 316′ | 6 | N | N | 6 | N | 120 |
| 318′ | 9 | 8 | N | 8 | N | 120 |
| 319′ | 5 | 5 | N | 6 | N | 120 |
| 320 | 6 | 6 | N | 5 | N | 120 |
| 321 | 5 | 5 | N | 6 | N | 120 |
| 321′ | 9 | 6 | N | 7 | N | 120 |
| 323 | 5 | 5 | N | 6 | N | 120 |
| 324 | 7 | 7 | N | 9 | N | 120 |
| 326′ | 8 | 7 | N | N | 0 | 120 |
| 329′ | 7 | 6 | N | N | N | 120 |
| 330 | 6 | 5 | N | N | N | 120 |
| 331 | 9 | 7 | N | 9 | N | 120 |
| 332 | 5 | N | N | N | N | 120 |
| 335′ | 7 | N | N | N | N | 120 |
| 338′ | 9 | 6 | N | N | 0 | 120 |
| 339 | 9 | N | 7 | 6 | N | 120 |
| 342 | 5 | 5 | N | N | N | 120 |
| 343 | 6 | 5 | N | 5 | N | 120 |
| 353′ | 9 | 7 | N | 9 | N | 120 |
| 355 | 5 | N | N | N | N | 120 |
| 356′ | 5 | N | N | N | N | 120 |
| 357 | 9 | 9 | N | 9 | N | 120 |
| 359 | N | 7 | N | N | 0 | 120 |
| 360′ | 7 | N | N | N | N | 120 |
| 361 | 7 | N | N | N | 0 | 120 |
| 367′ | 9 | N | N | N | 1 | 120 |
| 378′ | 9 | 6 | N | N | 0 | 120 |
| 379′ | 9 | 6 | N | N | 0 | 120 |
| 381′ | 9 | 6 | N | N | 0 | 120 |
| 384′ | 6 | N | N | N | 0 | 120 |
| 385′ | 9 | 9 | N | N | 2 | 120 |
| 386′ | 9 | 6 | N | N | 0 | 120 |
| 388′ | 9 | 6 | N | N | 0 | 120 |
| 389′ | 9 | 9 | N | N | 0 | 120 |
| 391′ | 9 | 9 | N | N | 0 | 120 |
| 393′ | 9 | 9 | N | N | N | 120 |
| 394′ | 9 | 6 | N | N | 0 | 120 |
| 395′ | 9 | 9 | N | N | N | 120 |
| 399′ | 9 | 9 | N | N | 0 | 120 |
| 403′ | 9 | 9 | N | N | N | 120 |
| 404′ | 9 | 9 | N | N | N | 120 |
| 406′ | 9 | 9 | N | N | 2 | 120 |
| 407′ | 9 | 9 | N | N | 0 | 120 |
| 408′ | 9 | 9 | N | N | 0 | 120 |
| 409′ | 9 | 7 | N | N | 0 | 120 |
| 413′ | 9 | 9 | N | N | 0 | 120 |
| 414′ | 9 | 9 | N | N | N | 120 |
| 415′ | 9 | 9 | N | N | N | 120 |
| 416′ | 9 | 6 | N | N | 0 | 120 |
| 418′ | 9 | 9 | N | N | N | 120 |
| 419′ | 9 | 9 | N | N | 0 | 120 |
| 420′ | 9 | 9 | N | N | 0 | 120 |
| 421′ | 9 | N | N | N | 0 | 120 |
| 422′ | 9 | 9 | N | N | 1 | 120 |
| 423′ | 9 | 6 | N | N | 0 | 120 |
| 425′ | 9 | 9 | N | N | 0 | 120 |
| 426′ | 9 | 9 | N | N | 0 | 120 |
| 427′ | 9 | 9 | N | N | 2 | 120 |
| 428′ | 6 | N | N | N | 0 | 120 |
| 429′ | 9 | 9 | N | N | N | 120 |
| 431′ | 9 | 9 | N | N | N | 120 |
| 432′ | 9 | 9 | N | N | 0 | 120 |
| 433′ | 9 | 6 | N | N | 0 | 120 |
| 434′ | 9 | 9 | N | N | N | 120 |
| 438′ | 9 | 9 | N | N | 2 | 120 |
| 439′ | 9 | 9 | N | N | 2 | 120 |
| 440′ | 9 | 9 | N | N | 0 | 120 |
| 441′ | 9 | 9 | N | N | 0 | 120 |
| 442′ | 8 | 8 | N | N | 0 | 120 |
| 443′ | 9 | 8 | N | N | 0 | 120 |
| 444′ | 9 | 9 | N | N | N | 120 |
| 445′ | 9 | 9 | N | N | 0 | 120 |
| 446′ | 9 | 9 | N | N | 0 | 120 |
| 447′ | 9 | 9 | N | N | 0 | 120 |
| 448′ | 9 | 9 | N | N | 0 | 120 |
| 449′ | 6 | N | N | N | 0 | 120 |
| 450′ | 9 | 9 | N | N | 0 | 120 |
| 451′ | 8 | 7 | N | N | 0 | 120 |
| 452′ | 9 | 9 | N | N | 0 | 120 |
| 453′ | 9 | 9 | N | N | 2 | 120 |
| 456 | 7 | N | 9 | N | 0 | 120 |
| 457 | N | N | 9 | N | 0 | 120 |
| 460 | N | N | 7 | N | N | 120 |
| 462′ | 9 | 9 | N | N | 0 | 120 |
| 463′ | 6 | N | N | N | 0 | 120 |
| 465′ | 9 | 9 | N | N | 2 | 120 |
| 466′ | 9 | 9 | N | N | N | 120 |
| 468′ | 9 | 9 | N | N | 0 | 120 |
| 469 | 6 | N | N | N | 0 | 120 |
| 471′ | 9 | N | N | N | 0 | 120 |
| 475 | 9 | 9 | N | N | 0 | 120 |
| 477 | 9 | N | N | N | 0 | 120 |
| 478′ | 9 | 9 | N | N | 0 | 120 |
| 479′ | 9 | 9 | N | N | N | 120 |
| 480′ | 9 | 9 | N | N | 1 | 120 |
| 482′ | 9 | N | N | N | 0 | 120 |
| 483′ | 9 | 8 | N | N | 0 | 120 |
| 485′ | 9 | 9 | N | N | 0 | 120 |
| 486′ | 9 | 9 | N | N | 2 | 120 |
| 487′ | 9 | 9 | N | N | 2 | 120 |
| 488 | 9 | 9 | N | N | N | 120 |
| 489′ | 9 | 9 | N | N | 0 | 120 |
| 490′ | 9 | N | N | N | 0 | 120 |
| 494′ | 9 | 9 | N | N | 2 | 120 |
| 495′ | 9 | 7 | N | N | 0 | 120 |
| 496′ | 9 | 9 | N | N | 0 | 120 |
| 498′ | 9 | 9 | N | N | 0 | 120 |
| 499 | 8 | N | N | N | 0 | 120 |
| 501′ | 9 | 7 | N | N | 0 | 120 |
| 502′ | 9 | 7 | N | N | 0 | 120 |
| 504′ | 9 | 9 | N | N | 0 | 120 |
| 505′ | 9 | 9 | N | N | 2 | 120 |
| 506′ | 9 | 9 | N | N | 0 | 120 |
| 507′ | 9 | 9 | N | N | 0 | 120 |
| 508 | 9 | 9 | N | N | N | 120 |
| 509 | 9 | 9 | N | N | 2 | 120 |
| 510′ | 9 | 9 | N | N | 2 | 120 |
| 511′ | 9 | 6 | N | N | 0 | 120 |
| 512′ | 9 | 7 | N | N | 0 | 120 |
| 513′ | 9 | N | N | N | 0 | 120 |
| 514′ | 9 | 6 | N | N | 0 | 120 |
| 515′ | 9 | 9 | N | N | N | 120 |
| 516′ | 9 | 9 | N | N | 0 | 120 |
| 517 | 9 | 9 | N | N | 0 | 120 |
| 518′ | 9 | 9 | N | N | 0 | 120 |
| 519′ | 9 | 9 | N | N | 1 | 120 |
| 520′ | 9 | 7 | N | N | 0 | 120 |
| 523′ | 9 | 6 | 9 | N | 0 | 120 |
| 524′ | 9 | 9 | 9 | N | 2 | 120 |
| 525′ | 9 | 9 | 9 | N | 2 | 120 |
| 526′ | 9 | 6 | 9 | N | 0 | 120 |
| 527′ | 9 | 6 | 9 | N | 0 | 120 |
| 528′ | 9 | 7 | 9 | N | N | 120 |
| 530′ | 9 | N | N | N | 0 | 120 |
| 531′ | 9 | 9 | 9 | N | 0 | 120 |
| 532′ | 9 | 6 | 9 | N | 0 | 120 |
| 533′ | 9 | 9 | 9 | N | 0 | 120 |
| 535′ | 9 | 9 | 9 | N | 0 | 120 |
| 536′ | 9 | 9 | 9 | N | N | 120 |
| 538′ | 9 | 9 | 9 | N | N | 120 |
| 539′ | 9 | N | 6 | N | 0 | 120 |
| 540′ | 9 | 9 | 9 | N | 0 | 120 |
| 544′ | 9 | 9 | 9 | N | N | 120 |
| 546′ | 9 | 9 | 9 | N | 0 | 120 |
| 547′ | 9 | 9 | 9 | N | 0 | 120 |
| 548′ | 9 | 6 | 9 | N | 0 | 120 |
| 549′ | 9 | 9 | 9 | N | N | 120 |
| 551′ | 9 | N | N | N | 0 | 120 |
| 552′ | 9 | N | 9 | N | 0 | 120 |
| 553′ | 9 | N | 9 | N | 2 | 120 |
| 554′ | 9 | 9 | 9 | N | 0 | 120 |
| 555′ | 9 | 9 | 9 | N | 0 | 120 |
| 556′ | 9 | 9 | 9 | N | 2 | 120 |
| 557′ | 9 | 6 | 9 | N | N | 120 |
| 558′ | 9 | 9 | 9 | N | 2 | 120 |
| 560′ | 9 | 9 | 9 | N | 0 | 120 |
| 561′ | 9 | 9 | 9 | N | 1 | 120 |
| Control | 0 | 0 | 0 | N | 0 | 120 |
| compound | | | | | | |
| A | | | | | | |

Note: N represents no data; Control compound A:

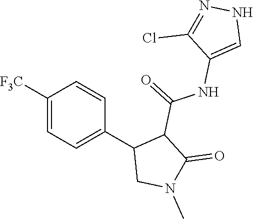 [SMILES: CN1CC(c2ccc(C(F)(F)F)cc2)C(C(=O)Nc2c[nH]nc2Cl)C1=O]

### (2) Safety Evaluation on Transplanted Paddy Rice and Control Effect Evaluation on Weeds in Paddy Fields:

After paddy field soil was loaded into a 1/1,000,000 ha pot, the seeds of Echinochloa crusgalli and Echinochloa phyllopogon were sowed and gently covered with soil, then left to stand in a greenhouse with water storage of 0.5-1 cm deep. The water storage was kept at a depth of 3-4 cm thereafter. When the Echinochloa crusgalli and Echinochloa phyllopogon reached their 0.5-leaf stages, WP or SC aqueous dilutions of a compound of the present invention formulated according to customary formulation methods were homogeneously dripped for treatment to the specified effective amount using pipettes.

In addition, after being loaded into the 1/1,000,000 ha pot, the paddy field soil was leveled to keep the water storage at a depth of 3-4 cm. The paddy rice (japonica rice) at 3-leaf stage was transplanted at a transplanting depth of 3 cm on the next day. The compound of the present invention was treated in the same manner as above on the 5^{th} day after the transplantation.

The developmental conditions of Echinochloa crusgalli and Echinochloa phyllopogon on the 14^{th} day after the drug treatment and the developmental conditions of paddy rice on the 21^{st} day after the drug treatment were observed with the naked eye, respectively. The effects were evaluated according to the above-mentioned criteria for activity levels. Many compounds exhibited excellent activity and selectivity.

### (3) Fungicidal Activity Assay:

Petri dish method: The drug agent was dissolved with acetone, diluted with sterile water, and set to different levels of mass concentrations according to its activity. Under aseptic operation conditions, the pre-melted sterilized medium was quantitatively added into sterilized flasks according to the test treatment. The drug solutions were quantitatively aspirated in order from low to high concentrations, added into the above-mentioned flasks, and shaken well, respectively. Next, equal amounts of the solution were poured into 3 petri dishes with a diameter of 6 cm to prepare drug-containing plates with corresponding concentrations. In the test, a treatment without drugs was set up as the blank control and each treatment was repeated 3 times. A fungus cake was cut out from the edge of the colony of the cultured pathogens using a sterile hole puncher with a diameter of 3 mm under sterile conditions. The fungus cake was inoculated into the center of a drug-containing plate using an inoculator, with the mycelia facing up, covered with a plate cover, and cultured in an incubator at 25° C. The growth of pathogen mycelia was investigated according to the fungal growth in the blank control petri dishes after 4 days. The diameters of the colonies were measured with a caliper in millimeters (mm). Each colony was vertically measured for diameters once each by the cross method and then averaged. Based on the investigation results, the mycelium growth inhibition rate of each treatment concentration on the test target fungi was calculated in accordance with Equation (1) and (2). The unit was percentage (%).

$$D = {D}_{1} - {D}_{2} \qquad ( 1 )$$

wherein: D—Increase of colony diameter; D₁—Diameter of colony; D₂—Diameter of fungus cake.

$$I = \frac{D 0 - Dt}{D 0} \times 100 \qquad ( 2 )$$

wherein: I—Mycelium growth inhibition rate; D₀—Increase of colony diameter of blank control; Dₜ—Increase of colony diameter of drug treatment.

TABLE 4
Results of fungicidal activity assay

| | Tomato | Rice | Apple | |
|---|---|---|---|---|
| | gray | sheath | ring | Concentration/ |
| No. | mold/% | blight/% | rot/% | ppm |
| 117′ | N | 66 | 55 | 400 |
| 250′ | N | N | 51 | 400 |
| 259′ | 80 | 59 | 51 | 400 |
| 270′ | 51 | 61 | N | 400 |
| 356′ | 57 | N | 83 | 400 |
| 357′ | 80 | 50 | 65 | 400 |
| 418′ | 96 | N | N | 500 |
| 425′ | 68 | 56 | 55 | 400 |
| 504′ | N | 83 | 80 | 400 |

### (4) Insecticidal Activity Assay:

Spraying method: the technical material was weighed and dissolved with acetone, and the drug liquid was diluted with distilled water to different concentrations. The 2^{nd} instar Spodoptera frugiperda test insects or the 3^{rd} instar Mythimna separata test insects reared indoors with consistent physiological conditions were selected. Ten test insects were picked into each petri dish. Two 2 cm-long corn leaves were placed in the petri dish and sprayed using a spray tower. Each dosage was repeated 3 times, with acetone of the corresponding concentration as a control. After drug application, the insects were transferred to and reared in a condition where the temperature was 25° C. and the humidity was 60%. The results were investigated 48 h after drug application and the death number per treatment was collected, respectively. The mortality was calculated in accordance with the following equation: Mortality (%)=(Number of dead insects/Number of test insects)×100. Representative data are as shown in Table 5.

TABLE 5
Results of insecticidal activity assay

| | Spodoptera | Mythimna | Concentration/ |
|---|---|---|---|
| No. | frugiperda/% | separata/% | ppm |
| 270′ | 55 | 75 | 500 |
| 277′ | N | 95 | 100 |
| 281′ | N | 60 | 500 |
| 340′ | 87 | 100 | 500 |
| 420′ | 75 | 90 | 500 |
| 421′ | 65 | 75 | 500 |
| 491′ | 60 | 70 | 500 |
| 500′ | 60 | 70 | 500 |
| 522′ | 60 | 70 | 500 |

### (5) Activity Assay of Compositions:

The required active ingredient B was purchased from a reagent company or a technical material manufacturer or synthesized by a conventional method. The technical materials were all dissolved with acetone solvents and diluted with aqueous solution of 0.1% Tween® 80 emulsifier, which should be used immediately after dilution.

### (A) Soil Sealing Treatment (S):

The weeds were cultured in a controllable sunlight greenhouse at 20-30° C., in natural light and with relative humidity of 57%-72%. The soil was loam with an organic matter content of 1.63%, a pH value of 7.1, alkali-hydrolyzable nitrogen of 84.3 mg/kg, rapidly available phosphorus of 38.5 mg/kg, and rapidly available potassium of 82.1 mg/kg. The test soil was quantitatively filled to ¾ of a pot, and then watered from the bottom of the pot to completely wet the soil to saturation. The seeds of the test weeds were accelerated to Just germinate, and then evenly and quantitatively broadcasted on the soil surface, covered with 0.5-1 cm soil according to the sizes of the seeds, and ready for use 72 hours after broadcasting.

Each treatment was repeated 4 times. Four pots were treated each time with 20 weed seeds per pot.

The seeds were planted in a 20 cm*30 cm square box. Water was added to 2-3 cm deep after sowing, then injected with the drug agent using a syringe, stirred evenly, and left to stand for 2 days to dry naturally. The soil was kept moist afterwards.

### (B) Post-Emergence Spray Treatment of Stems and Leaves (F):

Weeds were cultivated by the pot culture method. A 180×140 mm plastic nutritional bowl, containing air-dried and sieved topsoil (taking up 4/5 of the bowl) collected from the field, was placed in an enamel pan, wherein the soil had an initial moisture content of 20%. Weed seeds with plump and uniform grains were selected, soaked in lukewarm water at 25° C. for 6 hours, and accelerated to germinate in a 28° C. biochemical incubator (in darkness). The weed seeds that had just germinated were evenly placed on the surface of the soil and then covered with 0.5-1 cm soil according to the sizes of seeds.

The weeds were cultured in a controllable sunlight greenhouse at 20-30° C., in natural light and with relative humidity of 57%-72%. The soil was loam with an organic matter content of 1.63%, a pH value of 7.1, alkali-hydrolyzable nitrogen of 84.3 mg/kg, rapidly available phosphorus of 38.5 mg/kg, and rapidly available potassium of 82.1 mg/kg.

Each treatment was repeated 4 times. Three pots with 20 weed seeds sowed per pot were treated each time.

The drug was applied once in total in the experiment. When at 1.5-2 leaf stage, the weeds were thinned out to maintain 10 strains per pot. Thirty strains were kept in each treatment and then continued to be cultured until the 3-4 leaf stage for further treatment.

The well-cultured test materials were evenly placed on a 0.5 m² platform and foliage-sprayed by the 3WP-2000-type walking spray tower at a dosage of 450 kg/ha and a spray pressure of 0.3 MPa. After all the drug solution was sprayed, the air valve was closed. And 30 seconds later, the door of the spray tower was opened, and the nutritional bowl was taken out. Next, the air valve was opened, and the spray tube was cleaned by spraying 50 mL of water. The test materials were transferred to and routinely cultured in a greenhouse after the treatment.

### (C) Data Investigation and Statistical Analysis

A method for investigating absolute numbers was employed. The whole seedlings of the survival weeds were cut along the soil surface with a blade, and the fresh weight of the weeds was weighed by an analytical balance. For dead weeds, the fresh weight thereof was considered zero.

The investigation was performed 21 days after the treatment for once in total.

The theoretical fresh weight inhibition rate of a mixed combination of each treatment was calculated by the Gowing method (E0═X+Y−X*Y/100), then compared with the measured inhibition rate (E) to evaluate the type of the combined action of the combination of the two on weeds. The combined action was a synergistic effect when E−E0 >10%; an antagonistic effect when E−E0<−10%; an additional effect when −10%≤E−E0≤10%. An optimal ratio was determined according to factors such as actual control effects, characteristics of herbicides, balance of the formulation, etc. In the formula, X represents the fresh weight inhibition rate of the active ingredient A in a dosage of P; Y represents the fresh weight inhibition rate of the active ingredient B in a dosage of Q. The statistical results are shown in Table 6.

TABLE 6
Evaluation of actual control effect and combined action on weeds of Component A
(Compound 117′) combinations

| | | | | | Control | Control | | | |
|---|---|---|---|---|---|---|---|---|---|
| | | | | | effect of | effect of | | | |
| | | | | | A applied | B applied | Control | | |
| | | | | | alone | alone | effect | | |
| | | Treat- | Dosage | | in a corre- | in a corre- | of | | E − |
| | | ment | of A + B | | sponding | sponding | A + B | E0 | E0 |
| Component (A + B) | Weed | F/S | (ga.i./ha) | A:B | dose (%) | dose (%) | (%), E | (%) | (%) |
| A + Pretilachlor | Rotala | S | 15 + 75 | 1:5 | 37.6 | 54.2 | 82.9 | 71.4 | 11.5 |
| | indica | | 15 + 150 | 1:10 | 37.6 | 63.9 | 88.8 | 77.5 | 11.3 |
| A + Butachlor | Rotala | S | 15 + 150 | 1:10 | 37.6 | 42.4 | 78.5 | 64.1 | 14.4 |
| | indica | | 15 + 300 | 1:20 | 37.6 | 58.6 | 84.8 | 74.2 | 10.6 |
| A + Mefenacet | Rotala | S | 15 + 150 | 1:10 | 37.6 | 38.4 | 72.8 | 61.6 | 11.2 |
| | indica | | 15 + 300 | 1:20 | 37.6 | 58.3 | 85.6 | 74.0 | 11.6 |
| A + Acetochlor | Rotala | S | 15 + 150 | 1:10 | 37.6 | 46.2 | 79.8 | 66.4 | 13.4 |
| | indica | | 15 + 300 | 1:20 | 37.6 | 66.5 | 90.4 | 79.1 | 11.3 |
| A + Anilofos | Rotala | S | 15 + 75 | 1:5 | 37.6 | 22.4 | 62.8 | 51.6 | 11.2 |
| | indica | | 15 + 150 | 1:10 | 37.6 | 31.8 | 69.6 | 57.4 | 12.2 |
| A + | Rotala | S | 15 + 7.5 | 2:1 | 37.6 | 47.1 | 79.3 | 67.0 | 12.3 |
| 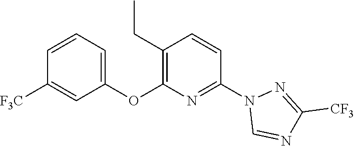 [SMILES: CCc1ccc(-n2cnc(C(F)(F)F)n2)nc1Oc1cccc(C(F)(F)F)c1] | indica | | 15 + 15 | 1:1 | 37.6 | 63.6 | 87.6 | 77.3 | 10.3 |
| A + | Rotala | S | 15 + 15 | 1:1 | 37.6 | 35.8 | 71.8 | 59.9 | 11.9 |
| 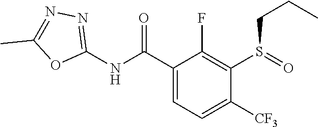 [SMILES: CCC[S@@](=O)c1c(C(F)(F)F)ccc(C(=O)Nc2nnc(C)o2)c1F] | indica | | 15 + 30 | 1:2 | 37.6 | 46.6 | 79.2 | 66.7 | 12.5 |
| A + Tefuryltrione | Rotala | S | 15 + 15 | 1:1 | 37.6 | 30.8 | 71.1 | 56.8 | 14.3 |
| | indica | | 15 + 30 | 1:2 | 37.6 | 45.2 | 78.5 | 65.8 | 12.7 |
| A + Benzobicyclon | Rotala | S | 15 + 15 | 1:1 | 37.6 | 32.8 | 73.8 | 58.1 | 15.7 |
| | indica | | 15 + 30 | 1:2 | 37.6 | 49.6 | 83.5 | 68.6 | 14.9 |
| A + Bipyrazone | Rotala | S | 15 + 15 | 1:1 | 37.6 | 24.5 | 64.5 | 52.9 | 11.6 |
| | indica | | 15 + 30 | 1:2 | 37.6 | 38.8 | 73.9 | 61.8 | 12.1 |
| A + Oxadiazon | Rotala | S | 15 + 30 | 1:2 | 37.6 | 50.5 | 80.1 | 69.1 | 11.0 |
| | indica | | 15 + 60 | 1:4 | 37.6 | 68.7 | 93.4 | 80.5 | 12.9 |
| A + Pyraclonil | Rotala | S | 15 + 30 | 1:2 | 37.6 | 48.2 | 81.7 | 67.7 | 14.0 |
| | indica | | 15 + 60 | 1:4 | 37.6 | 63.4 | 91.0 | 77.2 | 13.8 |
| A + Oxyfluorfen | Rotala | S | 15 + 7.5 | 2:1 | 37.6 | 51.0 | 84.1 | 69.4 | 14.7 |
| | indica | | 15 + 15 | 1:1 | 37.6 | 69.3 | 94.6 | 80.8 | 13.8 |
| A + Oxadiargyl | Rotala | S | 15 + 7.5 | 2:1 | 37.6 | 47.2 | 81.8 | 67.1 | 14.7 |
| | indica | | 15 + 15 | 1:1 | 37.6 | 58.6 | 88.2 | 74.2 | 14.0 |
| A + Pentoxazone | Rotala | S | 15 + 30 | 1:2 | 37.6 | 55.3 | 82.2 | 72.1 | 10.1 |
| | indica | | 15 + 60 | 1:4 | 37.6 | 62.7 | 89.4 | 76.7 | 12.7 |
| A + | Rotala | S | 15 + 7.5 | 2:1 | 37.6 | 47.9 | 79.4 | 67.5 | 11.9 |
| 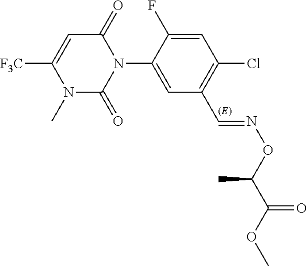 [SMILES: COC(=O)[C@@H](C)O/N=C/c1cc(-n2c(=O)cc(C(F)(F)F)n(C)c2=O)c(F)cc1Cl] | indica | | 15 + 15 | 1:1 | 37.6 | 56.6 | 85.0 | 72.9 | 12.1 |
| A + | Rotala | S | 15 + 7.5 | 2:1 | 37.6 | 45.3 | 78.1 | 65.9 | 12.2 |
| 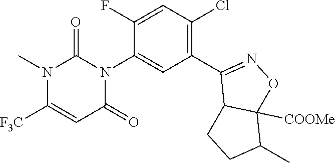 | indica | | 15 + 15 | 1:1 | 37.6 | 57.1 | 86.3 | 73.2 | 13.1 |
| A + Halauxifen-methyl | Alisma | S | 15 + 3.75 | 4:1 | 29.6 | 58.4 | 81.1 | 70.7 | 10.4 |
| | plantago- | | 15 + 7.5 | 2:1 | 29.6 | 68.9 | 91.5 | 78.1 | 13.4 |
| | aquatica | | | | | | | | |
| A + Florpyrauxifen-benzyl | Alisma | S | 15 + 3.75 | 4:1 | 29.6 | 62.8 | 85.8 | 73.8 | 12.0 |
| | plantago- | | 15 + 7.5 | 2:1 | 29.6 | 74.6 | 94.5 | 82.1 | 12.4 |
| | aquatica | | | | | | | | |
| A + | Alisma | F | 30 + 3.75 | 8:1 | 24.8 | 43.5 | 70.7 | 57.5 | 13.2 |
| 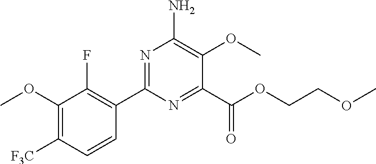 | plantago- aquatica | | 30 + 7.5 | 4:1 | 24.8 | 57.2 | 81.2 | 67.8 | 13.4 |
| A + Fluroxypyr | Alisma | F | 30 + 30 | 1:1 | 24.8 | 52.5 | 74.9 | 64.3 | 10.6 |
| | plantago- | | 30 + 60 | 1:2 | 24.8 | 65.8 | 85.6 | 74.3 | 11.3 |
| | aquatica | | | | | | | | |
| A + | Alisma | F | 30 + 30 | 1:1 | 24.8 | 38.7 | 67.2 | 53.9 | 13.3 |
| 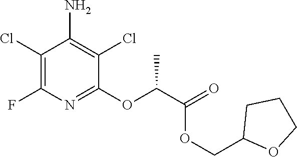 [SMILES: C[C@@H](Oc1nc(F)c(Cl)c(N)c1Cl)C(=O)OCC1CCCO1] | plantago- aquatica | | 30 + 60 | 1:2 | 24.8 | 45.6 | 70.2 | 59.1 | 11.1 |
| A + Propanil | Paspalum | F | 30 + 300 | 1:10 | 42.6 | 48.3 | 81.4 | 70.3 | 11.1 |
| | distichum | | 30 + 600 | 1:20 | 42.6 | 56.1 | 86.8 | 74.8 | 12.0 |
| A + Bentazone | Paspalum | F | 30 + 375 | 2:25 | 42.6 | 29.2 | 69.8 | 59.4 | 10.4 |
| | distichum | | 30 + 750 | 1:25 | 42.6 | 36.7 | 74.8 | 63.7 | 11.1 |
| A + Simetryn | Paspalum | S | 15 + 75 | 1:5 | 45.7 | 45.5 | 81.6 | 70.4 | 11.2 |
| | distichum | | 15 + 150 | 1:10 | 45.7 | 54.8 | 89.0 | 75.5 | 13.5 |
| A + Clomazone | Paspalum | S | 15 + 30 | 1:2 | 45.7 | 44.3 | 81.4 | 69.8 | 11.6 |
| | distichum | | 15 + 60 | 1:4 | 45.7 | 51.5 | 84.7 | 73.7 | 11.0 |
| A + | Paspalum | S | 15 + 30 | 1:2 | 45.7 | 37.8 | 78.5 | 66.2 | 12.3 |
| 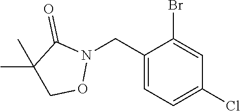 [SMILES: CC1(C)CON(Cc2ccc(Cl)cc2Br)C1=O] | distichum | | 15 + 60 | 1:4 | 45.7 | 51.3 | 84.7 | 73.6 | 11.1 |
| A + Bixlozone | Paspalum | S | 15 + 30 | 1:2 | 45.7 | 41.7 | 79.4 | 68.3 | 11.1 |
| | distichum | | 15 + 60 | 1:4 | 45.7 | 52.8 | 85.0 | 74.4 | 10.6 |
| A + Cinmethylin | Paspalum | S | 15 + 7.5 | 2:1 | 45.7 | 34.8 | 75.7 | 64.6 | 11.1 |
| | distichum | | 15 + 15 | 1:1 | 45.7 | 47.6 | 84.5 | 71.5 | 13.0 |
| A + | Paspalum | S | 15 + 7.5 | 2:1 | 45.7 | 46.3 | 84.6 | 70.8 | 13.8 |
| 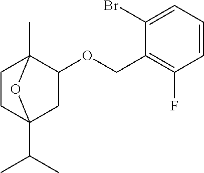 [SMILES: CC(C)C12CCC(C)(O1)C(OCc1c(F)cccc1Br)C2] | distichum | | 15 + 15 | 1:1 | 45.7 | 57.4 | 89.8 | 76.9 | 12.9 |
| A + Oxaziclomefone | Paspalum | S | 15 + 7.5 | 2:1 | 45.7 | 36.9 | 76.7 | 65.7 | 11.0 |
| | distichum | | 15 + 15 | 1:1 | 45.7 | 48.5 | 83.6 | 72.0 | 11.6 |

Meanwhile, it has been found by extensive experimentations that many of the compounds of the present invention and compositions thereof have good selectivity to gramineae grasses such as zoysia grass, bermuda grass, tall fescue, bluegrass, ryegrass, seashore paspalum, etc., and can prevent or eliminate many key gramineous weeds and broad-leaf weeds. The experiments on sugarcane, soybean, cotton, oil sunflower, potato, fruit trees, vegetables and the like using different drug application methods also demonstrated excellent selectivity and commercial values. Furthermore, many of the compounds of the present invention and compositions thereof have good control activity against fungi of different varieties including the orders Ascomycetes, Basidiomycetes, Deuteromycetes, Oomycetes and the like and also have good control activity against agricultural pests such as lepidopterans (e.g., Spodoptera frugiperda, Mythimna separata, etc.), which have certain commercial values.

Finally, it should be noted that the above embodiments are only to illustrate the technical solutions of the present invention buy not to set limitations. Although the present invention has been described in detail with reference to preferred embodiments, it should be understood by one of ordinary skill in the art that the technical solutions of the present invention can be modified or equivalently replaced without departing from the spirit and the scope of the technical solutions of the present invention.

1. A substituted pyrrolidone compound is as shown in the general formula I. 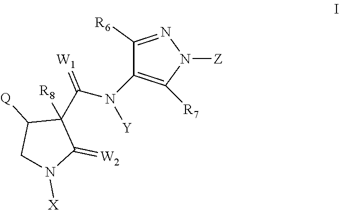
wherein, Q represents 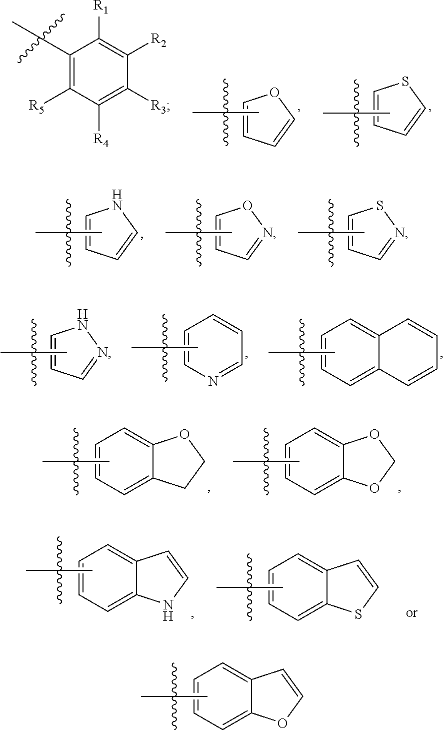
that is unsubstituted or substituted by at least one R₁₁;
X and Y each independently represent hydrogen, alkyl, alkenyl, alkynyl, haloalkyl, haloalkenyl, haloalkynyl, cycloalkyl, cycloalkylalkyl, aryl, arylalkyl, heterocyclyl, heterocyclylalkyl, hydroxyl, hydroxylalkyl, mercapto, mercaptoalkyl, nitro, cyanoalkyl, formyl, trialkylsilyl, —N(R₂₁)₂, —OR₂₂, —SR₂₂, —(CO)R₂₂, —(CO)OR₂₂, —(SO₂)R₂₂, —(SO)R₂₂, —(SO₂)N(R₂₁)₂, -alkylene-N(R₂₁)₂, -alkylene-OR₂₂, -alkylene-SR₂₂, -alkylene-(CO)R₂₂, -alkylene-(CO)OR₂₂, -alkylene-(SO₂)R₂₂, or -alkylene-(SO₂)N(R₂₁)₂;
Z represents alkyl, alkenyl, alkynyl, haloalkyl, haloalkenyl, haloalkynyl, cycloalkyl, cycloalkylalkyl, aryl, arylalkyl, heterocyclyl, heterocyclylalkyl, hydroxyl, hydroxylalkyl, mercapto, mercaptoalkyl, nitro, cyanoalkyl, formyl, trialkylsilyl, —N(R₂₁)₂, —OR₂₂, —SR₂₂, —(SO)R₂₂, —(CO)R₂₂, —(CO)OR₂₂, —(SO₂)R₂₂, —(SO₂)N(R₂₁)₂, -alkylene-N(R₂₁)₂, -alkylene-OR₂₂, -alkylene-SR₂₂, -alkylene-(CO)R₂₂, -alkylene-(CO)OR₂₂, -alkylene-(SO₂)R₂₂, or -alkylene-(SO₂)N(R₂₁)₂;
W₁ and W₂ each independently represent O or S;
R₆ represents hydrogen, hydroxyl, halogen, alkyl, cycloalkyl, alkoxy, or haloalkyl;
R₇ represents hydrogen, halogen, nitro, cyano, formyl, carboxyl, hydroxyl, hydroxylalkyl, mercapto, mercaptoalkyl, alkyl, alkenyl, alkynyl, haloalkyl, haloalkenyl, haloalkynyl, —OR₂₂, —SR₂₂, —(SO)R₂₂, —(SO₂)R₂₂, —CR₂₃═N—O—R₂₂, -alkylene-OR₂₂, -alkylene-SR₂₂, -alkylene-(SO₂)R₂₂, cycloalkyl, cycloalkenyl, aryl, heterocyclyl, —(CO)R₂₂, —(CO)N(R₂₁)₂, —(CO)OR₂₂, —N(R₂₁)₂, -alkylene-(CO)R₂₂, -alkylene-(CO)N(R₂₁)₂, -alkylene-(CO)OR₂₂, or -alkylene-N(R₂₁)₂;
R₈ represents hydrogen, alkyl, alkenyl, alkynyl, haloalkyl, haloalkenyl, haloalkynyl, cycloalkyl, or cycloalkylalkyl;
R₁, R₂, R₃, R₄ and R₅ each independently represent hydrogen, halogen, nitro, cyano, cyanothio, hydroxyl, mercapto, carboxyl, sulfo, formyl, haloformyl, azido, alkyl, alkenyl, alkynyl, cycloalkyl, cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —PO(OR₂₂)₂, —OR₂₂, —(CO)R₂₂, —SR₂₂, —(SO)R₂₂, —(SO₂)R₂₂, —Si(R₂₂)₃, —O(CO)R₂₂, —O—(SO₂)R₂₂, —S(CO)R₂₂, —(SO₂)OR₂₂, —O(CO)OR₂₂, —(CO)(CO)OR₂₂, —(CO)OR₂₂, —O—N═C(R₂₃)₂, —CR₂₃═N—OH, or —CR₂₃═N—O—R₂₂; wherein, the “alkyl”, “alkenyl” or “alkynyl” is optionally substituted by at least one group selected from halogen, nitro, cyano, hydroxyl, mercapto, carboxyl, cycloalkyl, cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —OR₂₂, —(CO)R₂₂, —SR₂₂, —(SO)R₂₂, —(SO₂)R₂₂, —O(CO)H, —O(CO)R₂₂, —O—(SO₂)R₂₂, —(CO)OR₂₂, —O(CO)OR₂₂, —Si(R₂₂)₃, —O(CO)(CO)OH, —O(CO)(CO)OR₂₂, —O— alkylene-(CO)OH, and —O-alkylene-(CO)OR₂₂;
R₁₁ each independently represents halogen, nitro, cyano, cyanothio, hydroxyl, mercapto, carboxyl, sulfo, formyl, haloformyl, azido, alkyl, alkenyl, alkynyl, cycloalkyl, cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —PO(OR₂₂)₂, —OR₂₂, —(CO)R₂₂, —SR₂₂, —(SO)R₂₂, —(SO₂)R₂₂, —Si(R₂₂)₃, —O(CO)R₂₂, —O—(SO₂)R₂₂, —S(CO)R₂₂, —(SO₂)OR₂₂, —O(CO)OR₂₂, —(CO)(CO)OR₂₂, —(CO)OR₂₂, —O—N═C(R₂₃)₂, —CR₂₃═N—OH, or —CR₂₃═N—O—R₂₂; wherein, the “alkyl”, “alkenyl” or “alkynyl” is optionally substituted by at least one group selected from halogen, nitro, cyano, hydroxyl, mercapto, carboxyl, cycloalkyl, cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —OR₂₂, —(CO)R₂₂, —SR₂₂, —(SO)R₂₂, —(SO₂)R₂₂, —O(CO)H, —O(CO)R₂₂, —O—(SO₂)R₂₂, —(CO)OR₂₂, —O(CO)OR₂₂, —Si(R₂₂)₃, —O(CO)(CO)OH, —O(CO)(CO)OR₂₂, —O-alkylene-(CO)OH, and —O— alkylene-(CO)OR₂₂;
R₂₁ each independently represents hydrogen, alkyl, alkenyl, alkynyl, cycloalkyl, cycloalkenyl, aryl, heterocyclyl, —OR₂₂, —(CO)R₂₂, —(CO)OR₂₂, -alkylene-(CO)OR₂₂, —(SO₂)R₂₂, —(SO₂)OR₂₂, -alkylene-(SO₂)R₂₂, —(CO)N(R₂₄)₂, or —(SO₂)N(R₂₄)₂;
R₂₂ each independently represents alkyl, alkenyl, alkynyl, cycloalkyl, cycloalkenyl, aryl, or heterocyclyl, wherein, the “alkyl”, “alkenyl” or “alkynyl” is optionally substituted by at least one group selected from halogen, cyano, trialkylsilyl, cycloalkyl, cycloalkenyl, aryl, heterocyclyl, —OR₂₅, —SR₂₅, —O(CO)R₂₅, —(CO)R₂₅, —(CO)OR₂₅, and —O(CO)OR₂₅;
R₂₃ each independently represents hydrogen, halogen, alkoxy, alkoxyalkyl, alkyl, alkenyl, alkynyl, cycloalkyl, cycloalkylalkyl, cycloalkenyl, cycloalkenylalkyl, aryl, arylalkyl, heterocyclyl, or heterocyclylalkyl;
R₂₄ each independently represents hydrogen, alkyl, alkenyl, alkynyl, alkoxy, alkylsulfonyl, cycloalkyl, cycloalkylalkyl, cycloalkenyl, or cycloalkenylalkyl;
or N(R₂₁)₂ and N(R₂₄)₂ each independently represent heterocyclyl with a nitrogen atom at 1-position;
R₂₅ each independently represents hydrogen, alkyl, alkenyl, alkynyl, cycloalkyl, haloalkyl, haloalkenyl, haloalkynyl, phenyl or phenyl that is substituted by at least one group selected from the following: halogen, cyano, nitro, alkyl, haloalkyl, alkoxy, haloalkoxy, alkoxycarbonyl, alkylthio, alkylsulfonyl, phenoxy and phenoxy that is substituted by at least one group selected from halogen, cyano, nitro, alkyl, haloalkyl, alkoxy and haloalkoxy;
the foregoing “cycloalkyl”, “cycloalkenyl”, “heterocyclyl” or “aryl” is optionally substituted by at least one group selected from oxo, halogen, cyano, nitro, alkyl, alkenyl, alkynyl, cycloalkyl, haloalkyl, haloalkenyl, haloalkynyl, halocycloalkyl, alkyl-substituted cycloalkyl, —OR₁₀, —SR₁₀, —(CO)OR₁₀, —(SO₂)R₁₀, —N(R₁₀)₂ and —O-alkylene-(CO)OR₁₀, or two adjacent carbon atoms form a fused ring with —OCH₂CH₂— or —OCH₂O— that is unsubstituted or substituted by halogen;
R₁₀ each independently represents hydrogen, alkyl, haloalkyl, phenyl, or phenyl that is substituted by at least one group selected from halogen, cyano, nitro, alkyl, haloalkyl, alkoxycarbonyl, alkylthio, alkylsulfonyl, alkoxy, and haloalkoxy.

2. The substituted pyrrolidone compound according to claim 1, wherein it is characterized in that,
X and Y each independently represent hydrogen, C1-C8 alkyl, C2-C8 alkenyl, C2-C8 alkynyl, halo C1-C8 alkyl, halo C2-C8 alkenyl, halo C2-C8 alkynyl, C3-C8 cycloalkyl, C3-C8 cycloalkyl C1-C8 alkyl, aryl, aryl C1-C8 alkyl, heterocyclyl, heterocyclyl C1-C8 alkyl, hydroxyl, hydroxyl C1-C8 alkyl, mercapto, mercapto C1-C8 alkyl, nitro, cyano C1-C8 alkyl, formyl, tri C1-C8 alkylsilyl, —N(R₂₁)₂, —OR₂₂, —SR₂₂, —(SO)R₂₂, —(CO)R₂₂, —(CO)OR₂₂, —(SO₂)R₂₂, —(SO₂)N(R₂₁)₂, —(C1-C8 alkylene)-N(R₂₁)₂, —(C1-C8 alkylene)-OR₂₂, —(C1-C8 alkylene)-SR₂₂, —(C1-C8 alkylene)-(CO)R₂₂, —(C1-C8 alkylene)-(CO)OR₂₂, —(C1-C8 alkylene)-(SO₂)R₂₂, or —(C1-C8 alkylene)-(SO₂)N(R₂₁)₂;
Z represents C1-C8 alkyl, C2-C8 alkenyl, C2-C8 alkynyl, halo C1-C8 alkyl, halo C2-C8 alkenyl, halo C2-C8 alkynyl, C3-C8 cycloalkyl, C3-C8 cycloalkyl C1-C8 alkyl, aryl, aryl C1-C8 alkyl, heterocyclyl, heterocyclyl C1-C8 alkyl, hydroxyl, hydroxyl C1-C8 alkyl, mercapto, mercapto C1-C8 alkyl, nitro, cyano C1-C8 alkyl, formyl, tri C1-C8 alkylsilyl, —N(R₂₁)₂, —OR₂₂, —SR₂₂, —(SO)R₂₂, —(CO)R₂₂, —(CO)OR₂₂, —(SO₂)R₂₂, —(SO₂)N(R₂₁)₂, —(C1-C8 alkylene)-N(R₂₁)₂, —(C1-C8 alkylene)-OR₂₂, —(C1-C8 alkylene)-SR₂₂, —(C1-C8 alkylene)-(CO)R₂₂, —(C1-C8 alkylene)-(CO)OR₂₂, —(C1-C8 alkylene)-(SO₂)R₂₂, or —(C1-C8 alkylene)-(SO₂)N(R₂₁)₂;
R₆ represents hydrogen, hydroxyl, halogen, C1-C8 alkyl, C3-C8 cycloalkyl, C1-C8 alkoxy, or halo C1-C8 alkyl;
R₇ represents hydrogen, halogen, nitro, cyano, formyl, carboxyl, hydroxyl, hydroxyl C1-C8 alkyl, mercapto, mercapto C1-C8 alkyl, C1-C8 alkyl, C2-C8 alkenyl, C2-C8 alkynyl, halo C1-C8 alkyl, halo C2-C8 alkenyl, halo C2-C8 alkynyl, —OR₂₂, —SR₂₂, —(SO)R₂₂, —(SO₂)R₂₂, —CR₂₃═N—O—R₂₂, —(C1-C8 alkylene)-OR₂₂, —(C1-C8 alkylene)-SR₂₂, —(C1-C8 alkylene)-(SO₂)R₂₂, C3-C8 cycloalkyl, C3-C8 cycloalkenyl, aryl, heterocyclyl, —(CO)R₂₂, —(CO)N(R₂₁)₂, —(CO)OR₂₂, —N(R₂₁)₂, —(C1-C8 alkylene)-(CO)R₂₂, —(C1-C8 alkylene)-(CO)N(R₂₁)₂, —(C1-C8 alkylene)-(CO)OR₂₂, or —(C1-C8 alkylene)-N(R₂₁)₂;
R₈ represents hydrogen, C1-C8 alkyl, C2-C8 alkenyl, C2-C8 alkynyl, halo C1-C8 alkyl, halo C2-C8 alkenyl, halo C2-C8 alkynyl, C3-C8 cycloalkyl, or C3-C8 cycloalkyl C1-C8 alkyl;
R₁, R₂, R₃, R₄ and R₅ each independently represent hydrogen, halogen, nitro, cyano, cyanothio, hydroxyl, mercapto, carboxyl, sulfo, formyl, haloformyl, azido, C1-C8 alkyl, C2-C8 alkenyl, C2-C8 alkynyl, C3-C8 cycloalkyl, C3-C8 cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —PO(OR₂₂)₂, —OR₂₂, —(CO)R₂₂, —SR₂₂, —(SO)R₂₂, —(SO₂)R₂₂, —Si(R₂₂)₃, —O(CO)R₂₂, —O—(SO₂)R₂₂, —S(CO)R₂₂, —(SO₂)OR₂₂, —O(CO)OR₂₂, —(CO)(CO)OR₂₂, —(CO)OR₂₂, —O—N═C(R₂₃)₂, —CR₂₃═N—OH, or —CR₂₃═N—O—R₂₂; wherein, the “C1-C8 alkyl”, “C2-C8 alkenyl” or “C2-C8 alkynyl” is optionally substituted by at least one group selected from halogen, nitro, cyano, hydroxyl, mercapto, carboxyl, C3-C8 cycloalkyl, C3-C8 cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —OR₂₂, —(CO)R₂₂, —SR₂₂, —(SO)R₂₂, —(SO₂)R₂₂, —O(CO)H, —O(CO)R₂₂, —O—(SO₂)R₂₂, —(CO)OR₂₂, —O(CO)OR₂₂, —Si(R₂₂)₃, —O(CO)(CO)OH, —O(CO)(CO)OR₂₂, —O—(C1-C8 alkylene)-(CO)OH, and —O—(C1-C8 alkylene)-(CO)OR₂₂;
R₁₁ each independently represents halogen, nitro, cyano, cyanothio, hydroxyl, mercapto, carboxyl, sulfo, formyl, haloformyl, azido, C1-C8 alkyl, C2-C8 alkenyl, C2-C8 alkynyl, C3-C8 cycloalkyl, C3-C8 cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —PO(OR₂₂)₂, —OR₂₂, —(CO)R₂₂, —SR₂₂, —(SO)R₂₂, —(SO₂)R₂₂, —Si(R₂₂)₃, —O(CO)R₂₂, —O—(SO₂)R₂₂, —S(CO)R₂₂, —(SO₂)OR₂₂, —O(CO)OR₂₂, —(CO)(CO)OR₂₂, —(CO)OR₂₂, —O—N═C(R₂₃)₂, —CR₂₃═N—OH, or —CR₂₃═N—O—R₂₂; wherein, the “C1-C8 alkyl”, “C2-C8 alkenyl” or “C2-C8 alkynyl” is optionally substituted by at least one group selected from halogen, nitro, cyano, hydroxyl, mercapto, carboxyl, C3-C8 cycloalkyl, C3-C8 cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —OR₂₂, —(CO)R₂₂, —SR₂₂, —(SO)R₂₂, —(SO₂)R₂₂, —O(CO)H, —O(CO)R₂₂, —O—(SO₂)R₂₂, —(CO)OR₂₂, —O(CO)OR₂₂, —Si(R₂₂)₃, —O(CO)(CO)OH, —O(CO)(CO)OR₂₂, —O—(C1-C8 alkylene)-(CO)OH, and —O—(C1-C8 alkylene)-(CO)OR₂₂;
R₂₁ each independently represents hydrogen, C1-C8 alkyl, C2-C8 alkenyl, C2-C8 alkynyl, C3-C8 cycloalkyl, C3-C8 cycloalkenyl, aryl, heterocyclyl, —OR₂₂, —(CO)R₂₂, —(CO)OR₂₂, —(C1-C8 alkylene)-(CO)OR₂₂, —(SO₂)R₂₂, —(SO₂)OR₂₂, —(C1-C8 alkylene)-(SO₂)R₂₂, —(CO)N(R₂₄)₂, or —(SO₂)N(R₂₄)₂;
R₂₂ each independently represents C1-C8 alkyl, C2-C8 alkenyl, C2-C8 alkynyl, C3-C8 cycloalkyl, C3-C8 cycloalkenyl, aryl, or heterocyclyl, wherein, the “C1-C8 alkyl”, “C2-C8 alkenyl” or “C2-C8 alkynyl” is optionally substituted by at least one group selected from halogen, cyano, tri C1-C8 alkylsilyl, C3-C8 cycloalkyl, C3-C8 cycloalkenyl, aryl, heterocyclyl, —OR₂₅, —SR₂₅, —O(CO)R₂₅, —(CO)R₂₅, —(CO)OR₂₅, and —O(CO)OR₂₅;
R₂₃ each independently represents hydrogen, halogen, C1-C8 alkoxy, C1-C8 alkoxy C1-C8 alkyl, C1-C8 alkyl, C2-C8 alkenyl, C2-C8 alkynyl, C3-C8 cycloalkyl, C3-C8 cycloalkyl C1-C8 alkyl, C3-C8 cycloalkenyl, C3-C8 cycloalkenyl C1-C8 alkyl, aryl, aryl C1-C8 alkyl, heterocyclyl, or heterocyclyl C1-C8 alkyl;
R₂₄ each independently represents hydrogen, C1-C8 alkyl, C2-C8 alkenyl, C2-C8 alkynyl, C1-C8 alkoxy, C1-C8 alkylsulfonyl, C3-C8 cycloalkyl, C3-C8 cycloalkyl C1-C8 alkyl, C3-C8 cycloalkenyl, or C3-C8 cycloalkenyl C1-C8 alkyl;
or N(R₂₁)₂ and N(R₂₄)₂ each independently represent heterocyclyl with a nitrogen atom at 1-position;
R₂₅ each independently represents hydrogen, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, phenyl or phenyl that is substituted by at least one group selected from the following: halogen, cyano, nitro, C1-C8 alkyl, halo C1-C8 alkyl, C1-C8 alkoxy, halo C1-C8 alkoxy, C1-C8 alkoxycarbonyl, C1-C8 alkylthio, C1-C8 alkylsulfonyl, phenoxy and phenoxy that is substituted by at least one group selected from halogen, cyano, nitro, C1-C8 alkyl, halo C1-C8 alkyl, C1-C8 alkoxy and halo C1-C8 alkoxy;
the foregoing “C3-C8 cycloalkyl”, “C3-C8 cycloalkenyl”, “heterocyclyl” or “aryl” is optionally substituted by at least one group selected from oxo, halogen, cyano, nitro, C1-C8 alkyl, C2-C8 alkenyl, C2-C8 alkynyl, C3-C8 cycloalkyl, halo C1-C8 alkyl, halo C2-C8 alkenyl, halo C2-C8 alkynyl, halo C3-C8 cycloalkyl, C1-C8 alkyl-substituted C3-C8 cycloalkyl, —OR₁₀, —SR₁₀, —(CO)OR₁₀, —(SO₂)R₁₀, —N(R₁₀)₂ and —O—(C1-C8 alkylene)-(CO)OR₁₀, or two adjacent carbon atoms form a fused ring with —OCH₂CH₂— or —OCH₂O— that is unsubstituted or substituted by halogen;
R₁₀ each independently represents hydrogen, C1-C8 alkyl, halo C1-C8 alkyl, phenyl, or phenyl that is substituted by at least one group selected from halogen, cyano, nitro, C1-C8 alkyl, halo C1-C8 alkyl, C1-C8 alkoxycarbonyl, C1-C8 alkylthio, C1-C8 alkylsulfonyl, C1-C8 alkoxy, and halo C1-C8 alkoxy.

3. The substituted pyrrolidone compound according to claim 1, wherein it is characterized in that,
X and Y each independently represent hydrogen, C1-C6 alkyl, C2-C6 alkenyl, C2-C6 alkynyl, halo C1-C6 alkyl, halo C2-C6 alkenyl, halo C2-C6 alkynyl, C3-C6 cycloalkyl, C3-C6 cycloalkyl C1-C6 alkyl, aryl, aryl C1-C6 alkyl, heterocyclyl, heterocyclyl C1-C6 alkyl, hydroxyl, hydroxyl C1-C6 alkyl, mercapto, mercapto C1-C6 alkyl, nitro, cyano C1-C6 alkyl, formyl, tri C1-C6 alkylsilyl, —N(R₂₁)₂, —OR₂₂, —SR₂₂, —(SO)R₂₂, —(CO)R₂₂, —(CO)OR₂₂, —(SO₂)R₂₂, —(SO₂)N(R₂₁)₂, —(C1-C6 alkylene)-N(R₂₁)₂, —(C1-C6 alkylene)-OR₂₂, —(C1-C6 alkylene)-SR₂₂, —(C1-C6 alkylene)-(CO)R₂₂, —(C1-C6 alkylene)-(CO)OR₂₂, —(C1-C6 alkylene)-(SO₂)R₂₂, or —(C1-C6 alkylene)-(SO₂)N(R₂₁)₂;
Z represents C1-C6 alkyl, C2-C6 alkenyl, C2-C6 alkynyl, halo C1-C6 alkyl, halo C2-C6 alkenyl, halo C2-C6 alkynyl, C3-C6 cycloalkyl, C3-C6 cycloalkyl C1-C6 alkyl, aryl, aryl C1-C6 alkyl, heterocyclyl, heterocyclyl C1-C6 alkyl, hydroxyl, hydroxyl C1-C6 alkyl, mercapto, mercapto C1-C6 alkyl, nitro, cyano C1-C6 alkyl, formyl, tri C1-C6 alkylsilyl, —N(R₂₁)₂, —OR₂₂, —SR₂₂, —(SO)R₂₂, —(CO)R₂₂, —(CO)OR₂₂, —(SO₂)R₂₂, —(SO₂)N(R₂₁)₂, —(C1-C6 alkylene)-N(R₂₁)₂, —(C1-C6 alkylene)-OR₂₂, —(C1-C6 alkylene)-SR₂₂, —(C1-C6 alkylene)-(CO)R₂₂, —(C1-C6 alkylene)-(CO)OR₂₂, —(C1-C6 alkylene)-(SO₂)R₂₂, or —(C1-C6 alkylene)-(SO₂)N(R₂₁)₂;
R₆ represents hydrogen, hydroxyl, halogen, C1-C6 alkyl, C3-C6 cycloalkyl, C1-C6 alkoxy, or halo C1-C6 alkyl;
R₇ represents hydrogen, halogen, nitro, cyano, formyl, carboxyl, hydroxyl, hydroxyl C1-C6 alkyl, mercapto, mercapto C1-C6 alkyl, C1-C6 alkyl, C2-C6 alkenyl, C2-C6 alkynyl, halo C1-C6 alkyl, halo C2-C6 alkenyl, halo C2-C6 alkynyl, —OR₂₂, —SR₂₂, —(SO)R₂₂, —(SO₂)R₂₂, —CR₂₃═N—O—R₂₂, —(C1-C6 alkylene)-OR₂₂, —(C1-C6 alkylene)-SR₂₂, —(C1-C6 alkylene)-(SO₂)R₂₂, C3-C6 cycloalkyl, C3-C6 cycloalkenyl, aryl, heterocyclyl, —(CO)R₂₂, —(CO)N(R₂₁)₂, —(CO)OR₂₂, —N(R₂₁)₂, —(C1-C6 alkylene)-(CO)R₂₂, —(C1-C6 alkylene)-(CO)N(R₂₁)₂, —(C1-C6 alkylene)-(CO)OR₂₂, or —(C1-C6 alkylene)-N(R₂₁)₂;
R₈ represents hydrogen, C1-C6 alkyl, C2-C6 alkenyl, C2-C6 alkynyl, halo C1-C6 alkyl, halo C2-C6 alkenyl, halo C2-C6 alkynyl, C3-C6 cycloalkyl, or C3-C6 cycloalkyl C1-C6 alkyl;
R₁, R₂, R₃, R₄ and R₅ each independently represent hydrogen, halogen, nitro, cyano, cyanothio, hydroxyl, mercapto, carboxyl, sulfo, formyl, haloformyl, azido, C1-C6 alkyl, C2-C6 alkenyl, C2-C6 alkynyl, C3-C6 cycloalkyl, C3-C6 cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —PO(OR₂₂)₂, —OR₂₂, —(CO)R₂₂, —SR₂₂, —(SO)R₂₂, —(SO₂)R₂₂, —Si(R₂₂)₃, —O(CO)R₂₂, —O—(SO₂)R₂₂, —S(CO)R₂₂, —(SO₂)OR₂₂, —O(CO)OR₂₂, —(CO)(CO)OR₂₂, —(CO)OR₂₂, —O—N═C(R₂₃)₂, —CR₂₃═N—OH, or —CR₂₃═N—O—R₂₂; wherein, the “C1-C6 alkyl”, “C2-C6 alkenyl” or “C2-C6 alkynyl” is optionally substituted by at least one group selected from halogen, nitro, cyano, hydroxyl, mercapto, carboxyl, C3-C6 cycloalkyl, C3-C6 cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —OR₂₂, —(CO)R₂₂, —SR₂₂, —(SO)R₂₂, —(SO₂)R₂₂, —O(CO)H, —O(CO)R₂₂, —O—(SO₂)R₂₂, —(CO)OR₂₂, —O(CO)OR₂₂, —Si(R₂₂)₃, —O(CO)(CO)OH, —O(CO)(CO)OR₂₂, —O—(C1-C6 alkylene)-(CO)OH, and —O—(C1-C6 alkylene)-(CO)OR₂₂;
R₁₁ each independently represents halogen, nitro, cyano, cyanothio, hydroxyl, mercapto, carboxyl, sulfo, formyl, haloformyl, azido, C1-C6 alkyl, C2-C6 alkenyl, C2-C6 alkynyl, C3-C6 cycloalkyl, C3-C6 cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —PO(OR₂₂)₂, —OR₂₂, —(CO)R₂₂, —SR₂₂, —(SO)R₂₂, —(SO₂)R₂₂, —Si(R₂₂)₃, —O(CO)R₂₂, —O—(SO₂)R₂₂, —S(CO)R₂₂, —(SO₂)OR₂₂, —O(CO)OR₂₂, —(CO)(CO)OR₂₂, —(CO)OR₂₂, —O—N═C(R₂₃)₂, —CR₂₃═N—OH, or —CR₂₃═N—O—R₂₂; wherein, the “C1-C6 alkyl”, “C2-C6 alkenyl” or “C2-C6 alkynyl” is optionally substituted by at least one group selected from halogen, nitro, cyano, hydroxyl, mercapto, carboxyl, C3-C6 cycloalkyl, C3-C6 cycloalkenyl, heterocyclyl, aryl, —N(R₂₁)₂, —(CO)N(R₂₁)₂, —O(CO)N(R₂₁)₂, —O(CS)N(R₂₁)₂, —(SO₂)N(R₂₁)₂, —O(SO₂)N(R₂₁)₂, —OR₂₂, —(CO)R₂₂, —SR₂₂, —(SO)R₂₂, —(SO₂)R₂₂, —O(CO)H, —O(CO)R₂₂, —O—(SO₂)R₂₂, —(CO)OR₂₂, —O(CO)OR₂₂, —Si(R₂₂)₃, —O(CO)(CO)OH, —O(CO)(CO)OR₂₂, —O—(C1-C6 alkylene)-(CO)OH, and —O—(C1-C6 alkylene)-(CO)OR₂₂;
R₂₁ each independently represents hydrogen, C1-C6 alkyl, C2-C6 alkenyl, C2-C6 alkynyl, C3-C6 cycloalkyl, C3-C6 cycloalkenyl, aryl, heterocyclyl, —OR₂₂, —(CO)R₂₂, —(CO)OR₂₂, —(C1-C6 alkylene)-(CO)OR₂₂, —(SO₂)R₂₂, —(SO₂)OR₂₂, —(C1-C6 alkylene)-(SO₂)R₂₂, —(CO)N(R₂₄)₂, or —(SO₂)N(R₂₄)₂;
R₂₂ each independently represents C1-C6 alkyl, C2-C6 alkenyl, C2-C6 alkynyl, C3-C6 cycloalkyl, C3-C6 cycloalkenyl, aryl, or heterocyclyl, wherein, the “C1-C6 alkyl”, “C2-C6 alkenyl” or “C2-C6 alkynyl” is optionally substituted by at least one group selected from halogen, cyano, tri C1-C6 alkylsilyl, C3-C6 cycloalkyl, C3-C6 cycloalkenyl, aryl, heterocyclyl, —OR₂₅, —SR₂₅, —O(CO)R₂₅, —(CO)R₂₅, —(CO)OR₂₅, and —O(CO)OR₂₅;
R₂₃ each independently represents hydrogen, halogen, C1-C6 alkoxy, C1-C6 alkoxy C1-C6 alkyl, C1-C6 alkyl, C2-C6 alkenyl, C2-C6 alkynyl, C3-C6 cycloalkyl, C3-C6 cycloalkyl C1-C6 alkyl, C3-C6 cycloalkenyl, C3-C6 cycloalkenyl C1-C6 alkyl, aryl, aryl C1-C6 alkyl, heterocyclyl, or heterocyclyl C1-C6 alkyl;
R₂₄ each independently represents hydrogen, C1-C6 alkyl, C2-C6 alkenyl, C2-C6 alkynyl, C1-C6 alkoxy, C1-C6 alkylsulfonyl, C3-C6 cycloalkyl, C3-C6 cycloalkyl C1-C6 alkyl, C3-C6 cycloalkenyl, or C3-C6 cycloalkenyl C1-C6 alkyl;
or N(R₂₁)₂ and N(R₂₄)₂ each independently represent 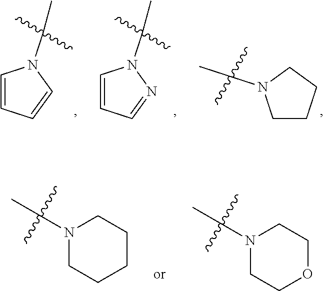 [SMILES: *n1cccc1 ; *n1cccn1 ; *N1CCCC1 ; *N1CCCCC1 ; *N1CCOCC1]
that is unsubstituted or substituted by at least one group selected from oxo, C1-C6 alkyl, and C1-C6 alkoxycarbonyl;
R₂₅ each independently represents hydrogen, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, phenyl or phenyl that is substituted by at least one group selected from the following: halogen, cyano, nitro, C1-C6 alkyl, halo C1-C6 alkyl, C1-C6 alkoxy, halo C1-C6 alkoxy, C1-C6 alkoxycarbonyl, C1-C6 alkylthio, C1-C6 alkylsulfonyl, phenoxy and phenoxy that is substituted by at least one group selected from halogen, cyano, nitro, C1-C6 alkyl, halo C1-C6 alkyl, C1-C6 alkoxy and halo C1-C6 alkoxy;
the foregoing “C3-C6 cycloalkyl”, “C3-C6 cycloalkenyl”, “heterocyclyl” or “aryl” is optionally substituted by at least one group selected from oxo, halogen, cyano, nitro, C1-C6 alkyl, C2-C6 alkenyl, C2-C6 alkynyl, C3-C6 cycloalkyl, halo C1-C6 alkyl, halo C2-C6 alkenyl, halo C2-C6 alkynyl, halo C3-C6 cycloalkyl, C1-C6 alkyl-substituted C3-C6 cycloalkyl, —OR₁₀, —SR₁₀, —(CO)OR₁₀, —(SO₂)R₁₀, —N(R₁₀)₂ and —O—(C1-C6 alkylene)-(CO)OR₁₀, or two adjacent carbon atoms form a fused ring with —OCH₂CH₂— or —OCH₂O— that is unsubstituted or substituted by halogen;
R₁₀ independently represents hydrogen, C1-C6 alkyl, halo C1-C6 alkyl, phenyl, or phenyl substituted by at least one group selected from halogen, cyano, nitro, C1-C6 alkyl, halo C1-C6 alkyl, C1-C6 alkoxycarbonyl, C1-C6 alkylthio, C1-C6 alkylsulfonyl, C1-C6 alkoxy, and halo C1-C6 alkoxy;
preferably, the compound is selected from any one in Table 1.

4. A substituted pyrrolidone compound with chiral centers as set forth in Formula I′: 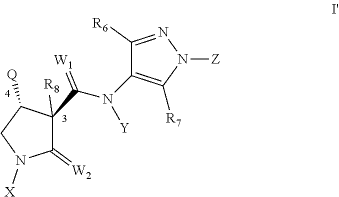
wherein, the definitions of substituents Q, R₆, R₇, R₈, W₁, W₂, X, Y and Z are as described according to claim 1;
preferably, based on the contents of stereoisomers that have R- and S-configuration at position 3, it has a stereochemical purity (S) of 60%-100%, preferably 70%-100%, more preferably 80%-100%, further preferably 90%-100%, even further preferably 95%-100%; and based on the contents of stereoisomers that have R- and S-configuration at position 4, it has a stereochemical purity (S or R) of 60%-100%, preferably 70%-100%, more preferably 80%-100%, further preferably 90%-100%, even further preferably 95%-100%;
more preferably, the compound is selected form any one in Table A.

5. A method for preparing the substituted pyrrolidone compound according to claim 1, wherein it is characterized in that, it includes the following steps:
subjecting the compound shown in the general formula II and the compound shown in the general formula III to reaction to produce the compound as shown in the general formula I, and the reaction equation is as follows: 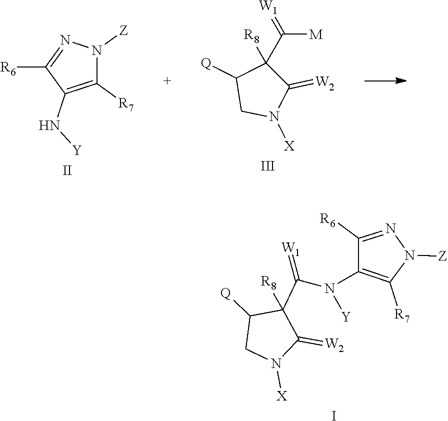
wherein, M represents OH or halogen, and the definitions of substituents Q, R₆, R₇, R₈, W₁, W₂, X, Y and Z are as described according to claim 1;
preferably, the reaction is carried out in the presence of a base and a solvent, with or without a condensing agent, or without a base; more preferably, the base is selected from at least one of inorganic bases and organic bases; more preferably, the solvent is selected from at least one of DMF, DMA, methanol, ethanol, acetonitrile, dichloroethane, DMSO, dioxane, dichloromethane, and ethyl acetate; more preferably, the condensing agent is selected from at least one of Py-BOP, Py-AOP, EDCI, HOBT, DCC, HBTU, and HATU.

6. A herbicidal composition, wherein it is characterized in that, it comprises (i) a herbicidally effective amount of at least one of the substituted pyrrolidone compounds according to claim 1; preferably, also comprises (ii) a formulation auxiliary; more preferably, further comprises (iii) a herbicidally effective amount of one or more additional herbicides and/or safeners;
the additional herbicide is selected from one or more of the following compounds:
(1) VLCFA inhibitors: pretilachlor, butachlor, mefenacet, acetochlor, anilofos;
(2) HPPD inhibitors: 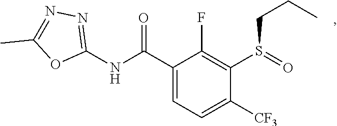 [SMILES: CCC[S@@](=O)c1c(C(F)(F)F)ccc(C(=O)Nc2nnc(C)o2)c1F]
tefuryltrione, benzobicyclon, bipyrazone;
(3) PPO inhibitors: oxadiazon, pyraclonil, oxyfluorfen, oxadiargyl, pentoxazone, 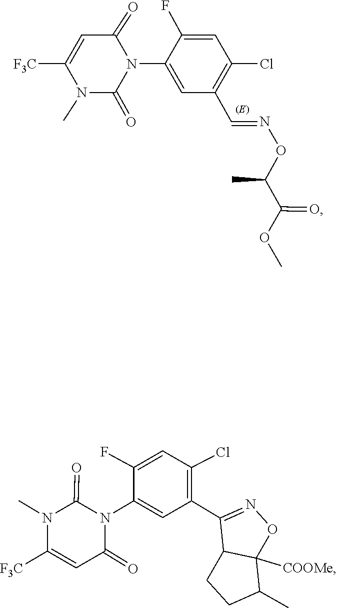 [SMILES: COC(=O)[C@@H](C)O/N=C/c1cc(-n2c(=O)cc(C(F)(F)F)n(C)c2=O)c(F)cc1Cl ; COC(=O)C12ON=C(c3cc(-n4c(=O)cc(C(F)(F)F)n(C)c4=O)c(F)cc3Cl)C1CCC2C]
(4) Synthetic hormones: halauxifen-methyl, florpyrauxifen-benzyl, 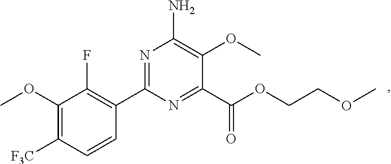
fluroxypyr, 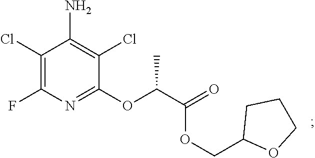 [SMILES: C[C@@H](Oc1nc(F)c(Cl)c(N)c1Cl)C(=O)OCC1CCCO1]
(5) PSII inhibitors: propanil, bentazone, simetryn;
(6) DOXP inhibitors: clomazone, 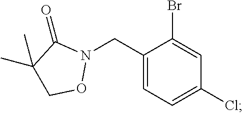 [SMILES: CC1(C)CON(Cc2ccc(Cl)cc2Br)C1=O]
(7) PDS inhibitors: bixlozone, 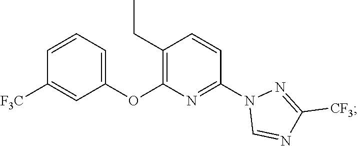 [SMILES: CCc1ccc(-n2cnc(C(F)(F)F)n2)nc1Oc1cccc(C(F)(F)F)c1]
(8) FAT inhibitors: cinmethylin, 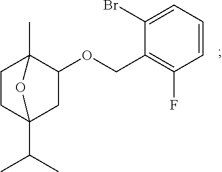 [SMILES: CC(C)C12CCC(C)(O1)C(OCc1c(F)cccc1Br)C2]
(9) Other herbicides: oxaziclomefone;
the weight ratio of the effective ingredient (i) and the additional herbicide in (iii) in the composition is 1:100-100:1, 1:80-80:1, 1:50-50:1, 1:30-30:1, 1:20-20:1, 1:10-10:1, 1:5-1:1, or 1:1-5:1.

7. A fungicidal composition, wherein it is characterized in that, it comprises a disease-inhibiting and phytologically acceptable amount of at least one of the substituted pyrrolidone compounds according to claim 1; preferably, also comprises a formulation auxiliary; more preferably, further comprises another active ingredient.

8. An insecticidal composition, wherein it is characterized in that, it comprises a biologically effective amount of at least one of the substituted pyrrolidone compounds according to claim 1; preferably, also comprises a formulation auxiliary; more preferably, further comprises another active ingredient.

9. A method for controlling a weed, wherein it is characterized in that, it comprises applying a herbicidally effective amount of at least one of the substituted pyrrolidone compounds according to claim 1 or a herbicidal composition to a plant or a weedy area; wherein the herbicidal composition comprises a herbicidally effective amount of at least one of the substituted pyrrolidone compounds according to claim 1.

10. A method for preventing and controlling a harmful fungus, comprising treating the fungus or a material, a plant, soil or a seed to be protected against the fungal infestation with a disease-inhibiting and phytologically acceptable amount of at least one of the substituted pyrrolidone compounds according to claim 1 or a fungicidal composition which comprises a disease-inhibiting and phytologically acceptable amount of at least one of the substituted pyrrolidone compounds according to claim 1.

11. A method for preventing and controlling a pest insect, wherein it is characterized in that, it comprises exposing the pest insect or its environment to a biologically effective amount of at least one of the substituted pyrrolidone compounds according to claim 1 or the insecticidal composition which comprises a biologically effective amount of at least one of the substituted pyrrolidone compounds according to claim 1.

12. (canceled)

13. An intermediate, as shown in Formula II or Formula III according to claim 5.

14. A method for preventing or eliminating the weed among a useful crop, and the useful crop is a transgenic crop or a crop treated by a genome editing technique; wherein the method is characterized in that, it comprises applying a herbicidally effective amount of at least one of the substituted pyrrolidone compounds according to claim 1 or a herbicidal composition to the weed or the useful crop; wherein the herbicidal composition comprises a herbicidally effective amount of at least one of the substituted pyrrolidone compounds according to claim 1.

15. A method for preventing and controlling a phytopathogenic fungus, including protecting a plant from infestation by a phytopathogenic microorganism or treating a plant which is attacked by a phytopathogenic microorganism; wherein the method is characterized in that, it comprises applying a disease-inhibiting and phytologically acceptable amount of at least one of the substituted pyrrolidone compounds according to claim 1 or a herbicidal composition to soil, a plant, a part of a plant, leaves, and/or roots; wherein the herbicidal composition comprises a herbicidally effective amount of at least one of the substituted pyrrolidone compounds according to claim 1.
